(19) United States
(12) Patent Application Publication
(10) Pub. No.: US 2026/0109737 A1
(43) Pub. Date: Apr. 23, 2026

(54) ANTI-CRISPR DELIVERY COMPOSITIONS AND METHODS

(71) Applicants: The Brigham and Women's Hospital, Inc., Boston, MA (US); Massachusetts Institute of Technology, Cambridge, MA (US)

(72) Inventors: Amit Choudhary, Boston, MA (US); Axel O. Vera, Cambridge, MA (US); Bradley Pentelute, Cambridge, MA (US); Nicholas Truex, Cambridge, MA (US); Ronald T. Raines, Cambridge, MA (US)

(21) Appl. No.: 19/304,906

(22) Filed: Aug. 20, 2025

Related U.S. Application Data
(63) Continuation of application No. PCT/US2024/016743, filed on Feb. 21, 2024.
(60) Provisional application No. 63/447,235, filed on Feb. 21, 2023.

(51) Int. Cl. C07K 14/005; C07K 14/32; C07K 14/34; C12N 9/99

(52) U.S. Cl. CPC C07K 14/005; C07K 14/32; C07K 14/34; C12N 9/99; C07K 2319/09; C07K 2319/55; C12N 2795/00022

(57) ABSTRACT

Described in certain exemplary embodiments herein are engineered Anti-CRISPR (Acr) polypeptides and delivery systems engineered for delivery to the cytosol and/or nucleus of cells. In certain embodiments, an engineered Acr polypeptide comprises an Acr polypeptide operatively coupled to a cargo delivery molecule, wherein the cargo delivery molecule is capable of binding or otherwise interacting with a pore-forming polypeptide. Also described in certain exemplary embodiments are methods of Acr delivery to cells via the engineered Acr polypeptides and delivery systems of the present disclosure.

### CROSS-REFERENCE TO RELATED APPLICATIONS

This application is a continuation application of PCT/US2024/016743, filed Feb. 21, 2024, which claims the benefit of and priority to U.S. Provisional Patent Application No. 63/447,235, filed on Feb. 21, 2023, the contents of which are incorporated by reference herein in its entirety.

### STATEMENT REGARDING FEDERALLY SPONSORED RESEARCH

This invention was made with government support under Grant No. GM137606 awarded by the National Institutes of Health. The government has certain rights in the invention.

### SEQUENCE LISTING

This application contains a sequence listing filed in electronic form as an XML file entitled “BROD-5645US_ST26_revised.xml”, created on Aug. 26, 2025, and having a size of 144,557 bytes. The content of the sequence listing is incorporated herein in its entirety.

### TECHNICAL FIELD

The subject matter disclosed herein is generally directed to delivery compositions and methods, particularly for delivering anti-CRISPR systems and components.

### BACKGROUND

Clustered regularly interspaced short palindromic repeats (CRISPR)-associated systems (Cas) are in development for laboratory research, agricultural engineering, various biotechnology endeavors, and treatment of various genetic disorders. In particular, Class 2 systems (e.g., Cas9 and Cas12a) have been engineered to treat blood and muscle disorders, such as Sickle cell disease and Duchenne muscular dystrophy. Despite the rapid progress in basic research and clinical tests, some underlying problems present continuous, significant challenges, such as editing efficiency, relative difficulty in delivery, off-target effects, and immunogenicity, among others. Off-target effects observed with Cas systems include chromosomal rearrangements and disruption of essential genes in non-target cells. It has been observed that Cas systems may be more prone to these off-target effects than other conventional gene-editing methods because the Cas protein is a monomer that can facilitate recognition of shorter target sequences in comparison with transcription activator-like effector nucleases (TALENs) or Zinc Finger Nucleases (ZFNs), whose assemblies are dimeric.

Several methods have been utilized to counter the off-target effects, including optimization of the RNA guide sequences and engineering Cas proteins to improve system specificity and minimize the chances of causing off-target activity. Another approach is inhibiting Cas protein activity, particularly by anti-CRISPR (Acr) proteins, small molecule inhibitors, and nucleic acid-based inhibitors. Acrs are being intensely developed as they offer a highly potent approach due to possessing several CRISPR-Cas interaction sites and function through various mechanisms, such as binding Cas with or without the target nucleic acid or modifying Cas to prevent its nuclease activity. These features of Acrs may make it possible to control Cas activity and improve targeting specificity. However, like Cas proteins, delivery of Acrs has proved to be a significant challenge due to, among other things, their ability to stimulate a host immune response and, due to their size, an inability to diffuse through a cell membrane freely. As such, there exists a need for alternative delivery compositions and methods of Acrs.

Citation or identification of any document in this application is not an admission that such a document is available as prior art to the present invention.

### SUMMARY

Described in specific example embodiments herein are engineered Anti-CRISPR (Acr) polypeptides comprising an Acr polypeptide operatively coupled to a cargo delivery molecule, wherein the cargo delivery molecule is capable of binding or otherwise interacting with a pore-forming polypeptide.

In an example embodiment, the cargo delivery molecule is a bacterial exotoxin, optionally a Bacillus anthracis lethal factor (LF) or edema factor (EF) or a derivative thereof or a Corynebacterium diphtheriae catalytic domain or derivative thereof.

In an example embodiment, the cargo delivery molecule is engineered to comprise a pore-forming polypeptide interaction molecule or domain, optionally where the pore-forming polypeptide interaction molecule or domain is operatively coupled to the N-terminus, the C-terminus, at a location between the N- and C-terminus, or any combination thereof of the cargo delivery molecule.

In an embodiment, the cargo delivery molecule is cleavably coupled to the Acr polypeptide. In an embodiment, the cargo delivery molecule is cleavably coupled to the Acr polypeptide, so cleavage occurs via protease, deubiquitinase, small molecule, reducing agent, metabolite, etc.). In an embodiment, the cargo delivery molecule comprises a cleavable domain. In an embodiment, the cargo delivery molecule is linked via a cleavable linker to the Acr polypeptide. In an embodiment, the protease, deubiquitinase, small molecule, reducing agent, and metabolite are native to the cytosol. In an embodiment, the protease, deubiquitinase, small molecule, thickening agent, metabolite, etc., is exogenous to the cytosol.

In an example embodiment, the pore-forming polypeptide is an alpha pore-forming polypeptide, beta pore-forming polypeptide, or both.

In an example embodiment, the pore-forming polypeptide is a Bacillus anthracis protective antigen polypeptide or a derivative thereof or is a Corynebacterium diphtheriae translocation polypeptide or a derivative thereof.

In an example embodiment, the Acr polypeptide inhibits a Type I, Type II, Type III, Type V, or Type VI CRISPR-Cas system, component, or activity thereof.

In an example embodiment, the Acr polypeptide is a Type I Acr polypeptide, a Type II Acr polypeptide, a Type III Acr polypeptide, a Type V Acr polypeptide, a Type VI Acr polypeptide, or any combination thereof.

In an example embodiment, the Acr polypeptide is selected from an AcrIE8.2, AcrIE9, AcrIF1, AcrIF2, AcrIF3, AcrIF4, AcrIF5, AcrIF6, ArcIF7, AcrIF8, AcrIF9, AcrIF10, AcrIF11, AcrIF11.1, AcrIF11.2, AcrIF12, AcrIF13, AcrIF14, AcrIF15, AcrIF16, AcrIF17, AcrIF18, AcrIF19, AcrIF20, AcrIF21, AcrIF22, AcrIF23, AcrIF24, AcrIE4-F7, AcrIAI, AcrIB1, AcrIC1, AcrIF2/C2, AcrIC3, AcrIC4, AcrIC5, AcrIC6, AcrIC7, AcrIC8, AcrIC9, AcrIC10, AcrID1, AcrIIA1, AcrIIA2, AcrIIA2-1, AcrIIA2-2, AcrIIA2b, AcrIIA3, AcrIIA4, AcrIIA4-2, AcrIIA4-3, AcrIIA4 variant Ins. 5, AcrIIA4 variant N39A, AcrIIA4 variant D14A/G38A, AcrIIA5, AcrIIA5-2, AcrIIA6, AcrIIA7, AcrIIA8, AcrIIA9, AcrIIA10, AcrIIA11, AcrIIA12, AcrIIA13, AcrIIA13b, AcrIIA14, AcrIIA15, AcrIIA16, AcrIIA17, AcrIIA18, AcrIIA19, AcrIIA20, AcrIIA21, AcrIIA22, AcrIIA23, AcrIIA24, AcrIIA25, AcrIIA26, AcrIIA27, AcrIIA28, AcrIIA29, AcrIIA30, AcrIIA31, AcrIIA32, AcrIIC1, AcrIIC1-1, AcrIIC2, AcrIIC2-1, AcrIIC2-2, AcrIIC3, AcrIIC4, AcrIIC5, AcrIIC6, AcrIII-1, AcrIIIB1, AcrVA1, AcrVA2, AcrVA3, AcrVA3.1, AcrVA4, AcrVA5, AcrVIA1(Lse), AcrVIA1(Lwa) AcrVIA2, AcrVIA3, AcrVIA4, AcrVIA5, AcrVIA6, AcrVIA7, AcrVIB, Csx27, a homologue thereof, or any combination thereof.

In an example embodiment, the engineered Acr polypeptide further comprises a reporter molecule operatively coupled to the cargo delivery molecule, the Acr polypeptide, or both.

In an example embodiment, the pore-forming polypeptide comprises a targeting moiety or targeting domain.

In an example embodiment, the pore-forming polypeptide is operatively coupled to a targeting moiety, optionally, wherein the targeting moiety is an antibody or fragment thereof.

In an example embodiment, the pore-forming polypeptide interaction molecule or domain is a charged polypeptide, optionally a polybasic polypeptide.

Described in an example embodiment herein are engineered Acr polypeptide delivery systems comprising a plurality of pore-forming polypeptides, wherein one or more of the pore-forming polypeptides are operatively coupled to a targeting moiety, and an engineered Acr polypeptide of the present invention, wherein the cargo delivery molecule of the engineered Acr polypeptide is capable of binding or otherwise interacting with the pore-forming polypeptide thereby transporting the Acr polypeptide through a pore formed from the pore-forming polypeptide.

In an embodiment, the targeting moiety is an antibody or fragment.

In an example embodiment, the pore-forming polypeptide is an alpha or beta pore-forming polypeptide.

In an embodiment, the pore-forming polypeptide is a Bacillus anthracis protective antigen polypeptide or a derivative thereof, or is a Corynebacterium diphtheriae translocation polypeptide or a derivative thereof.

An embodiment herein describes polynucleotides encoding an engineered Acr polypeptide of the present invention, an engineered Acr polypeptide delivery system or component thereof.

Described in an embodiment herein are vector systems that may comprise one or more vectors comprising one or more polynucleotides of the present invention encoding an engineered Acr polypeptide of the present invention and/or an engineered Acr polypeptide delivery system or component thereof of the present invention; and optionally one or more regulatory elements operatively coupled to the one or more polynucleotides.

A delivery vehicle may comprise (a) an engineered Acr polypeptide of the present invention; (b) an engineered Acr polypeptide delivery system or component thereof of the present invention; (c) one or more polynucleotides of the present invention; (d) one or more vectors of the present invention; or any combination of (a)-(d).

A cell or cell population may comprise (a) an engineered Acr polypeptide of the present invention; (b) an engineered Acr polypeptide delivery system or component thereof of the present invention; (c) one or more polynucleotides of the present invention; (d) one or more vectors of the present invention; (e) a delivery vehicle of the present invention; or (f) any combination of (a)-(e).

Pharmaceutical formulations may comprise (a) an engineered Acr polypeptide of the present invention; (b) an engineered Acr polypeptide delivery system or component thereof of the present invention; (c) one or more polynucleotides of the present invention; (d) one or more vectors of the present invention; (e) a delivery vehicle of the present invention; (f) a cell or cell population of the present invention; or any combination of (a)-(f); and a pharmaceutically acceptable carrier.

A kit may comprise (a) an engineered Acr polypeptide of the present invention; (b) an engineered Acr polypeptide delivery system or component thereof of the present invention; (c) one or more polynucleotides of the present invention; (d) one or more vectors of the present invention; (e) a delivery vehicle of the present invention; (f) a cell or cell population of the present invention; (g) a pharmaceutical formulation of the present invention; or (h) any combination of (a)-(g).

Methods of delivering an anti-CRISPR (Acr) polypeptide to cells may comprise providing, to a cell or cell population, (a) an engineered Acr polypeptide of the present invention; (b) an engineered Acr polypeptide delivery system or component thereof of the present invention; (c) one or more polynucleotides of the present invention; (d) one or more vectors of the present invention; (e) a delivery vehicle of the present invention; (f) a cell or cell population of the present invention; (g) a pharmaceutical formulation of the present invention; or (h) any combination of (a)-(g).

The cell may comprise a targeting moiety-binding partner on the cell membrane surface.

The method of delivering an Arc to cells may further comprise binding the targeting moiety or targeting domain of a plurality of pore-forming polypeptides of an engineered Acr delivery system of the present invention to the targeting moiety binding partner on the cell surface, thereby tethering the pore-forming protein to the cell surface; and forming a pre-pore at the cell membrane surface formed from a plurality of the pore-forming polypeptides tethered to the cell membrane surface.

In an embodiment, the method further comprises coupling the engineered Acr polypeptide to one or more pore-forming polypeptides in the pre-pore or pore-forming polypeptide complex via binding of the cargo delivery molecule to the pore-forming polypeptide in the pre-pore or pore-forming polypeptide complex.

In an embodiment, the method further comprises transporting the pre-pore and the engineered Acr polypeptide coupled to it into the cell via endocytosis, whereby the pre-pore becomes a pore.

In an embodiment, the method comprises the formation of a pore from the pre-pore or pore-forming polypeptide complex.

In an embodiment, the method further releases the engineered Acr polypeptide from the pre-pore, pore-forming complex, or pore.

In an embodiment, the method further comprises releasing the engineered Acr polypeptide from an endosome into the cell's cytosol.

In an embodiment, the method comprises releasing the engineered Acr polypeptide from the pre-pore, pore-forming complex, or pore and releasing the engineered Acr polypeptide from an endosome into the cell's cytosol and/or nucleus.

Described herein are methods of inhibiting the activity of a CRISPR-Cas system in a cell comprising delivering an anti-CRISPR (Acr) polypeptide to the cell by the method of delivery described herein, whereby the Acr polypeptide inhibits the activity of a CRISPR-Cas system or a component thereof in the cell. In an embodiment, the method increases the specificity of a CRISPR-Cas system.

These and other aspects, objects, features, and advantages of the example embodiments will become apparent to those with ordinary skill in the art upon considering the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1A-1B—Shows exemplary embodiments of a method that uses protective antigen (PA) to deliver an anti-CRISPR (Acr) polypeptide into the cytosol of a target cell. (1) Wild-type PA (e.g., PA₈₃) or a PA variant with (FIG. 1B) or without (FIG. 1A), an engineered targeting moiety, binds to its binding partner in the cell surface. (2) PA bound to its partner becomes active (e.g., via proteolytic cleavage). (3) The activated PA (e.g., PA₆₃) oligomerizes into a pre-pore. (4) An engineered Acr polypeptide, consisting of an Acr polypeptide (e.g., AcrIIA4) operatively coupled to a cargo delivery molecule (e.g., LF_{N}), binds to the PA pre-pore (5) The entire complex is endocytosed and the engineered Acr polypeptide translocates into the cytosol (e.g. after acidification of the endosome promotes the PA pre-pore to change into a transmembrane pore for translocating the engineered Acr polypeptide into the cytosol). The PA or PA variant is also referred to herein as a pore-forming polypeptide.

FIG. 2—(SEQ ID NO: 1) An exemplary construct for producing and purifying engineered Acr delivery polypeptides. A protein fusion between a polyhistidine-tagged maltose-binding protein (MBP, for solubility and purification), a TEV protease cleavage site, a glycine-cysteine-serine linker (G₄CG₄S (SEQ ID NO: 1), for flexibility and optional bioconjugation), the N-terminus of lethal factor (LF_{N}, for binding to PA), AcrIIA4 (for Cas9 inhibition), a glycine-serine (GS) linker (for flexibility), and an SV40 nuclear localization sequence (NLS). After isolating the 10×His-MBP-TEV-LF_{N}-G₄CG₄S-AcrIIA4-GS-NLS protein via metal affinity chromatography. The MBP tag is cleaved using TEV protease and separated from LF_{N}-G₄CG₄S-AcrIIA4-GS-NLS (hereafter referred to as LF_{N}-AcrIIA4) via affinity chromatography, ion exchange chromatography, and size-exclusion chromatography.

FIG. 3A-3C—Purification and characterization of a representative Acr polypeptide that is operatively coupled to a cargo delivery molecule (LF_{N}-AcrIIA4) for delivery using the PA method. FIG. 3A—Sodium dodecyl sulfate-polyacrylamide gel electrophoresis (SDS-PAGE) demonstrating the purification of the LF_{N}-AcrIIA4 polypeptide. FIG. 3B—(SEQ ID NO: 1) A cartoon of LF_{N}-AcrIIA4 and some of its physical properties determined using the Expasy ProtParam tool. FIG. 3C—Liquid chromatography-mass spectrometry (LC-MS) of LF_{N}-AcrIIA4.

FIG. 4—Assessing the delivery of an engineered Acr (e.g., LF_{N}-AcrIIA4) via the EGFP disruption assay. A CRISPR-Cas system (e.g., a Streptococcus pyogenes Cas9, SpCas9, ribonucleoprotein, RNP, complex) targeting the EGFP gene is delivered via nucleofection into U2OS-EGFP.PEST cells (300,000 cells). The nucleofected cells are resuspended in DMEM media (10% FBS) and transferred to a 96-well plate (100 μL final volume, 100,000-200,000 cells/mL), where they are incubated with the Acr delivery system (e.g., LF_{N}-AcrIIA4 and PA) for 24-48 h. Live cells are stained with Hoechst 33342 and imaged via confocal microscopy. Cas-mediated cleavage disrupts EGFP production. Successful delivery of the Acr prevents Cas-mediated EGFP disruption.

FIG. 5—The EGFP disruption assay confirms that the PA method delivers an engineered Acr polypeptide (e.g., LF_{N}-AcrIIA4) into human cells in a dose-dependent manner. C-NLS-SpCas9 RNP (20 μmol) targeting the EGFP gene is delivered via nucleofection into U2OS-EGFP.PEST cells, followed by resuspension in DMEM media (10% FBS), incubation with LF_{N}-AcrIIA4 (250 fM-250 nM) and PA (20 nM) 2 h post-nucleofection/resuspension, and imaging after 43 h. Positive controls include nucleofection of apo-Cas9 and co-nucleofection of Cas9 RNP and LF_{N}-AcrIIA4 (50 μmol, inhibited-Cas9 complex (IC)). Negative controls include nucleofection of Cas9 RNP, nucleofection of Cas9 RNP followed by incubation with LF_{N}-AcrIIA4 (250 fM-250 nM) 2 h post-nucleofection/resuspension, and nucleofection of Cas9 RNP followed by incubation with LF_{N}-AcrIIA4 (250 fM-250 nM) and the translocation-deficient PA [F427H] variant (PAH, 20 nM) 2 h post-nucleofection/resuspension. Data are the mean±s.d. of two independent replicates. The significance of Cas9 RNP+LF_{N}-AcrIIA4/PA additions was determined with an unpaired, two-tailed t-test versus Cas9 RNP, where *, **, ***, and **** stand for P≤0.05, P≤0.01, P≤0.001, and P≤0.0001, respectively.

FIG. 6—Representative fluorescent microscopic images from the EGFP disruption assay of FIG. 5, demonstrating the dose-dependent delivery of an engineered Acr polypeptide (e.g., LF_{N}-AcrIIA4) into human cells.

FIG. 7—The EGFP disruption assay confirms that timing the delivery of an engineered Acr polypeptide (e.g., LF_{N}-AcrIIA4) using the PA method controls Cas9 activity in human cells. C-NLS-SpCas9 RNP (20 μmol) targeting the EGFP gene is delivered via nucleofection into U2OS-EGFP.PEST cells, followed by resuspension in DMEM media (10% FBS), incubation with LF_{N}-AcrIIA4 (25 fM-250 nM), and PA (20 nM) 2 or 4 h post-nucleofection/resuspension (LF_{N}-AcrIIA4 dosing time=2 or 4 h), and imaging after 48 h. Positive controls include nucleofection of apo-Cas9 and co-nucleofection of Cas9 RNP and LF_{N}-AcrIIA4 (50 μmol, inhibited-Cas9 complex (IC)). Negative controls include nucleofection of Cas9 RNP and nucleofection of Cas9 RNP followed by incubation with LF_{N}-AcrIIA4 (25 fM-250 nM) 2 h or 4 h post-nucleofection/resuspension. Data are the mean±s.d. of three independent replicates. The significance of Cas9 RNP+LF_{N}-AcrIIA4/PA additions was determined with an unpaired, two-tailed t-test versus Cas9 RNP, where *, **, ***, and **** stand for P≤0.05, P≤0.01, P≤0.001, and P≤0.0001, respectively.

FIG. 8—Representative fluorescent microscopic images from the EGFP disruption assay of FIG. 7, demonstrating the dose-dependent delivery of an engineered Acr polypeptide (e.g., LF_{N}-AcrIIA4) with a two-hour dosing time.

FIG. 9—Representative fluorescent microscopic images from the EGFP disruption assay of FIG. 7, demonstrating the dose-dependent delivery of an engineered Acr polypeptide (e.g., LF_{N}-AcrIIA4) with a four-hour dosing time.

FIG. 10—Assessing the delivery of an engineered Acr (e.g., LF_{N}-AcrIIA4) via the HiBiT assay. A CRISPR-Cas system (e.g., SpCas9 RNP) targeting the GAPDH gene and a single-stranded oligo donor nucleotide (ssODN) encoding for the HiBiT tag are delivered via nucleofection into HEK293T cells (300,000 cells). The nucleofected cells are resuspended in DMEM media (10% FBS) and transferred to a 96-well plate (100 μL final volume, 135,000-290,000 cells/mL), where they are incubated with the Acr delivery system (e.g., LF_{N}-AcrIIA4 and PA) for 72 h. Cell viability is measured with the PrestoBlue reagent, followed by cell lysis, incubation with LgBiT, and quantification of luminescence. Cas-mediated cleavage and HiBIT tag insertion via homology-directed repair led to the formation of NanoLuc after LgBiT addition. Successful delivery of the Acr prevents Cas-mediated cleavage, HiBiT tag insertion, and NanoLuc formation after LgBiT addition.

FIG. 11—The HiBiT assay confirms that the PA method delivers an engineered Acr polypeptide (e.g., LF_{N}-AcrIIA4) into human cells in a dose-dependent manner. C-NLS-SpCas9 RNP (20 μmol) targeting the GAPDH gene and the HiBIT ssODN (80 μmol) are co-delivered via nucleofection into HEK293T cells, followed by resuspension in DMEM media (10% FBS), incubation with LF_{N}-AcrIIA4 (2.5-250 nM) and PA (20 nM), and quantification of luminescence after 72 h. Positive controls include nucleofection of apo-Cas9, ssODN, and Cas9 RNP separately and co-nucleofection of Cas9 RNP, ssODN, and LF_{N}-AcrIIA4 (50 μmol, inhibited-Cas9 complex (IC)). Negative controls include co-nucleofection of Cas9 RNP and ssODN and co-nucleofection of Cas9 RNP and ssODN followed by incubation with LF_{N}-AcrIIA4 (2.5-250 nM). The data were normalized to Cas9 RNP+ssODN and are the mean±s.d. of three independent replicates. The significance of Cas9 RNP+ssODN+LEN-AcrIIA4/PA additions was determined with an unpaired, two-tailed t-test versus Cas9 RNP+ssODN, where *, **, ***, and **** stand for P≤0.05, P≤0.01, P≤0.001, and P≤0.0001, respectively.

FIG. 12—The HiBiT assay confirms that timing the delivery of an engineered Acr polypeptide (i.e., LF_{N}-AcrIIA4) using the PA method controls Cas9 activity in human cells. C-NLS-SpCas9 RNP (20 μmol) targeting the GAPDH gene and the HiBIT ssODN (80 μmol) are co-delivered via nucleofection into HEK293T cells, followed by resuspension in DMEM media (10% FBS), incubation with LF_{N}-AcrIIA4 (250 nM) and PA (20 nM) 0-48 h post-nucleofection/resuspension (dosing time (DT) 0-48 h), and quantification of luminescence after 72 h. Positive controls include nucleofection of apo-Cas9, ssODN, and Cas9 RNP separately and co-nucleofection of Cas9 RNP, ssODN, and LF_{N}-AcrIIA4 (50 μmol, inhibited-Cas9 complex-IC). Negative controls include co-nucleofection of Cas9 RNP and ssODN and co-nucleofection of Cas9 RNP and ssODN followed by incubation with LF_{N}-AcrIIA4 (250 nM) 0-48 h post-nucleofection/resuspension. The data were normalized to Cas9 RNP+ssODN and are the mean±s.d. of three independent replicates. The significance of Cas9 RNP+ssODN+LF_{N}-AcrIIA4/PA timepoint additions was determined with an unpaired, two-tailed t-test versus Cas9 RNP+ssODN, where *, **, ***, and **** stand for P<0.05, P≤0.01, P≤0.001, and P≤0.0001, respectively.

FIG. 13—General strategy for a T7 endonuclease 1 (T7E1) mismatch detection assay to validate the delivery of an engineered Acr polypeptide (e.g., LF_{N}-AcrIIA4). A CRISPR-Cas system (e.g., SpCas9 RNP) targeting a gene of interest (e.g., the EMX1 gene) is delivered via nucleofection into HEK293T cells (300,000 cells). The nucleofected cells are resuspended in DMEM media (10% FBS) and transferred to a 24-well plate (600 μL final volume, 240,000 cells/mL), where they are incubated with the Acr delivery system (e.g., LF_{N}-AcrIIA4 and PA) for 72 h. Genomic DNA is extracted from the cells, and the target site is PCR amplified. After rehybridization, the amplicon is incubated with T7E1, and the cleavage products are visualized via agarose gel electrophoresis. Cas-mediated cleavage of the genomic DNA target leads to insertions and deletions (indels) that cause mismatches after rehybridization of the amplicon. T7E1 recognizes and cleaves these mismatches. Successful delivery of the Acr prevents Cas-mediated cleavage, indels, and mismatches; therefore, T7E1 does not cleave the amplicon.

FIG. 14—The T7E1 assay confirms that the PA method delivers an engineered Acr polypeptide (e.g., LF_{N}-AcrIIA4) into human cells in a dose- and time-dependent manner. C-NLS-SpCas9 RNP (20 μmol) targeting the EMX1 gene is delivered via nucleofection into HEK293T cells, followed by resuspension in DMEM media (10% FBS) and incubation with LF_{N}-AcrIIA4 (0.025-250 nM) and PA (20 nM) 0-24 h post-nucleofection/resuspension (dosing time (DT) 0-24 h) for 72 h. Genomic DNA is extracted, and the target is PCR amplified, rehybridized, incubated with T7E1, and visualized via agarose gel electrophoresis. Positive controls include nucleofection of apo-Cas9 and co-nucleofection of Cas9 RNP and LF_{N}-AcrIIA4 (50 μmol, inhibited-Cas9 complex (IC)). Negative controls include nucleofection of Cas9 RNP and nucleofection of Cas9 RNP followed by incubation with LF_{N}-AcrIIA4 (250 nM). The first lane contains an E-Gel 50 bp DNA ladder for reference.

FIG. 15A-15B-Next-generation sequencing (NGS) confirms that delivering an engineered Acr polypeptide (e.g., LF_{N}-AcrIIA4) using the PA method increases genome-editing specificity in human cells in a dose-dependent manner. FIG. 15A—NLS-SpCas9-NLS RNP (80 μmol) targeting the EMX1 gene is delivered via nucleofection into HEK293T cells (300,000 cells). The nucleofected cells are resuspended in DMEM media (10% FBS) and transferred to a 24-well plate (600 μL final volume, 240,000 cells/mL), where they are incubated with LF_{N}-AcrIIA4 (2.5-250 nM) and PA (20 nM) for 72 h. Genomic DNA is extracted, and the target and off-target sites are PCR amplified and barcoded. The amplicons are sequenced via NGS and analyzed using the CRISPResso2 software pipeline to determine insertions, deletions, and substitutions (% modification). Positive controls include nucleofection of apo-Cas9 and co-nucleofection of Cas9 RNP and LF_{N}-AcrIIA4 (200 μmol, inhibited-Cas9 complex (IC)). Negative controls include nucleofection of Cas9 RNP followed by incubation with LF_{N}-AcrIIA4 (250 nM), nucleofection of Cas9 RNP followed by incubation with LF_{N}-AcrIIA4 (250 nM) and the translocation-deficient PA [F427H] variant (PAH, 20 nM), and nucleofection of Cas9 RNP. The data were normalized to Cas9 RNP (On-target) and are the mean±s.d. of three independent replicates. The significance of Cas9 RNP+LF_{N}-AcrIIA4/PA additions was determined with an unpaired, two-tailed t-test versus Cas9 RNP, where *, **, ***, and **** stand for P≤0.05, P≤0.01, P<0.001, and P≤0.0001, respectively. FIG. 15B—The on-target/off-target ratio (i.e., the specificity) of Cas9 RNP increases with the concentration of LF_{N}-AcrIIA4 while PA is kept constant. The specificity was calculated as a ratio of on-target to off-target % modification of FIG. 15A, normalizing to Cas9 RNP (specificity=1). The significance of the calculated specificity of Cas9 RNP+LF_{N}-AcrIIA4/PA additions was determined with an unpaired, two-tailed t-test versus Cas9 RNP, where *, **, ***, and **** stand for P<0.05, P≤0.01, P≤0.001, and P≤0.0001, respectively.

FIG. 16A-16B—NGS confirms that timing the delivery of an engineered Acr polypeptide (e.g., LF_{N}-AcrIIA4) using the PA method increases genome-editing specificity in human cells. FIG. 16A—NLS-SpCas9-NLS RNP (80 μmol) targeting the EMX1 gene is delivered via nucleofection into HEK293T cells (300,000 cells). The nucleofected cells are resuspended in DMEM media (10% FBS) and transferred to a 24-well plate (600 μL final volume, 240,000 cells/mL), where they are incubated with LF_{N}-AcrIIA4 (250 nM) and PA (20 nM) 0-48 h post-nucleofection/resuspension (dosing time (DT) 0-48 h) for 72 h. Genomic DNA is extracted, and the target and off-target sites are PCR amplified and barcoded. The amplicons are sequenced via NGS and analyzed using the CRISPResso2 software pipeline to determine insertions, deletions, and substitutions (% modification). Positive controls include nucleofection of apo-Cas9 and co-nucleofection of Cas9 RNP and LF_{N}-AcrIIA4 (200 μmol, inhibited-Cas9 complex (IC)). Negative controls include nucleofection of Cas9 RNP followed by incubation with LF_{N}-AcrIIA4 (250 nM), nucleofection of Cas9 RNP followed by incubation with LF_{N}-AcrIIA4 (250 nM) and the translocation-deficient PA [F427H] variant (PAH, 20 nM), and nucleofection of Cas9 RNP. The data were normalized to Cas9 RNP (On-target) and are the mean±s.d. of three independent replicates. The significance of Cas9 RNP+LF_{N}-AcrIIA4/PA timepoint additions was determined with an unpaired, two-tailed t-test versus Cas9 RNP, where *, **, ***, and **** stand for P≤0.05, P≤0.01, P≤0.001, and P≤0.0001, respectively. FIG. 16B—The on-target/off-target ratio (i.e., the specificity) of Cas9 RNP can be optimized by timing the delivery of LF_{N}-AcrIIA4 using the PA method. The specificity was calculated as a ratio of on-target to off-target % modification of FIG. 16A, normalizing to Cas9 RNP (specificity=1). The significance of the calculated specificity of Cas9 RNP+LEN-AcrIIA4/PA timepoint additions was determined with an unpaired, two-tailed t-test versus Cas9 RNP, where *, **, ***, and **** stand for P≤0.05, P≤0.01, P≤0.001, and P≤0.0001, respectively.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of standard terms and techniques in molecular biology may be found in Molecular Cloning: A Laboratory Manual, 2nd edition (1989) (Sambrook, Fritsch, and Maniatis); Molecular Cloning: A Laboratory Manual, 4th edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2nd edition 2013 (E. A. Greenfield ed.); Animal Cell Culture (1987) (R. I. Freshney, ed.); Benjamin Lewin, Genes IX, published by Jones and Bartlett, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2nd edition (2011).

As used herein, the singular forms “a,” “an,” and “the” include both singular and plural referents unless the context dictates otherwise.

The terms “optional” or “optionally” means that the subsequently described step or element may or may not occur. The description includes instances where the step occurs, instances where it does not, or where the element is present or not present.

The recitation of numerical ranges by endpoints includes all numbers and fractions subsumed within the respective ranges and the recited endpoints.

The terms “about” or “approximately,” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is understood that the value to which the modifier “about” or “approximately” refers is also specifically and preferably disclosed.

As used herein, a “biological sample” may contain whole cells and/or live cells and/or cell debris. The biological sample may have (or be derived from) a “bodily fluid.” The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, and cell cultures from bodily fluids. Bodily fluids may be obtained from a mammal organism, for example, by puncture or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells, and the progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment,” “an embodiment,” and “an example embodiment” means that a particular feature, structure, or characteristic described in connection with the embodiment is included in at least one embodiment of the present invention. Furthermore, the specific features, structures, or characteristics may be combined in any suitable manner, as would be apparent to a person skilled in the art from this disclosure in one or more embodiments. Furthermore, while an embodiment described herein includes some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each publication, published patent document, or patent application was specifically and individually indicated as being incorporated by reference.

### Overview

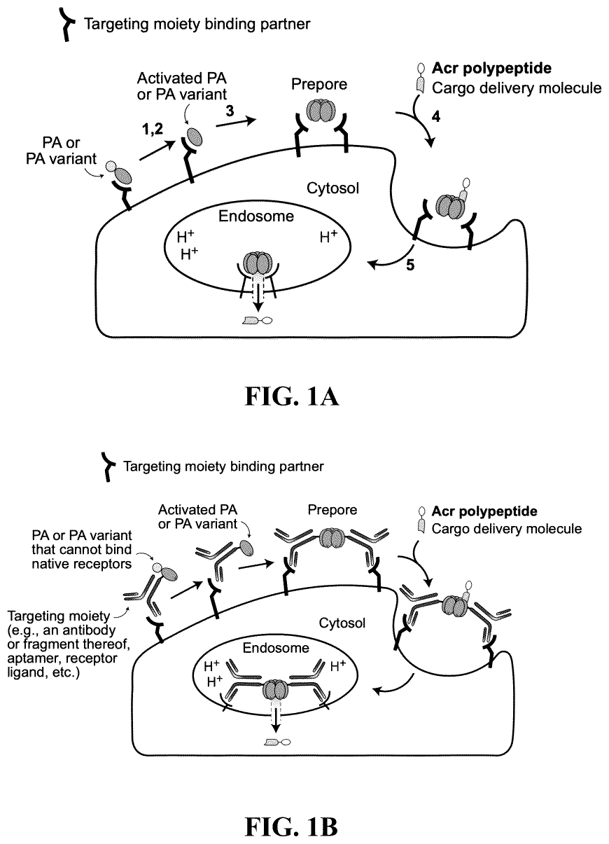

The present disclosure provides methods and compositions for regulating the activity of a CRISPR-Cas system. Described herein are engineered compositions and methods of Acr-based delivery. Described in an example embodiment herein are engineered Acr polypeptides that include an Acr polypeptide that is operatively coupled to a cargo delivery molecule, where the cargo delivery molecule is capable of binding or otherwise interacting with a pore-forming polypeptide. In an example embodiment, the cargo delivery molecule is a bacterial exotoxin or is otherwise engineered to comprise a pore-forming polypeptide interaction molecule or domain. Without being bound by theory, as shown in FIG. 1A-1B, during delivery, a plurality of pore-forming polypeptides, of which one or more comprise a targeting moiety or targeting domain (FIG. 1A) or are optionally operatively coupled to a targeting moiety (FIG. 1B), are targeted to a target cell via binding of the targeting moiety(ies) to a binding partner on the surface of the target cell such that a pre-pore is formed on/at the surface of a target cell. The engineered Acr polypeptide then binds or otherwise interacts with the pre-pore, and the complex is taken up by the cell by an endocytic mechanism, which results in the delivery of the engineered Acr polypeptide to the target cell. Without being bound by theory, delivery can occur via the translocation mechanism utilized by a native PA. See, e.g., Young and Collier. Annu. Rev. Biochem. (2007) 76:243-265. In an embodiment, the target cell is a cell in which it is desirable to inhibit the activity of a CRISPR-Cas system.

Other compositions, compounds, methods, features, and advantages of the present disclosure will be or become apparent to one having ordinary skill in the art upon examination of the following drawings, detailed descriptions, and examples. It is intended that all such additional compositions, compounds, methods, features, and advantages be included within this description within the scope of the present disclosure.

### Acr Delivery Compositions and Systems

### Engineered Acr Delivery Polypeptides

Described in an example embodiment herein are engineered Anti-CRISPR (Acr) polypeptides comprising an Acr polypeptide operatively coupled to a cargo delivery molecule (e.g., LF_{N}), wherein the cargo delivery molecule is capable of binding or otherwise interacting with a pore-forming polypeptide (e.g., PA or PA variant). See, e.g., FIG. 1A-1B. Without being bound by theory, binding or other interaction with a pore-forming polypeptide, optionally contained in a pre-pore, that is operatively coupled to a target cell results in translocation of the engineered Acr polypeptide into the cytosol of the cell by an endocytic mechanism. In an embodiment, an Acr polypeptide is cleaved from the cargo delivery molecule in the endosome. The engineered Acr polypeptide or component thereof (e.g., Acr polypeptide and/or cargo delivery molecule) is then released from the endosome via an endosomal escape mechanism. In an embodiment, the engineered Acr polypeptide is cleaved from the cargo delivery molecule after release from an endosome. In an embodiment, the engineered polypeptide or one or more components thereof (e.g., Acr polypeptide, cargo delivery molecule, etc., or any combination thereof) are unfolded in the endosome before release from the endosome. In an embodiment, the Acr polypeptide is not cleaved from the cargo delivery molecule. In an embodiment, the Acr polypeptide is cleaved from the cargo delivery molecule. See, e.g., FIG. 1A-1B. As used in this context herein, “operatively coupled” refers to a direct coupling (e.g., in-frame fusion), indirect coupling (e.g., via a linker molecule), or other interaction (e.g., hydrostatic, van der Waals interaction), that effectively attaches, links, brings into effective proximity, or otherwise results in a functional interaction between the molecules, compounds, and/or the like that are operatively coupled.

As used herein, “effective proximity” refers to the distance, region, or area surrounding a reference point, molecule, compound, or object in which a desired effect or activity occurs. The effective proximity can be determined by measuring the desired effect or activity in a representative number of species surrounding the reference point or object. By way of non-limiting examples, an agent can be delivered to a specific point in a subject's tissue, diffused through the surrounding tissue, and cause effects in cells at a distance from the initial point of delivery. Cells that are affected by the agent can be determined, and thus, the region of effective proximity can be determined. Cells within that region are said to be within effective proximity to the initial delivery point. Similarly, if a cell is engineered to produce a product and secretes it into the surrounding environment, cells in the surrounding environment that are affected by the secreted product are said to be within effective proximity to the producing cell (or reference point). Likewise, if two (or more) molecules, compounds, compositions, objects, and/or the like are in effective proximity to one another, such a distance, region, or area can be defined and/or determined by measuring a change in one or more of the molecules, compounds, compositions, objects, and/or the like, a product produced from the molecules, compounds, compositions, objects, and/or the like (e.g., light, heat, or product compound, composition and/or the like). The molecules, compounds, compositions, objects, and/or the like are in “effective proximity” at the physical distance(s), position(s), etc., where a change, reaction, product, and/or the like is produced. In an embodiment, effective proximity ranges from 0 to 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1100, 1110, 1120, 1130, 1140, 1150, 1160, 1170, 1180, 1190, 1200, 1210, 1220, 1230, 1240, 1250, 1260, 1270, 1280, 1290, 1300, 1310, 1320, 1330, 1340, 1350, 1360, 1370, 1380, 1390, 1400, 1410, 1420, 1430, 1440, 1450, 1460, 1470, 1480, 1490, 1500, 1510, 1520, 1530, 1540, 1550, 1560, 1570, 1580, 1590, 1600, 1610, 1620, 1630, 1640, 1650, 1660, 1670, 1680, 1690, 1700, 1710, 1720, 1730, 1740, 1750, 1760, 1770, 1780, 1790, 1800, 1810, 1820, 1830, 1840, 1850, 1860, 1870, 1880, 1890, 1900, 1910, 1920, 1930, 1940, 1950, 1960, 1970, 1980, 1990, 2000 angstroms, pm, microns, or mm away from the reference point. In an embodiment, direct contact or covalent bonding (i.e., effective proximity is 0).

### Acr Polypeptides

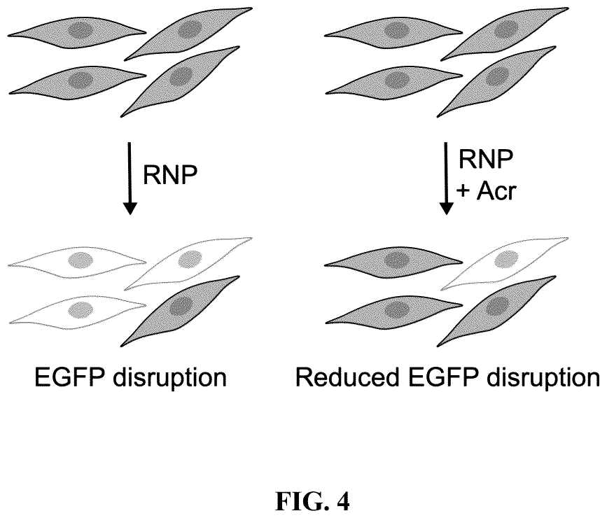

The engineered Acr polypeptides contain one or more Acr polypeptides with one or more modifications. Acr polypeptides can inhibit Cas system activity by various mechanisms, including but not limited to inhibiting target nucleic acid binding, target nucleic acid cleavage, gRNA binding, and/or Cas complex formation. In an embodiment, the Acr polypeptide(s) prevent or inhibit target nucleic acid binding by the CRISPR-Cas system or component thereof. In an embodiment, the Acr polypeptide(s) prevent or inhibit Cas nuclease or nickase activity. In an embodiment, the Acr polypeptide(s) prevent or inhibit target nucleic acid cleavage. In an embodiment, the Acr polypeptide(s) prevent or inhibit target nucleic acid (e.g., DNA or RNA) binding or interaction with a Cas or Cas complex. In an embodiment, the Acr polypeptide(s) prevent or inhibit crRNA or gRNA loading into a Cas. In an embodiment, the Acr polypeptide(s) prevent or inhibit Cascade complex recruitment, formation, activity, or both. For example, AcrIE2 inhibits Cas3 recruitment to the Cascade complex (see, e.g., Mejdani et al., J. Mol. Biol. 433 (3): 166759 (2021)). In an embodiment, the Acr polypeptide is capable of cleaving gRNAs. See, e.g., Knott et al., Nat. Struct. Mol. Biol. 26, 315-321 (2019), Wang et al., Nuc. Acid. Res., 50, 512-521 (2022). In an embodiment, the Acr polypeptide is capable of homo or heterodimerization (see, e.g., Wiegand et al., Annu. Rev. Microbiol. 2020. 74:21-37, particularly at FIG. 4). In an embodiment, the Acr polypeptide(s) inhibit or prevent Cas system activity by allosteric or steric inhibition. In an embodiment, the Acr polypeptide(s) inhibit or prevent Cas system activity by preventing or inhibiting Cas system enzyme recycling (see, e.g., Peng et al., Proc. Natl. Acad. Sci. 116 (38) 18928-18936).

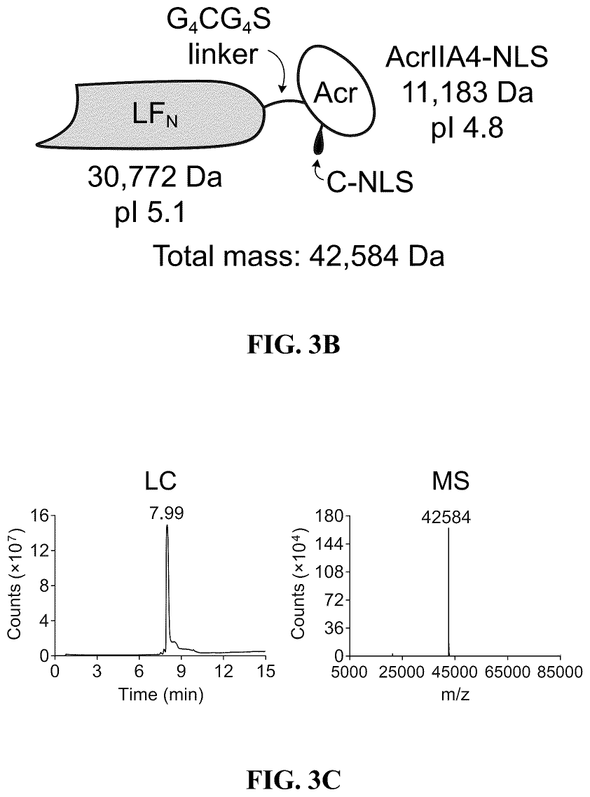

In an embodiment, the Acr polypeptide(s) binds a Cas protein or a domain thereof. In an embodiment, the Acr polypeptide(s) bind an HNH domain of a Cas (see, e.g., Liu et al., Nuc. Acid. Res. 2021. 46 (11): 6587-6595 and Harrington et al., Cell. 2017. 170 (6): 1224-1223.e15). In an embodiment, the Acr polypeptide(s) bind a RuvC domain of a Cas (see, e.g., Song et al., 2019. Cell Rep. 29:2579-2589; Kim et al., Sci. Report. (2018) 8:3883; Knott et al., eLife. 2019, 8, e49110 doi.org/10.7554/eLife.49110.001). In an embodiment, the Acr polypeptide(s) bind or interact with a bridge-helix domain of a Cas (see, e.g., Knott et al., eLife. 2019. doi.org/10.7554/eLife.49110.001). In an embodiment, the Acr polypeptide(s) bind one or more nucleic acid binding sites in a Cas or Cas complex (see, e.g., Zhang et al., 2020. Proc. Natl. Acad. Sci. U.S.A. Lett. Doi/10.1073/pnas. 1922638117). In and embodiments, the Acr polypeptide(s) bind and/or interact with one or more REC domains (see, e.g., Harrington et al., Cell. 2017. 170 (6): 1224-1223.e15). In an embodiment, the Acr polypeptide(s) bind and/or interact with a PAM-interacting domain (see e.g., Dong et al. Nature 2017, 546 (7658), 436-439; Yang et al. Mol. Cell 2017, 67 (1), 117-127 e5; and Zhang et al. Cell Host Microbe 2019, 25 (6), 815-826 e4) In an embodiment, the Acr polypeptide(s) mimic dsDNA (see e.g., Wiegand et al., Annu. Rev. Microbiol. 2020. 74:21-37, particularly at FIG. 3). Without being bound by theory, mimicking dsDNA structure facilitates the interaction of the Acr with a Cas and inhibits Cas activity on a dsDNA target.

In an embodiment, the Acr polypeptide inhibits or can inhibit a Type I, Type II, Type III, Type V, or Type VI CRISPR-Cas system, component thereof, activity thereof, or both. CRISPR-Cas systems that the Acr polypeptide can inhibit also include derivatives and variants thereof, such as, without limitation, CAST systems, prime editor systems, and base editors systems. Exemplary CRISPR-Cas systems that the Acr polypeptide can inhibit are described in greater detail elsewhere herein, such as in connection with co-therapies. In an example embodiment, the Acr polypeptide inhibits or can inhibit a catalytically inactive Cas polypeptide. In an embodiment, the Acr polypeptide can inhibit a Cas lacking double-stranded nucleic acid cleavage activity. In an embodiment, the Acr polypeptide can inhibit a Cas lacking nucleic acid single-stranded cleavage activity. In an embodiment, the Acr polypeptide can inhibit a Cas lacking nuclease activity. See, e.g., Liu et al. Cell 2018, 172 (5), 979-992 e6. In an embodiment, the Acr polypeptide is capable of inhibiting a Cas lacking nuclease activity but having nickase activity (see, e.g., Liang et al. Cells 2020, 9, 1786 and Song et al. Cell Reports 2019 29, 2579-2589). In an embodiment, the Acr polypeptide inhibits or is capable of inhibiting a dead Cas polypeptide (dCas) (see, e.g., Knott et al. Nat. Struct. Mol. Biol. 26, 315-321 (2019)).

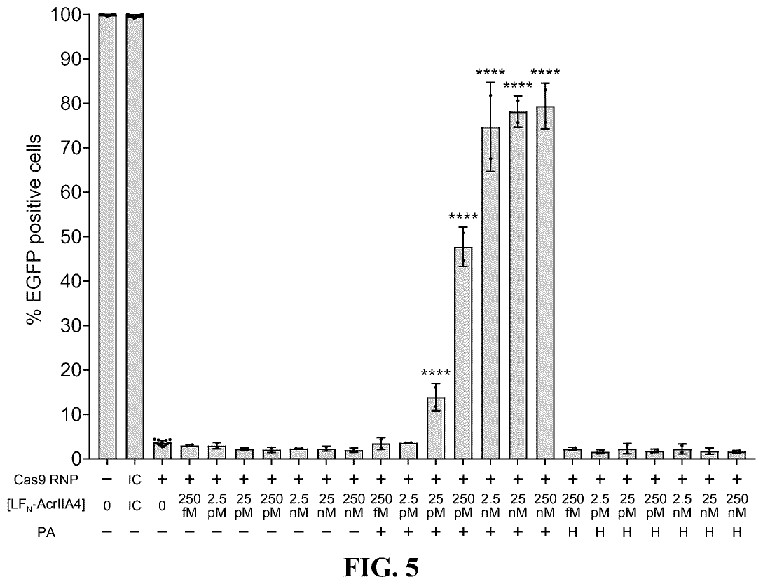

In an embodiment, the Acr polypeptide is a Type I Acr polypeptide. In an embodiment, the Acr polypeptide is a Type II Acr polypeptide. In an embodiment, the Acr polypeptide is a Type III Acr polypeptide. In an embodiment, the Acr polypeptide is a Type V Acr polypeptide. In an embodiment, the Acr polypeptide is a Type VI Acr polypeptide. See also, e.g., Marshall et al., 2018, Molecular Cell 69, 146-157, particularly at FIG. 5b.

In an example embodiment, the one or more Acr polypeptides are selected from an AcrIE8.2, AcrIE9, AcrIF1, AcrIF2, AcrIF3, AcrIF4, AcrIF5, AcrIF6, ArcIF7, AcrIF8, AcrIF9, AcrIF10, AcrIF11, AcrIF11.1, AcrIF11.2, AcrIF12, AcrIF13, AcrIF14, AcrIF15, AcrIF16, AcrIF17, AcrIF18, AcrIF19, AcrIF20, AcrIF21, AcrIF22, AcrIF23, AcrIF24, AcrIE4-F7, AcrIAI, AcrIB1, AcrIC1, AcrIF2/C2, AcrIC3, AcrIC4, AcrIC5, AcrIC6, AcrIC7, AcrIC8, AcrIC9, AcrIC10, AcrID1, AcrIIA1, AcrIIA2, AcrIIA2-1, AcrIIA2-2, AcrIIA2b, AcrIIA3, AcrIIA4, AcrIIA4-2, AcrIIA4-3, AcrIIA4 variant Ins. 5, AcrIIA4 variant N39A, AcrIIA4 variant D14A/G38A, AcrIIA5, AcrIIA5-2, AcrIIA6, AcrIIA7, AcrIIA8, AcrIIA9, AcrIIA10, AcrIIA11, AcrIIA12, AcrIIA13, AcrIIA13b, AcrIIA14, AcrIIA15, AcrIIA16, AcrIIA17, AcrIIA18, AcrIIA19, AcrIIA20, AcrIIA21, AcrIIA22, AcrIIA23, AcrIIA24, AcrIIA25, AcrIIA26, AcrIIA27, AcrIIA28, AcrIIA29, AcrIIA30, AcrIIA31, AcrIIA32, AcrIIC1, AcrIIC1-1, AcrIIC2, AcrIIC2-1, AcrIIC2-2, AcrIIC3, AcrIIC4, AcrIIC5, AcrIIC6, AcrIII-1, AcrIIIB1, AcrVA1, AcrVA2, AcrVA3, AcrVA3.1, AcrVA4, AcrVA5, AcrVIA1 (Lse), AcrVIA1 (Lwa) AcrVIA2, AcrVIA3, AcrVIA4, AcrVIA5, AcrVIA6, AcrVIA7, AcrVIB, Csx27, a homologue thereof, or any combination thereof. The terms “orthologue” (also referred to as “ortholog” herein) and “homologue” (also referred to as “homolog” herein) are well known in the art. By means of further guidance, a “homologue” of a protein as used herein is a protein of the same species which performs the same or a similar function as the protein it is a homologue of. Homologous proteins may but need not be structurally related, or are only partially structurally related. An “orthologue” of a protein as used herein is a protein of a different species which performs the same or a similar function as the protein it is an orthologue of. Orthologous proteins may but need not be structurally related, or are only partially structurally related. Homologs and orthologs may be identified by homology modelling (see, e.g., Greer, Science vol. 228 (1985) 1055, and Blundell et al. Eur J Biochem vol 172 (1988), 513) or “structural BLAST” (Dey F, Cliff Zhang Q, Petrey D, Honig B. Toward a “structural BLAST”: using structural relationships to infer function. Protein Sci. 2013 April; 22 (4): 359-66. doi: 10.1002/pro.2225). Homologous proteins may but need not be structurally related, or are only partially structurally related.

In an embodiment, the one or more Acr polypeptides comprise or contain only the functional domain(s) of one or more Acr polypeptides selected from an AcrIE8.2, AcrIE9, AcrIF1, AcrIF2, AcrIF3, AcrIF4, AcrIF5, AcrIF6, ArcIF7, AcrIF8, AcrIF9, AcrIF10, AcrIF11, AcrIF11.1, AcrIF11.2, AcrIF12, AcrIF13, AcrIF14, AcrIF15, AcrIF16, AcrIF17, AcrIF18, AcrIF19, AcrIF20, AcrIF21, AcrIF22, AcrIF23, AcrIF24, AcrIE4-F7, AcrIAI, AcrIB1, AcrIC1, AcrIF2/C2, AcrIC3, AcrIC4, AcrIC5, AcrIC6, AcrIC7, AcrIC8, AcrIC9, AcrIC10, AcrID1, AcrIIA1, AcrIIA2, AcrIIA2-1, AcrIIA2-2, AcrIIA2b, AcrIIA3, AcrIIA4, AcrIIA4-2, AcrIIA4-3, AcrIIA4 variant Ins. 5, AcrIIA4 variant N39A, AcrIIA4 variant D14A/G38A, AcrIIA5, AcrIIA5-2, AcrIIA6, AcrIIA7, AcrIIA8, AcrIIA9, AcrIIA10, AcrIIA11, AcrIIA12, AcrIIA13, AcrIIA13b, AcrIIA14, AcrIIA15, AcrIIA16, AcrIIA17, AcrIIA18, AcrIIA19, AcrIIA20, AcrIIA21, AcrIIA22, AcrIIA23, AcrIIA24, AcrIIA25, AcrIIA26, AcrIIA27, AcrIIA28, AcrIIA29, AcrIIA30, AcrIIA31, AcrIIA32, AcrIIC1, AcrIIC1-1, AcrIIC2, AcrIIC2-1, AcrIIC2-2, AcrIIC3, AcrIIC4, AcrIIC5, AcrIIC6, AcrIII-1, AcrIIIB1, AcrVA1, AcrVA2, AcrVA3, AcrVA3.1, AcrVA4, AcrVA5, AcrVIA1 (Lse), AcrVIA1 (Lwa) AcrVIA2, AcrVIA3, AcrVIA4, AcrVIA5, AcrVIA6, AcrVIA7, AcrVIB, Csx27, a homologue thereof, or any combination thereof.

In an embodiment, the one or more Acr polypeptides are variants, derivatives, homologs, orthologs, or paralogues of one or more Acr polypeptides selected from an AcrIE8.2, AcrIE9, AcrIF1, AcrIF2, AcrIF3, AcrIF4, AcrIF5, AcrIF6, ArcIF7, AcrIF8, AcrIF9, AcrIF10, AcrIF11, AcrIF11.1, AcrIF11.2, AcrIF12, AcrIF13, AcrIF14, AcrIF15, AcrIF16, AcrIF17, AcrIF18, AcrIF19, AcrIF20, AcrIF21, AcrIF22, AcrIF23, AcrIF24, AcrIE4-F7, AcrIAI, AcrIB1, AcrIC1, AcrIF2/C2, AcrIC3, AcrIC4, AcrIC5, AcrIC6, AcrIC7, AcrIC8, AcrIC9, AcrIC10, AcrID1, AcrIIA1, AcrIIA2, AcrIIA2-1, AcrIIA2-2, AcrIIA2b, AcrIIA3, AcrIIA4, AcrIIA4-2, AcrIIA4-3, AcrIIA4 variant Ins. 5, AcrIIA4 variant N39A, AcrIIA4 variant D14A/G38A, AcrIIA5, AcrIIA5-2, AcrIIA6, AcrIIA7, AcrIIA8, AcrIIA9, AcrIIA10, AcrIIA11, AcrIIA12, AcrIIA13, AcrIIA13b, AcrIIA14, AcrIIA15, AcrIIA16, AcrIIA17, AcrIIA18, AcrIIA19, AcrIIA20, AcrIIA21, AcrIIA22, AcrIIA23, AcrIIA24, AcrIIA25, AcrIIA26, AcrIIA27, AcrIIA28, AcrIIA29, AcrIIA30, AcrIIA31, AcrIIA32, AcrIIC1, AcrIIC1-1, AcrIIC2, AcrIIC2-1, AcrIIC2-2, AcrIIC3, AcrIIC4, AcrIIC5, AcrIIC6, AcrIII-1, AcrIIIB1, AcrVA1, AcrVA2, AcrVA3, AcrVA3.1, AcrVA4, AcrVA5, AcrVIA1 (Lse), AcrVIA1 (Lwa) AcrVIA2, AcrVIA3, AcrVIA4, AcrVIA5, AcrVIA6, AcrVIA7, AcrVIB, Csx27, a homologue thereof, or any combination thereof.

Table 1 provides exemplary GenBank Accession numbers, polypeptide sequences, and/or references for Acr polypeptides suitable for use in the engineered Acr polypeptides of the present invention.

TABLE 1
Exemplary Acrs

| Acr | |
|---|---|
| polypeptide | Representative GenBank Accession No(s)., Sequence, and/or Reference |
| AcrIE1 | YP_007392738.1 |
| AcrIE2 | YP_007392439.1 |
| AcrIE3 | YP_950454.1 |
| AcrIE4 | NP_938238.1 |
| AcrIE5 | WP_074973300.1 |
| AcrIE6 | WP_087937214.1 |
| AcrIE7 | WP_087937215.1 |
| AcrIE4-IF7 | Marino, Zhang, Borges, 2018, Science Oct 12; 362(6411): 240-242. |
| | MSTQYTYQQIAEDFRLWSEYVDTAGEMSKDEFNSLSTEDKVRLQVEAFGEEKSPKFS |
| | TKVTTKPDFDGFQFYIEAGRDFDGDAYTEAYGVAVPTNIAARIQAQAAELNAGEWL |
| | LVEHEA (SEQ ID NO: 2) |
| AcrIE8 | Pinilla-Redondo et al., Nat. Comm. 2020. 11: 562 |
| | doi.org/10.1038/s41467-020-19415-3. |
| | MTTITINTYDPEARFNMSGEEAKEFFAFVEEQAKVSGFDVYYDSCTYVDEESE |
| | RFVEKCFQNY (SEQ ID NO: 3) |
| AcrIE8.1 | WP_038434996.1 |
| AcrIE8.2 | WP_117085605.1 |
| AcrIE9 | WP_101192668.1; Leon, 2021, Nucleic Acids Research. Volume 49, Issue |
| | 4, 26 Feb. 2021, Pages 2114-2125 |
| AcrIF1 | YP_007392342.1; Bondy-Denomy, 2013, Nature. 493, pages 429-432. |
| AcrIF2 | NP_938237, YP_002332454.1, WP_015972868.1; Bondy-Denomy, 2013, |
| | Nature. 493, pages 429-432. |
| AcrIF3 | YP_007392440.1, YP_007392739.1; Bondy-Denomy, 2013, Nature. 493, |
| | pages 429-432. |
| AcrIF4 | WP_016068584.1, YP_007392799.1; Bondy-Denomy, 2013, Nature. 493, |
| | pages 429-432. |
| AcrIF5 | YP_007392740.1; Bondy-Denomy, 2013, Nature. 493, pages 429-432. |
| AcrIF6 | WP_043884810, WP_034001826.1, WP_031691692.1, WP_019933870.1, |
| | WP_014702809.1; Pawluk, 2016, Nature Microbiology. Jun 13; 1(8): 16085 |
| ArcIF7 | ACD38920.1; Pawluk, 2016, Nature Microbiology. Jun 13; 1(8): 16085 |
| AcrIF8 | AFC22483.1, KEH13790.1; Pawluk, 2016, Nature Microbiology. Jun |
| | 13; 1(8): 16085 |
| AcrIF9 | WP_031500045.1, EEG86164.1; Pawluk, 2016, Nature Microbiology. Jun |
| | 13; 1(8): 16085 |
| AcrIF10 | KEK29119; Pawluk, 2016, Nature Microbiology. Jun 13; 1(8): 16085 |
| AcrIF11 | WP_038819808.1, WP_102394900.1, WP_087698854.1, |
| | WP_049175110.1, WP_004681960.1, WP_062681378.1, KTG25401.1, |
| | WP_059284897.1, WP_107732478.1, WP_071971444.1, |
| | WP_086652143.1, OHU91773.1, WP_064700809.1, WP_064702655.1, |
| | WP_066478200.1, WP_068370878.1, WP_057083778.1, |
| | WP_074032235.1, WP_039494318.1, WP_077457760.1, |
| | WP_064369479.1, WP_041946990.1, WP_036292019.1, |
| | WP_017725053.1, WP_061524032.1, WP_004824702.1, |
| | WP_049556453.1, WP_109055423.1, WP_097468739.1, OZT63688.1, |
| | PKT06451.1, WP_084913096.1, WP_050090803.1, WP_050879812.1, |
| | WP_050296286.1, WP_079326564.1, WP_003671754.1, |
| | WP_026949101.1, WP_092828131.1, WP_027705017.1, SMF80656.1, |
| | WP_016360505.1, SMC32303.1, WP_051420249.1, WP_060561196.1, |
| | WP_004247747.1, WP_086368795.1, WP_078005047.1, |
| | WP_018125160.1, OYL21963.1, PAY74230.1, CFQ72446.1; Marino, |
| | Zhang, Borges, 2018, Science Oct 12; 362(6411): 240-242. |
| AcrIF11.1 | WP_033936089.1 |
| AcrIF11.2 | EGE18857.1 |
| AcrIF12 | ABR13388.1; Marino, Zhang, Borges, 2018, Science Oct |
| | 12; 362(6411): 240-242. |
| AcrIF13 | EGE18854.1; Marino, Zhang, Borges, 2018, Science Oct |
| | 12; 362(6411): 240-242. |
| AcrIF14 | AKI27193.1; Marino, Zhang, Borges, 2018, Science Oct |
| | 12; 362(6411): 240-242. |
| AcrIF15 | WP_117085604.1; Pinilla-Redondo et al. 2020, Nature Communications. 11, |
| | Article number: 5652 |
| AcrIF16 | WP_121296237.1; Pinilla-Redondo et al. 2020, Nature Communications. |
| | 11, Article number: 5652 |
| AcrIF17 | WP_102117861.1; Pinilla-Redondo et al. 2020, Nature Communications. |
| | 11, Article number: 5652 |
| AcrIF18 | WP_049300010.1, WP_060431798.1; Pinilla-Redondo et al. 2020, Nature |
| | Communications. 11, Article number: 5652 |
| AcrIF19 | WP_119870654.1; Pinilla-Redondo et al. 2020, Nature Communications. |
| | 11, Article number: 5652 |
| AcrIF20 | WP_119870655.1, WP_121268706.1; Pinilla-Redondo et al. 2020, Nature |
| | Communications. 11, Article number: 5652 |
| AcrIF21 | WP_102117862.1; Pinilla-Redondo et al. 2020, Nature Communications. |
| | 11, Article number: 5652 |
| AcrIF22 | WP_109463511.1; Pinilla-Redondo et al. 2020, Nature Communications. |
| | 11, Article number: 5652 |
| AcrIF23 | WP_052155777.1; Pinilla-Redondo et al. 2020, Nature Communications. |
| | 11, Article number: 5652 |
| AcrIF24 | WP_043084540.1; Pinilla-Redondo et al. 2020, Nature Communications. |
| | 11, Article number: 5652 |
| AcrIE4-F7 | WP_064584002.1; Marino, Zhang, Borges, 2018, Science Oct |
| | 12; 362(6411): 240-242. |
| AcrIA1 | Zhang, 2019, J Bacteriol. May 22; 201(12): e00747-18. |
| | MRSKMIKKEEKDNKIYITVKDEETGIEWTAVVEKVEFEWCVKQKEELEVEDAEKSV |
| | MLDYALFGNCAIPKVTAEEYKNSLTKYTGEKMSRLLHILYNYEIVSQNDTKNIWVTE |
| | LSRCLRRSYLMRKEGKTKVGLNEAMKMHIGSGLHMRLQSLLRKHGFETEVRVQRK |
| | TALGFQIVGRIDVYDKEENVIYELKYTHNDKLDSVRLNNYLRQLNYYIEMANAMKG |
| | YLVIVHADGSVEEIKRDWAETDLEKRANAFGIYVEENTLPPKKSRPDAECIECPFYNF |
| | CWGKL (SEQ ID NO: 4) |
| AcrIB1 | ACV38859.1 Lin, 2020, Molecular Cell. 78, 850-861 |
| AcrIC1 | AKG19229.1 Marino, Zhang, Borges, 2018, Science Oct 12; |
| | 362(6411): 240-242 |
| AcrIF2/C2 | Leon et al., 2020. bioRxiv. 2020.2006.2015.151498; |
| | Leon, 2021, Nucleic Acids Research. |
| | Volume 49, Issue 4, 26 Feb. 2021, Pages 2114-2125; |
| | MATKTAQMIAQQHKDTVAACEAAEAIAIAKDQVWDGEGYTKYTFDDNSVLIQSGT |
| | TQYAMDADDADSIKGYADWLDDEARSAEASEIERLLESVEEE (SEQ ID NO: 5) |
| AcrIC3 | WP_058130594.1, KSR23770.1 Leon, 2021, Nucleic Acids Research. |
| | Volume 49, Issue 4, 26 Feb. 2021, Pages 2114-2125 |
| AcrIC4 | WP_153575361.1, CD085538.1 Leon, 2021, Nucleic Acids Research. |
| | Volume 49, Issue 4, 26 Feb. 2021, Pages 2114-2125 |
| AcrIC5 | SDK41378.1, WP_089394111.1 Leon, 2021, Nucleic Acids Research. |
| | Volume 49, Issue 4, 26 Feb. 2021, Pages 2114-2125 |
| AcrIC6 | WP_080050315.1 Leon, 2021, Nucleic Acids Research. Volume 49, Issue |
| | 4, 26 Feb. 2021, Pages 2114-2125 |
| AcrIC7 | WP_003294373.1, EWC40192.1 Leon, 2021, Nucleic Acids Research. |
| | Volume 49, Issue 4, 26 Feb. 2021, Pages 2114-2125 |
| AcrIC8 | WP_074202337.1 Leon, 2021, Nucleic Acids Research. Volume 49, Issue |
| | 4, 26 Feb. 2021, Pages 2114-2125 |
| AcrVA3 | Marino, Zhang, Borges, 2018, Science Oct 12; 362(6411): 240-242; |
| | MVGKSKIDWQSIDWTKTNAQIAQECGRAYNTVCKMRGKLG |
| | KSHQGAKSPRKDKGISRPQPHLNRLEYQALATAKAKASPKA |
| | GRFETNTKAKTWTLKSPDNKTYTFTNLMHFVRTNPHLFDPD |
| | DVVWRTKSNGVEWCRASSGLALLAKRKKAPLSWKGWRLIS |
| | LTKDNK (SEQ ID NO: 6) |
| AcrIC9 | ETD02882.1; Gussow, 2020, Nature Communications 11, |
| | Article number: 3784 (2020) |
| AcrIC10 | WP_017907426.1, WP_058195519.1; Gussow, 2020, Nature |
| | Communications 11, Article number: 3784 (2020) |
| AcrID1 | NP_666537.1; He, 2018, Nature Microbiology. 3: 461-469 |
| AcrIIA1 | WP_003722518.1, AEO04364.1, AGR27297.1, EEW20426.1, EZH69029.1, KHK04755.1, |
| | KID25720.1, KKB87492.1, KTA28092.1, EAL06505.1, EEW22374.1, EFG00298.1, |
| | KTA33667.1, KTA68177.1, ALU78083.1, KHK19909.1, KHK17523.1, KID20145.1, |
| | KID21568.1, KID27662.1, KEU69221.1, KTA45326.1, KTA50988.1, KES96881.1, |
| | KET73262.1, KET94692.1, KEV69929.1, KEV93281.1, KEW08182.1, KEW09555.1, |
| | KEW17020.1, KEW65181.1, KEX05984.1, KHK12400.1, KJJ91612.1, KJQ94314.1, |
| | KJQ95812.1, KJR51140.1, KJR60209.1, KKD43688.1, KTA63900.1, KXF66381.1, |
| | AGR07061.1; Rauch, 2017, Cell. Jan 12; 168(1-2): 150-158.e10 |
| AcrIIA2 | WP_003722517.1, WP_167811084.1AE004363.1, AKI52062.1, |
| | EZH71062.1, KEU52814.1, KID23650.1, KID25721.1, KKB87491.1, |
| | KKB89544.1, KXS58607.1, EAL06504.1, EFG00297.1, KTA33666.1, |
| | KXS56902.1, KXW85500.1, KID20146.1, KID21567.1, KID27661.1, |
| | KXX34834.1, KXX34219.1, KES29690.1, KES36190.1, KEU69222.1, |
| | KEX13879.1, KEX45732.1, KEX49272.1, KLI12475.1, KNX95906.1, |
| | KES96882.1, KET73263.1, KET94691.1, KEV69928.1, KEV93282.1, |
| | KEW08181.1, KEW09554.1, KEW17021.1, KEW65182.1, KEX05985.1, |
| | KXF66382.1; Rauch, 2017, Cell. Jan 12; 168(1-2): 150-158.e10. |
| AcrIIA2-1 | Marshall et al., 2018, Molecular Cell 69, 146-157 |
| AcrIIA2-2 | Marshall et al., 2018, Molecular Cell 69, 146-157 |
| AcrIIA2b | Jiang et al., (2019). Mol Cell 73, 601-610 e605 |
| AcrIIA3 | WP_014930691, EXL25968.1; Rauch, 2017, Cell. Jan 12; 168(1-2): 150- |
| | 158.e10 |
| AcrIIA4 | WP_003723290.1, AE004689.1, EAL05809.1, EEW23439.1, EFG00182.1, |
| | KTA31189.1, AMD24318.1; Rauch, 2017, Cell. Jan 12; 168(1-2): 150- |
| | 158.e10 |
| AcrIIA4-2, | Marshall et al., 2018, Molecular Cell 69, 146-157 |
| AcrIIA4-3 | |
| AcrIIA4 | e.g., AcrIIA4 variant Ins. 5, AcrIIA4 variant N39A, AcrIIA4 variant |
| Variants | D14A/G38A, See e.g., Aschenbrenner et al., Science Advances. 6(6): |
| | eaay0187 (2020) |
| AcrIIA5 | D4276_028, ASD50988.1; Hynes, 2017, Nature Microbiology. 2, pages 1374-1380 (2017) |
| AcrIIA5-2 | Marshall et al., 2018, Molecular Cell 69, 146-157 |
| AcrIIA6 | D1811_026, AVO22749.1, AVO22721.1; Hynes, 2018, Nature Communications. 9, Article |
| | number: 2919 (2018) |
| AcrIIA7 | VDB32354.1; Uribe, 2019, Cell Host & Microbe. 2019 Feb 13; 25(2): 233-241.e5 |
| AcrIIA8 | VDB32352.1; Uribe, 2019, Cell Host & Microbe. 2019 Feb 13; 25(2): 233- |
| | 241.e5 |
| AcrIIA9 | VDB32351.1; Uribe, 2019, Cell Host & Microbe. 2019 Feb 13; 25(2): 233-241.e5 |
| AcrIIA10 | VDB32353.1; Uribe, 2019, Cell Host & Microbe. 2019 Feb 13; 25(2): 233-241.e5 |
| AcrIIA11 | QEH00205.1, WP_064786071.1, OHE28210.1, OHE43765.1, |
| | WP_006572312.1, WP_009258904.1, WP_054338718.1, |
| | WP_016321673.1, WP_023346767.1, WP_055271317.1, |
| | WP_118651841.1 Forsberg et al., 2019. eLife 8: e46540. |
| AcrIIA12 | Osuna et al., 2020. Cell Host & Microbe 28, 31-40. |
| | MSKTMYKNDVIELIKNAKTNNEELLFTSVERNTREAATQYFRCPEKHVSDAGVYYG |
| | EDFEFDGFEIFEDDLIYTRSYDKEELN (SEQ ID NO: 7) |
| AcrIIA13 | WP_050337628.1; Watters et al., 2020. PNAS USA, 117(12): 6531-6539 |
| AcrIIA13b | WP_053038109.1, Watters et al., 2020. PNAS USA, 117(12): 6531-6539 |
| AcrIIA14 | Liu et al., Nucleic Acids Res. 2021 Jun 21; 49(11): 6587-6595; Watters et |
| | al., 2020. PNAS USA, 117(12): 6531-6539 |
| AcrIIA15 | Watters et al., 2020. PNAS USA, 117(12): 6531-6539; |
| | MRKTIERLLNSELSSNSIAVRTGVSQAVISKLRNGKKELGNLTLNSAEKLFEYQKEME |
| | KVDTWIVYRGRTADMNKSYIAEGSTYEEVYNNFVDKYGYDVLDEDIYEIQLLKKNG |
| | ENLDDYDVDSDGINNYDKLDEFRESDYVDLEDYDYRELFENSSSQVYYHEFEITHE |
| | SEQ ID NO: 8) |
| AcrIIA16 | Mahendra et al., Nat Microbiol. 2020 Apr; 5(4): 620-629; |
| | MGYIGTKRSERSQDAIEDYEVPLNHFNKDLIQAFIDENEAYDTLKTKKVRLWKFVAP |
| | RAGATSWHHTGTYYNKTDHYSLEKVADELLQNGDEWEEQFKAYVKEEQETATSEP |
| | VFLSVIKVQIWGGSMKRPKLVGHEVVMGVKKEGWLHAVSKATQSKYKLSANKVE |
| | MQKHYSLEDYSALTKDFPEFKAQKRAINKKMKEMYN (SEQ ID NO: 9) |
| AcrIIA17 | WP_002401839.1, WP_074626943.1; Mahendra et al., Nat Microbiol. |
| | 2020 Apr; 5(4): 620-629 |
| AcrIIA18 | WP_099390844.1, WP_074627086.1; Mahendra et al., Nat Microbiol. |
| | 2020 Apr; 5(4): 620-629 |
| AcrIIA19 | WP_107591702.1, WP_100006909.1; Mahendra et al., Nat Microbiol. |
| | 2020 Apr; 5(4): 620-629 |
| AcrIIA20 | Eitzinger et al., Nuc. Acid. Res. 2020. 48(9): 4698-4708; |
| | MKNYEVTNEVKNLNTQVETIGQAVDLYKEYGSNTIVWSIDKNEDLIDEVTELVAEY |
| | AEKGTVIK (SEQ ID NO: 10) |
| AcrIIA21 | Eitzinger et al., Nuc. Acid. Res. 2020. 48(9): 4698-4708; |
| | MDYDNENYLIPKILLQDDFYSSLSAKDILVYAVLKDRQIEALEKGWIDTDGSIYLNFK |
| | LIELAKMFSCSRTTMIDVMQRLEEVNLIERERVDVFYGYSLPYKTYINEV (SEQ ID |
| | NO: 11) |
| AcrIIA22 | QEH00216.1; Forsberg, 2020, bioRxiv. https://doi.org/10.1101/2020.09.28.317578; |
| | MVVEETRDLAETADCVVIEAILVDDGLRYRQLSVGIKDENGDIIRIVPISTVLI (SEQ |
| | ID NO: 12) |
| AcrIIA23 | Varble et al., 2020. bioRxiv, 2020.2010.2009.333658; |
| | MFIYVIRRNKMEYGNKIFEIYNKPFKYRNSSSTNYNKVRASGIEPNTKFVVNKTANIN |
| | CAVYPRHGSIEKVFYWGDRKITQATAEKRCGYFKG (SEQ ID NO: 13) |
| AcrIIA24 | Song, 2022, Nucleic Acids Research, Nucleic Acids Research, Volume 50, |
| | Issue 5, 21 March 2022, Pages 2836-285 |
| | MKKAQQLLKEIKTNNVSYAIMDEDNEIYCNKETNNIMDIYGYDNENG |
| | HFYGVYGDVVDGQIDSRYFSDDAILNAIDKLLFLGDPIKRTDLPSDAD |
| | FKRTFFFEE (SEQ ID NO: 14) |
| AcrIIA25 | Song, 2022, Nucleic Acids Research, in press; |
| | MKNRLLGSRYTDAIKNDCGTANKMSNIYNKLNKDSLREIHSALYGLLTAGYDISNM |
| | RNIEELEKYVNLKKSRGQLLNVSSDDIKLYHKLFVIRFGK (SEQ ID NO: 15) |
| AcrIIA26 | Song, 2022, Nucleic Acids Research, in press; |
| | MKKLYIQTNQFANGELQVENTSYELCDTFKELYSVASNLVDENTLNFVEDNFIEQNY |
| | KDEYNGVYENDGDTGEFVGQVFENKVTEEQFKELLEQLEITYTEFDPEEELAKCIAN |
| | KNRKSEFYGNGLKVIAEYLESISHEDALAVVTYYYFYFGFGYEDQLISDIKDDQEDG |
| | VKFEHVERSETI (SEQ ID NO: 16) |
| AcrIIA27 | Song, 2022, Nucleic Acids Research, in press; |
| | MKTFNIIVSESANLKEHSSELVDNIIYKVEAKNRREAFKKAREEYSFSSKWKFNMRD |
| | LTAIDNTHRRAWGRRYLRVEEA(SEQ ID NO: 17) |
| AcrIIA28 | Song, 2022, Nucleic Acids Research, in press; |
| | MKTIFTKKQTEELLNDISIEKQKELFNSMHDFRSQHAKEARIPGWSDKYNKLEKKML |
| | SDFEEVTGIKYDTLESELIWDNLSNKFLYNS (SEQ ID NO: 18) |
| AcrIIA29 | Song, 2022, Nucleic Acids Research, in press; |
| | MKPSQKIKWLLTATGITTYKIGKDIEESTQFLDRYKNDPEKIGGMRLEKAEKLLEYIS |
| | NLRQEDVIKTNWNNQQILVQNSTEKEITKYFNSYPFAIKLNWIKPHKEMFIVNFDTTS |
| | NKTFRKYPYDLKNLYFLVDKNRDKMSQFAEFLIICGRKSHFGGSRVLYEVEGKKYQI |
| | IFSIKRPSELGPTIRLINVVETDTYRDDLVPKISEEESILRSEDLDLKGKRVSIKDS |
| | ELLELMSIIDN (SEQ ID NO: 19) |
| AcrIIA30 | Song, 2022, Nucleic Acids Research, in press; |
| | MITANEIVKTHKGIRLVQRKNESWEEFKERIQEVIAKQGDNYLTQTKPVHEIKNKGT |
| | RNIRRTYVNILLKEGA (SEQ ID NO: 20) |
| AcrIIA31 | Song, 2022, Nucleic Acids Research, in press; |
| | MVTEEQLKEVLVGIYETEYKDEQTFEEYADGWDFWIDKDGDILIEGRGMKPIDGVQ |
| | KVGHVDNGVIYAY (SEQ ID NO: 21) |
| AcrIIA32 | Song, 2022, Nucleic Acids Research, in press; |
| | MKNEDGKLVVSKAHFGNMIRNCQSVEDFKKSFERLTYYSSENRESTVRQRLKIAEKE |
| | YNFKAGVKEDLEIKNTTDKEILDYVRNELSKIDSKKQADKNWSEKNREHRNYLSKR |
| | SSARSFINNNATHEDLLELKKIIEEKLK (SEQ ID NO: 22) |
| AcrIIC1 | WP_049360089.1; Pawluk et al., Cell. 2016. 167(7): 1829-1838 |
| AcrIIC2 | WP_042743678.1; Pawluk et al., Cell. 2016. 167(7): 1829-1838 |
| AcrIIC3 | WP_042743676.1; Pawluk et al., Cell. 2016. 167(7): 1829-1838 |
| AcrIIC4 | WP_049372635.1; Lee et al., 2018. mBio. 9(6): e02321-18 |
| AcrIIC5 | WP_002642161.1; Lee et al., 2018. mBio. 9(6): e02321-18 |
| AcrIIC6 | MKTLKTNLFVLEQTSQNTFNVYRNHYDINISTPYGIVKLSDEVIAGLEKKPHSGYWS |
| | EVVRQTVEQNGALYEKHKI (SEQ ID NO: 23) |
| AcrID1 | YP_009272954.1, YP_003728.1, NP_445679.1 |
| AcrIII1 | NP_666617.1; Athukoralage, 2020, Nature. 577, pages 572-575 (2020) |
| AcrIIIB1 | NP_666582.1; Bhoobalan-Chitty, 2019, Cell. 179: 448-458 |
| AcrVIA1 | ERK51680.1; Lin, 2020, Molecular Cell. 78, 850-861; Meeske et al., |
| | Science. 369 (6499): 54-59 |
| AcrVIA2 | ERK51681.1; Lin, 2020, Molecular Cell. 78, 850-861 |
| AcrVIA3 | ERK48335.1; Lin, 2020, Molecular Cell. 78, 850-861 |
| AcrVIA4 | ERK48333.1; Lin, 2020, Molecular Cell. 78, 850-861 |
| AcrVIA5 | ERK48092.1; Lin, 2020, Molecular Cell. 78, 850-861 |
| AcrVIA6 | ETD74580.1; Lin, 2020, Molecular Cell. 78, 850-861 |
| AcrVIA7 | ACV38861.1; Lin, 2020, Molecular Cell. 78, 850-861 |
| AcrVIB | WP_034985946.1, EKB54194.1; Wandera et al., 2022, MolCell. |
| | Jul 21; 82(14): 2714-2726.e4 |
| AcrVA1 | WP_046701302.1, AKG19227.1, WP_046699156.1; Marino, Zhang, |
| | Borges, 2018, Science Oct 12; 362(6411): 240-242 |
| AcrVA2 | AKG19228.1, AKG12143.1; Marino, Zhang, Borges, 2018, Science Oct |
| | 12; 362(6411): 240-242 |
| AcrVA3 | AKG19230.1, 00R90252.1; Marino, Zhang, Borges, 2018, Science Oct |
| | 12; 362(6411): 240-242 |
| AcrVA3.1 | B0181_04965, Marino, Zhang, Borges, 2018, Science Oct |
| | 12; 362(6411): 240-242 |
| AcrVA4 | AKG19230.1; Watters et al., 2018. Science. 362(6411): 236-239 |
| AcrVA5 | WP_046699157.1, AKG12174.1; Watters et al., 2018. Science. |
| | 362(6411): 236-239 |
| Csx27 | Marino et al., 2020. Nat Meth. 17(5): 471-479 |

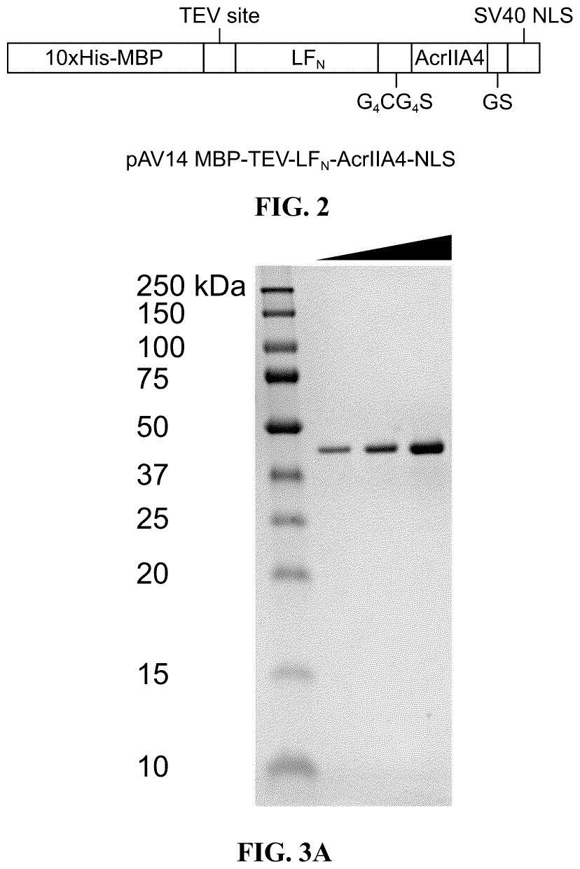

Additional exemplary Acr polypeptides and/or anti-CRISPR functional domains that can be included in the engineered Acr polypeptides of the present invention are discussed, e.g., Zhang and Marchisio. RNA Biol. 2021 August; 18 (8): 1085-1098; Liu et al., FEBS J. 2020 February; 287 (4): 626-644, particularly at Table 1; Yu and Marchisio. Front Bioeng Biotechnol. 2020 Sep. 30; 8:575393, particularly at Table 2; Zhu et al., BMC Biol. 2018 Mar. 19; 16 (1): 32; an Acr in any one or more of the following databases: AcrDB (Acr Database) (Huang et al., Nucleic Acids Research, Volume 49, Issue D1, 8 Jan. 2021, Pages D622-D629), Anti-CRISPRdb (see, e.g., Dong et al., Nucl Acid Res. 2018. 46: D393-D398, Anti-CRISPRdb V2.2 (available at http://guolab.whu.edu.cn/anti-CRISPRdb/), the Acr Registry (anti-CRISPR assembly spreadsheet, available at https://tinyurl.com/anti-CRISPR), CRISPRimer (see e.g., Zhang et al., Commun Biol. 2018. 1:180, Bondy-Denomy et al., Acr nomenclature (CRISPR J. 2018; 1:304-305, ArcCatalog (see e.g., Gussow et al., Nat. Commun. 2020; 11:3784, AcRanker (see, e.g., Eitzinger et al., Nucleic Acids Res. 2020; 48:4698-4708 and those identified using the method described therein), AcrFinder (see, e.g., Yi et al., Nucleic Acids Res. 2020; 48: W358-W365), PaCRISPR (see, e.g., Wang et al., Nucleic Acids Res. 2020; 48: W348-W357), AcrDetector (see, e.g., Dong et al., Precise detection of Acrs in prokaryotes using only six features. 2020; bioRxiv doi: 26 May 2020, https://doi.org/10.1101/2020.05.23.112011), AcrHub (see e.g., Wang et al., Nuc Acid Res. 2021. 49 (D1): D630-D638); Rauch et al., Cell. 2017; 168:150-158; Hwang and Maxwell. 2019. The CRISPR Journal. 2 (1), DOI: 10.1089/crispr.2018.0052, particularly in Table 1; Pinilla-Redondo. 202. Nat. Commun. 11 (5652), particularly at FIGS. 1 and 2; Forsberg et al., 2021. PLOS Biol 19 (10): e3001428. https://doi.org/10.1371/journal.pbio.3001428; Meeske et al., 2020. Science. 369 (6499): 54-59; Davidson et al., 2020. Ann. Rev. Biochem. Vol. 89:309-332; Stanley et al., 2019. Cell. 178 (6): 1452-1464; Pawluk et al., Nature Reviews Microbiology volume 16, pages 12-17 (2018); Peng et al., 2020. Trends Microbiol. 28 (11): 913-921; Osuna et al., 2020. Cell Host & Microbe 28, 31-40; Trasanidou et al., 2019. Nat. Comm. 10:2806; Liu et al., Nucleic Acids Res. 2021 Jun. 21; 49 (11): 6587-6595; Watters et al., 2020. PNAS USA, 117 (12): 6531-6539; Mahendra et al., Nat Microbiol. 2020 April; 5 (4): 620-629; Varble et al., 2020. bioRxiv, 2020.2010.2009.333658; Leon et al., 2020. bioRxiv. 2020.2006.2015.151498; Jiang et al., (2019). Temperature-Responsive Competitive Inhibition of CRISPR-Cas9. Mol Cell 73, 601-610 e605; Liu, et al. (2019). Mol Cell 73, 611-620 e613; Dong et al. Nature. 546:429-432; Stanley, S. Y. (2018). An Investigation of Bacteriophage Anti-CRISPR and Anti-CRISPR Associated Proteins. In Department of Molecular Genetics (http://hdl.handle.net/1807/97883: University of Toronto), pp. 120; Yang et al., Nat Comm 13:1931 (2022); EP3615552 A1; WO/2018/197495; US20200190492; WO/2018/197520; US20200040328; EP3615665; US20190382741; WO/2017/160689; EP3429635; US20210198328; WO/2019/185751; WO/2020/059708; WO/2020/043148; WO/2019/076651; US20210317480; US20210095004; WO/2021/108442, the disclosures of which can be adapted for use with the present invention.

In an embodiment, the Acr polypeptide is an engineered polypeptide with improved potency. In an embodiment, the engineered Acr polypeptide with improved potency is any of those set forth, as described in Mathony et al. Nat. Chem. Biol. 16:725-730 (2020). In an embodiment, the Acr polypeptide is engineered to be responsive to an external stimulus (e.g., a chemical stimulus, light stimulus, radiation stimulus, magnetic stimulus, temperature stimulus, or other physical or energy stimulus. Such engineered Acr polypeptides can allow for further temporal or spatial control of Acr activity. In some example embodiments, the engineered Acr polypeptide is coupled to a light-sensitive molecule. See, e.g., Bubeck et al., Nature Methods volume 15, pages 924-927 (2018). In some example embodiments, the engineered Acr polypeptide is coupled to a molecule or polypeptide degraded in a particular cell cycle phase. See, e.g., Matsumoto et al., Communications Biology 3:601 (2020).

In an embodiment, the Acr polypeptide is about 12 to about 1,000 amino acids in length. See e.g., Cui et al., Genome Biol. 21:51 (2020). In an embodiment, the Acr polypeptide is about 12, to/or 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000 or more amino acids in length.

In an embodiment, the Acr polypeptide(s) reduce one or more CRISPR-Cas system activities by 1 to 1,000 fold or more. In an embodiment, the Acr polypeptide(s) reduce one or more CRISPR-Cas system activities by 1 to/or 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000 fold or more.

In an embodiment, the Acr polypeptide(s) reduce one or more CRISPR-Cas system activities by any non-zero number to/or 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%.

### Cargo Delivery Molecules

The engineered Acr polypeptide contains a cargo delivery molecule operatively coupled to the Acr polypeptide. Without being bound by theory and shown in, e.g., FIG. 1A-1B, the cargo delivery molecule can bind, attach, or otherwise interact with one or more pore-forming polypeptides. In an embodiment, the cargo delivery molecule is operatively coupled to the Acr polypeptide by fusing, in-frame, the Acr polypeptide and the cargo delivery molecule. In an embodiment, the cargo delivery molecule is operatively coupled to the Acr polypeptide via a linker. In an embodiment, the linker is a peptide linker. In an embodiment, the peptide linker is flexible. In an embodiment, the peptide linker is rigid. In an embodiment, the linker is a cleavable linker. In an embodiment, the cleavable linker is cleaved by an enzyme, light, radiation, a chemical reaction, and/or the like. In an embodiment, the linker is configured or otherwise capable of bioconjugation. For example, the linker can be used with or include a cysteine, which can allow for bioconjugation. In an embodiment, the cargo delivery molecule is operatively coupled to the Acr polypeptide via a GS linker with a cysteine between the GS linker and the cargo delivery molecule and/or Acr polypeptide. In an embodiment, the linker includes one or more residues for bioconjugation reactions.

In an embodiment, the linker is a non-cleavable linker.

In an embodiment, the peptide linker has a sequence of GGGLLK (SEQ ID NO: 24). In an embodiment, the peptide linker has a sequence of GGGLLK (SEQ ID NO: 25), wherein L4 and/or L5 are D-Leu. In an embodiment, the peptide linker has a sequence of GGG[GGS]₇K (SEQ ID NO: 26). In an embodiment, the peptide linker has a sequence of GGG[GGS]₇K (SEQ ID NO: 27), where S is L-Ser and/or K is L-Lys. In an embodiment, the peptide linker contains an N^{ε}-linked α-bromoacetyl group. In an embodiment, the peptide linker contains an N^{ε}-linked maleimide group. In an embodiment, the peptide linker is linker peptide 1, 2, or 3 of Lu et al., ACS Cent. Sci. 2021. 7:365-378. In an embodiment, the peptide linker comprises LPSTGGK (SEQ ID NO: 28). In an embodiment, the linker is or comprises GGGGGGGGGS (SEQ ID NO: 29). In an embodiment, the linker is a GlySer linker. Additional exemplary linkers include those set forth in Chen et al., Adv Drug Deliv Rev. 2013 Oct. 15; 65 (10): 1357-1369; Rosmalen et al., Biochem. 2017, 56, 50, 6565-6574; a Proline 9 (P9) linker, GAAPAAAPAKQEAAAPAPAAKAEAPAAAPAAKA (SEQ ID NO: 30), (GGGGS)₃ (SEQ ID NO: 31), (G)₈ (SEQ ID NO: 32), (G)₆ (SEQ ID NO: 33), (EAAAK)₃ (SEQ ID NO: 34), (EAAAK)ₙ (n=1-3) (SEQ ID NO: 35-36, 34), A(EAAAK)₄ALEA(EAAAK)₄A (SEQ ID NO: 37), GGGGS (SEQ ID NO: 38), PAPAP (SEQ ID NO: 39), AEAAAKEAAAKA (SEQ ID NO: 40), (GGGGS)ₙ (n=1-10) (SEQ ID NO: 38, 41, 31, 42-48), (Ala-Pro)n (n=10-32) (SEQ ID NO: 49-71), disulfide, VSQTSKLTR↓AETVFPDV (SEQ ID NO: 72), PLG↓LWA (SEQ ID NO: 73), RVL↓AEA (SEQ ID NO: 74); EDVVCC↓SMSY (SEQ ID NO: 75); GGIEGR↓GS (SEQ ID NO: 76), TRHRQPR↓GWE (SEQ ID NO: 77); AGNRVRR↓SVG (SEQ ID NO: 78); RRRRRRR↓R↓R (SEQ ID NO: 79), GFLG↓ (SEQ ID NO: 80), LE, LEAGCKNFFPR↓SFTSCGSLE (SEQ ID NO: 81), CRRRRRREAEAC (SEQ ID NO: 82), a TEV site linker, e.g., ENLYFQ↓(S, G, A, M, C, or H) (SEQ ID NO: 83), (Protease-sensitive cleavage sites are indicated with “↓”) or any combination thereof. In an embodiment, the linker is or comprises LPSTGGK (SEQ ID NO: 28). Other suitable linkers will be appreciated by those of ordinary skill in the art in view of the description herein.

In an embodiment, the Acr polypeptide is operatively coupled to the N-terminus, the C-terminus, or between the N- and C-terminus of a cargo delivery molecule. In an embodiment, the cargo delivery molecule is cleavably coupled to the Acr polypeptide. In an embodiment, the cargo delivery molecule is cleavably coupled to the Acr polypeptide such that cleavage occurs via protease, deubiquitinase, small molecule, reducing agent, metabolite, etc. In an embodiment, the protease, deubiquitinase, small molecule, reducing agent, or metabolite, is native to a cytosol. In an embodiment, the protease, deubiquitinase, small molecule, reducing agent, or metabolite, is exogenous to a cytosol. In an embodiment, the cargo delivery molecule comprises a cleavable domain. For example, LF_{N} (an exemplary cargo delivery molecule) has been engineered to include a cleavable domain. See e.g., Rabideau and Pentelute ACS Cent. Sci. 2015, 1, 423-430 and Bachran et al., MBio. 2013, 4 (3), e00201-13. In an embodiment, a cargo delivery molecule that contains a cleavable domain is fused, in frame, to the Acr polypeptide. In an embodiment, the cargo delivery molecule is linked via a cleavable linker to the Acr polypeptide.

In an embodiment, the cargo delivery molecule is a native molecule capable of binding, attaching, or otherwise interacting with one or more pore-forming polypeptides. In an example embodiment, the cargo delivery molecule is or comprises a bacterial exotoxin, optionally a Bacillus anthracis lethal factor (LF) or edema factor (EF), a fragment thereof, or a derivative thereof or a Corynebacterium diphtheriae catalytic domain or derivative thereof. In an embodiment, the cargo delivery molecule is or comprises the N-terminal domain of Bacillus anthracis LF (LF_{N}) (see e.g., Feld et al., 2010. Nat. Struct. Mol. Biol. 17:1383-1390). In an embodiment, the cargo delivery molecule is or comprises botulinum neurotoxin (BoNT), a fragment thereof, or a derivative thereof. See e.g., Tian et al., Cell Rep. 2022. 38 (10): 110476. In an embodiment, the cargo delivery molecule is capable of binding the pre-pore or a pre-pore polypeptide forming the pre-pore at one or more domains or regions. In an embodiment, an LF or EF cargo delivery molecule is capable of binding a pre-pore comprising one or more protective antigen (PA) polypeptides. In an embodiment, an LF or EF cargo delivery molecule is capable of binding a pre-pore comprising PA₆₃ polypeptides. PA₆₃ refers to the portion of the full-length PA (PA₈₃) after cleavage of its N-terminal 20 kDa portion (PA₂₀). In an embodiment, an LF or EF cargo delivery molecule is capable of binding a pre-pore comprising PA₆₃ polypeptides. In an embodiment, the LF or EF cargo delivery molecule is capable of binding the pre-pore comprising PA polypeptides via ˜30 kDa homologous N-terminal domains. See e.g., Pimental et al., 2004. Biochem. Biophys. Res. Commun. 322:258-262. 10.1016/j.bbrc.2004.07.105, and Drum et al., J Biol. Chem. 275 (46): 36334-36340 (2000).

In an embodiment, the cargo delivery molecule comprises or is engineered to comprise a pore-forming polypeptide interaction molecule or domain. In an embodiment, the pore-forming polypeptide interaction molecule or domain is capable of binding or otherwise interacting with a pore-forming polypeptide. In an embodiment, the pore-forming polypeptide interaction molecule or domain is capable of directing the engineered Acr polypeptide to a pore-forming polypeptide that is part of a pre-pore or pore. In an example embodiment, the cargo delivery molecule comprises an N-terminal, C-terminal, or N- and C-terminal pore-forming polypeptide interaction molecule or domain. In an embodiment, the cargo delivery molecule comprises a pore-forming polypeptide interaction molecule or domain located between the N- and C-terminals of the cargo delivery molecule. In an example embodiment, the pore-forming polypeptide interaction molecule or domain can be operatively coupled (e.g., fused in frame or linked via a linker) to the N-terminus, the C-terminus, or between the N- and C-terminus of a cargo delivery molecule.

In an embodiment as previously discussed, the pore-forming polypeptide interaction molecule or domain is an LF or EF polypeptide. In an embodiment, the pore-forming polypeptide interaction molecule or domain is a heterologous (relative to the cargo delivery molecule and/or pore-forming polypeptide) LF or EF polypeptide. In an embodiment, the pore-forming polypeptide interaction molecule or domain is a heterologous (relative to the cargo delivery molecule and/or pore-forming polypeptide) LF_{N} polypeptide. In an embodiment, a cargo delivery molecule comprises a heterologous (relative to the cargo delivery molecule and/or pore-forming polypeptide) N-terminal EF polypeptide. In an embodiment, the pore-forming polypeptide interaction molecule or domain is a polypeptide. In an embodiment, the pore-forming polypeptide interaction molecule or domain is a positively charged polypeptide. In an embodiment, the pore-forming polypeptide interaction molecule or domain is a polybasic polypeptide. In an embodiment, the pore-forming polypeptide interaction molecule comprises a purification or identification tag or reporter. In an embodiment, the pore-forming polypeptide interaction molecule or domain is or comprises a polyhistidine tag (His-tag). Other exemplary purification and identification tags and reporters are described elsewhere herein and can be used with the pore-forming interaction molecule. In an embodiment, the pore-forming polypeptide interaction molecule or domain comprises K and/or R residues such that the peptide is basic. In an embodiment, the number of K and/or R residues is such that the total percent of K and/or R residues in the polypeptide interaction molecule or domain is 0.01 to 100%. In an embodiment, the pore-forming polypeptide interaction molecule or domain is a negatively charged polypeptide. In an embodiment, the pore-forming polypeptide interaction molecule or domain is a polyacid polypeptide.

In an embodiment, the pore-forming interaction molecule or domain is capable of binding or interacting with a pre-pore polypeptide or domain thereof, a pre-pore complex, or pore, which are described in greater detail elsewhere herein.

### Pore-Forming Polypeptides

The engineered Acr polypeptide is capable of binding, attaching, or otherwise interacting with one or more pore-forming polypeptides. In an embodiment, the pore-forming polypeptide is a prokaryotic pore-forming polypeptide. In an embodiment, the pore-forming polypeptide is a bacteria pore-forming polypeptide. In an embodiment, the pore-forming polypeptide is a eukaryotic pore-forming polypeptide. In an embodiment, the eukaryotic pore-forming polypeptide is a eukaryotic immune system component (see e.g., Szczesny et al., PLOS ONE 6, e20349 (2011); Galinier et al., PLOS Pathog. 9, e1003216 (2013); Xiang et al., Proc. Natl. Acad. Sci. USA 111:6702-6707 (2014)).

In an embodiment, the pore-forming polypeptide is a pore-forming toxin, such as a bacterial pore-forming toxin. In an example embodiment, the pore-forming polypeptide is an alpha pore-forming polypeptide or a beta pore-forming polypeptide. In an example embodiment, the pore-forming polypeptide is an alpha pore-forming polypeptide. In an example embodiment, the pore-forming polypeptide is a beta pore-forming toxin. As used herein, “alpha”, in connection with the pore-forming polypeptide, refers to pore-forming polypeptides in which the membrane-spanning domain(s) are alpha helices (see e.g., Lesieur et al., Mol. Membrane Biol. 14:45-64 (1997); Iacovache et al., Curr. Opin. Struct. Biol. 20:241-246 (2010); Gouaux et al., Curr. Opin. Struct. Biol. 7:566-573 (1997)). As used herein, “beta”, in connection with the pore-forming polypeptide, refers to pore-forming polypeptides in which the membrane-spanning domain(s) are beta helices (see e.g., Lesieur et al., Mol. Membrane Biol. 14:45-64 (1997); Iacovache et al., Curr. Opin. Struct. Biol. 20:241-246 (2010); Gouaux et al., Curr. Opin. Struct. Biol. 7:566-573 (1997)).

In an embodiment, the pore-forming polypeptide is a colicin family pore-forming polypeptide. In an embodiment, the colicin family pore-forming polypeptide is an Escherichia coli pore-forming polypeptide (see e.g., Lakey et al., Toxicology. 87:85-108 (1994)). Exemplary colicin family pore-forming polypeptides include but are not limited to, Colicin E1, Colicin Ia, Colicin A, Colicin N, and derivatives thereof.

In an embodiment, the pore-forming polypeptide comprises a diphtheria toxin (DT), a diphtheria toxin translocation domain, a diphtheria toxin catalytic domain, a botulinum neurotoxin (BoNT), a deactivated botulinum neurotoxin-like toxin enzymatic domain (dBoNT/X-LC), a deactivated botulinum neurotoxin-like toxin translocation domain (BoNT/X-H_{N}), or any combination thereof. See e.g., Murphy, J. Toxins. 2011. 3(3), 294-308 and Tian et al., Cell Reports. 38(10): 110476 (2022).

In an embodiment, the pore-forming polypeptide is an Actinoporin family pore-forming polypeptide. In an embodiment, the Actinoporin family pore-forming polypeptide is from A. equina. In an embodiment, the Actinoporin family pore-forming polypeptide is from S. helianthus. In an embodiment, the Actinoporin family pore-forming polypeptide is from A. fragacea. Exemplary Actinoporin family pore-forming polypeptides include, but are not limited to, Equinatoxin II (EqtII), Sticholysin II (StnII), Fragaceatoxin C (FraC), and derivatives thereof.

In an embodiment, the pore-forming polypeptide is a ClyA family pore-forming polypeptide. In an embodiment, the ClyA family pore-forming polypeptide is from E. coli, S. enterica, S. flexneri, or B. cereus. Exemplary ClyA family pore-forming polypeptides include but are not limited to, Cyolysin A (ClyA, also known as HlyE), non-hemolytic tripartite enterotoxin (Nhe), Haemolysin BL (Hbl), and derivatives thereof.

In an embodiment, the pore-forming polypeptide is a Haemolysin family pore-forming polypeptide. In an embodiment, the Haemolysin family pore-forming polypeptide is from S. aureus, C. perfringens, V. cholerae, or V. vulnificus. Exemplary Haemolysin family pore-forming polypeptides include but are not limited to, alpha-haemolysin (Hla), gamma-haemolysin (Hlg), leukocidins (e.g., HlgACB, LukED), necrotic enteritis toxin B (NetB), delta-toxin, V. cholerae cytolysin (VCC), V. vulnificus haemolysin (VVH), and derivatives thereof.

In an embodiment, the pore-forming polypeptide is an Aerolysin family pore-forming polypeptide. In an embodiment, the Aerolysin pore-forming polypeptide is from an Aeromonas spp., Clostridium spp., Cnidaria spp., C. perfringens, L. sulphureus, or E. fetida. Exemplary Aerolysin family pore-forming polypeptides include but are not limited to, Aerolysin, alpha-toxin, hydralysin, ε-toxin, enterotoxin (CPE), Haemolytic lectin (LSL), Kysenin, and derivatives thereof.

In an embodiment, the pore-forming polypeptide is a cholesterol-dependent cytolysin (CDC) family pore-forming polypeptide. In an embodiment, the CDC family pore-forming polypeptide is from C. perfringens, S. suis, S. intermedius, L. monocytogenes, S. mitis, B. anthracis, or S. pyogenes. Exemplary CDC family pore-forming polypeptides include, but are not limited to, Perfringolysin O (PFO), Suilysin (SLY), Intermedilysin (ILY), Listeriolysin O (LLO), Lectinolysin (LLY), Anthrolysin O (ALO), Streptolysin O (SLO), and derivatives thereof.

In an embodiment, the pore-forming polypeptide is a membrane attack complex component/perforin (MACPF) pore-forming polypeptide. In an embodiment, the MACPF family pore-forming polypeptide is from P. luminescens or B. thetaiotamicron. Exemplary MACPF family pore-forming polypeptide Plu-MACPF, Bth-MACPF (BT 3439), and derivatives thereof.

In an embodiment, the pore-forming polypeptide is a repeats-in-toxin (RTX) family pore-forming polypeptide. In an embodiment, the RTX family pore-forming polypeptide is from E coli, B. pertussis, or A. hydrophila. Exemplary RTX family pore-forming polypeptides include but are not limited to, HylA, bifunctional haemolysin-adenylyl cyclase toxin (CyA), MARTX, and derivatives thereof.

In an example embodiment, the pore-forming polypeptide is or comprises a Bacillus anthracis protective antigen (PA) polypeptide or a derivative thereof. In an embodiment, the pore-forming polypeptide is or comprises a full-length PA polypeptide (e.g., a PA₈₃ polypeptide). In an embodiment, the pore-forming polypeptide is or comprises a PA₆₃ polypeptide.

In an embodiment the pore-forming polypeptide is or comprises a stabilized Bacillus anthracis protective antigen polypeptide as in Becker et al., BMC Biology volume 18, Article number: 100 (2020).

In an embodiment, the pore-forming polypeptide is or comprises a mutated PA. In an embodiment, 1-10 or more amino acids of the PA are mutated. In an embodiment, the mutated PA has an altered function or activity as compared to the non-mutated PA. In an embodiment, the PA is engineered so that its capability to form a pre-pore in the cell membrane is activated by a target protease or by other stimuli (e.g., radiation, chemical, molecule, etc.), and is different from wild-type PA₈₃ which requires activation by a cell-surface furin family protease. In an embodiment, the mutated PA lacks or has reduced binding at its native receptor and/or has binding at a non-native receptor. In an embodiment, the PA comprises a double mutation in domain 4 that ablates its ability to bind the native receptor. In an embodiment, the PA comprises a double mutation in domain 4 as described in Mechaley et al., Changing the receptor specificity of anthrax toxin. MBio 2012, 3 (3), e00088-12. In an embodiment, the PA variant is a protease-activated PA. In an embodiment, the PA comprises an N682A mutation, a D683A mutation, a K563C mutation, or any combination thereof. In an embodiment, the PA comprises an N682A mutation and a D683A mutation. In an embodiment, the PA comprises an N682A mutation, D683A mutation, and a K563C mutation. In an embodiment, the PA comprises a targeting moiety fused or otherwise coupled to the C-terminus, N-terminus, or both. In an embodiment, the targeting moiety is ZHER2, scFv, EGF, DARPin, an antibody, or a fragment thereof. Targeting moieties that can be fused or otherwise coupled to the PA or other pore-forming polypeptide are described in greater detail elsewhere herein.

Table 2 provides additional cell-targeting Anthrax proteins that can be used in the context of the present invention.

TABLE 2

| | | | Biological | |
|---|---|---|---|---|
| Year | PA modification | Targeting moiety | target | DOI (or reference) |
| 2000 | PA (protease activated) | matrix | MMP tumor | Cancer Res (2000) 60 (21): |
| | | metalloproteinase | cells | 6061-6067 |
| | | (MMP) | | |
| 2001 | PA (protease activated) | urokinase plasminogen | uPA tumor | 10.1074/jbc.M011085200 |
| | | activator (uPA) | cells | |
| 2003 | PA (protease activated) | urokinase plasminogen | uPA tumor | 10.1073/pnas.0236849100 |
| | | activator (uPA) | cells (lung, | |
| | | | colon, | |
| | | | breast, | |
| | | | stomach, | |
| | | | pancreas, | |
| | | | head and | |
| | | | neck, skin, | |
| | | | uterus, | |
| | | | ovaries, and | |
| | | | brain, | |
| | | | melanoma, | |
| | | | hard and soft | |
| | | | tissue | |
| | | | sarcoma, and | |
| | | | monocytic | |
| | | | and | |
| | | | myelogenous | |
| | | | leukemia) | |
| 2006 | PA (protease activated) | urokinase plasminogen | uPA tumor | 10.1158/1535-7163.Mct-06- |
| | | activator (uPA) | cells | 0315 |
| 2008 | PA (protease activated) | matrix | MMP tumor | 10.1074/jbc.M707419200 |
| | | metalloproteinase | cells | |
| | | (MMP) | | |
| 2012 | PA (N682A/D683A); | (1) Epidermal growth | (1) EGFR | 10.1128/mBio.00088-12 |
| | prepared with receptor- | factor (EGF); | (A431 cells) | |
| | binding protein fused to | (2) receptor-binding | (2) DTR | |
| | the C terminus | domain of diptheria | (CHO-K1 | |
| | | | cells) | |
| 2013 | PA (N682A/D683A); | ZHER2 affibody | HER2 (BT- | 10.1016/j.molonc.2012.12.003 |
| | prepared with receptor- | | 474) | |
| | binding protein (ZHER2) | | | |
| | fused to the C terminus | | | |
| 2020 | PA (N682A/D683A); | (1) anti-EGFR scFv | EGFR & | 10.1002/cbic.202000201 |
| | prepared with receptor- | (epthilial growth factor | CEA | |
| | binding scFv | receptor) | | |
| | | (2) anti-CEA scFv | | |
| | | (carcinoembryonic | | |
| | | antigen) | | |
| 2020 | PA (N682A/D683A); | EGF (EGFR ligand) | EGFR/HER2 | 10.1002/ijc.32719 |
| | prepared with receptor- | | tumors | |
| | binding protein (EGF) | | | |
| | fused to the C terminus | | | |
| 2020 | PA (N682A/D683A); | DARPin protein that | EpCAM | 10.1186/s12915-020-00827-y |
| | prepared with receptor- | binds to EpCAM | cells | |
| | binding protein | | | |
| | (DARPin) fused to the C | | | |
| | terminus | | | |
| 2021 | PA | Trastuzumab (Tmab), | HER2, | 10.1021/acscentsci.0c01670 |
| | (K563C/N682A/D683A), | cetuximab (Cmab), | EGFR, uPA, | |
| | conjugated with IgG | urokinase plasminogen | MMP tumor | |
| | alone, and with protease- | activator (uPA), | cells | |
| | activator sequences | matrixmetalloproteinase | | |
| | | activator (MMP) | | |

In an embodiment, the pore-forming protein is engineered to comprise a targeting moiety or targeting domain (see e.g., FIG. 1B). In some of these embodiments, the pore-forming protein is fused to a targeting domain or targeting moiety. In an embodiment, a pore-forming polypeptide or fragment thereof is mutated such that its ability to bind its native receptor is reduced or eliminated and is further fused to a targeting domain or targeting moiety that targets a non-native target. In an embodiment, a PA polypeptide or fragment thereof is mutated such that its ability to bind its native receptor is reduced or eliminated and is further fused to a targeting domain or targeting moiety that targets a non-native target. Exemplary targeting moieties and domains are described in greater detail elsewhere herein.

In an embodiment, the pore-forming protein is engineered to comprise one or more domains that bind(s) a bacterial endotoxin, such as any one or more of those previously mentioned. In an embodiment, the pore-forming protein is engineered to comprise one or more domains that bind to Bacillus anthracis LF or EF, a fragment thereof, a variant thereof, and/or a derivative thereof. In an embodiment, the pore-forming protein is engineered to comprise a domain that binds to a polypeptide that is homologous to the N-terminal region of Bacillus anthracis LF or EF. In an embodiment, a pore-forming protein comprises a heterologous (relative to the pore-forming polypeptide) PA polypeptide, fragment thereof, or derivative thereof. In an embodiment, a pore-forming protein comprises a heterologous (relative to the pore-forming polypeptide) PA₈₃ polypeptide, fragment thereof, or derivative thereof. In an embodiment, a pore-forming protein comprises a heterologous (relative to the pore-forming polypeptide) PA₆₃ polypeptide, fragment thereof, or derivative thereof.

In an embodiment, the targeting moiety recognizes a cell receptor that triggers endocytosis. In an embodiment, such a targeting moiety is a receptor-binding domain of diphtheria toxin. In an embodiment, such a targeting moiety is the receptor binding domain of BONT type A (BoNt/A-H_{c}). See e.g., Tian et al., Cell Reports. (2022) 38 (10): 110476.

### Pre-Pores and Pore-Forming Polypeptide Complexes

Without being bound by theory, in an embodiment, the pore-forming polypeptide(s) are capable of oligomerization with one or more other pore-forming polypeptides. In an embodiment, oligomerization of one or more pore-forming polypeptides forms a pre-pore. In an embodiment, oligomerization forms a pore-forming polypeptide complex. In an embodiment, a pore-forming polypeptide complex is formed by tethering or otherwise linking two or more pore-forming polypeptides together. In an embodiment 1, 2, 3, 4, 5, 6, 7, 8, 9 or 10 or more pore-forming polypeptides oligomerize to form a pre-pore or pore-forming polypeptide complex. In an embodiment, the pre-pore or pore-forming polypeptide complex binds one or more engineered Acr polypeptides and/or cargo delivery molecules thereof. In an embodiment, the pre-pore or pore-forming polypeptide complex binds 1, 2, 3, 4, or 5 or more cargo-engineered Acr polypeptides and/or cargo delivery molecules thereof. In an embodiment, the pre-pore or pore-forming polypeptide complex is formed at the surface of a cell membrane or in a cell membrane.

In an example embodiment, engineered Acr polypeptide is operatively coupled or is capable of operatively coupling to one or more pore-forming polypeptides in the pre-pore or pore-forming polypeptide complex via binding of the cargo delivery molecule to the pore-forming polypeptide in the pre-pore or pore-forming polypeptide complex. In an embodiment, the engineered Acr polypeptide is operatively coupled or is capable of operatively coupling to two or more pore-forming polypeptides in the pre-pore or pore-forming polypeptide complex such as adjacent pore-forming polypeptides in the pre-pore. In an embodiment, the pore-forming polypeptide is or comprises a targeting moiety or targeting domain (i.e., a domain that can function as a targeting moiety). For example, PA₈₃ polypeptide or fragment thereof is capable of targeting cell surface molecules without being coupled to a separate targeting moiety. See e.g., Rabideau and Pentelute. ACS Chem. Biol. 2016, 11, 1490-1501.

In an embodiment, the pore-forming polypeptide is or comprises a targeting domain or targeting moiety. In an example embodiment, the pore-forming polypeptide is operatively coupled to a targeting moiety. Exemplary targeting moieties are described in greater detail elsewhere herein. In an embodiment, the pore-forming polypeptide is linked to a targeting moiety via a linker In an embodiment, the linker is a peptide linker. In an embodiment, the peptide linker is a flexible peptide linker. In an embodiment, the peptide linker is a rigid peptide linker. In an embodiment, the linker is a cleavable linker. In an embodiment, the cleavable linker is cleaved by an enzyme, light, radiation, a chemical reaction, and/or the like. In an embodiment, the linker is configured or otherwise capable of bioconjugation. For example, the linker can be used with or include a cysteine, which can allow for bioconjugation. In an embodiment, the pore-forming polypeptide is operatively coupled to the targeting moiety via a GS linker with a cysteine between the GS linker and the pore-forming polypeptide and/or targeting moiety.

In an embodiment, the linker is a non-cleavable linker.

In an embodiment, the peptide linker has a sequence of GGGLLK (SEQ ID NO: 24). In an embodiment, the peptide linker has a sequence of GGGLLK (SEQ ID NO: 25), wherein L4 and/or L5 are D-Leu. In an embodiment, the peptide linker has a sequence of GGG[GGS]₇K (SEQ ID NO: 26). In an embodiment, the peptide linker has a sequence of GGG[GGS]₇K (SEQ ID NO: 27), where S is L-Ser and/or K is L-Lys. In an embodiment, the peptide linker contains an N^{ε}-linked α-bromoacetyl group. In an embodiment, the peptide linker contains an N^{ε}-linked maleimide group. In an embodiment, the peptide linker is linker peptide 1, 2, or 3 of Lu et al., ACS Cent. Sci. 2021. 7:365-378. In an embodiment, the peptide linker comprises LPSTGGK (SEQ ID NO: 28). Additional exemplary linkers include those set forth in Chen et al., Adv Drug Deliv Rev. 2013 Oct. 15; 65 (10): 1357-1369; Rosmalen et al., Biochem. 2017, 56, 50, 6565-6574; a Proline 9 (P9) linker, GAAPAAAPAKQEAAAPAPAAKAEAPAAAPAAKA (SEQ ID NO: 30), (GGGGS)₃ (SEQ ID NO: 31), (G)₈ (SEQ ID NO: 32), (G)₆ (SEQ ID NO: 33), (EAAAK)₃ (SEQ ID NO: 34), (EAAAK)ₙ (n=1-3) (SEQ ID NO: 35-36, 34), A(EAAAK)₄ALEA(EAAAK)₄A (SEQ ID NO: 37), GGGGS (SEQ ID NO: 38), PAPAP (SEQ ID NO: 39), AEAAAKEAAAKA (SEQ ID NO: 40), (GGGGS)ₙ (n=1-10) (SEQ ID NO: 38, 41, 31, 42-48), (Ala-Pro)n (n=10-32) (SEQ ID NO: 49-71), disulfide, VSQTSKLTR↓AETVFPDV (SEQ ID NO: 72), PLG↓LWA (SEQ ID NO: 73), RVL↓AEA (SEQ ID NO: 74); EDVVCC↓SMSY (SEQ ID NO: 75); GGIEGR↓GS (SEQ ID NO: 76), TRHRQPR↓GWE (SEQ ID NO: 77); AGNRVRR↓SVG (SEQ ID NO: 78); RRRRRRR↓R↓R (SEQ ID NO: 79), GFLG↓ (SEQ ID NO: 80), LE, LEAGCKNFFPR↓SFTSCGSLE (SEQ ID NO: 81), CRRRRRREAEAC (SEQ ID NO: 82), and a TEV site linker, e.g., ENLYFQ↓(S, G, A, M, C, or H) (SEQ ID NO: 83) (Protease-sensitive cleavage sites are indicated with “1”) or any combination thereof. In an embodiment, the linker is or comprises LPSTGGK (SEQ ID NO: 28). Other suitable linkers will be appreciated by those of ordinary skill in the art in view of the description herein.

### Targeting Moieties

As previously discussed, in an embodiment, the pore-forming polypeptide contains or is operatively coupled (e.g., fused to or linked) to a targeting moiety, which includes without limitation targeting molecules and targeting domains. Without being bound by theory and as shown in FIG. 1A-1B, the targeting moiety is capable of recognizing, binding, attaching to, or otherwise interacting with a binding partner that can be present on the surface of a target cell. This can direct where the engineered Acr polypeptide is delivered. In an embodiment, the target cell contains a CRISPR-Cas system or a component thereof. In an embodiment, the targeting moiety targets a receptor that is present on all cell types. In an embodiment, the targeting moiety targets a receptor that is present on multiple cell types. In an embodiment, the targeting moiety targets a receptor that is present on a single cell type. In an embodiment, the targeting moiety targets a specific cell or tissue type and/or cell state. As used herein, “cell state” is used to describe transient elements of a cell's identity. Cell state can be thought of as the transient characteristic profile or phenotype of a cell. Cell states arise transiently during time-dependent processes, either in a temporal progression that is unidirectional (e.g., during differentiation, or following an environmental stimulus) or in a state vacillation that is not necessarily unidirectional and in which the cell may return to the origin state. Vacillating processes can be oscillatory (e.g., cell-cycle or circadian rhythm) or can transition between states with no predefined order (e.g., due to stochastic or environmentally controlled molecular events). These time-dependent processes may occur transiently within a stable cell type (as in a transient environmental response), or may lead to a new, distinct type (as in differentiation). See e.g., Wagner et al., 2016. Nat Biotechnol. 34 (11): 1145-1160. Exemplary targeting moieties and binding partners are discussed below.

In an embodiment, the targeting moiety is or includes a peptide or a polypeptide. In an embodiment, the targeting moiety is or includes an antibody or fragment thereof. In an embodiment, the targeting moiety is or includes an aptamer. In an embodiment, the targeting moiety is or includes a small molecule. In an embodiment, the targeting moiety is or includes a nucleic acid (e.g., DNA or RNA). In an embodiment, the targeting moiety is or includes a receptor. In an embodiment, the targeting moiety is or includes a receptor ligand. In an embodiment, the targeting moiety is or includes a carbohydrate (e.g., sugar). In an embodiment, the targeting moiety is or includes a lipid. In an embodiment, the targeting moiety is an engineered protein scaffold. In an embodiment, the targeting moiety is an affibody. In an embodiment, the targeting moiety is an antibody mimetic. In an embodiment, the targeting moiety is an engineered binding protein, such as a designed ankyrin repeat proteins (DARPins) (see e.g., Plückthun et al., Annu. Rev. Pharmacol. Toxicol. (2015) 55 (1): 489-511), avimers (Silverman et al., Nat. Biotechnol. (2005) 23 (12): 1556-1561 and Jeong et al. Nat. Biotechnol. (2005) 23 (12): 1493-1494), or affibodies (see e.g., Nord et al., Nat. Biotechnol. (1997) 15 (8): 772-777). In an embodiment, the targeting moiety is a receptor ligand or binding protein.

In an embodiment, the targeting moiety targets an anthrax receptor. In an embodiment, the targeting moiety targets a cell adhesion molecule, selectin, or syndecan. In an embodiment, the targeting moiety targets an integrin.

The term “antibody” is used interchangeably with the term “immunoglobulin” herein, and includes intact antibodies, fragments of antibodies, e.g., Fab, F(ab′) 2 fragments, and intact antibodies and fragments that have been mutated either in their constant and/or variable region (e.g., mutations to produce chimeric, partially humanized, or fully humanized antibodies, as well as to produce antibodies with a desired trait, e.g., enhanced binding and/or reduced Immunoglobulin Fc receptor (FcR) binding). “Antibody” includes monovalent and multivalent antibodies. The term “fragment” refers to a part or portion of an antibody or antibody chain comprising fewer amino acid residues than an intact or complete antibody or antibody chain. Fragments can be obtained via chemical or enzymatic treatment of an intact or complete antibody or antibody chain. Fragments can also be obtained by recombinant means. Exemplary fragments include Fab, Fab′, F(ab′)2, Fabc, Fd, dAb, V_{HH}, and scFv and/or Fv fragments.

As used herein, a preparation of antibody protein having less than about 50% of non-antibody protein (also referred to herein as a “contaminating protein”) or of chemical precursors is considered to be “substantially free.” In an embodiment, a preparation of antibody protein having less than about 40%, 30%, 20%, 10%, and more preferably 5% (by dry weight) of non-antibody protein or of chemical precursors is considered to be substantially free. When the antibody protein or biologically active portion thereof is recombinantly produced, it is also preferably substantially free of culture medium, i.e., culture medium represents less than about 30%, preferably less than about 20%, more preferably less than about 10%, and most preferably less than about 5% of the volume or mass of the protein preparation.

As used herein, “nanobody” refers to a single-domain antibody fragment that is capable of specifically binding an antigen. Nanobodies can be engineered to have desired antigen-binding capabilities. Nanobodies can be based on heavy-chain or light-chain domains. See e.g. Arbabi Ghahroudi M, Desmyter A, Wyns L, Hamers R, Muyldermans S (September 1997). “Selection and identification of single domain antibody fragments from camel heavy-chain antibodies”. FEBS Letters. 414 (3): 521-6. doi: 10.1016/S0014-5793 (97) 01062-4; Ward E S, Gussow D, Griffiths A D, Jones P T, Winter G (October 1989). “Binding activities of a repertoire of single immunoglobulin variable domains secreted from Escherichia coli”. Nature. 341 (6242): 544-6. doi: 10.1038/341544a0; Holt L J, Herring C, Jespers L S, Woolven B P, Tomlinson I M (November 2003). “Domain antibodies: proteins for therapy”. Trends in Biotechnology. 21 (11): 484-90. doi: 10.1016/j.tibtech.2003.08.007; Borrebaeck C A, Ohlin M (December 2002). “Antibody evolution beyond Nature”. Nature Biotechnology. 20 (12): 1189-90. doi: 10.1038/nbt1202-1189; Van de Broek B, Devoogdt N, D'Hollander A, Gijs H L, Jans K, Lagae L, et al. (June 2011). “Specific cell targeting with nanobody conjugated branched gold nanoparticles for photothermal therapy”. ACS Nano. 5 (6): 4319-28. doi: 10.1021/nn1023363.

As used herein, the term “antigen-binding fragment” refers to a polypeptide fragment of an immunoglobulin or antibody that binds antigen or competes with intact antibody (i.e., with the intact antibody from which they were derived) for antigen binding (i.e., specific binding). As such these antibodies or fragments thereof are included in the scope of the invention, provided that the antibody or fragment binds specifically to a target molecule.

It is intended that the term “antibody” encompass any Ig class or any Ig subclass (e.g., the IgG1, IgG2, IgG3, and IgG4 subclasses of IgG) obtained from any source (e.g., humans and non-human primates, and in rodents, lagomorphs, caprines, bovines, equines, ovines, etc.).

The term “Ig class” or “immunoglobulin class”, as used herein, refers to the five classes of immunoglobulin that have been identified in humans and higher mammals, IgG, IgM, IgA, IgD, and IgE. The term “Ig subclass” refers to the two subclasses of IgM (H and L), three subclasses of IgA (IgA1, IgA2, and secretory IgA), and four subclasses of IgG (IgG1, IgG2, IgG3, and IgG4) that have been identified in humans and higher mammals. The antibodies can exist in monomeric or polymeric form; for example, lgM antibodies exist in pentameric form, and IgA antibodies exist in monomeric, dimeric, or multimeric form.

The term “IgG subclass” refers to the four subclasses of immunoglobulin class IgG—IgG1, IgG2, IgG3, and IgG4 that have been identified in humans and higher mammals by the heavy chains of the immunoglobulins, VI-γ4, respectively. The term “single-chain immunoglobulin” or “single-chain antibody” (used interchangeably herein) refers to a protein having a two-polypeptide chain structure consisting of a heavy and a light chain, said chains being stabilized, for example, by interchain peptide linkers, which has the ability to specifically bind the antigen. The term “domain” refers to a globular region of a heavy or light chain polypeptide comprising peptide loops (e.g., comprising 3 to 4 peptide loops) stabilized, for example, by a β pleated sheet and/or intrachain disulfide bond. Domains are further referred to herein as “constant” or “variable”, based on the relative lack of sequence variation within the domains of various class members in the case of a “constant” domain, or the significant variation within the domains of various class members in the case of a “variable” domain. Antibody or polypeptide “domains” are often referred to interchangeably in the art as antibody or polypeptide “regions”. The “constant” domains of an antibody light chain are referred to interchangeably as “light chain constant regions”, “light chain constant domains”, “CL” regions or “CL” domains. The “constant” domains of an antibody heavy chain are referred to interchangeably as “heavy chain constant regions”, “heavy chain constant domains”, “CH” regions or “CH” domains). The “variable” domains of an antibody light chain are referred to interchangeably as “light chain variable regions”, “light chain variable domains”, “VL” regions or “VL” domains). The “variable” domains of an antibody heavy chain are referred to interchangeably as “heavy chain variable regions”, “heavy chain variable domains”, “VH” regions or “VH” domains). In an embodiment, the VH domain is a human VH domain.

The term “region” can also refer to a part or portion of an antibody chain or antibody chain domain (e.g., a part or portion of a heavy or light chain or a part or portion of a constant or variable domain, as defined herein), as well as more discrete parts or portions of said chains or domains. For example, light and heavy chains or light and heavy chain variable domains include “complementarity determining regions” or “CDRs” interspersed among “framework regions” or “FRs”, as defined herein.

The term “conformation” refers to the tertiary structure of a protein or polypeptide (e.g., an antibody, antibody chain, domain or region thereof). For example, the phrase “light (or heavy) chain conformation” refers to the tertiary structure of a light (or heavy) chain variable region, and the phrase “antibody conformation” or “antibody fragment conformation” refers to the tertiary structure of an antibody or fragment thereof.

As used herein, “affibody” refers to small (typically around 6.5 kDa) non-immunoglobulin-engineered proteins based on a three-helix bundle domain framework that is based on a 58-amino-acid Z-domain scaffold, derived from one of the IgG-binding domains of staphylococcal protein A and can be engineered for desired target recognition. See e.g., Frejd and Kim. 2017. Exp. Mol. Med. 49 (3): e306; Löfblom J, et al. FEBS Lett. 2010 Jun. 18; 584 (12): 2670-80. doi: 10.1016/j.febslet.2010.04.014. Epub 2010 Apr. 11; and Nygren, P. A. FEBS J. 2008 June; 275 (11): 2668-76.

The term “antibody-like protein scaffolds” or “engineered protein scaffolds” broadly encompasses proteinaceous non-immunoglobulin specific-binding agents, typically obtained by combinatorial engineering (such as site-directed random mutagenesis in combination with phage display or other molecular selection techniques). Usually, such scaffolds are derived from robust and small soluble monomeric proteins (such as Kunitz inhibitors or lipocalins) or from a stably folded extra-membrane domain of a cell surface receptor (such as protein A, fibronectin, or the ankyrin repeat).

Such scaffolds have been extensively reviewed in Binz et al. Engineering novel binding proteins from nonimmunoglobulin domains. Nat Biotechnol 2005, 23:1257-1268; Gebauer and Skerra. Engineered protein scaffolds as next-generation antibody therapeutics. Curr Opin Chem Biol. 2009, 13:245-55; Gill and Damle. Biopharmaceutical drug discovery using novel protein scaffolds. Curr Opin Biotechnol 2006, 17:653-658; Skerra. Engineered protein scaffolds for molecular recognition. J Mol Recognit 2000, 13:167-187; and Skerra. Alternative non-antibody scaffolds for molecular recognition. Curr Opin Biotechnol 2007, 18:295-304; and include without limitation affibodies, based on the Z-domain of staphylococcal protein A, a three-helix bundle of 58 residues providing an interface on two of its alpha-helices (Nygren, Alternative binding proteins: Affibody binding proteins developed from a small three-helix bundle scaffold. FEBS J 2008, 275:2668-2676); engineered Kunitz domains based on a small (ca. 58 residues) and robust, disulfide-crosslinked serine protease inhibitor, typically of human origin (e.g., LACI-D1), which can be engineered for different protease specificities (Nixon and Wood, Engineered protein inhibitors of proteases. Curr Opin Drug Discov Dev 2006, 9:261-268); monobodies or adnectins based on the 10th extracellular domain of human fibronectin III (10Fn3), which adopts an Ig-like beta-sandwich fold (94 residues) with 2-3 exposed loops, but lacks the central disulfide bridge (Koide and Koide, Monobodies: antibody mimics based on the scaffold of the fibronectin type III domain. Methods Mol Biol 2007, 352:95-109); anticalins derived from the lipocalins, a diverse family of eight-stranded beta-barrel proteins (ca. 180 residues) that naturally form binding sites for small ligands by means of four structurally variable loops at the open end, which are abundant in humans, insects, and many other organisms (Skerra, Alternative binding proteins: Anticalins—harnessing the structural plasticity of the lipocalin ligand pocket to engineer novel binding activities. FEBS J 2008, 275:2677-2683); DARPins, designed ankyrin repeat domains (166 residues), which provide a rigid interface arising from typically three repeated beta-turns (Stumpp et al., DARPins: a new generation of protein therapeutics. Drug Discov Today 2008, 13:695-701); avimers (multimerized LDLR-A module) (Silverman et al., Multivalent avimer proteins evolved by exon shuffling of a family of human receptor domains. Nat Biotechnol 2005, 23:1556-1561); and cysteine-rich knottin peptides (Kolmar, Alternative binding proteins: biological activity and therapeutic potential of cystine-knot miniproteins. FEBS J 2008, 275:2684-2690).

In certain embodiments, the targeting moiety is an aptamer. Nucleic acid aptamers are nucleic acid species that have been engineered through repeated rounds of in vitro selection or equivalently, SELEX (systematic evolution of ligands by exponential enrichment) to bind to various molecular targets such as small molecules, proteins, nucleic acids, cells, tissues, and organisms. Nucleic acid aptamers have a specific binding affinity to molecules through interactions other than classic Watson-Crick base pairing. Aptamers are useful in biotechnological and therapeutic applications as they offer molecular recognition properties similar to antibodies. In addition to their discriminate recognition, aptamers offer advantages over antibodies as they can be engineered completely in a test tube, are readily produced by chemical synthesis, possess desirable storage properties, and elicit little or no immunogenicity in therapeutic applications. In certain embodiments, RNA aptamers may be expressed from a DNA construct. In other embodiments, a nucleic acid aptamer may be linked to another polynucleotide sequence. The polynucleotide sequence may be a double-stranded DNA polynucleotide sequence. The aptamer may be covalently linked to one strand of the polynucleotide sequence. The aptamer may be ligated to the polynucleotide sequence. The polynucleotide sequence may be configured, such that the polynucleotide sequence may be linked to a solid support or ligated to another polynucleotide sequence.

Aptamers, like peptides generated by phage display or monoclonal antibodies (“mAbs”), are capable of specifically binding to selected targets and modulating the target's activity, e.g., through binding, aptamers may block their target's ability to function. A typical aptamer is 10-15 kDa in size (30-45 nucleotides), binds its target with sub-nanomolar affinity, and discriminates against closely related targets (e.g., aptamers will typically not bind other proteins from the same gene family). Structural studies have shown that aptamers are capable of using the same types of binding interactions (e.g., hydrogen bonding, electrostatic complementarity, hydrophobic contacts, steric exclusion) that drives affinity and specificity in antibody-antigen complexes.

Aptamers have a number of desirable characteristics for use in research and as therapeutics and diagnostics including high specificity and affinity, biological efficacy, and excellent pharmacokinetic properties. In addition, they offer specific competitive advantages over antibodies and other protein biologics. Aptamers are chemically synthesized and are readily scaled as needed to meet production demand for research, diagnostic or therapeutic applications. Aptamers are chemically robust. They are intrinsically adapted to regain activity following exposure to factors such as heat and denaturants and can be stored for extended periods (>1 yr) at room temperature as lyophilized powders. Not being bound by theory, aptamers bound to a solid support or beads may be stored for extended periods.

Oligonucleotides in their phosphodiester form may be quickly degraded by intracellular and extracellular enzymes such as endonucleases and exonucleases. Aptamers can include modified nucleotides conferring improved characteristics on the ligand, such as improved in vivo stability or improved delivery characteristics. Examples of such modifications include chemical substitutions at the ribose and/or phosphate and/or base positions. SELEX-identified nucleic acid ligands containing modified nucleotides are described, e.g., in U.S. Pat. No. 5,660,985, which describes oligonucleotides containing nucleotide derivatives chemically modified at the 2′ position of ribose, 5 position of pyrimidines, and 8 position of purines, U.S. Pat. No. 5,756,703 which describes oligonucleotides containing various 2′-modified pyrimidines, and U.S. Pat. No. 5,580,737 which describes highly specific nucleic acid ligands containing one or more nucleotides modified with 2′-amino (2′-NH₂), 2′-fluoro (2′-F), and/or 2′-O-methyl (2′-OMe) substituents. Modifications of aptamers may also include, modifications at exocyclic amines, substitution of 4-thiouridine, substitution of 5-bromo or 5-iodo-uracil; backbone modifications, phosphorothioate or allyl phosphate modifications, methylations, and unusual base-pairing combinations such as the isobases isocytidine and isoguanosine. Modifications can also include 3′ and 5′ modifications such as capping. As used herein, the term phosphorothioate encompasses one or more non-bridging oxygen atoms in a phosphodiester bond replaced by one or more sulfur atoms. In further embodiments, the oligonucleotides comprise modified sugar groups, for example, one or more of the hydroxyl groups is replaced with halogen, aliphatic groups, or functionalized as ethers or amines. In one embodiment, the 2′-position of the furanose residue is substituted by any of an O-methyl, O-alkyl, O-allyl, S-alkyl, S-allyl, or halo group. Methods of synthesis of 2′-modified sugars are described, e.g., in Sproat, et al., Nucl. Acid Res. 19:733-738 (1991); Cotten, et al, Nucl. Acid Res. 19:2629-2635 (1991); and Hobbs, et al, Biochemistry 12:5138-5145 (1973). Other modifications are known to one of ordinary skill in the art. In certain embodiments, aptamers include aptamers with improved off-rates as described in International Patent Publication No. WO 2009012418, “Method for generating aptamers with improved off-rates,” incorporated herein by reference in its entirety. In certain embodiments, aptamers are chosen from a library of aptamers. Such libraries include, but are not limited to, those described in Rohloff et al., “Nucleic Acid Ligands With Protein-like Side Chains: Modified Aptamers and Their Use as Diagnostic and Therapeutic Agents,” Molecular Therapy Nucleic Acids (2014) 3, e201. Aptamers are also commercially available (see, e.g., SomaLogic, Inc., Boulder, Colorado). In certain embodiments, the present invention may utilize any aptamer containing any modification as described herein.

In an embodiment, the targeting moiety is a small molecule, such as a small molecule receptor ligand. The term “small molecule” refers to compounds, preferably organic compounds, with a size comparable to those organic molecules generally used in pharmaceuticals. The term excludes biological macromolecules (e.g., proteins, peptides, nucleic acids, etc.). Preferred small organic molecules range in size up to about 5000 Da, e.g., up to about 4000, preferably up to 3000 Da, more preferably up to 2000 Da, even more preferably up to about 1000 Da, e.g., up to about 900, 800, 700, 600 or up to about 500 Da. In certain embodiments, the small molecule may act as an antagonist or agonist (e.g., blocking an enzyme active site or activating a receptor by binding to a ligand binding site).

The targeting moiety can be capable of specifically binding, attaching, or otherwise interacting with a binding partner (also referred to herein as a “specific binding partner”) on a target cell. In an embodiment, the specific binding partner, and thus the target cell, is predetermined. Thus, in an embodiment, the engineered Acr polypeptide is engineered to interact with a pore-forming polypeptide that is operatively coupled to a targeting moiety selected to target a specific cell via its binding partner.

As used herein, the term “specific binding” refers to non-covalent physical association of a first and a second moiety wherein the association between the first and second moieties is at least 2 times as strong, at least 5 times as strong as, at least 10 times as strong as, at least 50 times as strong as, at least 100 times as strong as, or stronger than the association of either moiety with most or all other moieties present in the environment in which binding occurs. The binding of two or more entities may be considered specific if the equilibrium dissociation constant, Kd, is 10⁻³ M or less, 10⁻⁴ M or less, 10⁻⁵ M or less, 10⁻⁶ M or less, 10⁻⁷ M or less, 10⁻⁸ M or less, 10⁻⁹ M or less, 10⁻¹⁰ M or less, 10⁻¹¹ M or less, or 10⁻¹² M or less under the conditions employed, e.g., under physiological conditions such as those inside a cell or consistent with cell survival. In an embodiment, specific binding can be accomplished by a plurality of weaker interactions (e.g., a plurality of individual interactions, wherein each individual interaction is characterized by a Kd of greater than 10⁻³ M). In an embodiment, specific binding, which can be referred to as “molecular recognition,” is a saturable binding interaction between two entities that is dependent on complementary orientation of functional groups on each entity. Examples of specific binding interactions include primer-polynucleotide interaction, aptamer-aptamer target interactions, antibody-antigen interactions, avidin-biotin interactions, ligand-receptor interactions, metal-chelate interactions, hybridization between complementary nucleic acids, etc.

Exemplary target cells include, but are not limited to liver cells, pancreatic cells, muscle cells (e.g., skeletal, cardiac, and/or smooth muscle cells), brain cells, neurons, nerve support cells (e.g., glial cells, Schwann cells, astrocytes, dendrites, etc.), immune cells (T-cells, B-cells, monocytes, macrophages, dendritic cells, NK cells, neutrophils, plasma cells, etc.), kidney cells, thyroid cells, bone cells, gastrointestinal tract cells, auditory cells (e.g., hair cells), eye cells (e.g., retinal cells, corneal cells, etc.), skin cells, lung cells, adipocytes, bladder cells, olfactory cells, vasculature cells, cancer cells, tumor cells, cancer stem cells, and/or the like. In an embodiment, the target cells are diseased. In an embodiment, the target cells are normal (non-diseased). In an embodiment, the target cells are progenitor cells. In an embodiment, the target cells are differentiated cells. In an embodiment, the target cells contain a CRISPR-Cas system or component thereof. In an embodiment, the target cell is also a target cell of a CRISPR-Cas system (i.e., a cell in which it is desirable that a CRISPR-Cas system be active in). In an embodiment, the target cell is not an intended target cell of a CRISPR-Cas system (i.e., a cell in which it is not desirable that a CRISPR-Cas system be active in). Exemplary targeting moieties are described in Table 3. Other suitable targeting moieties will be appreciated by those of skill in the art in view of the description herein.

TABLE 3
Exemplary Targeting Moieties

| Targeting Moiety or Targeting | | | Exemplary target cell(s) |
|---|---|---|---|
| domain | Binding Partner (target) | Reference a | and/or references |
| LF, EF, or fragment thereof, variant | TEM8, CMG2 (anthrax receptors) | Protein Atlas entry | The anthrax receptors are |
| thereof, or derivative thereof | | for ANTXR1 and | expressed on most human |
| | | ANTXR2 and | cells including U2OS and |
| | | available at | HEK293T (cell lines used |
| | | https://www.proteinatlas.org/ | for example embodiments |
| | | ENSG000001 | herein). |
| | | 69604- | TEM8 (ANTXR1) |
| | | ANTXR1/cell + line | expression in U2OS (52.3 |
| | | and | nTPM) and HEK293 (24.4 |
| | | https://www.proteinatlas.org/ | nTPM) |
| | | ENSG000001 | CMG2 (ANTXR2) |
| | | 63297- | expression in U2OS (6.6 |
| | | ANTXR2/cell + line | nTPM) and HEK293 (4.2 |
| | | (accessed on Jan. | nTPM) |
| | | 25, 2023) | nTPM is a normalized |
| | | Abi-Habib et al. Mol. | transcript expression value |
| | | Cancer Ther. 4, | and is proportional to the |
| | | 1303-1310. | expression of each |
| | | | receptor in each cell. See |
| | | | https://www.proteinatlas.org/ |
| | | | about/assays + annotation |
| | | | cell + line |
| Integrins (e.g., VLA-1, VLA-2, VLA-3, | Various ligands, including RGD peptide, | | Various, integrin |
| VLA-4, VLA-5, VLA-6, LFA-1, Mac- | fibronectin, vitronectin, collagens, | | dependent. Including but |
| 1, fibrinogen receptor, vitronectin | laminins, and proteinases, including but | | not limited to muscle cells, |
| receptor, α₇β₁, αᵥβ₁, αᵥβ₅, αᵥβ₆, αᵥβ₈, | not limited to laminin-5, VCAM-1, | | glioma cells, T- |
| α6β4 | ICAM-1, ICAM-2, osteopontin, | | lymphocytes, neutrophils, |
| | fibrinogen, Cyr61, throxine, TETRAC, | | monocytes, platelets, |
| | adnovirus, TGFβ1 + 3, etc. | | neurotumor cells, |
| | | | activated endothelia cells, |
| | | | melanoma cells, |
| | | | glioblastoma cells, |
| | | | fibroblasts, epithelial cells, |
| | | | neural cells (CNS and |
| | | | PNS), etc. |
| Folate, anti-folate receptor molecule | Folate receptor | | |
| (e.g., antibody, affibody, aptamer, etc.) | | | |
| transferrin | transferrin receptor | | |
| Anti-CC52 molecule (e.g., antibody, | rat CC531 and homologs | | |
| affibody, aptamer, etc.) | | | |
| anti-HER2 molecule (e.g., antibody, | HER2 | Lu and Truex et al. | |
| affibody, aptamer, etc.) | | ACS Cent. Sci. 2021, | |
| | | 7, 365-378 | |
| anti-GD2 molecule (e.g., antibody, | GD2 | | |
| affibody, aptamer, etc.) | | | |
| anti-EGFR molecule (e.g., antibody, | EGFR | Mechaly et al. | Pancreas cells |
| affibody, aptamer, etc.) | | Changing the receptor | |
| | | specificity of anthrax | |
| | | toxin. MBio 2012, | |
| | | 3(3), e00088-12. | |
| pH-dependent fusogenic peptide diINF-7 | | | |
| anti-VEGFR molecule (e.g., antibody, | VEGF Receptor | | |
| affibody, aptamer, etc.), | | | |
| anti-CD19 molecule (e.g., antibody, | CD19 (B cell marker) | | |
| affibody, aptamer, etc.) | | | |
| RGD peptides | Integrins | Bernhagen et al. ACS | Muscle cells |
| | | Comb. Sci. 2019. | |
| | | 21(3): 198-206 | |
| Anti-Actin molecule (e.g., antibody, | Smooth muscle cell actin | Gown et al., J Cell | Smooth muscle cells and |
| affibody, aptamer, etc.) | | Biol. 1985 | myoepithelial cells |
| | | March; 100(3): 807-13 | |
| Anti-desmin molecule (e.g., antibody, | Desmin | | Smooth, skeletal, cardiac |
| affibody, aptamer, etc.) | | | muscle cells; |
| Anti-T-tubule molecule (e.g., antibody, | T-tubule | Malouf et al., J | Skeletal muscle |
| affibody, aptamer, etc.) | | Histochem | |
| | | Cytochem. 1986 | |
| | | March; 34(3): 347-55. | |
| | | doi: | |
| | | 10.1177/34.3.3950385 | |
| Anti-myosin molecule (e.g., antibody, | Muscle myosin | Lindskog et al., BMC | Various muscle types |
| affibody, aptamer, etc.) | | Genomics. 2015; | (e.g., cardiac, skeletal, |
| | | 16(1): 475; | smooth) |
| | | Schiaffino, S. FEBS | |
| | | J. 2018 | |
| | | October; 285(20): 3688- | |
| | | 3694; Gambke et al., | |
| | | J Biol Chem. 1984 | |
| | | October | |
| | | 10; 259(19): 12092- | |
| | | 100; Sartore et al., | |
| | | Eur J Biochem. 1989 | |
| | | January 15; 179(1): 79-85. | |
| Anti-NG2 molecule (e.g., antibody, | NG2, a membrane chondroitin sulfate | | Oligodendrocyte precursor |
| affibody, aptamer, etc.) | proteoglycan | | cells |
| Anti-PDGFRA molecule (e.g., | Platelet derived growth factor receptor A | | Oligodendrocyte precursor |
| antibody, affibody, aptamer, etc.) | (PDGFRA), a cell surface tyrosine kinase | | cells |
| | receptor | | |
| Anti-MOG molecule (e.g., antibody, | MOG, a glycoprotein found on the surface | | oligodendrocytes |
| affibody, aptamer, etc.) | of oligodendrocytes | | |
| Anti-EAAT2/GLT-1 molecule (e.g., | EAAT2, is a glutamate transporter | Lee et al., | astrocytes |
| antibody, affibody, aptamer, etc.) | | Transcription | |
| | | Chromatin, and | |
| | | Epigenetics. | |
| | | 283(19): P13116- | |
| | | 13123 (2008). | |
| Anti-myelin protein zero (MPZ) | MPZ, a structural component of the | | Schwann cell precursors, |
| molecule (e.g., antibody, affibody, | myelin sheath | | myelinating Schwann cells |
| aptamer, etc.) | | | |
| Anti-NCAM molecule (e.g., antibody, | NCAM, a cell adhesion glycoprotein | | Non-myelinating Schwann |
| affibody, aptamer, etc.) | | | cells |
| Anti-P75NTR molecule (e.g., antibody, | p75 NGF receptor | | |
| affibody, aptamer, etc.) | | | |
| Nerve Growth Factor (NGF, Brain- | p75 NGF receptor | | Schwan cells, particularly |
| derived neurotrophic factor (BDNF, | | | Schwann cell precursors |
| neurotrophins 3 and 4 | | | and non-myelinating |
| | | | Schwann cells |
| Anti-myelin basic protein) molecule | Anti-myelin basic protein, most abundant | | Myelinating Schwann |
| (e.g., antibody, affibody, aptamer, etc.) | protein of the myelin membrane | | cells |
| Anti-TMEM119 molecule (e.g., | TMEM119 cell-surface protein | | Microglia cells |
| antibody, affibody, aptamer, etc.) | | | |
| Anti-IBA1 molecule (e.g., antibody, | Ionized calcium-binding adaptor molecule | | Microglia cells and |
| affibody, aptamer, etc.) | 1 (IBA1) | | macrophages |
| Anti-GAP43 molecule (e.g., antibody, | GAP43, which is a major component of | | neurons |
| affibody, aptamer, etc.) | “growth cones” of axons | | |
| Anti-NMDA receptor subunit molecule | NMDA receptor subunits/receptors are | | Glutamatergic neurons |
| (e.g., antibody, affibody, aptamer, etc.); | components of NMDA receptors on | | |
| exemplary subunits GluN1, GluN2, | GABAergic neurons | | |
| GluN3, some of which have variants | | | |
| (GluN2A-D; GluN3A-B) | | | |
| Anti-GAT-1 molecule (e.g., antibody, | GAT-1, a cell membrane GABA | | GABAergic neurons |
| affibody, aptamer, etc.) | transporter | | |
| Anti-DAT molecule (e.g., antibody, | Dopamine Transporter (DAT) | | Dopaminergic neurons |
| affibody, aptamer, etc.) | | | |
| Anti-synapsin I molecule (e.g., | Synapsin I, present in neuron synapses | | Presynaptic neurons |
| antibody, affibody, aptamer, etc.) | | | |
| Anti-synapsin II molecule (e.g., | Synapsin II, present in neuron synapses | | Presynaptic neurons |
| antibody, affibody, aptamer, etc.) | | | |
| Anti-synaptotagmin molecule (e.g., | Synaptotagmins, which are integral | | Presynaptic neurons |
| antibody, affibody, aptamer, etc.) | membrane proteins of synaptic vesicles | | |
| Anti-CD24 molecule (e.g., antibody, | CD24 | | neurons |
| affibody, aptamer, etc.) | | | |
| Anti-hepatocyte Specific Antigen | Hepatocyte Specific Antigen | | Hepatocytes |
| molecule (e.g., antibody, affibody, | | | |
| aptamer, etc.) | | | |
| Anti-Alpha 1 antitrypsin (AAT) | Alpha 1 antitrypsin | | Hepatocytes |
| molecule (e.g., antibody, affibody, | | | |
| aptamer, etc.) | | | |
| Anti-insulin receptor molecule (e.g., | Insulin receptor | | Pancreas cells, kidney |
| antibody, affibody, aptamer, etc.) | | | tubules |
| Anti-insulin-like growth receptor | IGFR | | Pancreas cells |
| molecule (e.g., antibody, affibody, | | | |
| aptamer, etc.) | | | |
| Anti-GPR40 molecule (e.g., antibody, | G-Protein coupled receptor 40 | | Pancreas cells |
| affibody, aptamer, etc.) | | | |
| Anti-IL-1R molecule (e.g., antibody, | Interleukin-1 receptor | | Pancreas cells |
| affibody, aptamer, etc.) | | | |
| Anti-GLUT1 molecule (e.g., antibody, | GLUT1 transporter | | Pancreas cells |
| affibody, aptamer, etc.) | | | |
| Anti-GLUT2 molecule (e.g., antibody, | GLUT2 transporter | | Pancreas cells |
| affibody, aptamer, etc.) | | | |
| Anti-GLUT3 molecule (e.g., antibody, | GLUT3 transporter | | |
| affibody, aptamer, etc.) | | | |
| Anti-GLUT4 molecule (e.g., antibody, | GLUT4 transporter | | |
| affibody, aptamer, etc.) | | | |
| Anti-GLUT5 molecule (e.g., antibody, | GLUT5 transporter | | |
| affibody, aptamer, etc.) | | | |
| Anti-GLUT6 molecule (e.g., antibody, | GLUT6 transporter | | |
| affibody, aptamer, etc.) | | | |
| Anti-GLUT7 molecule (e.g., antibody, | GLUT7 transporter | | |
| affibody, aptamer, etc.) | | | |
| Anti-GLUT8 molecule (e.g., antibody, | GLUT8 transporter | | |
| affibody, aptamer, etc.) | | | |
| Anti-GLUT9 molecule (e.g., antibody, | GLUT9 transporter | | |
| affibody, aptamer, etc.) | | | |
| Anti-GLUT10 molecule (e.g., antibody, | GLUT10 transporter | | |
| affibody, aptamer, etc.) | | | |
| Anti-GLUT11 molecule (e.g., antibody, | GLUT11 transporter | | |
| affibody, aptamer, etc.) | | | |
| Anti-GLUT12 molecule (e.g., antibody, | GLUT12 transporter | | |
| affibody, aptamer, etc.) | | | |
| Anti-GLUT13 molecule (e.g., antibody, | GLUT13 transporter | | |
| affibody, aptamer, etc.) | | | |
| Anti-GLUT14 molecule (e.g., antibody, | GLUT14 transporter | | |
| affibody, aptamer, etc.) | | | |
| Anti-HMIT molecule (e.g., antibody, | HMIT transporter | | |
| affibody, aptamer, etc.) | | | |
| Glucose | GLUT1-14, SGLT1, SGLT3, SGLT5, | | |
| | SGLT6 | | |
| Fructose | GLUT2, 5, 7, 11, SGLT5 | | |
| Dehydro-ascorbic acid | GLUT1, 3, 4 | | |
| glucosamine | GLUT2 | | |
| Myo-inositol | HMIT, SGLT6, SMIT | | |
| Anti-PEPT1 molecule (e.g., antibody, | PWPT1, a di- and tri-peptide transporter | | enterocytes |
| affibody, aptamer, etc.) | and di- and tri-peptide mimetics | | |
| Anti-SGLT1 molecule (e.g., antibody, | SGLT1, sodium dependent glucose | | enterocytes |
| affibody, aptamer, etc.) | transporter (SGLT) 1 | | |
| Anti-SGLT2 molecule (e.g., antibody, | SGLT2 | | |
| affibody, aptamer, etc.) | | | |
| Anti-SGLT3 molecule (e.g., antibody, | SGLT3 | | |
| affibody, aptamer, etc.) | | | |
| Anti-SGLT4 molecule (e.g., antibody, | SGLT4 | | |
| affibody, aptamer, etc.) | | | |
| Anti-SGLT5 molecule (e.g., antibody, | SGLT5 | | |
| affibody, aptamer, etc.) | | | |
| Anti-SGLT6 molecule (e.g., antibody, | SGLT6 | | |
| affibody, aptamer, etc.) | | | |
| Anti-SMIT molecule (e.g., antibody, | | | |
| affibody, aptamer, etc.) | | | |
| mannose | SGLT4, SGLT5 | | |
| galactose | SGLT1, SGLT2, SGLT5 | | |
| mannose | SGLT4 | | |
| Anti-EAAT3 molecule (e.g., antibody, | EAAT3, a glutamate, aspartate, cystine | | enterocytes |
| affibody, aptamer, etc.) | transporter | | |
| Anti-EAAT2 molecule (e.g., antibody, | EAAT2, an aspartate, glutamate, | | |
| affibody, aptamer, etc.) | transporter | | |
| Anti-EAAT1 molecule (e.g., antibody, | EAAT1, an aspartate, glutamate, | | |
| affibody, aptamer, etc.) | transporter | | |
| Anti-ASCT1 molecule (e.g., antibody, | ASCT1, an alanine, serine, cysteine, | | |
| affibody, aptamer, etc.) | transporter | | |
| Anti-ASCT2 molecule (e.g., antibody, | ASCT2, an alanine, serine, cysteine, | | |
| affibody, aptamer, etc.) | threonine, glutamine, transporter | | |
| Anti-EAAT4 molecule (e.g., antibody, | EAAT4, a glutamate, aspartate transporter | | |
| affibody, aptamer, etc.) | | | |
| Anti-GAT1 molecule (e.g., antibody, | GAT1, a gamma-aminobutyric acid | | |
| affibody, aptamer, etc.) | (GABA)transporter | | |
| Anti-NET molecule (e.g., antibody, | NET, a dopamine, norepinephrine | | |
| affibody, aptamer, etc.) | transporter | | |
| Anti-DA transporter molecule (e.g., | DA transporter, a dopamine transporter | | |
| antibody, affibody, aptamer, etc.) | | | |
| Anti-SERT molecule (e.g., antibody, | SERT, a serotonin transporter | | |
| affibody, aptamer, etc.) | | | |
| Anti-GLY2 molecule (e.g., antibody, | GLY2, a glycine transporter | | |
| affibody, aptamer, etc.) | | | |
| Anti-PROT molecule (e.g., antibody, | PROT, a proline transporter | | |
| affibody, aptamer, etc.) | | | |
| Anti-CT1 molecule (e.g., antibody, | CT1, a creatine transporter | | |
| affibody, aptamer, etc.) | | | |
| Anti-GAT3 molecule (e.g., antibody, | GAT3, a GABA transporter | | |
| affibody, aptamer, etc.) | | | |
| Anti-GAT2 molecule (e.g., antibody, | GAT2, a GABA transporter | | |
| affibody, aptamer, etc.) | | | |
| Anti-CAT-2 molecule (e.g., antibody, | CAT-2, an arginine, lysine, ornithine | | |
| affibody, aptamer, etc.) | transporter | | |
| Anti-CAT-3 molecule (e.g., antibody, | CAT-3, a homoarginine, arginine, lysine, | | |
| affibody, aptamer, etc.) | ornithine transporter | | |
| Anti-Asc-1//4f2hc molecule (e.g., | Asc-1//4f2hc, a glycine, alanine, serine, | | |
| antibody, affibody, aptamer, etc.) | cysteine, threonine transporter | | |
| Anti-XCT/4f2hc molecule (e.g., | XCT/4f2hc, an aspartic acid, glutamic | | |
| antibody, affibody, aptamer, etc.) | acid, cysteine transporter | | |
| Anti-TAT1 molecule (e.g., antibody, | TAT1, a tryptophan, tyrosine, | | |
| affibody, aptamer, etc.) | phenylalanine transporter | | |
| Anti-SNAT-1 molecule (e.g., antibody, | SNAT-1, a glycine, alanine, asparagine, | | |
| affibody, aptamer, etc.) | cysteine, glutamine, histidine, methionine | | |
| Anti-SNAT-3 molecule (e.g., antibody, | SNAT-3, a glutamine, asparagine, | | |
| affibody, aptamer, etc.) | histidine transporter | | |
| Anti-LAT4 molecule (e.g., antibody, | LAT4, a leucine, isoleucine, methionine, | | |
| affibody, aptamer, etc.) | phenylalanine, valine transporter | | |
| Anti-TautT molecule (e.g., antibody, | TautT, a taurine, beta-alanine transporter | | |
| affibody, aptamer, etc.) | | | |
| Anti-ATB^{0, +} molecule (e.g., antibody, | ATB^{0, +}, a neutral amino acid and cationic | | |
| affibody, aptamer, etc.) | amino acid transporter | | |
| Anti-IMINO molecule (e.g., antibody, | IMINO, a proline, hydroxy-proline, | | |
| affibody, aptamer, etc.) | betaine transporter | | |
| Anti-Y⁺ (CAT-1) molecule (e.g., | Y⁺ (CAT-1), a lysine, arginine, ornithine, | | |
| antibody, affibody, aptamer, etc.) | histidine transporter | | |
| Anti-LAT1/4f2hc molecule (e.g., | LAT1/4f2hc, a histidine, methionine, | | |
| antibody, affibody, aptamer, etc.) | leucine, isoleucine, valine, phenylalanine, | | |
| | tryptophan transporter | | |
| Anti-Y⁺LAT2/4f2hc molecule (e.g., | Y⁺LAT2/4f2hc, a lysine, arginine, | | |
| antibody, affibody, aptamer, etc.) | glutamine, histidine, methionine, leucine | | |
| | transporter | | |
| Anti-Y⁺LAT1/4f2hc molecule (e.g., | Y⁺LAT1/4f2hc, a lysine, arginine, | | |
| antibody, affibody, aptamer, etc.) | glutamine, histidine, methionine, leucine, | | |
| | alanine, cysteine transporter | | |
| Anti-b^{0, +}AT molecule (e.g., antibody, | b^{0, +}AT, a neutral and cationic amino acid | | |
| affibody, aptamer, etc.) | transporter | | |
| Anti-PAT1 molecule (e.g., antibody, | PAT1, a glycine, proline, alanine | | |
| affibody, aptamer, etc.) | transporter | | |
| Anti-SNAT2 molecule (e.g., antibody, | SNAT2, a glycine, proline, alanine, | | |
| affibody, aptamer, etc.) | serine, cysteine, glutamine, asparagine, | | |
| | histidine, methionine transporter | | |
| Anti-SNAT5 molecule (e.g., antibody, | SNAT5, a glutamine, asparagine, | | |
| affibody, aptamer, etc.) | histidine, alanine transporter | | |
| Anti-LAT3 molecule (e.g., antibody, | LAT3, a leucine, isoleucine, methionine, | | |
| affibody, aptamer, etc.) | phenylalanine, valine transporter | | |
| Anti-B(0)AT2 molecule (e.g., antibody, | B(0)AT2, a proline, leucine, valine, | | |
| affibody, aptamer, etc.) | isoleucine, methionine transporter | | |
| Anti-B(0)AT3 molecule (e.g., antibody, | B(0)AT3, a glycine, alanine, methionine, | | |
| affibody, aptamer, etc.) | serine, cysteine transporter | | |
| Anti-B(0)AT1 molecule (e.g., antibody, | B(0)AT1, a neutral amino acid transporter | | |
| affibody, aptamer, etc.) | | | |
| Anti-CAT-4 molecule (e.g., antibody, | CAT4, an arginine transporter | | |
| affibody, aptamer, etc.) | | | |
| Anti-PEPT2 molecule (e.g., antibody, | PEPT2, a di- and tri-peptide transporter | | |
| affibody, aptamer, etc.) | and di- and tri-peptide mimetics | | |
| Anti-PAT2 molecule (e.g., antibody, | PAT2, a glycine, alanine, proline | | |
| affibody, aptamer, etc.) | transporter | | |
| Anti-PAT4 molecule (e.g., antibody, | PAT4, a proline, tryptophan, alanine | | |
| affibody, aptamer, etc.) | transporter | | |
| Anti-SNAT4 molecule (e.g., antibody, | SNAT4, a glycine, alanine, serine, | | |
| affibody, aptamer, etc.) | cysteine, glutamine, asparagine, | | |
| | methionine transporter | | |
| Anti-FGFR molecule (e.g., antibody, | Fibroblast Growth Factor Receptor | | |
| affibody, aptamer, etc.) | (FGFR) | | |
| Fibroblast Growth Factor | Fibroblast Growth Factor Receptor | | |
| | (FGFR) | | |
| Anti-HGFR molecule (e.g., antibody, | Hepatocyte Growth Factor Receptor | | |
| affibody, aptamer, etc.) | (HGFR) | | |
| Hepatocyte Growth Factor (HGF) | Hepatocyte Growth Factor Receptor | | |
| | (HGFR | | |
| An Anti-Olfactory Receptor Class I | Olfactory Receptor (OR) Class I (OR | | Olfactory neurons |
| molecule (e.g., antibody, affibody, | families 51-56) | | |
| aptamer, etc.) | | | |
| An Anti-Olfactory Receptor Class II | Olfactory Receptor (OR) Class II (OR | | Olfactory neurons |
| molecule (e.g., antibody, affibody, | families 1-13) | | |
| aptamer, etc.) | | | |
| An Anti-adrenoreceptor (e.g., alpha-1, | Adrenoreceptors | | |
| alpha-2, beta-1, beta-2, beta-3 | | | |
| adrenoreceptor) molecule (e.g., | | | |
| antibody, affibody, aptamer, etc.) | | | |
| Norepinephrine, epinephrine, | adrenoreceptor (e.g., alpha-1, alpha-2, | | |
| isoprenaline beta-receptor blocker | beta-1, beta-2, and/or beta-3 | | |
| agents, adrenoreceptor agonists, alpha | adrenoreceptor) | | |
| receptor blocker agents | | | |
| An Anti-TrkA, TrkB, or TrkC) | Tropomyosin receptor kinase A, B, or C, | | |
| molecule (e.g., antibody, affibody, | a tyrosine kinase receptor | | |
| aptamer, etc.) | | | |
| An Anti-Eph Receptor molecule (e.g., | Ephrin Receptor (EPH Receptor) | | |
| antibody, affibody, aptamer, etc.) | | | |
| Ephrin | Ephrin Receptor (EPH Receptor) | | |
| An Anti-Eph Receptor molecule (e.g., | | | |
| antibody, affibody, aptamer, etc.) | | | |
| An Anti-CD3 molecule (e.g., antibody, | CD3 | | T-cells |
| affibody, aptamer, etc.) | | | |
| An Anti-T cell receptor alpha chain | TCR-alpha subunit | | T-cells |
| molecule (e.g., antibody, affibody, | | | |
| aptamer, etc.) | | | |
| An Anti-T cell receptor beta chain | TCR-beta subunit | | T-cells |
| molecule (e.g., antibody, affibody, | | | |
| aptamer, etc.) | | | |
| An Anti-T cell receptor gamma chain | TCR-gamma subunit | | T-cells |
| molecule (e.g., antibody, affibody, | | | |
| aptamer, etc.) | | | |
| An Anti-CD28 molecule (e.g., antibody, | CD28 | | T-Cells |
| affibody, aptamer, etc.) | | | |
| An Anti-SCIMP molecule (e.g., | SLP65/SLP76, Csk-interacting membrane | | B-cells, bone marrow- |
| antibody, affibody, aptamer, etc.) | protein (SCIMP) | | derived dendritic cells, |
| | | | macrophages |
| An Anti-toll like receptor (e.g., TLR1, | Toll-like receptors (TLRs), e.g., (e.g., | | |
| TLR2, TLR3, TLR4, TLR5, TLR6, | TLR1, TLR2, TLR3, TLR4, TLR5, | | |
| TLR7, TLR8, TLR9, TLR10, TLR11, | TLR6, TLR7, TLR8, TLR9, TLR10, | | |
| | TLR11, TLR12, and TLR13) | | |
| TLR12, and TLR13) molecule (e.g., | | | |
| antibody, affibody, aptamer, etc.) | | | |
| Anti-ATP-binding cassette sub-family | ATP-binding cassette sub-family A | | |
| A member 1 molecule (e.g., antibody, | member 1, a cholesterol transporter | | |
| affibody, aptamer, etc.) | | | |
| cholesterol | ATP-binding cassette sub-family A | | |
| | member 1, ATP-binding cassette sub- | | |
| | family G member 5, ATP-binding cassette | | |
| | sub-family G member 8, a cholesterol | | |
| | transporter | | |
| An Anti-FATP-1 molecule (e.g., | FATP-1, a long and very long chain fatty | | |
| antibody, affibody, aptamer, etc.) | acid transporter (e.g., C18:1, C20:4, | | |
| | C16:0, C24:0) transporter | | |
| An Anti-FATP-2 molecule (e.g., | FATP-2, a C16:0, C24:0, bile acid, and | | |
| antibody, affibody, aptamer, etc.) | other long chain fatty acids transporter | | |
| An Anti-FATP-3 molecule (e.g., | FATP-3, a long chain fatty acid | | |
| antibody, affibody, aptamer, etc.) | transporter | | |
| An Anti-FATP-4 molecule (e.g., | FATP-4, a long chain fatty acid | | |
| antibody, affibody, aptamer, etc.) | transporter, particularly C18:1, C20:4 | | |
| An Anti-FATP-5 molecule (e.g., | FATP-5, a long chain fatty acid | | |
| antibody, affibody, aptamer, etc.) | transporter | | |
| An Anti-FATP-6 molecule (e.g., | FATP-6, a long chain fatty acid | | |
| antibody, affibody, aptamer, etc.) | transporter, particularly C16:0, C18:0 | | |
| | (LCFAs > C10) | | |
| Anti-ATP-binding cassette sub-family | ATP-binding cassette sub-family G | | |
| G member 5 molecule (e.g., antibody, | member 5, a cholesterol transporter | | |
| affibody, aptamer, etc.) | | | |
| Anti-ATP-binding cassette sub-family | ATP-binding cassette sub-family G | | |
| G member 8 molecule (e.g., antibody, | member 8, a cholesterol transporter | | |
| affibody, aptamer, etc.) | | | |
| Anti-FAT molecule (e.g., antibody, | FAT, a very long chain fatty acid, HDL, | | |
| affibody, aptamer, etc.) | LDL, VLDL, phospholipid, advanced | | |
| | glycation end product, GHRP, hexarelin, | | |
| | EP 80317, vitamin D, transporter | | |
| Anti-FABPpm molecule (e.g., antibody, | FABpm, a long chain fatty acid | | |
| affibody, aptamer, etc.) | transporter | | |
| Anti-Niemann-Pick C1-like protein 1 | Niemann-Pick C1-like protein 1, a | | |
| molecule (e.g., antibody, affibody, | cholesterol, cholestanol, ampesterol, | | |
| aptamer, etc.) | sitosterol, vitamin E, vitamin D | | |
| | transporter | | |
| Anti-scavenger receptor class B, | scavenger receptor class B, member 1, an | | |
| member 1 molecule (e.g., antibody, | HDL-cholesterol transporter | | |
| affibody, aptamer, etc.) | | | |
| Anti-SMVT molecule (e.g., antibody, | SMVT, a pantothenic acid, biotin | | |
| affibody, aptamer, etc.) | transporter | | |
| Anti-RFT/reduced folate carrier (RFC) | RFT/reduced folate carrier (RFC), a 5- | | |
| molecule (e.g., antibody, affibody, | methyl THFm thiamin-mono- and di- | | |
| aptamer, etc.) | phosphates, but not free thiamin | | |
| | transporter | | |
| Anti-ThTr1 molecule (e.g., antibody, | ThTr1, a thiamin, thiamin-mono- and di- | | |
| affibody, aptamer, etc.) | phosphate transporter | | |
| Anti-ThTr2 molecule (e.g., antibody, | ThTr2, a thiamin, thiamin-mono- and di- | | |
| affibody, aptamer, etc.) | phosphate transporter | | |
| Anti-Vitamin D transporter molecule | Vitamin D transporter | | |
| (e.g., antibody, affibody, aptamer, etc.) | | | |
| Anti-Folate Receptor (FR) (e.g., | FR, (transporter, binds 5- | | |
| FOLR1, FOLR3) molecule (e.g., | Methyltetrahydrofolate, folate) | | |
| antibody, affibody, aptamer, etc.) | | | |
| Anti-cobalamin transporter molecule | Cobalamin transporter, a B12 transporter | | |
| (e.g., antibody, affibody, aptamer, etc.) | | | |
| Anti-SVCT1 molecule (e.g., antibody, | SVCT1, a L-ascorbic acid transporter | | |
| affibody, aptamer, etc.) | | | |
| Anti-SVCT2 molecule (e.g., antibody, | SVCT2, a L-ascorbic acid transporter | | |
| affibody, aptamer, etc.) | | | |
| Anti-RFT1 molecule (e.g., antibody, | RFT1, a riboflavin transporter | | |
| affibody, aptamer, etc.) | | | |
| Anti-RFT2 molecule (e.g., antibody, | RFT2, a riboflavin transporter | | |
| affibody, aptamer, etc.) | | | |
| Anti-Vitamin A transporter molecule | Vitamin A transporter, transports Vitamin | | |
| (e.g., antibody, affibody, aptamer, etc.) | A (retinol) | | |
| Anti-Vitamin E transporter molecule | Vitamin E transporter, transports Vitamin | | |
| (e.g., antibody, affibody, aptamer, etc.) | E | | |
| Anti-SMCT1 molecule (e.g., antibody, | SMCT1, an iodine, lactate, short chain | | |
| affibody, aptamer, etc.) | fatty acid, niacin transporter | | |
| Anti-RFT3 molecule (e.g., antibody, | RFT3, a riboflavin transporter | | |
| affibody, aptamer, etc.) | | | |
| Anti-Cadherin 9 molecule (e.g., | Cadherin-9 | | Kidney cells |
| antibody, affibody, aptamer, etc.) | | | |
| Anti-Slc5a2 molecule (e.g., antibody, | Slc5a2 | | Kidney proximal tubule |
| affibody, aptamer, etc.) | | | cells |
| Anti-Slc12a3 molecule (e.g., antibody, | Slc12a3 | | Distal convoluted tubule |
| affibody, aptamer, etc.) | | | |
| Anti-CD40b molecule (e.g., antibody, | CD40b | | Retinal pigment epithelial |
| affibody, aptamer, etc.) | | | cells |
| Anti-ASC-1 molecule (e.g., antibody, | ASC-1 | | adipocyte |
| affibody, aptamer, etc.) | | | |
| Anti-PAT2 molecule (e.g., antibody, | PAT2 | | adipocyte |
| affibody, aptamer, etc.) | | | |
| Anti-P2RX5 molecule (e.g., antibody, | P2RX5 | | adipocyte |
| affibody, aptamer, etc.) | | | |
| Anti-CD16 molecule (e.g., antibody, | CD16 | | Natural killer cells |
| affibody, aptamer, etc.) | | | |
| Anti-NK1.1 molecule (e.g., antibody, | NK1.1 | | Natural killer cells |
| affibody, aptamer, etc.) | | | |
| Anti-CD177 molecule (e.g., antibody, | CD177 | | neutrophils |
| affibody, aptamer, etc.) | | | |
| Anti-GR-1 molecule (e.g., antibody, | GR-1 | | neutrophils |
| affibody, aptamer, etc.) | | | |
| Anti-FcγIII receptor molecule (e.g., | FcγIII receptor | | neutrophils |
| antibody, affibody, aptamer, etc.) | | | |
| Anti-CD90 molecule (e.g., antibody, | CD90 | | T cells. Liver cancer stem |
| affibody, aptamer, etc.) | | | cells |
| Anti-CD45 molecule (e.g., antibody, | CD45 | | T cells |
| affibody, aptamer, etc.) | | | |
| Anti-CD7 molecule (e.g., antibody, | CD7 | | T cells |
| affibody, aptamer, etc.) | | | |
| Anti-CD3 molecule (e.g., antibody, | CD3 | | T cells |
| affibody, aptamer, etc.) | | | |
| Anti-PD1 molecule (e.g., antibody, | PD1 | | T cells |
| affibody, aptamer, etc.) | | | |
| Anti-OX40 molecule (e.g., antibody, | OX40 | | T cells |
| affibody, aptamer, etc.) | | | |
| Anti-CD4 molecule (e.g., antibody, | CD4 | | T cells |
| affibody, aptamer, etc.) | | | |
| Anti-CD8 molecule (e.g., antibody, | CD8 | | T cells |
| affibody, aptamer, etc.) | | | |
| Anti-CD11b molecule (e.g., antibody, | CD11b | | monocytes |
| affibody, aptamer, etc.) | | | |
| Anti-beta glucan receptor molecule | beta glucan receptor | | monocytes |
| (e.g., antibody, affibody, aptamer, etc.) | | | |
| Anti-mannose receptor molecule (e.g., | Mannose receptor | | monocytes |
| antibody, affibody, aptamer, etc.) | | | |
| Anti-Fc receptor molecule (e.g., | Fc receptor | | monocytes |
| antibody, affibody, aptamer, etc.) | | | |
| Anti-DC-SIGN molecule (e.g., | DC-SIGN | | monocytes |
| antibody, affibody, aptamer, etc.) | | | |
| Anti-PSA molecule (e.g., antibody, | PSA (prostate-specific antigen) | | Prostate cells and prostate |
| affibody, aptamer, etc.) molecule (e.g., | | | cancer cells |
| antibody, affibody, aptamer, etc.) | | | |
| Anti-αv integrins (e.g., αvβ3 and αvβ5) | αv integrins | | Blood vessels |
| (e.g., antibody, affibody, aptamer, etc.) | | | |
| molecule (e.g., antibody, affibody, | | | |
| aptamer, etc.) | | | |
| Anti-CLDN1 molecule (e.g., antibody, | CLDN1 | | Colorectal cancer cells |
| affibody, aptamer, etc.) | | | |
| Anti-LY6G6D/F molecule (e.g., | LY6G6D/F | | Colorectal cancer cells |
| antibody, affibody, aptamer, etc.) | | | |
| Anti-TLR4 molecule (e.g., antibody, | TLR4 | | Colorectal cancer cells |
| affibody, aptamer, etc.) | | | |
| Anti-CD133 molecule (e.g., antibody, | CD133 | | Brain tumor cells, liver |
| affibody, aptamer, etc.) | | | cancer stem cells, |
| Anti-CD13 molecule (e.g., antibody, | CD13 | | Myeloid cells |
| affibody, aptamer, etc.) | | | |
| Anti-CD44 molecule (e.g., antibody, | CD44 | | Lymphocytes, monocytes, |
| affibody, aptamer, etc.) | | | endothelial cells, liver |
| | | | cancer stem cells |
| Anti-EpCam molecule (e.g., antibody, | EpCam | | Liver stem cells, |
| affibody, aptamer, etc.) | | | hepatoblasts, liver cancer |
| | | | stem cells |
| Anti-DLK1 molecule (e.g., antibody, | Delta-like 1 non-canonical | | Fetal liver cells, liver |
| affibody, aptamer, etc.) | Notch ligand 1 (DLK1) | | cancer stem cells |
| Anti-Matrix Metalloprotease (MMP) | MMPs | | |
| molecule (e.g., antibody, affibody, | | | |
| aptamer, etc.) | | | |
| PR_b peptide | α5β1 integrin | | Cancer cells |
| AG86 peptide | α6β4 integrin | | Cancer cells |
| affinity peptide LN (YEVGHRC) | Aminopeptidase N (APN/CD13) | | Aminopeptidase N |
| | | | expressing cells |
| Anti-CD20 molecule (e.g., antibody, | CD20 | | B-lymphocytes |
| affibody, aptamer, etc.) | | | |
| Anti-CD30 molecule (e.g., antibody, | CD30 | | |
| affibody, aptamer, etc.) | | | |

### Additional Components of the Engineered Acr Polypeptides

### Reporter Molecules and Tags

In an example embodiment, the engineered Acr polypeptide further comprises a reporter molecule operatively coupled to the cargo delivery molecule, the Acr polypeptide, or both.

Exemplary reporter proteins and tags include, but are not limited to, affinity tags, such as chitin binding protein (CBP), maltose-binding protein (MBP), glutathione-S-transferase (GST), poly(His) tag; solubilization tags such as thioredoxin (TRX) and poly(NANP), MBP, and GST; chromatography tags such as those consisting of polyanionic amino acids, such as FLAG-tag; epitope tags such as V5-tag, Myc-tag, HA-tag and NE-tag; protein tags that can allow specific enzymatic modification (such as biotinylation by biotin ligase) or chemical modification (such as reaction with FLASH-EDT2 for fluorescence imaging), DNA and/or RNA segments that contain restriction enzyme or other enzyme cleavage sites; DNA segments that encode products that provide resistance against otherwise toxic compounds including antibiotics, such as spectinomycin, ampicillin, kanamycin, tetracycline, Basta, neomycin phosphotransferase II (NEO), hygromycin phosphotransferase (HPT) and the like; DNA and/or RNA segments that encode products that are otherwise lacking in the recipient cell (e.g., tRNA genes, auxotrophic markers); DNA and/or RNA segments that encode products which can be readily identified (e.g., phenotypic markers such as β-galactosidase, β-glucuronidase (GUS)); fluorescent proteins such as green fluorescent protein (GFP), cyan fluorescent protein (CFP), yellow fluorescent protein (YFP), red fluorescent protein (RFP), mCherry, or other optically active proteins e.g., luciferase, and cell surface proteins); optically active dyes (e.g., fluorescent, UV, IR, and NIR dyes), polynucleotides that can generate one or more new primer sites for PCR (e.g., the juxtaposition of two DNA sequences not previously juxtaposed), DNA sequences not acted upon or acted upon by a restriction endonuclease or other DNA modifying enzyme, chemical, etc.; epitope tags (e.g. GFP, FLAG- and His-tags), and, DNA sequences that make a molecular barcode or unique molecular identifier (UMI), DNA sequences required for a specific modification (e.g., methylation) that allows its identification. Other suitable markers will be appreciated by those of skill in the art.

### Nuclear Localization Signals

In an embodiment, the engineered Acr polypeptide includes one or more nuclear localization signals (NLSs) at the C-terminus, the N-terminus, or both the N- and C-terminus of the Acr polypeptide, cargo delivery molecule or both. Without being bound by theory, such sequences may increase the transport of the Acr polypeptide to the nucleus of a cell, increase transport of the Acr through a pore formed from the pore-forming polypeptides, or both. For example, a PA pore is negatively charged and can favorably interact with polybasic peptides so as to facilitate transport. See e.g., Biochemistry 2014, 53, 44, 6934-6940.

In an embodiment, the NLSs used in the context of the present disclosure are heterologous to the proteins. Non-limiting examples of NLSs include an NLS sequence derived from: the NLS of the SV40 virus large T-antigen, having the amino acid sequence PKKKRKV (SEQ ID NO: 84) or PKKKRKVEAS (SEQ ID NO: 85); the NLS from nucleoplasmin (e.g., the nucleoplasmin bipartite NLS with the sequence KRPAATKKAGQAKKKK (SEQ ID NO: 86)); the c-myc NLS having the amino acid sequence PAAKRVKLD (SEQ ID NO: 87) or RQRRNELKRSP (SEQ ID NO: 88); the hRNPAI M9 NLS having the sequence NQSSNFGPMKGGNFGGRSSGPYGGGGQYFAKPRNQGGY (SEQ ID NO: 89); the sequence RMRIZFKNKGKDTAELRRRRVEVSVELRKAKKDEQILKRRNV (SEQ ID NO: 90) of the IBB domain from importin-alpha; the sequences VSRKRPRP (SEQ ID NO: 91) and PPKKARED (SEQ ID NO: 92) of the myoma T protein; the sequence PQPKKKPL (SEQ ID NO: 93) of human p53; the sequence SALIKKKKKMAP (SEQ ID NO: 94) of mouse c-abl IV; the sequences DRLRR (SEQ ID NO: 95) and PKQKKRK (SEQ ID NO: 96) of the influenza virus NS1; the sequence RKLKKKIKKL (SEQ ID NO: 97) of the Hepatitis virus delta antigen; the sequence REKKKFLKRR (SEQ ID NO: 98) of the mouse Mx1 protein; the sequence KRKGDEVDGVDEVAKKKSKK (SEQ ID NO: 99) of the human poly(ADP-ribose) polymerase; and the sequence RKCLQAGMNLEARKTKK (SEQ ID NO: 100) of the steroid hormone receptors (human) glucocorticoid, TAT peptide (GRKKRRQRRRPQ (SEQ ID NO: 101)), and R10 or any other polyarginine peptides, or any combination thereof. Additional NLSs that are suitable for use with the present invention as described herein are any of those in Srivaths et al. Bioinformation 2018, 14 (3), 132; Böhmová et al. Physiol. Res. 67 (Suppl. 2): S267-S279, 2018; Lange et al. J. Biol. Chem. 2007, 282 (8), 5101-5105, and Negi et al., 2015. Database. 2015: bav003; doi: 10.1093/database/bav003.

### Engineered Acr Polypeptide Delivery Systems

Described in an example embodiment herein are engineered Acr polypeptide delivery systems comprising a plurality of pore-forming polypeptides, wherein one or more of the pore-forming polypeptides are operatively coupled to a targeting moiety, and an engineered Acr polypeptide of the present invention, wherein the cargo delivery molecule of the engineered Acr polypeptide is capable of binding or otherwise interacting with the pore-forming polypeptide thereby transporting the Acr polypeptide through a pore formed from the pore-forming polypeptide. In an example embodiment, the pore-forming polypeptide is an alpha pore-forming polypeptide or a beta pore-forming polypeptide. In an example embodiment, the pore-forming polypeptide is a Bacillus anthracis protective antigen polypeptide or a derivative thereof. In an embodiment, the targeting moiety is a peptide or polypeptide. In an example embodiment, the targeting moiety is an antibody or fragment thereof. Other exemplary targeting moieties are described elsewhere herein. Without being bound by theory, the system components interact, such as by the mechanism depicted in FIG. 1, thereby delivering the Acr polypeptide to a cell.

In an embodiment, one or more components of the delivery system are premixed and/or contained in the same formulation prior to delivery to a cell or cell population. In an embodiment, one or more pore-forming polypeptides and engineered Acr polypeptides are premixed prior to delivery to a cell. In an embodiment, all of the components of the delivery system are provided to a cell or cell population simultaneously. In an embodiment, one or more, but not all, of the components of the system are provided simultaneously to the cell. In an embodiment, one or more but not all, of the components of the system are provided sequentially to a cell or cell population.

In an embodiment, the components are not premixed and/or are not contained in the same formulation prior to delivery to a cell or cell population. In an embodiment, all of the components are provided separately and/or sequentially to the cell or cell population.

### Polynucleotides and Vectors

Described in an example embodiment herein are polynucleotides encoding an engineered Acr polypeptide of the present invention and/or an engineered Acr polypeptide delivery system or component thereof of the present invention. As used herein, “nucleic acid,” “nucleotide sequence,” and “polynucleotide” can be used interchangeably herein and can generally refer to a string of at least two base-sugar-phosphate combinations and refer to, among others, single double-stranded DNA, DNA that is a mixture of single- and double-stranded regions, single- and double-stranded RNA, and RNA that is a mixture of single- and double-stranded regions, hybrid molecules comprising DNA and RNA that may be single-stranded or, more typically, double-stranded or a mixture of single- and double-stranded regions. In addition, polynucleotide, as used herein, can refer to triple-stranded regions comprising RNA or DNA or both RNA and DNA. The strands in such regions can be from the same molecule or from different molecules. The regions may include all of one or more of the molecules, but more typically involve only a region of some of the molecules. One of the molecules of a triple-helical region often is an oligonucleotide. “Polynucleotide” and “nucleic acids” also encompass such chemically, enzymatically, or metabolically modified forms of polynucleotides, as well as the chemical forms of DNA and RNA characteristic of viruses and cells, including simple and complex cells, inter alia. For instance, the term polynucleotide, as used herein, can include DNAs or RNAs as described herein that contain one or more modified bases. Thus, DNAs or RNAs including unusual bases, such as inosine, or modified bases, such as tritylated bases, to name just two examples, are polynucleotides as the term is used herein. “Polynucleotide,” “nucleotide sequences,” and “nucleic acids” also include PNAs (peptide nucleic acids), phosphorothioates, phosphorodiamidate morpholino oligomers, and other variants of the phosphate backbone of native nucleic acids. Natural nucleic acids have a phosphate backbone; artificial nucleic acids can contain other types of backbones but contain the same bases. Thus, DNAs or RNAs with backbones modified for stability or for other reasons are “nucleic acids” or “polynucleotides” as that term is intended herein. As used herein, “nucleic acid sequence” and “oligonucleotide” also encompass nucleic acid and polynucleotide as defined elsewhere herein. As used herein “encode”, “encoding,” and/or the like refers to the general principle that DNA is transcribed into an RNA product, which in some cases is translated into amino acids that form polypeptides. Thus, a protein-encoding polynucleotide is a polynucleotide that encodes an RNA product that is translated into the protein. In an embodiment, the polynucleotides are codon-optimized. Codon optimization of polynucleotides is described elsewhere herein; see, e.g., below with respect to “vector polynucleotides”. In an embodiment, the polynucleotides are included in a vector or vector system. In an embodiment, the polynucleotides are not included in a vector or vector system. In an embodiment, the polynucleotides are contained in a delivery vehicle. Delivery vehicles are described in greater detail elsewhere herein.

### Vectors and Vector Systems

Described in an example embodiment herein are vector systems comprising one or more vectors comprising one or more polynucleotides of the present invention encoding an engineered Acr polypeptide of the present invention and/or an engineered Acr polypeptide delivery system or component thereof of the present invention; and optionally one or more regulatory elements operatively coupled to the one or more polynucleotides.

In certain embodiments, the vector can contain one or more polynucleotides encoding one or more elements of an engineered Acr polypeptide system or component thereof (e.g., an Acr polypeptide, cargo delivery molecule, pore-forming protein, etc.) described herein. The vectors can be useful in producing bacterial, fungal, yeast, plant cells, animal cells, and transgenic animals that can express one or more components of the engineered Acr polypeptide system described herein. Within the scope of this disclosure are vectors containing one or more of the polynucleotide sequences described herein. One or more of the polynucleotides that are part of the engineered Acr polypeptide system described herein can be included in a vector or vector system. The vectors and/or vector systems can be used, for example, to express one or more of the polynucleotides in a cell, such as a producer cell, to produce an engineered Acr polypeptide system containing virus particles described elsewhere herein. Other uses for the vectors and vector systems described herein are also within the scope of this disclosure. In general, and throughout this specification, the term “vector” refers to a tool that allows or facilitates the transfer of an entity from one environment to another. In some contexts which will be appreciated by those of ordinary skill in the art, “vector” can be a term of art to refer to a nucleic acid molecule capable of transporting another nucleic acid to which it has been linked. A vector can be a replicon, such as a plasmid, phage, or cosmid, into which another DNA segment may be inserted so as to bring about the replication of the inserted segment. Generally, a vector is capable of replication when associated with the proper control elements.

Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. One type of vector is a “plasmid,” which refers to a circular double-stranded DNA loop into which additional DNA segments can be inserted, such as by standard molecular cloning techniques. Another type of vector is a viral vector, wherein virally-derived DNA or RNA sequences are present in the vector for packaging into a virus (e.g. retroviruses, replication defective retroviruses, adenoviruses, replication defective adenoviruses, and adeno-associated viruses (AAVs)). Viral vectors also include polynucleotides carried by a virus for transfection into a host cell. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell and, thereby, are replicated along with the host genome. Moreover, certain vectors are capable of directing the expression of genes to which they are operatively linked. Such vectors are referred to herein as “expression vectors.” Common expression vectors of utility in recombinant DNA techniques are often in the form of plasmids.

Recombinant expression vectors can be composed of a nucleic acid (e.g., a polynucleotide) of the invention in a form suitable for expression of the nucleic acid in a host cell, which means that the recombinant expression vectors include one or more regulatory elements, which can be selected on the basis of the host cells to be used for expression, that is operatively-linked to the nucleic acid sequence to be expressed. Within a recombinant expression vector, “operably linked” and “operatively linked” are used interchangeably herein and further defined elsewhere herein. In the context of a vector, the term “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell). Advantageous vectors include lentiviruses and adeno-associated viruses, and types of such vectors can also be selected for targeting particular types of cells. These and other embodiments of the vectors and vector systems are described elsewhere herein.

In an embodiment, the vector can be a bicistronic vector. In an embodiment, a bicistronic vector can be used for one or more elements of the engineered Acr polypeptide system described herein. In an embodiment, the expression of element(s) of the engineered Acr polypeptide system described herein can be driven by a ubiquitous Pol II promoter, such as beta-actin, CMV, SV40, or another ubiquitous promoter. In an embodiment, the expression of element(s) of the engineered Acr polypeptide system described herein can be driven by a tissue-specific Pol II promoter. Where the element of the engineered Acr polypeptide system is an RNA, its expression can be driven by a Pol III promoter, such as a U6 promoter. In an embodiment, the two are combined.

These and others are further detailed and described elsewhere herein.

### Cell-Based Vector Amplification and Expression

Vectors may be introduced and propagated in a prokaryote or prokaryotic cell. In an embodiment, a prokaryote is used to amplify copies of a vector to be introduced into a eukaryotic cell or as an intermediate vector in the production of a vector to be introduced into a eukaryotic cell (e.g., amplifying a plasmid as part of a viral vector packaging system). The vectors can be viral-based or non-viral based. In an embodiment, a prokaryote is used to amplify copies of a vector and express one or more nucleic acids, such as to provide a source of one or more proteins for delivery to a host cell or host organism.

Vectors can be designed for expression of one or more elements of the engineered Acr polypeptide delivery system described herein (e.g., nucleic acid transcripts, proteins, enzymes, and combinations thereof) in a suitable host cell. In an embodiment, the suitable host cell is a prokaryotic cell. Suitable host cells include, but are not limited to, bacterial cells, yeast cells, insect cells, and mammalian cells. In an embodiment, the suitable host cell is a eukaryotic cell.

In an embodiment, the suitable host cell is a suitable bacterial cell. Suitable bacterial cells include, but are not limited to bacterial cells from the bacteria of the species Escherichia coli. Many suitable strains of E. coli are known in the art for expression of vectors. These include, but are not limited to Pir1, Stbl2, Stbl3, Stbl4, TOP10, XL1 Blue, XL10 Gold, Rosetta 2 (DE3) (Novagen), NEB® 5-alpha Competent E. coli (High Efficiency) (New England Biolabs), and BL21 (DE3) Competent E. coli (New England Biolabs). In an embodiment, the host cell is a suitable insect cell. Suitable insect cells include those from Spodoptera frugiperda. Suitable strains of S. frugiperda cells include, but are not limited to Sf9 and Sf21. In an embodiment, the host cell is a suitable yeast cell. In an embodiment, the yeast cell can be from Saccharomyces cerevisiae. In an embodiment, the host cell is a suitable mammalian cell. Many types of mammalian cells have been developed to express vectors. Suitable mammalian cells include, but are not limited to, HEK293, HEK293T, HEK293FT, Chinese Hamster Ovary Cells (CHOs), mouse myeloma cells, HeLa, U2OS, A549, HT1080, CAD, P19, NIH 3T3, L929, N2a, MCF-7, Y79, SO-Rb50, HepG G2, DIKX-X11, J558L, Baby hamster kidney cells (BHK), and chicken embryo fibroblasts (CEFs). Suitable host cells are discussed further in Goeddel, GENE EXPRESSION TECHNOLOGY: METHODS IN ENZYMOLOGY 185, Academic Press, San Diego, Calif. (1990).

In an embodiment, the vector can be a yeast expression vector. Examples of vectors for expression in yeast Saccharomyces cerevisiae include pYepSec1 (Baldari, et al., 1987. EMBO J. 6:229-234), pMFa (Kuijan and Herskowitz, 1982. Cell 30:933-943), pJRY88 (Schultz et al., 1987. Gene 54:113-123), pYES2 (Invitrogen Corporation, San Diego, Calif.), and picZ (InVitrogen Corp, San Diego, Calif.). As used herein, a “yeast expression vector” refers to a nucleic acid that contains one or more sequences encoding an RNA and/or polypeptide and may further contain any desired elements that control the expression of the nucleic acid(s), as well as any elements that enable the replication and maintenance of the expression vector inside the yeast cell. Many suitable yeast expression vectors and features thereof are known in the art; for example, various vectors and techniques are illustrated in Yeast Protocols, 2nd edition, Xiao, W., ed. (Humana Press, New York, 2007) and Buckholz, R. G. and Gleeson, M. A. (1991) Biotechnology (NY) 9 (11): 1067-72. Yeast vectors can contain, without limitation, a centromeric (CEN) sequence, an autonomous replication sequence (ARS), a promoter, such as an RNA Polymerase III promoter, operably linked to a sequence or gene of interest, a terminator such as an RNA polymerase III terminator, an origin of replication, and a marker gene (e.g., auxotrophic, antibiotic, or other selectable markers). Examples of expression vectors for use in yeast may include plasmids, yeast artificial chromosomes, 2μ plasmids, yeast integrative plasmids, yeast replicative plasmids, shuttle vectors, and episomal plasmids.

In an embodiment, the vector is a baculovirus vector or expression vector and can be suitable for expression of polynucleotides and/or proteins in insect cells. In an embodiment, the suitable host cell is an insect cell. Baculovirus vectors available for expression of proteins in cultured insect cells (e.g., SF9 cells) include the pAc series (Smith, et al., 1983. Mol. Cell. Biol. 3:2156-2165) and the pVL series (Lucklow and Summers, 1989. Virology 170:31-39). rAAV (recombinant Adeno-associated viral) vectors are preferably produced in insect cells, e.g., Spodoptera frugiperda Sf9 insect cells, grown in serum-free suspension culture. Serum-free insect cells can be purchased from commercial vendors, e.g., Sigma Aldrich (EX-CELL 405).

In an embodiment, the vector is a mammalian expression vector. In an embodiment, the mammalian expression vector is capable of expressing one or more polynucleotides and/or polypeptides in a mammalian cell. Examples of mammalian expression vectors include, but are not limited to, pCDM8 (Seed, 1987. Nature 329:840) and pMT2PC (Kaufman, et al., 1987. EMBO J. 6:187-195). The mammalian expression vector can include one or more suitable regulatory elements capable of controlling expression of the one or more polynucleotides and/or proteins in the mammalian cell. For example, commonly used promoters are derived from polyoma, adenovirus 2, cytomegalovirus, simian virus 40, and others disclosed herein and known in the art. More details on suitable regulatory elements are described elsewhere herein.

For other suitable expression vectors and vector systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In an embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1:268-277), lymphoid-specific promoters (Calame and Eaton, 1988. Adv. Immunol. 43:235-275), in particular promoters of T cell receptors (Winoto and Baltimore, 1989. EMBO) J. 8:729-733) and immunoglobulins (Baneiji, et al., 1983. Cell 33:729-740; Queen and Baltimore, 1983. Cell 33:741-748), neuron-specific promoters (e.g., the neurofilament promoter; Byrne and Ruddle, 1989. Proc. Natl. Acad. Sci. USA 86:5473-5477), pancreas-specific promoters (Edlund, et al., 1985. Science 230:912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249:374-379) and the α-fetoprotein promoter (Campes and Tilghman, 1989. Genes Dev. 3:537-546). With regards to these prokaryotic and eukaryotic vectors, mention is made of U.S. Pat. No. 6,750,059, the contents of which are incorporated by reference herein in their entirety. Other embodiments can utilize viral vectors, with regards to which mention is made of U.S. patent application Ser. No. 13/092,085, the contents of which are incorporated by reference herein in their entirety. Tissue-specific regulatory elements are known in the art and in this regard, mention is made of U.S. Pat. No. 7,776,321, the contents of which are incorporated by reference herein in their entirety. In an embodiment, a regulatory element can be operably linked to one or more elements of the engineered Acr polypeptide delivery system so as to drive the expression of one or more elements of the engineered Acr polypeptide delivery system described herein.

In an embodiment, the vector can be a fusion vector or fusion expression vector. In an embodiment, fusion vectors add a number of amino acids to a protein encoded therein, such as to the amino terminus, carboxy terminus, or both of a recombinant protein. Such fusion vectors can serve one or more purposes, such as (i) to increase the expression of recombinant protein, (ii) to increase the solubility of the recombinant protein, and (iii) to aid in the purification of the recombinant protein by acting as a ligand in affinity purification. In an embodiment, expression of polynucleotides (such as non-coding polynucleotides) and proteins in prokaryotes can be carried out in Escherichia coli with vectors containing constitutive or inducible promoters directing the expression of either fusion or non-fusion polynucleotides and/or proteins. In an embodiment, the fusion expression vector can include one or more proteolytic cleavage sites, which can be introduced at the junction of the fusion vector backbone or other fusion moiety and the recombinant polynucleotide or protein to enable separation of the recombinant polynucleotide or protein from the fusion vector backbone or another fusion moiety subsequent to purification of the fusion polynucleotide or protein. Such enzymes and their cognate recognition sequences include Factor Xa, thrombin and enterokinase, and TEV protease sites. Example fusion expression vectors include pGEX (Pharmacia Biotech Inc; Smith and Johnson, 1988. Gene 67:31-40), pMAL (New England Biolabs, Beverly, Mass.) and pRIT5 (Pharmacia, Piscataway, N.J.) that fuse glutathione S-transferase (GST), maltose-binding protein, or protein A, respectively, to the target recombinant protein. Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

In an embodiment, one or more vectors driving the expression of one or more elements of an engineered Acr polypeptide delivery system described herein are introduced into a host cell such that the expression of the elements of the engineered delivery system described herein direct formation of an engineered Acr polypeptide delivery complex at one or more target cells. For example, an engineered Acr polypeptide described herein and a pore-forming polypeptide component can each be operably linked to separate regulatory elements on separate vectors. DNAs and/or RNA(s) of different elements of an engineered Acr polypeptide delivery system described herein can be delivered to an animal, plant, microorganism or cell thereof to produce an animal (e.g., a mammal, reptile, avian, etc.), plant, microorganism or cell thereof that constitutively, inducibly, or conditionally expresses different elements of the engineered Acr polypeptide delivery system described herein that incorporates one or more elements of the engineered Acr polypeptide delivery system described herein or contains one or more cells that incorporate and/or express one or more elements of the engineered Acr polypeptide delivery system described herein.

In an embodiment, two or more of the elements expressed from the same or different regulatory element(s), can be combined in a single vector, with one or more additional vectors providing any components of the system not included in the first vector. Engineered Acr polypeptide delivery system polynucleotides that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In an embodiment, a single promoter drives the expression of a transcript encoding one or more engineered Acr polypeptide delivery system proteins embedded within one or more intron sequences (e.g., each in a different intron, two or more in at least one intron, or all in a single intron). In an embodiment, the engineered Acr polypeptide delivery system polynucleotides can be operably linked to and expressed from the same promoter.

### Cell-Free Vector and Polynucleotide Expression

In an embodiment, the polynucleotide encoding one or more features of the engineered Acr polypeptide delivery system can be expressed from a vector or suitable polynucleotide in a cell-free in vitro system. In other words, the polynucleotide can be transcribed and optionally translated in vitro. In vitro, transcription/translation systems and appropriate vectors are generally known in the art and commercially available. Generally, in vitro transcription and in vitro translation systems replicate the processes of RNA and protein synthesis, respectively, outside of the cellular environment. Vectors and suitable polynucleotides for in vitro transcription can include T7, SP6, and T3 promoters or other regulatory sequences that can be recognized and acted upon by an appropriate polymerase to transcribe the polynucleotide or vector.

In vitro translation can be stand-alone (e.g., translation of a purified polyribonucleotide) or linked/coupled to transcription. In an embodiment, the cell-free (or in vitro) translation system can include extracts from rabbit reticulocytes, wheat germ, and/or E. coli. The extracts can include various macromolecular components that are needed for the translation of exogenous RNA (e.g., 70S or 80S ribosomes, tRNAs, aminoacyl-tRNA, synthetases, initiation, elongation factors, termination factors, etc.). Other components can be included or added during the translation reaction, including but not limited to, amino acids, energy sources (ATP, GTP), energy regenerating systems (e.g., creatine phosphate and creatine phosphokinase for use in eukaryotic systems), and phosphoenol pyruvate and pyruvate kinase for use in bacterial systems), and other co-factors (e.g., Mg²⁺, K⁺, etc.). As previously mentioned, in vitro translation can be based on RNA or DNA starting material. Some translation systems can utilize an RNA template as starting material (e.g., reticulocyte lysates and wheat germ extracts). Some translation systems can utilize a DNA template as a starting material (e.g., E coli-based systems). In these systems, transcription and translation are coupled and DNA is first transcribed into RNA, which is subsequently translated. Suitable standard and coupled cell-free translation systems are generally known in the art and are commercially available.

### Vector Features

The vectors can include additional features that can confer one or more functionalities to the vector, the polynucleotide to be delivered, a virus particle produced therefrom, or a polypeptide expressed thereof. Such features include, but are not limited to, regulatory elements, selectable markers, molecular identifiers (e.g. molecular barcodes), stabilizing elements, and the like. It will be appreciated by those skilled in the art that the design of the expression vector and additional features included can depend on such factors as the choice of the host cell to be transformed, the level of expression desired, etc.

### Regulatory Elements

In certain embodiments, the polynucleotides and/or vectors thereof described herein (such as the engineered Acr polypeptide delivery system polynucleotides of the present invention) can include one or more regulatory elements that can be operatively linked to the polynucleotide. The term “regulatory element” is intended to include promoters, enhancers, internal ribosomal entry sites (IRES), other expression control elements (e.g., transcription termination signals, such as polyadenylation signals and poly-U sequences) and cellular localization signals (e.g. nuclear localization signals). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cells and those that direct expression of the nucleotide sequence only in certain host cells (e.g., tissue-specific regulatory sequences). A tissue-specific promoter can direct expression primarily in a desired tissue of interest, such as muscle, neuron, bone, skin, blood, specific organs (e.g., liver, pancreas), or particular cell types (e.g., lymphocytes). Regulatory elements may also direct expression in a temporal-dependent manner, such as in a cell cycle-dependent or developmental stage-dependent manner, which may or may not also be tissue or cell-type specific. In an embodiment, a vector comprises one or more pol III promoters (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) (see, e.g., Boshart et al, Cell, 41:521-530 (1985)), the SV40 promoter, the dihydrofolate reductase promoter, the β-actin promoter, the phosphoglycerol kinase (PGK) promoter, and the EF1α promoter. Also encompassed by the term “regulatory element” are enhancer elements, such as woodchuck hepatitis virus post-transcriptional regulator element (WPRE); CMV enhancers; the R-U5′ segment in the long terminal repeat (LTR) of HTLV-I (Mol. Cell. Biol., Vol. 8 (1), p. 466-472, 1988); SV40 enhancer; and the intron sequence between exons 2 and 3 of rabbit β-globin (Proc. Natl. Acad. Sci. USA., Vol. 78 (3), p. 1527-31, 1981).

In an embodiment, the regulatory sequence can be a regulatory sequence described in U.S. Pat. No. 7,776,321, U.S. Pat. Pub. No. 2011/0027239, and International Patent Publication No. WO 2011/028929, the contents of which are incorporated by reference herein in their entirety. In an embodiment, the vector can contain a minimal promoter. In an embodiment, the minimal promoter is the Mecp2 promoter, tRNA promoter, or U6. In a further embodiment, the minimal promoter is tissue-specific. In an embodiment, the length of the vector polynucleotide, the minimal promoters, and polynucleotide sequences is less than 4.4 kb.

To express a polynucleotide, the vector can include one or more transcriptional and/or translational initiation regulatory sequences, e.g. promoters, that direct the transcription of the gene and/or translation of the encoded protein in a cell. In an embodiment, a constitutive promoter may be employed. Suitable constitutive promoters for mammalian cells are generally known in the art and include, but are not limited to SV40, CAG, CMV, EF-1α, β-actin, RSV, and PGK. Suitable constitutive promoters for bacterial cells, yeast cells, and fungal cells are generally known in the art, such as a T7 promoter for bacterial expression and an alcohol dehydrogenase promoter for expression in yeast.

In an embodiment, the regulatory element can be a regulated promoter. “Regulated promoter” refers to promoters that direct gene expression not constitutively but in a temporally- and/or spatially-regulated manner and includes tissue-specific, tissue-preferred, and inducible promoters. Regulated promoters include conditional promoters and inducible promoters. In an embodiment, conditional promoters can be employed to direct the expression of a polynucleotide in a specific cell type, under certain environmental conditions, and/or during a specific state of development. Suitable tissue-specific promoters can include, but are not limited to, liver-specific promoters (e.g. APOA2, SERPIN A1 (hAAT), CYP3A4, and MIR122), pancreatic cell promoters (e.g. INS, IRS2, Pdx1, Alx3, Ppy), cardiac-specific promoters (e.g. Myh6 (alpha MHC), MYL2 (MLC-2v), TNI3 (cTnl), NPPA (ANF), Slc8a1 (Ncx1)), central nervous system cell promoters (SYN1, GFAP, INA, NES, MOBP, MBP, TH, FOXA2 (HNF3 beta)), skin cell-specific promoters (e.g. FLG, K14, TGM3), immune cell-specific promoters, (e.g. ITGAM, CD43 promoter, CD14 promoter, CD45 promoter, CD68 promoter), urogenital cell-specific promoters (e.g. Pbsn, Upk2, Sbp, Fer114), endothelial cell-specific promoters (e.g. ENG), pluripotent and embryonic germ layer cell-specific promoters (e.g. Oct4, NANOG, Synthetic Oct4, T brachyury, NES, SOX17, FOXA2, MIR122), and muscle cell-specific promoter (e.g. Desmin). Other tissue and/or cell-specific promoters are generally known in the art and are within the scope of this disclosure.

Inducible/conditional promoters can be positively inducible/conditional promoters (e.g. a promoter that activates transcription of the polynucleotide upon appropriate interaction with an activated activator, or an inducer compound, environmental condition, or another stimulus) or a negative/conditional inducible promoter (e.g. a promoter that is repressed by e.g., being bound by a repressor) until the repressor condition of the promotor is removed e.g., when inducer binds a repressor bound to the promoter, stimulating the release of the promoter by the repressor or removal of a chemical repressor from the promoter environment. The inducer can be a compound, environmental condition, or another stimulus. Thus, inducible/conditional promoters can be responsive to any suitable stimuli such as chemical, biological, or other molecular agents, temperature, light, and/or pH. Suitable inducible/conditional promoters include, but are not limited to, Tet-On, Tet-Off, Lac promoter, pBad, AlcA, LexA, Hsp70 promoter, Hsp90 promoter, pDawn, XVE/OlexA, GVG, and pOp/LhGR.

Where expression in a plant cell is desired, the components of the engineered Acr polypeptide delivery system described herein are typically placed under the control of a plant promoter, i.e., a promoter operable in plant cells. The use of different types of promoters is envisaged.

A constitutive plant promoter is a promoter that is able to express the open reading frame (ORF) that it controls in all or nearly all of the plant tissues during all or nearly all developmental stages of the plant (referred to as “constitutive expression”). One non-limiting example of a constitutive promoter is the cauliflower mosaic virus 35S promoter. Different promoters may direct the expression of a gene in different tissues or cell types, or at different stages of development, or in response to different environmental conditions. In particular embodiments, one or more of the engineered Acr polypeptide delivery system components are expressed under the control of a constitutive promoter, such as the cauliflower mosaic virus 35S promoter tissue-preferred promoters can be utilized to target enhanced expression in certain cell types within particular plant tissue, for instance, vascular cells in leaves or roots or in specific cells of the seed. Examples of particular promoters for use in the engineered Acr polypeptide delivery system are found in Kawamata et al., (1997) Plant Cell Physiol 38:792-803; Yamamoto et al., (1997) Plant J 12:255-65; Hire et al, (1992) Plant Mol Biol 20:207-18, Kuster et al, (1995) Plant Mol Biol 29:759-72, and Capana et al., (1994) Plant Mol Biol 25:681-91.

Examples of promoters that are inducible and that can allow for spatiotemporal control of gene editing or gene expression may use a form of energy. The form of energy may include but is not limited to sound energy, electromagnetic radiation, chemical energy, and/or thermal energy. Examples of inducible systems include tetracycline-inducible promoters (Tet-On or Tet-Off), small molecule two-hybrid transcription activation systems (FKBP, ABA, etc.), or light-inducible systems (Phytochrome, Light-oxygen-voltage-sensing (LOV) domains, or cryptochrome, such as a Light Inducible Transcriptional Effector (LITE) that directs changes in transcriptional activity in a sequence-specific manner. The components of a light-inducible system may include one or more elements of the engineered Acr polypeptide delivery system described herein, a light-responsive cytochrome heterodimer (e.g. from Arabidopsis thaliana), and a transcriptional activation/repression domain. In an embodiment, the vector can include one or more of the inducible DNA binding proteins provided in International Patent Publication No. WO 2014/018423 and US Patent Publication Nos. 2015/0291966, 2017/0166903, 2019/0203212, which describe, e.g., embodiments of inducible DNA binding proteins and methods of use and can be adapted for use with the present invention.

In an embodiment, transient or inducible expression can be achieved by including, for example, chemical-regulated promoters, i.e. whereby the application of an exogenous chemical induces gene expression. Modulation of gene expression can also be obtained by including a chemical-repressible promoter, where the application of the chemical represses gene expression. Chemical-inducible promoters include but are not limited to, the maize ln2-2 promoter, activated by benzene sulfonamide herbicide safeners (De Veylder et al., (1997) Plant Cell Physiol 38:568-77), the maize GST promoter (GST-11-27, WO93/01294), activated by hydrophobic electrophilic compounds used as pre-emergent herbicides, and the tobacco PR-1a promoter (Ono et al., (2004) Biosci Biotechnol Biochem 68:803-7) activated by salicylic acid. Promoters that are regulated by antibiotics, such as tetracycline-inducible and tetracycline-repressible promoters (Gatz et al., (1991) Mol Gen Genet 227:229-37; U.S. Pat. Nos. 5,814,618 and 5,789,156) can also be used herein.

In an embodiment, the polynucleotide, vector, or system thereof can include one or more elements capable of translocating and/or expressing an engineered Acr polypeptide delivery system polynucleotide to/in a specific cell component or organelle. Such organelles can include but are not limited to, the nucleus, ribosome, endoplasmic reticulum, Golgi apparatus, chloroplast, mitochondria, vacuole, lysosome, cytoskeleton, plasma membrane, cell wall, peroxisome, centrioles, etc. Such regulatory elements can include, but are not limited to, nuclear localization signals (examples of which are described in greater detail elsewhere herein), any such as those that are annotated in the LocSigDB database (see e.g., Negi et al., 2015. Database. 2015: bav003; doi: 10.1093/database/bav003), nuclear export signals (e.g., LXXXLXXLXL (SEQ ID NO: 102) and others described elsewhere herein), endoplasmic reticulum localization/retention signals (e.g. KDEL (SEQ ID NO: 103), KDXX, KKXX, KXX, and others described elsewhere herein; and see e.g. Liu et al. 2007 Mol. Biol. Cell. 18 (3): 1073-1082 and Gorleku et al., 2011. J. Biol. Chem. 286:39573-39584), mitochondria (see e.g. Cell Reports. 22:2818-2826, particularly at FIG. 2; Doyle et al. 2013. PLOS ONE 8, e67938; Funes et al. 2002. J. Biol. Chem. 277:6051-6058; Matouschek et al. 1997. PNAS USA 85:2091-2095; Oca-Cossio et al., 2003. 165:707-720; Waltner et al., 1996. J. Biol. Chem. 271:21226-21230; Wilcox et al., 2005. PNAS USA 102:15435-15440; Galanis et al., 1991. FEBS Lett 282:425-430), peroxisome (e.g. (S/A/C)-(K/R/H)-(L/A), SLK, (R/K)-(L/V/I)-XXXXX-(H/Q)-(L/A/F). Suitable protein targeting motifs can also be designed or identified using any suitable database or prediction tool, including but not limited to Minimotif Miner (http: minimotifminer.org, http://mitominer.mrc-mbu.cam.ac.uk/release-4.0/embodiment.do?name=Protein % 20MTS), LocDB (see above), PTSs predictor, TargetP-2.0 (http://www.cbs.dtu.dk/services/TargetP/), ChloroP (http://www.cbs.dtu.dk/services/ChloroP/); NetNES (http://www.cbs.dtu.dk/services/NetNES/), Predotar (https://urgi.versailles.inra.fr/predotar/), and SignalP (http://www.cbs.dtu.dk/services/SignalP/).

### Selectable Markers and Tags

One or more of the engineered Acr polypeptide delivery system polynucleotides can be operably linked, fused to, or otherwise modified to include a polynucleotide that encodes or is a selectable marker or tag, which can be a polynucleotide or polypeptide. In an embodiment, the polypeptide selectable marker can be incorporated in the engineered Acr polypeptide delivery system polynucleotide such that the selectable marker polypeptide, when translated, is inserted between two amino acids between the N- and C-terminus of the engineered Acr polypeptide delivery system polypeptide or at the N- and/or C-terminus of the engineered Acr polypeptide delivery system polypeptide. In an embodiment, the selectable marker or tag is a polynucleotide barcode or unique molecular identifier (UMI).

It will be appreciated that the polynucleotide encoding such selectable markers or tags can be incorporated into a polynucleotide encoding one or more components of the engineered Acr polypeptide delivery system described herein in an appropriate manner to allow expression of the selectable marker or tag. Such techniques and methods are described elsewhere herein and will be instantly appreciated by one of ordinary skill in the art in view of this disclosure. Many such selectable markers and tags are generally known in the art and are intended to be within the scope of this disclosure.

Suitable selectable markers and tags include, but are not limited to, affinity tags, such as chitin binding protein (CBP), maltose-binding protein (MBP), glutathione-S-transferase (GST), poly(His) tag; solubilization tags such as thioredoxin (TRX) and poly(NANP), MBP, and GST; chromatography tags such as those consisting of polyanionic amino acids, such as FLAG-tag; epitope tags such as V5-tag, Myc-tag, HA-tag and NE-tag; protein tags that can allow specific enzymatic modification (such as biotinylation by biotin ligase) or chemical modification (such as reaction with FLASH-EDT2 for fluorescence imaging), DNA and/or RNA segments that contain restriction enzyme or other enzyme cleavage sites; DNA segments that encode products that provide resistance against otherwise toxic compounds including antibiotics, such as, spectinomycin, ampicillin, kanamycin, tetracycline, Basta, neomycin phosphotransferase II (NEO), hygromycin phosphotransferase (HPT) and the like; DNA and/or RNA segments that encode products that are otherwise lacking in the recipient cell (e.g., tRNA genes, auxotrophic markers); DNA and/or RNA segments that encode products which can be readily identified (e.g., phenotypic markers such as β-galactosidase, GUS; fluorescent proteins such as green fluorescent protein (GFP), cyan (CFP), yellow (YFP), red (RFP), luciferase, and cell surface proteins); polynucleotides that can generate one or more new primer sites for PCR (e.g., the juxtaposition of two DNA sequences not previously juxtaposed), DNA sequences not acted upon or acted upon by a restriction endonuclease or other DNA modifying enzyme, chemical, etc.; epitope tags (e.g. GFP, FLAG- and His-tags), and, DNA sequences that make a molecular barcode or unique molecular identifier (UMI), DNA sequences required for a specific modification (e.g., methylation) that allows its identification. Other suitable markers will be appreciated by those of skill in the art.

Selectable markers and tags can be operably linked to one or more components of the engineered Acr polypeptide delivery system described herein via suitable linkers, such as a glycine or glycine serine linkers as short as GS or GG up to (GGGGG) 3 (SEQ ID NO: 104) or (GGGGS) 3 (SEQ ID NO: 31). and other linkers described elsewhere herein.

The vector or vector system can include one or more polynucleotides encoding one or more targeting moieties. In an embodiment, the targeting moiety encoding polynucleotides can be included in the vector or vector system, such as a viral vector system, such that they are expressed within and/or on the virus particle(s) produced such that the virus particles can be targeted to specific cells, tissues, organs, etc. In an embodiment, the targeting moiety encoding polynucleotides can be included in the vector or vector system such that the engineered Acr polypeptide delivery system polynucleotide(s) and/or products expressed therefrom include the targeting moiety and can be targeted to specific cells, tissues, organs, etc. In an embodiment, such as non-viral carriers, the targeting moiety can be attached to the carrier (e.g. polymer, lipid, inorganic molecule, etc.) and can be capable of targeting the carrier and any attached or associated engineered Acr polypeptide delivery system polynucleotide(s) to specific cells, tissues, organs, etc.

### Codon Optimization of Vector Polynucleotides

As described elsewhere herein, the polynucleotide encoding one or more embodiments of the engineered Acr polypeptide delivery system described herein can be codon optimized. In an embodiment, one or more polynucleotides contained in a vector (“vector polynucleotides”) that are not polynucleotides encoding an engineered Acr polypeptide or component thereof of the present invention are codon optimized. In general, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit a particular bias for certain codons of a particular amino acid. Codon bias (differences in codon usage between organisms) often correlates with the efficiency of translation of messenger RNA (mRNA), which is, in turn, believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, PA). In an embodiment, one or more codons (e.g., 1, 2, 3, 4, 5, 10, 15, 20, 25, 50, or more, or all codons) in a sequence encoding an engineered Acr polypeptide delivery system or component thereof of the present invention and any co-therapy (e.g., a DNA/RNA-targeting Cas protein) corresponds to the most frequently used codon for a particular amino acid. As to codon usage in yeast, reference is made to the online Yeast Genome database available at http://www.yeastgenome.org/community/codon_usage.shtml, or Codon selection in yeast, Bennetzen and Hall, J Biol Chem. 1982 Mar. 25; 257 (6): 3026-31. As to codon usage in plants including algae, reference is made to Codon usage in higher plants, green algae, and cyanobacteria, Campbell and Gowri, Plant Physiol. 1990 January; 92 (1): 1-11; as well as Codon usage in plant genes, Murray et al, Nucleic Acids Res. 1989 Jan. 25; 17 (2): 477-98; or Selection on the codon bias of chloroplast and cyanelle genes in different plant and algal lineages, Morton B R, J Mol Evol. 1998 April; 46 (4): 449-59.

The vector polynucleotide can be codon optimized for expression in a specific cell type, tissue type, organ type, and/or subject type. In an embodiment, a codon-optimized sequence is a sequence optimized for expression in a eukaryote, e.g., humans (i.e., being optimized for expression in a human or human cell), or for another eukaryote, such as another animal (e.g., a mammal or avian) as is described elsewhere herein. Such codon-optimized sequences are within the ambit of the ordinary skilled artisan in view of the description herein. In an embodiment, the polynucleotide is codon optimized for a specific cell type. Such cell types can include but are not limited to, epithelial cells (including skin cells, cells lining the gastrointestinal tract, cells lining other hollow organs), nerve cells (nerves, brain cells, spinal column cells, nerve support cells (e.g. astrocytes, glial cells, Schwann cells, etc.)), muscle cells (e.g. cardiac muscle cells, smooth muscle cells, and skeletal muscle cells), connective tissue cells (fat and other soft tissue padding cells, bone cells, tendon cells, cartilage cells), blood cells, stem cells and other progenitor cells, immune system cells, germ cells, and combinations thereof. Such codon-optimized sequences are within the ambit of the ordinary skilled artisan in view of the description herein. In an embodiment, the polynucleotide is codon optimized for a specific tissue type. Such tissue types can include, but are not limited to, muscle tissue, connective tissue, nervous tissue, and epithelial tissue. Such codon-optimized sequences are within the ambit of the ordinary skilled artisan in view of the description herein. In an embodiment, the polynucleotide is codon optimized for a specific organ. Such organs include but are not limited to, muscles, skin, intestines, liver, spleen, brain, lungs, stomach, heart, kidneys, gallbladder, pancreas, bladder, thyroid, bone, blood vessels, blood, and combinations thereof. Such codon-optimized sequences are within the ambit of the ordinary skilled artisan in view of the description herein.

In an embodiment, a vector polynucleotide is codon optimized for expression in particular cells, such as prokaryotic or eukaryotic cells. The eukaryotic cells may be those of or derived from a particular organism, such as a plant or a mammal, including but not limited to human, or non-human eukaryote or animal or mammal as discussed herein, e.g., mouse, rat, rabbit, dog, livestock, or non-human mammal or primate.

### Vector Construction

The vectors described herein can be constructed using any suitable process or technique. In an embodiment, one or more suitable recombination and/or cloning methods or techniques can be used to design the vector(s) described herein. Suitable recombination and/or cloning techniques and/or methods can include but are not limited to, those described in U.S. Patent Publication No. US 2004/0171156 A1. Other suitable methods and techniques are described elsewhere herein.

Construction of recombinant AAV vectors is described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989). Any of the techniques and/or methods can be used and/or adapted for constructing an AAV or other vector described herein. nullAAV (nAAV) vectors are discussed elsewhere herein.

In an embodiment, a vector comprises one or more insertion sites, such as a restriction endonuclease recognition sequence (also referred to as a “cloning site”). In an embodiment, one or more insertion sites (e.g., about or more than about 1, 2, 3, 4, 5, 6, 7, 8, 9, 10, or more insertion sites) are located upstream and/or downstream of one or more sequence elements of one or more vectors. When multiple different guide polynucleotides are used, such in the context of a CRISPR-Cas system, a single expression construct may be used to target multiple different, corresponding target sequences within a cell. For example, a single vector may comprise about or more than about 1, 2, 3, 4, 5, 6, 7, 8, 9, 10, 15, 20, or more guide polynucleotides. In an embodiment, about or more than about 1, 2, 3, 4, 5, 6, 7, 8, 9, 10, or more guide-polynucleotide-containing vectors may be provided, and optionally delivered to a cell.

Delivery vehicles, vectors, particles, nanoparticles, formulations, and components thereof for expression of one or more elements of an engineered Acr polypeptide delivery system described herein are as used in the foregoing documents, such as International Patent Publication No. WO 2014/093622 (PCT/US2013/074667) and are discussed in greater detail herein.

### Viral Vectors

In an embodiment, the vector is a viral vector. The term art “viral vector” as used herein in this context refers to polynucleotide-based vectors that contain one or more elements from or based upon one or more elements of a virus that can be capable of expressing and packaging a polynucleotide, such as an engineered Acr polypeptide delivery system polynucleotide of the present invention, into a virus particle and producing said virus particle when used alone or with one or more other viral vectors (such as in a viral vector system). Viral vectors and systems thereof can be used for producing viral particles for delivery of and/or expression of one or more components of the engineered Acr polypeptide delivery system described herein. The viral vector can be part of a viral vector system involving multiple vectors. In an embodiment, systems incorporating multiple viral vectors can increase the safety of these systems. Suitable viral vectors can include retroviral-based vectors, lentiviral-based vectors, adenoviral-based vectors, adeno-associated vectors, helper-dependent adenoviral (HdAd) vectors, hybrid adenoviral vectors, herpes simplex virus-based vectors, poxvirus-based vectors, and Epstein-Barr virus-based vectors. Other embodiments of viral vectors and viral particles produced therefrom are described elsewhere herein. In an embodiment, the viral vectors are configured to produce replication incompetent viral particles for improved safety of these systems.

In certain embodiments, the virus structural component, which can be encoded by one or more polynucleotides in a viral vector or vector system, comprises one or more capsid proteins including an entire capsid. In certain embodiments, such as wherein a viral capsid comprises multiple copies of different proteins, the delivery system can provide one or more of the same protein or a mixture of such proteins. For example, AAV comprises 3 capsid proteins, VP1, VP2, and VP3, thus delivery systems of the invention can comprise one or more of VP1, and/or one or more of VP2, and/or one or more of VP3. Accordingly, the present invention is applicable to a virus within the family Adenoviridae, such as Atadenovirus, e.g., Ovine atadenovirus D, Aviadenovirus, e.g., Fowl aviadenovirus A, Ichtadenovirus, e.g., Sturgeon ichtadenovirus A, Mastadenovirus (which includes adenoviruses such as all human adenoviruses), e.g., Human mastadenovirus C, and Siadenovirus, e.g., Frog siadenovirus A. Target-specific AAV capsid variants can be used or selected. Non-limiting examples include capsid variants selected to bind to chronic myelogenous leukemia cells, human CD34 PBPC cells, breast cancer cells, cells of the lung, heart, dermal fibroblasts, melanoma cells, stem cells, glioblastoma cells, coronary artery endothelial cells, and keratinocytes. See, e.g., Buning et al, 2015, Current Opinion in Pharmacology 24, 94-104. From teachings herein and knowledge in the art as to modifications of adenovirus (see, e.g., U.S. Pat. Nos. 9,410,129, 7,344,872, 7,256,036, 6,911,199, 6,740,525; Matthews, “Capsid-Incorporation of Antigens into Adenovirus Capsid Proteins for a Vaccine Approach,” Mol Pharm, 8 (1): 3-11 (2011)), as well as regarding modifications of AAV, the skilled person can readily obtain a modified adenovirus that has a large payload protein. Such modified adenovirus systems may be advantageous for embodiments of an engineered Acr polypeptide delivery system or one or more components thereof that may, when considered alone or together, be payload larger than the capacity of a native AAV. As to the viruses related to adenovirus mentioned herein, as well as to the viruses related to AAV mentioned elsewhere herein, the teachings herein as to modifying adenovirus and AAV, respectively, can be applied to those viruses without undue experimentation from this disclosure and the knowledge in the art.

In an embodiment, the viral vector is configured such that when the cargo is packaged the cargo(s) (e.g., one or more components of the engineered Acr polypeptide delivery system, including but not limited to an engineered Acr polypeptide or polynucleotide), is external to the capsid or virus particle. In the sense that it is not inside the capsid (enveloped or encompassed with the capsid) but is externally exposed so that it can contact the target cellular component (e.g., DNA, RNA, proteins). In an embodiment, the viral vector is configured such that all the cargo(s) are contained within the capsid after packaging.

### Split Viral Vector Systems

In an embodiment, the engineered Acr polypeptide delivery system viral vector or vector system (be it a retroviral (e.g., AAV) or lentiviral vector) is designed so as to position the cargo(s) (e.g., one or more engineered Acr polypeptide delivery system components) at the internal surface of the capsid. Once formed the cargo(s) will fill most or all of the internal volume of the capsid. In other embodiments, the engineered Acr polypeptide delivery system or component thereof may be modified or divided so as to occupy less of the capsid internal volume. Accordingly, in certain embodiments, the engineered Acr polypeptide delivery system or component thereof (e.g., an engineered Acr delivery polypeptide) can be divided into two portions, one portion comprised of one viral particle or capsid and the second portion comprised in a second viral particle or capsid. In certain embodiments, by splitting the engineered Acr polypeptide delivery system or component thereof in two portions, space is made available to link one or more additional domains to one or both of the engineered Acr polypeptide delivery system components (e.g., engineered Acr polypeptide and pore-forming polypeptide) portions. Such systems can be referred to as “split vector systems” or, in the context of the present disclosure, a “split system,” a “split protein,” and the like. This split protein approach is also described elsewhere herein. When the concept is applied to a vector system, it thus describes putting pieces of the split proteins on different vectors thus reducing the payload of any one vector. This approach can facilitate the delivery of systems where the total system size is close to or exceeds the packaging capacity of the vector. This is independent of any regulation of the engineered Acr polypeptide delivery system or component thereof that can be achieved with a split system or split protein design.

Split-engineered Acr delivery polypeptide system proteins that can be incorporated into the AAV or other vectors described herein are set forth elsewhere herein and in documents incorporated herein by reference in further detail herein. In certain embodiments, each part of a split-engineered Acr delivery polypeptide is attached to a member of a specific binding pair, and when bound with each other, the members of the specific binding pair maintain the parts of the engineered Acr delivery polypeptide in proximity. In certain embodiments, each part of a split-engineered Acr delivery polypeptide or pore-forming protein is associated with an inducible binding pair. An inducible binding pair is one that is capable of being switched “on” or “off” by a protein or small molecule that binds to both members of the inducible binding pair. In general, according to the invention, engineered Acr delivery system polypeptides may preferably split between domains, leaving domains intact. Exemplary engineered Acr delivery polypeptides and pore-forming proteins are described in greater detail elsewhere herein.

### Retroviral and Lentiviral Vectors

Retroviral vectors can be composed of cis-acting long terminal repeats (LTRs) with packaging capacity for up to 6-10 kb of foreign sequence. The minimum cis-acting LTRs are those sufficient for replication and packaging of the vectors, which are then used to integrate the therapeutic gene into the target cell to provide permanent transgene expression. Suitable retroviral vectors for the engineered Acr delivery polypeptide systems and/or components thereof can include those based upon murine leukemia virus (MuLV), gibbon ape leukemia virus (GaLV), Simian immunodeficiency virus (SIV), human immunodeficiency virus (HIV), and combinations thereof (see, e.g., Buchscher et al., J. Virol. 66:2731-2739 (1992); Johann et al., J. Virol. 66:1635-1640 (1992); Sommnerfelt et al., Virol. 176:58-59 (1990); Wilson et al., J. Virol. 63:2374-2378 (1989); Miller et al., J. Virol. 65:2220-2224 (1991); PCT/US94/05700). The selection of a retroviral gene transfer system may, therefore, depend on the target tissue.

The tropism of a retrovirus can be altered by incorporating foreign envelope proteins, expanding the potential target population of target cells. Lentiviral vectors are retroviral vectors that are able to transduce or infect non-dividing cells and are described in greater detail elsewhere herein. A retrovirus can also be engineered to allow for conditional expression of the inserted transgene, such that only certain cell types are infected by the lentivirus.

Lentiviruses are complex retroviruses that have the ability to infect and express their genes in both mitotic and post-mitotic cells. Advantages of using a lentiviral approach can include the ability to transduce or infect non-dividing cells and their ability to typically produce high viral titers, which can increase the efficiency or efficacy of production and delivery. Suitable lentiviral vectors include, but are not limited to, human immunodeficiency virus (HIV)-based lentiviral vectors, feline immunodeficiency virus (FIV)-based lentiviral vectors, simian immunodeficiency virus (SIV)-based lentiviral vectors, Moloney Murine Leukaemia Virus (Mo-MLV), Visna-Maedi virus (VMV)-based lentiviral vector, caprine arthritis-encephalitis virus (CAEV)-based lentiviral vector, bovine immune deficiency virus (BIV)-based lentiviral vector, and Equine infectious anemia (EIAV)-based lentiviral vector. In an embodiment, an HIV-based lentiviral vector system can be used. In an embodiment, an FIV-based lentiviral vector system can be used.

In an embodiment, the lentiviral vector is an EIAV-based lentiviral vector or vector system. EIAV vectors have been used to mediate expression, packaging, and/or delivery in other contexts, such as for ocular gene therapy (see, e.g., Balagaan, J Gene Med 2006; 8:275-285). In another embodiment, RetinoStat®, (see, e.g., Binley et al., HUMAN GENE THERAPY 23:980-991 (September 2012)), which describes an equine infectious anemia virus-based lentiviral gene therapy vector that expresses angiostatic proteins endostatin and angiostatin that is delivered via a subretinal injection for the treatment of the wet form of age-related macular degeneration. Any of these vectors described in these publications can be modified for the elements of the engineered Acr delivery polypeptide system described herein.

In an embodiment, the lentiviral vector or vector system thereof can be a first-generation lentiviral vector or vector system thereof. First-generation lentiviral vectors can contain a large portion of the lentivirus genome, including the gag and pol genes, other additional viral proteins (e.g. VSV-G), and other accessory genes (e.g. vif, vprm vpu, nef, and combinations thereof), regulatory genes (e.g. tat and/or rev) as well as the gene of interest between the LTRs. First-generation lentiviral vectors can result in the production of virus particles that can be capable of replication in vivo, which may not be appropriate for some instances or applications.

In an embodiment, the lentiviral vector or vector system thereof can be a second-generation lentiviral vector or vector system thereof. Second-generation lentiviral vectors do not contain one or more accessory virulence factors and do not contain all components necessary for virus particle production on the same lentiviral vector. This can result in the production of a replication-incompetent virus particle and thus increase the safety of these systems over first-generation lentiviral vectors. In an embodiment, the second-generation vector lacks one or more accessory virulence factors (e.g. vif, vprm, vpu, nef, and combinations thereof). Unlike the first-generation lentiviral vectors, no single second-generation lentiviral vector includes all the features necessary to express and package a polynucleotide into a virus particle. In an embodiment, the envelope and packaging components are split between two different vectors, with the gag, pol, rev, and tat genes being contained on one vector, and the envelope proteins (e.g., VSV-G) are contained on a second vector. The gene of interest, its promoter, and LTRs can be included on a third vector that can be used in conjunction with the other two vectors (packaging and envelope vectors) to generate a replication-incompetent virus particle.

In an embodiment, the lentiviral vector or vector system thereof can be a third-generation lentiviral vector or vector system thereof. Third-generation lentiviral vectors and vector systems thereof have increased safety over first- and second-generation lentiviral vectors and systems thereof because, for example, the various components of the viral genome are split between two or more different vectors but used together in vitro to make virus particles, they can lack the tat gene (when a constitutively active promoter is included upstream of the LTRs), and they can include one or more deletions in the 3′LTR to create self-inactivating (SIN) vectors having disrupted promoter/enhancer activity of the LTR. In an embodiment, a third-generation lentiviral vector system can include (i) a vector plasmid that contains the polynucleotide of interest and upstream promoters that are flanked by the 5′ and 3′ LTRs, which can optionally include one or more deletions present in one or both of the LTRs to render the vector self-inactivating; (ii) a “packaging vector(s)” that can contain one or more genes involved in packaging a polynucleotide into a virus particle that is produced by the system (e.g. gag, pol, and rev) and upstream regulatory sequences (e.g. promoter(s)) to drive expression of the features present on the packaging vector, and (iii) an “envelope vector” that contains one or more envelope protein genes and upstream promoters. In certain embodiments, the third-generation lentiviral vector system can include at least two packaging vectors, with the gag-pol being present on a different vector than the rev gene.

In an embodiment, self-inactivating lentiviral vectors with an siRNA targeting a common exon shared by HIV tat/rev, a nucleolar-localizing TAR decoy, and an anti-CCR5-specific hammerhead ribozyme (see, e.g., DiGiusto et al. (2010) Sci Transl Med 2:36ra43) can be used/and or adapted to the engineered Acr delivery polypeptide system of the present invention.

In an embodiment, the pseudotype and infectivity or tropism of a lentivirus particle can be tuned by altering the type of envelope protein(s) included in the lentiviral vector or system thereof. As used herein, an “envelope protein” or “outer protein” means a protein exposed at the surface of a viral particle that is not a capsid protein. For example, envelope or outer proteins typically comprise proteins embedded in the envelope of the virus. In an embodiment, a lentiviral vector or vector system thereof can include a VSV-G envelope protein. VSV-G mediates viral attachment to a low-density lipoprotein (LDL) receptor (LDLR) or an LDLR family member present on a host cell, which triggers endocytosis of the viral particle by the host cell. Because LDLR is expressed by a wide variety of cells, viral particles expressing the VSV-G envelope protein can infect or transduce a wide variety of cell types. Other suitable envelope proteins can be incorporated based on the host cell that a user desires to be infected by a virus particle produced from a lentiviral vector or system thereof described herein and can include, but are not limited to, feline endogenous virus envelope protein (RD114) (see e.g., Hanawa et al. Molec. Ther. 2002 5 (3) 242-251), modified Sindbis virus envelope proteins (see e.g., Morizono et al. 2010. J. Virol. 84 (14) 6923-6934; Morizono et al. 2001. J. Virol. 75:8016-8020; Morizono et al. 2009. J. Gene Med. 11:549-558; Morizono et al. 2006 Virology 355:71-81; Morizono et al J. Gene Med. 11:655-663, Morizono et al. 2005 Nat. Med. 11:346-352), baboon retroviral envelope protein (see e.g., Girard-Gagnepain et al. 2014. Blood. 124:1221-1231); Tupaia paramyxovirus glycoproteins (see e.g., Enkirch T. et al., 2013. Gene Ther. 20:16-23); measles virus glycoproteins (see e.g., Funke et al. 2008. Molec. Ther. 16 (8): 1427-1436), rabies virus envelope proteins, MLV envelope proteins, Ebola envelope proteins, baculovirus envelope proteins, filovirus envelope proteins, hepatitis E1 and E2 envelope proteins, gp41 and gp120 of HIV, hemagglutinin, neuraminidase, M2 proteins of influenza virus, and combinations thereof.

In an embodiment, the tropism of the resulting lentiviral particle can be tuned by incorporating cell-targeting peptides into a lentiviral vector such that the cell-targeting peptides are expressed on the surface of the resulting lentiviral particle. In an embodiment, a lentiviral vector can contain an envelope protein that is fused to a cell-targeting protein (see e.g., Buchholz et al. 2015. Trends Biotechnol. 33:777-790; Bender et al. 2016. PLOS Pathog. 12 (e1005461); and Friedrich et al. 2013. Mol. Ther. 2013. 21:849-859.

In an embodiment, a split-intein-mediated approach to target lentiviral particles to a specific cell type can be used (see e.g., Chamoun-Emaneulli et al. 2015. Biotechnol. Bioeng. 112:2611-2617, Ramirez et al. 2013. Protein. Eng. Des. Sel. 26:215-233). In these embodiments, a lentiviral vector can contain one-half of a splicing-deficient variant of the naturally split intein from Nostoc punctiforme fused to a cell-targeting peptide and the same or different lentiviral vector can contain the other half of the split intein fused to an envelope protein, such as a binding-deficient, fusion-competent virus envelope protein. This can result in production of a virus particle from the lentiviral vector or vector system that includes a split intein that can function as a molecular Velcro linker to link the cell-binding protein to the pseudotyped lentivirus particle. This approach can be advantageous for use where surface-incompatibilities can restrict the use of, e.g., cell-targeting peptides.

In an embodiment, a covalent-bond-forming protein-peptide pair can be incorporated into one or more of the lentiviral vectors described herein to conjugate a cell-targeting peptide to the virus particle (see e.g., Kasaraneni et al. 2018. Sci. Reports (8) No. 10990). In an embodiment, a lentiviral vector can include an N-terminal PDZ domain of InaD protein (PDZ1) and its pentapeptide ligand (TEFCA) (SEQ ID NO: 105) from NorpA, which can conjugate the cell-targeting peptide to the virus particle via a covalent bond (e.g., a disulfide bond). In an embodiment, the PDZ1 protein can be fused to an envelope protein, which can optionally be binding deficient and/or fusion competent virus envelope protein and included in a lentiviral vector. In an embodiment, the TEFCA (SEQ ID NO: 105) can be fused to a cell-targeting peptide and the TEFCA-CPT fusion construct can be incorporated into the same or a different lentiviral vector as the PDZ1-envelope protein construct. During virus production, specific interaction between the PDZ1 and TEFCA (SEQ ID NO: 105) facilitates producing virus particles covalently functionalized with the cell targeting peptide and thus capable of targeting a specific cell type based upon a specific interaction between the cell targeting peptide and cells expressing its binding partner. This approach can be advantageous for use where surface-incompatibilities can restrict the use of, e.g., cell-targeting peptides.

Lentiviral vectors have been disclosed as in the treatment for Parkinson's Disease, see, e.g., US Patent Publication No. 20120295960 and U.S. Pat. Nos. 7,303,910 and 7,351,585. Lentiviral vectors have also been disclosed for the treatment of ocular diseases, see e.g., US Patent Publication Nos. 20060281180, 20090007284, US20110117189; US20090017543; US20070054961, US20100317109. Lentiviral vectors have also been disclosed for delivery to the brain, see, e.g., US Patent Publication Nos. US20110293571; US20110293571, US20040013648, US20070025970, US20090111106, and U.S. Pat. No. 7,259,015. Any of these systems or a variant thereof can be used to deliver an engineered Acr delivery polypeptide system polynucleotide described herein to a cell.

In an embodiment, a lentiviral vector system can include one or more transfer plasmids. Transfer plasmids can be generated from various other vector backbones and can include one or more features that can work with other retroviral and/or lentiviral vectors in the system that can, for example, improve safety of the vector and/or vector system, increase virial titers, and/or increase or otherwise enhance expression of the desired insert to be expressed and/or packaged into the viral particle. Suitable features that can be included in a transfer plasmid can include, but are not limited to, 5′LTR, 3′LTR, SIN/LTR, origin of replication (Ori), selectable marker genes (e.g., antibiotic resistance genes), Psi (Y), RRE (rev response element), cPPT (central polypurine tract), promoters, WPRE (woodchuck hepatitis post-transcriptional regulatory element), SV40 polyadenylation signal, pUC origin, SV40 origin, F1 origin, and combinations thereof.

In another embodiment, Cocal vesiculovirus envelope pseudotyped retroviral or lentiviral vector particles are contemplated (see, e.g., US Patent Publication No. 20120164118 assigned to the Fred Hutchinson Cancer Research Center). Cocal virus is in the Vesiculovirus genus and is a causative agent of vesicular stomatitis in mammals. Cocal virus was originally isolated from mites in Trinidad (Jonkers et al., Am. J. Vet. Res. 25:236-242 (1964)), and infections have been identified in Trinidad, Brazil, and Argentina from insects, cattle, and horses. Many of the vesiculoviruses that infect mammals have been isolated from naturally infected arthropods, suggesting that they are vector-borne. Antibodies to vesiculoviruses are common among people living in rural areas where the viruses are endemic and laboratory-acquired; infections in humans usually result in influenza-like symptoms. The Cocal virus envelope glycoprotein shares 71.5% identity at the amino acid level with VSV-G Indiana, and phylogenetic comparison of the envelope gene of vesiculoviruses shows that the Cocal virus is serologically distinct from, but most closely related to, VSV-G Indiana strains among the vesiculoviruses. See e.g., Jonkers et al., Am. J. Vet. Res. 25:236-242 (1964) and Travassos da Rosa et al., Am. J. Tropical Med. & Hygiene 33:999-1006 (1984). The Cocal vesiculovirus envelope pseudotyped retroviral vector particles may include for example, lentiviral, alpharetroviral, betaretroviral, gammaretroviral, deltaretroviral, and epsilonretroviral vector particles that may comprise retroviral Gag, Pol, and/or one or more accessory protein(s) and a Cocal vesiculovirus envelope protein. In certain embodiments, the Gag, Pol, and accessory proteins are lentiviral and/or gammaretroviral. In an embodiment, a retroviral vector can contain encoding polypeptides for one or more Cocal vesiculovirus envelope proteins such that the resulting viral or pseudoviral particles are Cocal vesiculovirus envelope pseudotyped.

### Adenoviral Vectors, Helper-Dependent Adenoviral Vectors, and Hybrid Adenoviral Vectors

In an embodiment, the vector can be an adenoviral vector. In an embodiment, the adenoviral vector can include elements such that the virus particle produced using the vector or system thereof can be serotype 2 or serotype 5. In an embodiment, the polynucleotide to be delivered via the adenoviral particle can be up to about 8 kb. Thus, in an embodiment, an adenoviral vector can include a DNA polynucleotide to be delivered that can range in size from about 0.001 kb to about 8 kb. Adenoviral vectors have been used successfully in several contexts (see e.g. Teramato et al. 2000. Lancet. 355:1911-1912; Lai et al. 2002. DNA Cell. Biol. 21:895-913; Flotte et al., 1996. Hum. Gene. Ther. 7:1145-1159; and Kay et al. 2000. Nat. Genet. 24:257-261.

In an embodiment, the vector can be a helper-dependent adenoviral vector or system thereof. These are also referred to in the art as “gutless” or “gutted” vectors and are a modified generation of adenoviral vectors (see, e.g., Thrasher et al. 2006. Nature. 443: E5-7). In certain embodiments of the helper-dependent adenoviral vector system, one vector (the helper) can contain all the viral genes required for replication but contains a conditional gene defect in the packaging domain. The second vector of the system can contain only the ends of the viral genome, one or more engineered Acr delivery polypeptide system polynucleotides, and the native packaging recognition signal, which can allow selective packaged release from the cells (see e.g., Cideciyan et al. 2009. N Engl J Med. 361:725-727). Helper-dependent adenoviral vector systems have been successful for gene delivery in several contexts (see e.g., Simonelli et al. 2010. J Am Soc Gene Ther. 18:643-650; Cideciyan et al. 2009. N Engl J Med. 361:725-727; Crane et al. 2012. Gene Ther. 19 (4): 443-452; Alba et al. 2005. Gene Ther. 12:18-S27; Croyle et al. 2005. Gene Ther. 12:579-587; Amalfitano et al. 1998. J. Virol. 72:926-933; and Morral et al. 1999. PNAS. 96:12816-12821). The techniques and vectors described in these publications can be adapted for the inclusion and delivery of the engineered Acr delivery polypeptide system polynucleotides described herein. In an embodiment, the polynucleotide to be delivered via the viral particle produced from a helper-dependent adenoviral vector or system thereof can be up to about 37 kb. Thus, in an embodiment, an adenoviral vector can include a DNA polynucleotide to be delivered that can range in size from about 0.001 kb to about 37 kb (see e.g., Rosewell et al. 2011. J. Genet. Syndr. Gene Ther. Suppl. 5:001).

In an embodiment, the vector is a hybrid-adenoviral vector or system thereof. Hybrid adenoviral vectors are composed of the high transduction efficiency of a gene-deleted adenoviral vector and the long-term genome-integrating potential of adeno-associated retroviruses, lentiviruses, and transposon-based gene transfer. In an embodiment, such hybrid vector systems can result in stable transduction and limited integration sites. See e.g., Balague et al. 2000. Blood. 95:820-828; Morral et al. 1998. Hum. Gene Ther. 9:2709-2716; Kubo and Mitani. 2003. J. Virol. 77 (5): 2964-2971; Zhang et al. 2013. PloS One. 8 (10) e76771; and Cooney et al. 2015. Mol. Ther. 23 (4): 667-674), whose techniques and vectors described therein can be modified and adapted for use in the engineered Acr delivery polypeptide system of the present invention. In an embodiment, a hybrid-adenoviral vector can include one or more features of a retrovirus and/or an adeno-associated virus. In an embodiment, the hybrid-adenoviral vector can include one or more features of a spuma retrovirus or foamy virus (FV). See e.g., Ehrhardt et al. 2007. Mol. Ther. 15:146-156 and Liu et al. 2007. Mol. Ther. 15:1834-1841, whose techniques and vectors described therein can be modified and adapted for use in the Acr delivery system of the present invention. Advantages of using one or more features from the FVs in the hybrid-adenoviral vector or system thereof can include the ability of the viral particles produced therefrom to infect a broad range of cells, a large packaging capacity as compared to other retroviruses, and the ability to persist in quiescent (non-dividing) cells. See also e.g. Ehrhardt et al. 2007. Mol. Ther. 156:146-156 and Shuji et al. 2011. Mol. Ther. 19:76-82, whose techniques and vectors described therein can be modified and adapted for use in the Acr delivery system of the present invention.

### Adeno Associated Viral (AAV) Vectors

In an embodiment, the vector, such as a vector that can include an Acr delivery system of the present invention and/or a CRISPR-Cas system can be an adeno-associated virus (AAV) vector. See, e.g., West et al., Virology 160:38-47 (1987); U.S. Pat. No. 4,797,368; WO 93/24641; Kotin, Human Gene Therapy 5:793-801 (1994); and Muzyczka, J. Clin. Invest. 94:1351 (1994). Although similar to adenoviral vectors in some of their features, AAVs have some deficiency in their replication and/or pathogenicity and thus can be safer than adenoviral vectors. In an embodiment, the AAV can integrate into a specific site on chromosome 19 of a human cell with no observable side effects. In an embodiment, the capacity of the AAV vector, system thereof, and/or AAV particles can be up to about 4.7 kb. In an embodiment such as those where a CRISPR-Cas system is delivered as a co-therapy, utilizing homologs of the Cas effector protein that are shorter than e.g., SpCas9 (˜4104 bp) can be utilized, such as those in Table 4.

TABLE 4
Exemplary shorter Cas effector homologs.

| Species | Cas9 Size (bp) |
|---|---|
| Corynebacterium diphtheriae | 3252 |
| Eubacterium ventriosum | 3321 |
| Streptococcus pasteurianus | 3390 |
| Lactobacillus farciminis | 3378 |
| Sphaerochaeta globus | 3537 |
| Azospirillum B510 | 3504 |
| Gluconacetobacter diazotrophicus | 3150 |
| Neisseria cinerea | 3246 |
| Roseburia intestinalis | 3420 |
| Parvibaculum lavamentivorans | 3111 |
| Staphylococcus aureus | 3159 |
| Nitratifractor salsuginis DSM 16511 | 3396 |
| Campylobacter lari CF89-12 | 3009 |
| Campylobacter jejuni | 2952 |
| Streptococcus thermophilus LMD-9 | 3396 |

The AAV vector or system thereof can include one or more regulatory molecules. In an embodiment, the regulatory molecules can be promoters, enhancers, repressors, and the like, which are described in greater detail elsewhere herein. In an embodiment, the AAV vector or system thereof can include one or more polynucleotides that can encode one or more regulatory proteins. In an embodiment, the one or more regulatory proteins can be selected from Rep78, Rep68, Rep52, Rep40, variants thereof, and combinations thereof.

The AAV vector or system thereof can include one or more polynucleotides that can encode one or more capsid proteins. The capsid proteins can be selected from VP1, VP2, VP3, and combinations thereof. The capsid proteins can be capable of assembling into a protein shell of the AAV virus particle. In an embodiment, the AAV capsid can contain 60 capsid proteins. In an embodiment, the ratio of VP1:VP2:VP3 in a capsid can be about 1:1:10.

In an embodiment, the AAV vector or system thereof can include one or more adenovirus helper factors or polynucleotides that can encode one or more adenovirus helper factors. Such adenovirus helper factors can include, but are not limited to, E1A, E1B, E2A, E4ORF6, and VA RNAs. In an embodiment, a producing host cell line expresses one or more of the adenovirus helper factors.

The AAV vector or system thereof can be configured to produce AAV particles having a specific serotype. In an embodiment, the serotype can be AAV-1, AAV-2, AAV-3, AAV-4, AAV-5, AAV-6, AAV-7, AAV-8, AAV-9 or any combinations thereof. In an embodiment, the AAV can be AAV-1, AAV-2, AAV-5 or any combination thereof. One can select the AAV serotype of the AAV with regard to the cells to be targeted; e.g., one can select AAV serotypes 1, 2, 5 or a hybrid capsid AAV-1, AAV-2, AAV-5 or any combination thereof for targeting brain and/or neuronal cells; and one can select AAV-4 for targeting cardiac tissue; and one can select AAV-8 for delivery to the liver. Thus, in an embodiment, an AAV vector or system thereof capable of producing AAV particles capable of targeting the brain and/or neuronal cells can be configured to generate AAV particles having serotypes 1, 2, 5 or a hybrid capsid AAV-1, AAV-2, AAV-5 or any combination thereof. In an embodiment, an AAV vector or system thereof capable of producing AAV particles capable of targeting cardiac tissue can be configured to generate an AAV particle having an AAV-4 serotype. In an embodiment, an AAV vector or system thereof capable of producing AAV particles capable of targeting the liver can be configured to generate an AAV having an AAV-8 serotype. In an embodiment, the AAV vector is a hybrid AAV vector or system thereof. Hybrid AAVs are AAVs that include genomes with elements from one serotype that are packaged into a capsid derived from at least one different serotype. For example, if it is the recombinant AAV2/5 (rAAV2/5) that is to be produced, and if the production method is based on the helper-free, transient transfection method discussed elsewhere herein, all plasmids but the RepCap (pRepCap) plasmid will be the same. In the RepCap plasmid, called pRep2/Cap5, the Rep gene is still derived from AAV-2, while the Cap gene is derived from AAV-5. The production scheme is the same as the above-mentioned approach for AAV-2 production. The resulting rAAV is called rAAV2/5, in which the genome is based on recombinant AAV-2, while the capsid is based on AAV-5. It is assumed the cell or tissue-tropism displayed by this AAV2/5 hybrid virus should be the same as that of AAV-5. This can be applied to generate other hybrid serotypes.

A tabulation of certain AAV serotypes as to these cells can be found in Grimm, D. et al, J. Virol. 82:5887-5911 (2008) at Table 3.

In an embodiment, the AAV vector or system thereof is configured as a “gutless” vector, similar to that described in connection with a retroviral vector. In an embodiment, the “gutless” AAV vector or system thereof can have the cis-acting viral DNA elements involved in genome amplification and packaging in linkage with the heterologous sequences of interest (e.g., the engineered Acr delivery system or component thereof, a CRISPR-Cas system polynucleotide(s) co-therapy, or any combination thereof).

In an embodiment, the AAV vectors are produced in insect cells, e.g., Spodoptera frugiperda Sf9 insect cells, grown in serum-free suspension culture. Serum-free insect cells can be purchased from commercial vendors, e.g., Sigma Aldrich (EX-CELL 405).

In an embodiment, an AAV vector or vector system can contain or consists essentially of one or more polynucleotides encoding one or more components of a CRISPR system, such as when included as a co-therapy. In an embodiment, the AAV vector or vector system can contain a plurality of cassettes comprising or consisting a first cassette comprising or consisting essentially of a promoter, a nucleic acid molecule encoding a CRISPR-associated (Cas) protein (putative nuclease or helicase proteins), e.g., a Cas protein and a terminator, and a two, or more, advantageously up to the packaging size limit of the vector, e.g., in total (including the first cassette) five, cassettes comprising or consisting essentially of a promoter, nucleic acid molecule encoding guide RNA (gRNA) and a terminator (e.g., each cassette schematically represented as Promoter-gRNA1-terminator, Promoter-gRNA2-terminator, . . . Promoter-gRNA (N)-terminator; where N is a number that can be inserted that is at an upper limit of the packaging size limit of the vector), or two or more individual rAAVs, each containing one or more than one cassette of a CRISPR system, e.g., a first rAAV containing the first cassette comprising or consisting essentially of a promoter, a nucleic acid molecule encoding Cas, e.g., a Cas and a terminator, and a second rAAV containing a plurality of cassettes comprising or consisting essentially of a promoter, nucleic acid molecule encoding guide RNA (gRNA) and a terminator (e.g., each cassette schematically represented as Promoter-gRNA1-terminator, Promoter-gRNA2-terminator, . . . Promoter-gRNA (N)-terminator; where N is a number that can be inserted that is at an upper limit of the packaging size limit of the vector). As rAAV is a DNA virus, the nucleic acid molecules in the herein discussion concerning AAV or rAAV are advantageously DNA. In an embodiment, the promoter is a tissue-specific promoter or another tissue-specific regulatory element. Suitable tissue-specific regulatory elements, including promoters, are described in greater detail elsewhere herein.

In another embodiment, the invention provides a non-naturally occurring or engineered Acr delivery system or component(s) thereof and/or CRISPR-Cas system protein or polynucleotide associated with Adeno Associated Virus (AAV), e.g., an AAV comprising an engineered Acr delivery system or component(s) thereof and/or CRISPR-Cas system protein or polynucleotide as a fusion, with or without a linker, to or with an AAV capsid protein such as VP1, VP2, and/or VP3. Incorporation of proteins in viral capsids is described in e.g., Rybniker et al., “Incorporation of Antigens into Viral Capsids Augments Immunogenicity of Adeno-Associated Virus Vector-Based Vaccines,” J Virol. December 2012; 86 (24): 13800-13804, Lux K, et al. 2005; Green fluorescent protein-tagged adeno-associated virus particles allow the study of cytosolic and nuclear trafficking. J. Virol. 79:11776-11787; Munch R C, et al. 2012. “Displaying high-affinity ligands on adeno-associated viral vectors enables tumor cell-specific and safe gene transfer.” Mol. Ther. [doi: 10.1038/mt.2012.186 and Warrington K H, Jr, et al. 2004. Adeno-associated virus type 2 VP2 capsid protein is nonessential and can tolerate large peptide insertions at its N terminus. J. Virol. 78:6595-6609, which can each be adapted for use with the present invention. It will be understood by those skilled in the art that the modifications described herein, if inserted into the AAV capsid gene (cap gene), may result in modifications in the VP1, VP2 and/or VP3 capsid subunits. Alternatively, the capsid subunits can be expressed independently to achieve modification in only one or two of the capsid subunits (VP1, VP2, VP3, VP1+VP2, VP1+VP3, or VP2+VP3). One can modify the cap gene to have expressed at a desired location a non-capsid protein, advantageously a large payload protein, such as a CRISPR-protein. Likewise, these can be fusions, with the protein, e.g., a large payload protein such as a CRISPR-protein fused in a manner analogous to prior art fusions. See, e.g., US Patent Publication 20090215879; Nance et al., “Perspective on Adeno-Associated Virus Capsid Modification for Duchenne Muscular Dystrophy Gene Therapy,” Hum Gene Ther. 26 (12): 786-800 (2015) and documents cited therein, incorporated herein by reference. The skilled person, from this disclosure and the knowledge in the art can make and use modified AAV or AAV capsid as in the herein invention, and through this disclosure, one knows now that large payload proteins can be fused to the AAV capsid. In an embodiment, the AAV-capsid recombinant AAVs contain proteins and/or nucleic acid molecule(s) encoding or providing an engineered Acr delivery system or component thereof and/or a CRISPR-Cas system or component thereof co-therapy to a cell. In an embodiment, the engineered Acr delivery system and/or CRISPR-Cas system co-therapy is/are assembled from the nucleic acid molecule(s) contained in the AAV and a protein component on a surface of the capsid, such as outer or inner surface. The instant invention is also applicable to a virus in the genus Dependoparvovirus or in the family Parvoviridae, for instance, AAV, or a virus of Amdoparvovirus, e.g., Carnivore amdoparvovirus 1, a virus of Aveparvovirus, e.g., Galliform aveparvovirus 1, a virus of Bocaparvovirus, e.g., Ungulate bocaparvovirus 1, a virus of Copiparvovirus, e.g., Ungulate copiparvovirus 1, a virus of Dependoparvovirus, e.g., Adeno-associated dependoparvovirus A, a virus of Erythroparvovirus, e.g., Primate erythroparvovirus 1, a virus of Protoparvovirus, e.g., Rodent protoparvovirus 1, a virus of Tetraparvovirus, e.g., Primate tetraparvovirus 1. Thus, a virus within the family Parvoviridae or the genus Dependoparvovirus or any of the other foregoing genera within Parvoviridae is contemplated as within the invention with discussion herein as to AAV applicable to such other viruses.

In an embodiment, an engineered Acr delivery system component and/or CRISPR-Cas system co-therapy component is external to the capsid or virus particle in the sense that it is not inside the capsid (enveloped or encompassed with the capsid), but is externally exposed so that it can contact the target cellular component (e.g., DNA, RNA, and/or protein). In an embodiment, an engineered Acr delivery system component and/or CRISPR-Cas system co-therapy component is associated with the AAV VP2 domain by way of a fusion protein. In an embodiment, the association may be considered to be a modification of the VP2 domain. In an embodiment, the AAV VP2 domain may be associated (or tethered) to an engineered Acr delivery system component and/or CRISPR-Cas system co-therapy component via a connector protein, for example using a system such as the streptavidin-biotin system. In an embodiment, the engineered Acr delivery system component and/or CRISPR-Cas system co-therapy component and associated AAV VP2 domain are encoded by a polynucleotide. In one embodiment, the invention provides a non-naturally occurring modified AAV having a VP2-engineered Acr delivery system component and/or CRISPR-Cas system co-therapy component capsid protein, wherein the engineered Acr delivery system component and/or CRISPR-Cas system co-therapy component is part of or tethered to the VP2 domain. In an embodiment, the engineered Acr delivery system component and/or CRISPR-Cas system co-therapy component is fused to the VP2 domain to produce a modified AAV having a VP2-engineered Acr delivery system component and/or CRISPR-Cas system co-therapy component fusion capsid protein. In an embodiment, the VP2-engineered Acr delivery system component and/or CRISPR-Cas system co-therapy component capsid protein further comprises a linker, whereby the VP2-engineered Acr delivery system component and/or CRISPR-Cas system co-therapy component is distanced from the remainder of the AAV. In an embodiment, the VP2-engineered Acr delivery system component and/or CRISPR-Cas system co-therapy component capsid protein further comprises at least one protein complex, e.g., CRISPR complex, such as a CRISPR-Cas complex guide RNA that targets a particular cellular polynucleotide target (e.g., a DNA or an RNA molecule), such as in a co-therapy.

In one embodiment, the invention provides a non-naturally occurring or engineered composition comprising an engineered Acr delivery system component and/or CRISPR-Cas system co-therapy component. In some of such embodiments, the engineered Acr delivery system component and/or CRISPR-Cas system co-therapy is part of or tethered to an AAV capsid domain, i.e., VP1, VP2, or VP3 domain of Adeno-Associated Virus (AAV) capsid. In an embodiment, part of an engineered Acr delivery system component and/or CRISPR-Cas system co-therapy component tethered to an AAV capsid domain is associated with an AAV capsid domain. In an embodiment, an engineered Acr delivery system component and/or CRISPR-Cas system co-therapy component may be fused to the AAV capsid domain. In an embodiment, the fusion may be to the N-terminal end of the AAV capsid domain. As such, in an embodiment, the C-terminal end of the engineered Acr delivery system component and/or CRISPR-Cas system co-therapy component is fused to the N-terminal end of the AAV capsid domain. In an embodiment, an NLS and/or a linker (such as a GlySer linker) may be positioned between the C-terminal end of the engineered Acr delivery system component and/or CRISPR-Cas system co-therapy component and the N-terminal end of the AAV capsid domain. In an embodiment, the fusion may be to the C-terminal end of the AAV capsid domain. In an embodiment, this is not preferred due to the fact that the VP1, VP2, and VP3 domains of AAV are alternative splices of the same RNA and so a C-terminal fusion may affect all three domains. In an embodiment, the AAV capsid domain is truncated. In an embodiment, some or all of the AAV capsid domain is removed. In an embodiment, some of the AAV capsid domain is removed and replaced with a linker (such as a GlySer linker), typically leaving the N-terminal and C-terminal ends of the AAV capsid domain intact, such as the first 2, 5, or 10 amino acids. In this way, the internal (non-terminal) portion of the VP3 domain may be replaced with a linker. In an embodiment, the linker is fused to the engineered Acr delivery system component and/or CRISPR-Cas system co-therapy component. A branched linker may be used. In such embodiments, an engineered Acr delivery system component and/or CRISPR-Cas system co-therapy component is fused to the end of one of the branches. Without being bound by theory, this allows for some degree of spatial separation between the capsid and the Acr delivery system component(s) and/or CRISPR-Cas protein. In this way, the Acr delivery system component(s) and/or CRISPR-Cas protein is part of (or fused to) the AAV capsid domain.

In other embodiments, the engineered Acr delivery system component and/or CRISPR-Cas system co-therapy component may be fused in frame within, e.g., internal to, the AAV capsid domain. Thus, in an embodiment, the AAV capsid domain again preferably retains its N-terminal and C-terminal ends. In this case, a linker is preferred, in an embodiment, either at one or both ends of the engineered Acr delivery system component and/or CRISPR-Cas system co-therapy component. In this way, the engineered Acr delivery system component and/or CRISPR-Cas system co-therapy component is again part of (or fused to) the AAV capsid domain. In certain embodiments, the positioning of the CRISPR enzyme is such that the engineered Acr delivery system component and/or CRISPR-Cas system co-therapy component is at the external surface of the viral capsid once formed. In one embodiment, the invention provides a non-naturally occurring or engineered composition comprising an engineered Acr delivery system component and/or CRISPR-Cas system co-therapy component associated with an AAV capsid domain of the AAV capsid. In this context, “associated” refers to an embodiment to fused, or an embodiment bound to, or an embodiment tethered to. The engineered Acr delivery system component and/or CRISPR-Cas system co-therapy component may, in an embodiment, be tethered to the VP1, VP2, or VP3 domain. This may be via a connector protein or tethering system such as the biotin-streptavidin system. In one example, a biotinylation sequence (15 amino acids) could, therefore, be fused to an engineered Acr delivery system component and/or CRISPR-Cas system co-therapy component protein. When a fusion of the AAV capsid domain, especially the N-terminus of the AAV capsid domain, with streptavidin, is also provided, the two will, therefore, associate with very high affinity. Thus, in an embodiment, provided is a composition or system comprising an engineered Acr delivery system component and/or CRISPR-Cas system co-therapy component-biotin fusion and a streptavidin-AAV capsid domain arrangement, such as a fusion. The engineered Acr delivery system component and/or CRISPR-Cas system co-therapy component-biotin and streptavidin-AAV capsid domain forms a single complex when the two parts are brought together. NLSs may also be incorporated between the engineered Acr delivery system component and/or CRISPR-Cas system co-therapy component and the biotin; and/or between the streptavidin and the AAV capsid domain.

As such, provided is a fusion of an engineered Acr delivery system component and/or CRISPR-Cas system co-therapy component with a connector protein specific for a high-affinity ligand for that connector, whereas the AAV VP2 domain is bound to said high-affinity ligand. For example, streptavidin may be the connector fused to the engineered Acr delivery system component and/or CRISPR-Cas system co-therapy component, while biotin may be bound to the AAV VP2 domain. Upon co-localization, the streptavidin will bind to the biotin, thus connecting the engineered Acr delivery system component and/or CRISPR-Cas system co-therapy component to the AAV VP2 domain. The reverse arrangement is also possible. In an embodiment, a biotinylation sequence (15 amino acids) could, therefore, be fused to the AAV VP2 domain, especially the N-terminus of the AAV VP2 domain. A fusion of an engineered Acr delivery system component and/or CRISPR-Cas system co-therapy component with streptavidin is also preferred, in an embodiment. In an embodiment, the biotinylated AAV capsids with streptavidin-engineered Acr delivery system component(s) and/or CRISPR-Cas system co-therapy component(s) are assembled in vitro. This way, the AAV capsids should assemble in a straightforward manner, and the engineered Acr delivery system component and/or CRISPR-Cas system co-therapy component-streptavidin fusion can be added after the assembly of the capsid. In other embodiments, a biotinylation sequence (15 amino acids) could, therefore, be fused to the engineered Acr delivery system component and/or CRISPR-Cas system co-therapy component, together with a fusion of the AAV VP2 domain, especially the N-terminus of the AAV VP2 domain, with streptavidin. For simplicity, a fusion of the engineered Acr delivery system component and/or CRISPR-Cas system co-therapy component and the AAV VP2 domain is preferred in an embodiment. In an embodiment, the fusion may be to the N-terminal end of the engineered Acr delivery system component and/or CRISPR-Cas system co-therapy component. In other words, in an embodiment, the AAV and engineered Acr delivery system component and/or CRISPR-Cas system co-therapy component are associated via fusion. In an embodiment, the AAV and engineered Acr delivery system component and/or CRISPR-Cas system co-therapy component are associated via fusion including a linker. Suitable linkers are discussed herein but include Gly Ser linkers. Fusion to the N-terminus of AAV VP2 domain is preferred, in an embodiment. In an embodiment, an engineered Acr delivery system component and/or CRISPR-Cas system co-therapy component comprises at least one Nuclear Localization Signal (NLS). In a further embodiment, the present invention provides compositions comprising the engineered Acr delivery system component and/or CRISPR-Cas system co-therapy component and associated AAV VP2 domain or the polynucleotides or vectors described herein. Such compositions and formulations are discussed elsewhere herein.

An alternative tether may be to fuse or otherwise associate the AAV capsid domain to an adaptor protein that binds to or recognizes a corresponding RNA sequence or motif. In an embodiment, the adaptor is or comprises a binding protein that recognizes and binds (or is bound by) an RNA sequence specific to said binding protein. In an embodiment, a preferred example is the MS2 (see Konermann et al. Nature 517 (7536): 583-588 (2015), cited infra, incorporated herein by reference) binding protein which recognizes and binds (or is bound by) an RNA sequence specific for the MS2 protein. In an embodiment, the RNA sequence specific for a binding protein is a gRNA that can bind to a Cas protein.

With the AAV capsid domain associated with the adaptor protein, an engineered Acr delivery system component and/or CRISPR-Cas system co-therapy component may, in an embodiment, be tethered to the adaptor protein of the AAV capsid domain. The engineered Acr delivery system component and/or CRISPR-Cas system co-therapy component may, in an embodiment, be tethered to the adaptor protein of the AAV capsid domain via the engineered Acr delivery system component and/or CRISPR-Cas system co-therapy component being in a complex with a modified guide, see Konermann et al. Id. The modified guide is, in an embodiment, a sgRNA. In an embodiment, the modified guide comprises a distinct RNA sequence; see, e.g., International Patent Application No. PCT/US14/70175, incorporated herein by reference. In an embodiment, the distinct RNA sequence is an aptamer. Thus, corresponding aptamer-adaptor protein systems are preferred. One or more functional domains may also be associated with the adaptor protein. An example of a preferred arrangement would be: [AAV capsid domain-adaptor protein]-[modified guide-CRISPR protein and/or Acr delivery system component].

In certain embodiments, the positioning of the engineered Acr delivery system component and/or CRISPR-Cas system co-therapy component is such that the engineered Acr delivery system component and/or CRISPR-Cas system co-therapy component is at the internal surface of the viral capsid once formed. In one embodiment, the invention provides a non-naturally occurring or engineered composition comprising an engineered Acr delivery system component and/or CRISPR-Cas system co-therapy component associated with an internal surface of an AAV capsid domain. Here again, associated may mean in an embodiment fused, or in an embodiment bound to, or in an embodiment tethered to. The engineered Acr delivery system component and/or CRISPR-Cas system co-therapy component may, in an embodiment, be tethered to the VP1, VP2, or VP3 domain such that it is located on the internal surface of the viral capsid once formed. This may be via a connector protein or tethering system such as the biotin-streptavidin system as described above and/or elsewhere herein.

In one embodiment, a co-therapy can include a non-naturally occurring CRISPR-Cas system comprising an AAV-Cas protein and a guide RNA that targets a DNA molecule encoding a gene product in a cell, whereby the guide RNA targets the DNA molecule encoding the gene product and the Cas protein cleaves the DNA molecule encoding the gene product, whereby expression of the gene product is altered; and, wherein the Cas protein and the guide RNA do not naturally occur together. The invention comprehends the guide RNA comprising a guide sequence fused to a Trans-activating CRISPR (tracr) sequence. In a preferred embodiment, the Cas protein is a Cas9, a Cas13, or a Cas12 protein. Other suitable Cas proteins are described elsewhere herein. In an embodiment, the polynucleotide encoding the Cas protein is codon optimized for expression in a eukaryotic cell. In an embodiment, the eukaryotic cell is a mammalian cell, and in a more preferred embodiment, the mammalian cell is a human cell. In a further embodiment, the expression of the gene product is decreased.

In another embodiment, a co-therapy comprises a non-naturally occurring vector system comprising one or more vectors comprising a first regulatory element operably linked to a CRISPR-Cas system guide RNA that targets a DNA molecule encoding a gene product and an AAV-Cas protein. The components may be located on the same or different vectors of the system or may be the same vector whereby the AAV-Cas protein also delivers the RNA of the CRISPR system. The guide RNA targets the DNA molecule encoding the gene product in a cell and the AAV-Cas protein may cleave the DNA molecule encoding the gene product (it may cleave one or both strands or have substantially no nuclease activity), whereby expression of the gene product is altered; and, wherein the AAV-Cas protein and the guide RNA do not naturally occur together. The invention comprehends the guide RNA comprising a guide sequence fused to a tracr sequence. In an embodiment of the invention, the AAV-Cas protein is a type II AAV-CRISPR-Cas protein, and in an embodiment, the AAV-Cas protein is an AAV-Cas9, AAV-Cas12, or AAV-Cas13 protein. The invention further comprehends the coding for the AAV-Cas protein being codon optimized for expression in a eukaryotic cell. In a preferred embodiment, the eukaryotic cell is a mammalian cell and in a more preferred embodiment, the mammalian cell is a human cell. In a further embodiment of the invention, the expression of the gene product is decreased.

In one embodiment, the invention provides a vector system comprising one or more vectors. In an embodiment, the system comprises a CRISPR-Cas co-therapy that comprises: (a) a first regulatory element operably linked to a tracr mate sequence and one or more insertion sites for inserting one or more guide sequences upstream of the tracr mate sequence, wherein when expressed, the guide sequence directs sequence-specific binding of an AAV-CRISPR complex to a target sequence in a eukaryotic cell, wherein the CRISPR complex comprises a AAV-CRISPR enzyme complexed with (1) the guide sequence that is hybridized to the target sequence, and (2) the tracr mate sequence that is hybridized to the tracr sequence; and (b) said AAV-CRISPR enzyme comprising at least one nuclear localization sequence and/or at least one nuclear export signal (NES); wherein components (a) and (b) are located on or in the same or different vectors of the system. In an embodiment, component (a) further comprises the tracr sequence downstream of the tracr mate sequence under the control of the first regulatory element. In an embodiment, component (a) further comprises two or more guide sequences operably linked to the first regulatory element, wherein when expressed, each of the two or more guide sequences direct sequence-specific binding of an AAV-CRISPR complex to a different target sequence in a eukaryotic cell. In an embodiment, the system comprises the tracr sequence under the control of a third regulatory element, such as a polymerase III promoter. In an embodiment, the tracr sequence exhibits at least 50%, 60%, 70%, 80%, 90%, 95%, or 99% of sequence complementarity along the length of the tracr mate sequence when optimally aligned. Determining optimal alignment is within the purview of one of skill in the art. For example, there are publicly and commercially available alignment algorithms and programs such as, but not limited to, ClustalW, Smith-Waterman in Matlab, Bowtie, Geneious, Biopython, and SeqMan. In an embodiment, the AAV-CRISPR complex comprises one or more nuclear localization sequences of sufficient strength to drive the accumulation of said CRISPR complex in a detectable amount in the nucleus of a eukaryotic cell. Without wishing to be bound by theory, it is believed that a nuclear localization sequence is not necessary for AAV-CRISPR complex activity in eukaryotes but that including such sequences enhances the activity of the system, especially as to targeting nucleic acid molecules in the nucleus and/or having molecules exit the nucleus. In an embodiment, the AAV-CRISPR enzyme is an AAV-Cas enzyme. In an embodiment, the AAV-Cas enzyme is derived from S. pneumoniae, S. pyogenes, S. thermophiles, F. novicida or S. aureus Cas9, Cas12 (e.g., Cas12a), Cas13, etc. (e.g., a Cas protein of one of these organisms modified to have or be associated with at least one AAV) and may include further mutations or alterations or be a chimeric Cas9. The enzyme may be an AAV-Cas9 homolog or ortholog. In an embodiment, the AAV-CRISPR enzyme is codon-optimized for expression in a eukaryotic cell. In an embodiment, the AAV-CRISPR enzyme directs the cleavage of one or two strands at the location of the target sequence. In an embodiment, the AAV-CRISPR enzyme lacks DNA strand cleavage activity. In an embodiment, the first regulatory element is a polymerase III promoter. In an embodiment, the second regulatory element is a polymerase II promoter. In an embodiment, the guide sequence is at least 15, 16, 17, 18, 19, 20, 25 nucleotides, or between 10-30, or between 15-25, or between 15-20 nucleotides in length.

In general, in an embodiment, the AAV further comprises a repair template. It will be appreciated that comprises in the phrase “the virus comprises . . . ”, “the AAV comprises . . . ”, “the lentiviral vector LVV0”, “the LVV comprises”, and/or the like may mean encompassed within the viral capsid or that the virus encodes the comprised protein or polynucleotide such as a repair template, gRNA, mRNA, and/or the like. In an embodiment, one or more, preferably two or more guide RNAs, may be comprised/encompassed within the AAV vector. Two may be preferred, in an embodiment, as it allows for multiplexing or dual nickase approaches. Particularly for multiplexing, two or more guides may be used. In fact, in an embodiment, three or more, four or more, five or more, or even six or more guide RNAs may be comprised/encompassed within the AAV. More space has been freed up within the AAV by virtue of the fact that the AAV no longer needs to comprise/encompass the CRISPR enzyme. In each of these instances, a repair template may also be provided comprised/encompassed within the AAV. In an embodiment, the repair template corresponds to or includes the DNA target.

### Herpes Simplex Viral Vectors

In an embodiment, the vector can be a Herpes Simplex Viral (HSV)-based vector or system thereof. HSV systems can include the disabled infections single copy (DISC) viruses, which are composed of a glycoprotein H defective mutant HSV genome. When the defective HSV is propagated in complementing cells, virus particles can be generated that are capable of infecting subsequent cells, permanently replicating their own genome but are not capable of producing more infectious particles. See e.g., 2009. Trobridge. Exp. Opin. Biol. Ther. 9:1427-1436, whose techniques and vectors described therein can be modified and adapted for use in the engineered Acr delivery system and/or CRISPR-Cas co-therapy. In an embodiment where an HSV vector or system thereof is utilized, the host cell can be a complementing cell. In an embodiment, the HSV vector or system thereof can be capable of producing virus particles capable of delivering a polynucleotide cargo of up to 150 kb. Thus, in some embodiment, the engineered Acr delivery system and/or CRISPR-Cas co-therapy polynucleotide(s) included in the HSV-based viral vector or system thereof can sum from about 0.001 to about 150 kb. HSV-based vectors and systems thereof have been successfully used in several contexts including various models of neurologic disorders. See e.g., Cockrell et al. 2007. Mol. Biotechnol. 36:184-204; Kafri T. 2004. Mol. Biol. 246:367-390; Balaggan and Ali. 2012. Gene Ther. 19:145-153; Wong et al. 2006. Hum. Gen. Ther. 2002. 17:1-9; Azzouz et al. J. Neruosci. 22L10302-10312; and Betchen and Kaplitt. 2003. Curr. Opin. Neurol. 16:487-493, whose techniques and vectors described therein can be modified and adapted for use in the engineered Acr delivery system and/or CRISPR-Cas co-therapy.

### Poxvirus Vectors

In an embodiment, the vector can be a poxvirus vector or a system thereof. In an embodiment, the poxvirus vector can result in cytoplasmic expression of one or more engineered Acr delivery systems and/or CRISPR-Cas co-therapy polynucleotides described herein. In an embodiment, the capacity of a poxvirus vector or system thereof can be about 25 kb or more. In an embodiment, a poxvirus vector or system thereof can include one or more CRISPR-Cas system polynucleotides described herein.

### Viral Vectors for Delivery to Plants

The systems and compositions may be delivered to plant cells using viral vehicles. In particular embodiments, the compositions and systems may be introduced in the plant cells using a plant viral vector (e.g., as described in Scholthof et al. 1996, Annu Rev Phytopathol. 1996; 34:299-323). Such viral vector may be a vector from a DNA virus, e.g., geminivirus (e.g., cabbage leaf curl virus, bean yellow dwarf virus, wheat dwarf virus, tomato leaf curl virus, maize streak virus, tobacco leaf curl virus, or tomato golden mosaic virus) or nanovirus (e.g., Faba bean necrotic yellow virus). The viral vector may be a vector from an RNA virus, e.g., tobravirus (e.g., tobacco rattle virus, tobacco mosaic virus), potexvirus (e.g., potato virus X), or hordeivirus (e.g., barley stripe mosaic virus). The replicating genomes of plant viruses may be non-integrative vectors.

### Virus-Like Particles and Vectors

In an embodiment, the vector is a vector that is capable of generating virus-like particles (VLPs). VLPs is a term of art that refers to particles produced from virus proteins, such as capsid or other proteins, but that do not contain the native viral genetic materials. Exemplary VLPs and their production systems and vectors for delivery of an engineered Acr delivery system described herein are described in e.g., Bhat et al., Viruses 14 (2): 383 (2022) doi: 10.3390/v14020383; Hill et al., Curr Protein Pept Sci. (2018) 19 (1): 112-127; Schwarz B et al., Adv Virus Res. 2017. 97:1-60 doi: 10.1016/bs.aivir.2016.09.002; Banskota et al., Cell. 2022. 185 (2): 250-265; Ikwuagwu and Tullman-Ercek. Curr Opin Biotechnol. 2022. 78:102785 doi: 10.1016/j.copbio.2022.102785; Zdanowicz and Chroboczek. Acta Biochim Pol. 2016: 63 (3): 469-473; Suffian and Al-Jamal et al., Adv. Drug Deliv. Rev. 2022. 180:114030 doi: 10.1016/j.addr.2021.114030; and Segel et al., Science. 373:6557 (2021).

Virus Particle Production from Viral Vectors

### Retroviral Production

In an embodiment, one or more viral vectors and/or systems thereof can be delivered to a suitable cell line for the production of virus particles containing the polynucleotide or other payload to be delivered to a host cell. Suitable host cells for virus production from viral vectors and systems thereof described herein are known in the art and are commercially available. For example, suitable host cells include HEK 293 cells and their variants (HEK 293T and HEK 293TN cells). In an embodiment, the suitable host cell for virus production from viral vectors and systems thereof described herein can stably express one or more genes involved in packaging (e.g. pol, gag, and/or VSV-G) and/or other supporting genes.

In an embodiment, after delivery of one or more viral vectors to the suitable host cells for virus production from viral vectors and systems thereof, the cells are incubated for an appropriate length of time to allow for viral gene expression from the vectors, packaging of the polynucleotide to be delivered (e.g., an invention engineered Acr delivery system and/or CRISPR-Cas co-therapy polynucleotide), and virus particle assembly, and secretion of mature virus particles into the culture media. Various other methods and techniques are generally known to those of ordinary skill in the art.

Mature virus particles can be collected from the culture media by a suitable method. In an embodiment, this can involve centrifugation to concentrate the virus. The titer of the composition containing the collected virus particles can be obtained using a suitable method. Such methods can include transducing a suitable cell line (e.g. NIH 3T3 cells) and determining transduction efficiency and infectivity in that cell line by a suitable method. Suitable methods include PCR-based methods, flow cytometry, and antibiotic selection-based methods. Various other methods and techniques are generally known to those of ordinary skill in the art. The concentration of virus particles can be adjusted as needed. In an embodiment, the resulting composition containing virus particles can contain 1×10¹-1×10²⁰ particles/mL.

Lentiviruses may be prepared from any lentiviral vector or vector system described herein. In one example embodiment, after cloning pCasES10 (which contains a lentiviral transfer plasmid backbone), HEK293FT at low passage (p=5) can be seeded in a T-75 flask to 50% confluence the day before transfection in DMEM with 10% fetal bovine serum and without antibiotics. After 20 hours, the media can be changed to OptiMEM (serum-free) media, and transfection of the lentiviral vectors can be done 4 hours later. Cells can be transfected with 10 μg of lentiviral transfer plasmid (pCasES10) and the appropriate packaging plasmids (e.g., 5 μg of pMD2.G (VSV-g pseudotype), and 7.5 μg of psPAX2 (gag/pol/rev/tat)). Transfection can be carried out in 4 mL OptiMEM with a cationic lipid delivery agent (50 μL Lipofectamine 2000 and 100 μl Plus reagent). After 6 hours, the media can be changed to antibiotic-free DMEM with 10% fetal bovine serum. These methods can use serum during cell culture, but serum-free methods are preferred.

Following transfection and allowing the producing cells (also referred to as packaging cells) to package and produce virus particles with packaged cargo, the lentiviral particles can be purified. In an exemplary embodiment, virus-containing supernatants can be harvested after 48 hours. Collected virus-containing supernatants can first be cleared of debris and filtered through a 0.45 μm low protein binding (PVDF) filter. They can then be spun in an ultracentrifuge for 2 hours at 24,000 rpm. The resulting virus-containing pellets can be resuspended in 50 μl of DMEM overnight at 4 degrees C. They can be then aliquoted and used immediately or immediately frozen at −80 degrees C. for storage.

### AAV Particle Production

There are two main strategies for producing AAV particles from AAV vectors and systems thereof, such as those described herein, which depend on how the adenovirus helper factors are provided (helper- v. helper-free). In an embodiment, a method of producing AAV particles from AAV vectors and systems thereof can include adenovirus infection into cell lines that stably harbor AAV replication and capsid encoding polynucleotides along with AAV vector containing the polynucleotide to be packaged and delivered by the resulting AAV particle (e.g. the engineered Acr delivery system and/or CRISPR-Cas system polynucleotide(s)). In an embodiment, a method of producing AAV particles from AAV vectors and systems thereof can be a “helper-free” method, which includes co-transfection of an appropriate producing cell line with three vectors (e.g. plasmid vectors): (1) an AAV vector that contains a polynucleotide of interest (e.g. the engineered Acr delivery system and/or CRISPR-Cas system polynucleotide(s)) between 2 ITRs; (2) a vector that carries the AAV Rep-Cap encoding polynucleotides; and (3) a vector that carries helper polynucleotides. One of ordinary skill in the art will appreciated that various methods and variations thereof that are both helper- and helper-free and as well as the different advantages of each system.

### Non-Viral Vectors

In an embodiment, the vector is a non-viral vector or vector system. The term of art “Non-viral vector” and as used herein in this context refers to molecules and/or compositions that are vectors but that are not based on one or more components of a virus or virus genome (excluding any nucleotide to be delivered and/or expressed by the non-viral vector) that can be capable of incorporating engineered Acr delivery system polynucleotide(s) and/or CRISPR-Cas polynucleotide(s) and delivering said engineered Acr delivery system polynucleotide(s) and/or CRISPR-Cas polynucleotide(s) to a cell and/or expressing the polynucleotide in the cell. It will be appreciated that this does not exclude vectors containing a polynucleotide designed to target a virus-based polynucleotide that is to be delivered. For example, if a gRNA to be delivered is directed against a virus component and it is inserted or otherwise coupled to an otherwise non-viral vector or carrier, this would not make said vector a “viral vector”. Non-viral vectors can include, without limitation, naked polynucleotides and polynucleotide (non-viral) based vector and vector systems.

### Naked Polynucleotides

In an embodiment, one or more engineered Acr delivery system polynucleotide(s) and/or CRISPR-Cas system polynucleotides described elsewhere herein can be included in a naked polynucleotide. The term of art “naked polynucleotide” as used herein refers to polynucleotides that are not associated with another molecule (e.g., proteins, lipids, and/or other molecules) that can often help protect it from environmental factors and/or degradation. As used herein, associated with includes, but is not limited to, linked to, adhered to, adsorbed to, enclosed in, enclosed in or within, mixed with, and the like. Naked polynucleotides that include one or more of the engineered Acr delivery system polynucleotide(s) and/or CRISPR-Cas system polynucleotides described herein can be delivered directly to a host cell and optionally expressed therein. The naked polynucleotides can have any suitable two- and three-dimensional configurations. By way of non-limiting examples, naked polynucleotides can be single-stranded molecules, double-stranded molecules, circular molecules (e.g., plasmids and artificial chromosomes), molecules that contain portions that are single-stranded and portions that are double-stranded (e.g. ribozymes), and the like. In an embodiment, the naked polynucleotide contains only the engineered Acr delivery system polynucleotide(s) and/or CRISPR-Cas system polynucleotide(s) of the present invention. In an embodiment, the naked polynucleotide can contain other nucleic acids and/or polynucleotides in addition to the engineered Acr delivery system polynucleotide(s) and/or CRISPR-Cas system polynucleotide(s) of the present invention. The naked polynucleotides can include one or more elements of a transposon system. Transposons and systems thereof are described in greater detail elsewhere herein.

### Non-Viral Polynucleotide Vectors

In an embodiment, one or more of the engineered Acr delivery system polynucleotide(s) and/or CRISPR-Cas system polynucleotides can be included in a non-viral polynucleotide vector. Suitable non-viral polynucleotide vectors include, but are not limited to, transposon vectors and vector systems, plasmids, bacterial artificial chromosomes, yeast artificial chromosomes, AR (antibiotic resistance)-free plasmids and miniplasmids, circular covalently closed vectors (e.g. minicircles, minivectors, miniknots), linear covalently closed vectors (“dumbbell-shaped”), MIDGE (minimalistic immunologically defined gene expression) vectors, MiLV (micro-linear vector) vectors, Ministrings, mini-intronic plasmids, PSK systems (post-segregationally killing systems), ORT (operator repressor titration) plasmids, and the like. See e.g., Hardee et al. 2017. Genes. 8 (2): 65.

In an embodiment, the non-viral polynucleotide vector can have a conditional origin of replication. In an embodiment, the non-viral polynucleotide vector can be an ORT plasmid. In an embodiment, the non-viral polynucleotide vector can have a minimalistic immunologically defined gene expression. In an embodiment, the non-viral polynucleotide vector can have one or more post-segregationally killing system genes. In an embodiment, the non-viral polynucleotide vector is AR-free. In an embodiment, the non-viral polynucleotide vector is a minivector. In an embodiment, the non-viral polynucleotide vector includes a nuclear localization signal. In an embodiment, the non-viral polynucleotide vector can include one or more CpG motifs. In an embodiment, the non-viral polynucleotide vectors can include one or more scaffold/matrix attachment regions (S/MARs). See e.g. Mirkovitch et al. 1984. Cell. 39:223-232, Wong et al. 2015. Adv. Genet. 89:113-152, whose techniques and vectors can be adapted for use in the present invention. S/MARs are AT-rich sequences that play a role in the spatial organization of chromosomes through DNA loop base attachment to the nuclear matrix. S/MARs are often found close to regulatory elements such as promoters, enhancers, and origins of DNA replication. The inclusion of one or more S/MARs can facilitate a once-per-cell-cycle replication to maintain the non-viral polynucleotide vector as an episome in daughter cells. In certain embodiments, the S/MAR sequence is located downstream of an actively transcribed polynucleotide (e.g. one or more Acr delivery system polynucleotide(s) and/or CRISPR-Cas system polynucleotide(s) co-therapy of the present invention) included in the non-viral polynucleotide vector. In an embodiment, the S/MAR can be a S/MAR from the beta-interferon gene cluster. See e.g. Verghese et al. 2014. Nucleic Acid Res. 42: e53; Xu et al. 2016. Sci. China Life Sci. 59:1024-1033; Jin et al. 2016. 8:702-711; Koirala et al. 2014. Adv. Exp. Med. Biol. 801:703-709; and Nehlsen et al. 2006. Gene Ther. Mol. Biol. 10:233-244, whose techniques and vectors can be adapted for use in the present invention.

In an embodiment, the non-viral vector is a transposon vector or system thereof. As used herein, “transposon” (also referred to as transposable element) refers to a polynucleotide sequence that is capable of moving from one location in a genome to another. There are several classes of transposons. Transposons include retrotransposons and DNA transposons. Retrotransposons require the transcription of the polynucleotide that is moved (or transposed) in order to transpose the polynucleotide to a new genome or polynucleotide. DNA transposons are those that do not require reverse transcription of the polynucleotide that is moved (or transposed) in order to transpose the polynucleotide to a new genome or polynucleotide. In an embodiment, the non-viral polynucleotide vector can be a retrotransposon vector. In an embodiment, the retrotransposon vector includes long terminal repeats. In an embodiment, the retrotransposon vector does not include long terminal repeats. In an embodiment, the non-viral polynucleotide vector can be a DNA transposon vector. DNA transposon vectors can include a polynucleotide sequence encoding a transposase. In an embodiment, the transposon vector is configured as a non-autonomous transposon vector, meaning that the transposition does not occur spontaneously on its own. In some of these embodiments, the transposon vector lacks one or more polynucleotide sequences encoding proteins required for transposition. In an embodiment, the non-autonomous transposon vectors lack one or more Ac transposable elements.

In an embodiment, a non-viral polynucleotide transposon vector system can include a first polynucleotide vector that contains the Acr delivery system polynucleotide(s) and/or CRISPR-Cas system co-therapy polynucleotide(s) of the present invention flanked on the 5′ and 3′ ends by transposon terminal inverted repeats (TIRs) and a second polynucleotide vector that includes a polynucleotide capable of encoding a transposase coupled to a promoter to drive expression of the transposase. When both are expressed in the same cell the transposase can be expressed from the second vector and can transpose the material between the TIRs on the first vector (e.g. the Acr delivery system polynucleotide(s) and/or CRISPR-Cas system polynucleotide(s) of the present invention) and integrate it into one or more positions in the host cell's genome. In an embodiment, the transposon vector or system thereof can be configured as a gene trap. In an embodiment, the TIRs can be configured to flank a strong splice acceptor site followed by a reporter and/or another gene (e.g. one or more of the Acr delivery system polynucleotide(s) and/or CRISPR-Cas system polynucleotide(s) of the present invention) and a strong poly A tail. When transposition occurs while using this vector or system thereof, the transposon can insert into an intron of a gene, and the inserted reporter or another gene can provoke a mis-splicing process, and as a result, it inactivates the trapped gene.

Any suitable transposon system can be used. Suitable transposon and systems thereof can include, the Sleeping Beauty transposon system (Tcl/mariner superfamily) (see e.g. Ivics et al. 1997. Cell. 91 (4): 501-510), piggyBac (piggyBac superfamily) (see e.g. Li et al. 2013 110 (25): E2279-E2287 and Yusa et al. 2011. PNAS. 108 (4): 1531-1536), Tol2 (superfamily hAT), Frog Prince (Tcl/mariner superfamily) (see e.g. Miskey et al. 2003 Nucleic Acid Res. 31 (23): 6873-6881) and variants thereof.

### Delivery Vehicles

Described in an example embodiment herein are delivery vehicles comprising (a) an engineered Acr polypeptide of the present invention; (b) an engineered Acr polypeptide delivery system or component thereof of the present invention; (c) one or more polynucleotides of the present invention; (d) one or more vectors of the present invention; or any combination of (a)-(d). In an embodiment, the delivery vehicle comprises a co-therapy, including but not limited to a CRISPR-Cas system or component thereof. In an embodiment, the delivery vehicle comprises a ribonucleoprotein (RNP) complex of an engineered Acr delivery system and/or CRISPR-Cas system.

The delivery vehicles may deliver the engineered Acr polypeptide, encoding polynucleotides, vectors, etc., of the present invention into and/or within effective proximity of cells, tissues, organs, or organisms (e.g., animals or plants).

In connection with delivery vehicles herein, the engineered Acr polypeptide, encoding polynucleotides, vectors, etc., of the present invention that are carried by the delivery vehicle are referred to as “cargos” for simplicity, The cargos may be packaged, carried, or otherwise associated with the delivery vehicles. The delivery vehicles may be selected based on the types of cargo to be delivered, and/or the mode of delivery (e.g., in vitro and/or in vivo). Examples of delivery vehicles include vectors, viruses (e.g., virus particles), non-viral vehicles, and other delivery reagents described herein.

The delivery vehicles described herein can have the greatest dimension or greatest average dimension (e.g., diameter or greatest average diameter) of less than 100 microns (μm). In an embodiment, the delivery vehicles have the greatest dimension or greatest average dimension of less than 10 μm. In an embodiment, the delivery vehicles may have a greatest dimension or greatest average dimension of less than 2000 nanometers (nm). In an embodiment, the delivery vehicles may have a greatest dimension or greatest average dimension of less than 1000 nanometers (nm). In an embodiment, the delivery vehicles may have the greatest dimension or greatest average dimension (e.g., diameter or average diameter) of less than 900 nm, less than 800 nm, less than 700 nm, less than 600 nm, less than 500 nm, less than 400 nm, less than 300 nm, less than 200 nm, less than 150 nm, or less than 100 nm, less than 50 nm. In an embodiment, the delivery vehicles may have the greatest dimension or greatest average dimension ranging between 25 nm and 200 nm.

In an embodiment, the delivery vehicles may be or comprise particles. For example, the delivery vehicle may be or comprise nanoparticles (e.g., particles with the greatest dimension or greatest average dimension (e.g., diameter or greatest average diameter) no greater than 1000 nm. The particles may be provided in different forms, e.g., as solid particles (e.g., a metal such as silver, gold, iron, titanium), non-metal, lipid-based solids, polymers, suspensions of particles, or combinations thereof. Metal, dielectric, and semiconductor particles may be prepared, as well as hybrid structures (e.g., core-shell particles).

Nanoparticles may also be used to deliver the compositions and systems to cells, as described in WO 2008042156, US20130185823, and WO2015089419. In general, a “nanoparticle” refers to any particle having a diameter of less than 1000 nm. In certain embodiments, nanoparticles of the invention have the greatest dimension or greatest average dimension (e.g., diameter or average diameter) of 500 nm or less. In other embodiments, nanoparticles of the invention have the greatest dimension or greatest average dimension ranging between 25 nm and 200 nm. In other embodiments, nanoparticles of the invention have the greatest dimension or greatest average dimension of 100 nm or less. In other embodiments, nanoparticles of the invention have the greatest dimension or greatest average dimensions ranging between 35 nm and 60 nm. It will be appreciated that reference made herein to particles or nanoparticles can be interchangeable, where appropriate. Nanoparticles made of semiconducting material may also be labeled quantum dots if they are small enough (typically sub 10 nm) that quantization of electronic energy levels occurs. Such nanoscale particles are used in biomedical applications as drug carriers or imaging agents and may be adapted for similar purposes in the present invention. Semi-solid and soft nanoparticles have been manufactured and are within the scope of the present invention. Nanoparticles with one-half hydrophilic and the other half hydrophobic are termed Janus particles and are particularly effective for stabilizing emulsions. They can self-assemble at water/oil interfaces and act as solid surfactants.

Particle characterization (including e.g., characterizing morphology, dimension, etc.) is done using a variety of different techniques. Common techniques are electron microscopy (TEM, SEM), atomic force microscopy (AFM), dynamic light scattering (DLS), X-ray photoelectron spectroscopy (XPS), powder X-ray diffraction (XRD), Fourier transform infrared spectroscopy (FTIR), matrix-assisted laser desorption/ionization time-of-flight mass spectrometry (MALDI-TOF MS), ultraviolet-visible spectroscopy, dual polarization interferometry and nuclear magnetic resonance (NMR). Characterization (dimension measurements) may be made as to native particles (i.e., preloading) or after loading of the cargo (herein cargo refers to e.g., one or more components of the engineered Acr delivery system or any other system described herein e.g., CRISPR-Cas system e.g., CRISPR enzyme or mRNA or guide RNA, or any combination thereof, and may include additional carriers and/or excipients) to provide particles of an optimal size for delivery for any in vitro, ex vivo and/or in vivo application of the present invention. In certain preferred embodiments, particle dimension (e.g., diameter) characterization is based on measurements using dynamic laser scattering (DLS). Mention is made of U.S. Pat. Nos. 8,709,843; 6,007,845; 5,855,913; 5,985,309; 5,543,158; and the publication by James E. Dahlman and Carmen Barnes et al. Nature Nanotechnology (2014) published online 11 May 2014, doi: 10.1038/nnano.2014.84, describing particles, methods of making and using them, and measurements thereof.

### Vectors and Vector Systems

In an embodiment, the delivery vehicle is a vector or vector system. Vectors and vector systems of the present invention are described in greater detail elsewhere herein.

### Non-Vector Delivery Vehicles

The delivery vehicles may comprise non-viral vehicles. In general, methods and vehicles capable of delivering nucleic acids and/or proteins may be used for delivering the systems compositions herein. Examples of non-viral vehicles include lipid nanoparticles, cell-penetrating peptides (CPPs), DNA nanoclews, metal nanoparticles, streptolysin O, multifunctional envelope-type nanodevices (MENDs), lipid-coated mesoporous silica particles, and other inorganic nanoparticles, and those systems described in Hirschenberger et al. 2021. Front. Pharmacol. 12:770283. doi: 10.3389/fphar.2021.770283 and Tian et al., Cell. Rep. 38 (10): 110476 (2022)

### Lipid Particles

The delivery vehicles may comprise lipid particles, e.g., lipid nanoparticles (LNPs) and liposomes. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, International Patent Publication Nos. WO 91/17424 and WO 91/16024. The preparation of lipid: nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

### Lipid Nanoparticles (LNPs)

LNPs may encapsulate nucleic acids within cationic lipid particles (e.g., liposomes), and may be delivered to cells with relative ease. In some examples, lipid nanoparticles do not contain any viral components, which helps minimize safety and immunogenicity concerns. Lipid particles may be used for in vitro, ex vivo, and in vivo deliveries. Lipid particles may be used for various scales of cell populations.

In some examples. LNPs may be used for delivering DNA molecules (e.g., those comprising coding sequences of Cas and/or gRNA) and/or RNA molecules (e.g., mRNA of Cas, gRNAs). In an embodiment, LNPs can include and be used to deliver the engineered Acr delivery system or components thereof and/or a co-therapy, including but not limited to a CRISPR-Cas system or component thereof. In certain cases, LNPs may be used for delivering RNP complexes of the engineered Acr delivery system and/or Cas/gRNA.

Components in LNPs may comprise cationic lipids 1,2-dilineoyl-3-dimethylammonium-propane (DLinDAP), 1,2-dilinoleyloxy-3-N,N-dimethylaminopropane (DLinDMA), 1,2-dilinoleyloxyketo-N,N-dimethyl-3-aminopropane (DLinK-DMA), 1,2-dilinoleyl-4-(2-dimethylaminoethyl)-[1,3]-dioxolane (DLinKC2-DMA), (3-o-[2″-(methoxypolyethyleneglycol 2000) succinoyl]-1,2-dimyristoyl-sn-glycol (PEG-S-DMG), R-3-[(ro-methoxy-poly(ethylene glycol) 2000) carbamoyl]-1,2-dimyristyloxlpropyl-3-amine (PEG-C-DOMG), and any combination thereof. Preparation of LNPs and encapsulation may be adapted from Rosin et al, Molecular Therapy, vol. 19, no. 12, pages 1286-2200, December 2011.

In an embodiment, an LNP delivery vehicle can be used to deliver a virus particles, virus-like particles, proteins, and/or polynucleotides (e.g., DNA, RNA (e.g., mRNA), or ribonucleoprotein (RNP) complex, that encodes or contains an Acr delivery polypeptide, a CRISPR-Cas system co-therapy and/or component(s) thereof. In an embodiment, the virus particle(s), polynucleotide, and/or RNP can be adsorbed to the lipid particle, such as through electrostatic interactions, and/or can be attached to the liposomes via a linker.

In an embodiment, the LNP contains a nucleic acid, wherein the charge ratio of nucleic acid backbone phosphates to cationic lipid nitrogen atoms is about 1:1.5-7 or about 1:4.

In an embodiment, the LNP also includes a shielding compound, which is removable from the lipid composition under in vivo conditions. In an embodiment, the shielding compound is a biologically-inert compound. In an embodiment, the shielding compound does not carry any charge on its surface or on the molecule as such. In an embodiment, the shielding compounds are polyethylenglycoles (PEGs), hydroxyethylglucose (HEG) based polymers, polyhydroxyethyl starch (polyHES), and/or polypropylene. In an embodiment, the PEG, HEG, polyHES, and polypropylene weigh between about 500 to 10,000 Da or between about 2000 to 5000 Da. In an embodiment, the shielding compound is PEG2000 or PEG5000.

In an embodiment, the LNP can include one or more helper lipids. In an embodiment, the helper lipid can be a phospholipid or a steroid. In an embodiment, the helper lipid is between about 20 mol % to 80 mol % of the total lipid content of the composition. In an embodiment, the helper lipid component is between about 35 mol % to 65 mol % of the total lipid content of the LNP. In an embodiment, the LNP includes lipids at 50 mol % of the LNP, of which the helper lipid is present at 50 mol % of the total lipid content of the LNP.

Other non-limiting, exemplary LNP delivery vehicles are described in U.S. Patent Publication Nos. US20160174546, US20140301951, US20150105538, US20150250725, Wang et al., J. Control Release, 2017 Jan. 31. pii: S0168-3659 (17) 30038-X. doi: 10.1016/j.jconrel.2017.01.037. [Epub ahead of print]; Altιnoğlu et al., Biomater Sci., 4(12): 1773-80, Nov. 15, 2016; Wang et al., PNAS, 113(11):2868-73 Mar. 15, 2016; Wang et al., PloS One, 10(11): e0141860. doi: 10.1371/journal.pone.0141860. eCollection 2015 Nov. 3, 2015; Takeda et al., Neural Regen Res. 10 (5): 689-90, May 2015; Wang et al., Adv. Healthc Mater., 3 (9): 1398-403, September 2014; and Wang et al., Agnew Chem Int Ed Engl., 53 (11): 2893-8, Mar. 10, 2014; James E. Dahlman and Carmen Barnes et al. Nature Nanotechnology (2014) published online 11 May 2014, doi: 10.1038/nnano.2014.84; Coelho et al., N Engl J Med 2013; 369:819-29; Aleku et al., Cancer Res., 68 (23): 9788-98 (Dec. 1, 2008), Strumberg et al., Int. J. Clin. Pharmacol. Ther., 50 (1): 76-8 (January 2012), Schultheis et al., J. Clin. Oncol., 32 (36): 4141-48 (Dec. 20, 2014), and Fehring et al., Mol. Ther., 22 (4): 811-20 (Apr. 22, 2014); Novobrantseva, Molecular Therapy-Nucleic Acids (2012) 1, e4; doi: 10.1038/mtna.2011.3; WO2012135025; US20140348900; US20140328759; US20140308304; WO 2005/105152; WO 2006/069782; WO 2007/121947; US 2015/082080; US20120251618; U.S. Pat. Nos. 7,982,027; 7,799,565; 8,058,069; 8,283,333; 7,901,708; 7,745,651; 7,803,397; 8,101,741; 8,188,263; 7,915,399; 8,236,943 and 7,838,658 and European Pat. Nos 1766035; 1519714; 1781593 and 1664316.

### Liposomes

In an embodiment, a lipid particle may be a liposome. Liposomes are spherical vesicle structures composed of a uni- or multi-lamellar lipid bilayer surrounding internal aqueous compartments and a relatively impermeable outer lipophilic phospholipid bilayer. In an embodiment, liposomes are biocompatible, nontoxic, can deliver both hydrophilic and lipophilic drug molecules, protect their cargo from degradation by plasma enzymes, and transport their load across biological membranes and the blood-brain barrier (BBB).

Liposomes can be made from several different types of lipids, e.g., phospholipids. A liposome may comprise natural phospholipids and lipids such as 1,2-distearoryl-sn-glycero-3-phosphatidyl choline (DSPC), sphingomyelin, egg phosphatidylcholines, monosialoganglioside, or any combination thereof.

Several other additives may be added to liposomes in order to modify their structure and properties. For instance, liposomes may further comprise cholesterol, sphingomyelin, and/or 1,2-dioleoyl-sn-glycero-3-phosphoethanolamine (DOPE), e.g., to increase stability and/or to prevent the leakage of the liposomal inner cargo.

In an embodiment, a liposome delivery vehicle can be used to deliver a virus particle, vector, polynucleotide and/or protein, and/or complex thereof (e.g., an RNP) containing an engineered Acr delivery system or component(s) thereof and/or a CRISPR-Cas system and/or component(s) thereof. In an embodiment, the virus particle(s) can be adsorbed to the liposome, such as through electrostatic interactions, and/or can be attached to the liposomes via a linker.

In an embodiment, the liposome can be a Trojan Horse liposome (also known in the art as Molecular Trojan Horses), see e.g., http://cshprotocols.cshlp.org/content/2010/4/pdb.prot5407.long, the teachings of which can be applied and/or adapted to generate and/or deliver the engineered Acr delivery system or component(s) thereof and/or a CRISPR-Cas system and/or component(s) thereof described herein.

Other non-limiting, exemplary liposomes can be those as set forth in Wang et al., ACS Synthetic Biology, 1, 403-07 (2012); Wang et al., PNAS, 113 (11) 2868-2873 (2016); Spuch and Navarro, Journal of Drug Delivery, vol. 2011, Article ID 469679, 12 pages, 2011. doi: 10.1155/2011/469679; WO 2008/042973; U.S. Pat. No. 8,071,082; WO 2014/186366; 20160257951; US20160129120; US20160244761; 20120251618; WO2013/093648; Lipofectin (a combination of DOTMA and DOPE), Lipofectase, LIPOFECTAMINE® (e.g., LIPOFECTAMINE® 2000, LIPOFECTAMINE® 3000, LIPOFECTAMINE® RNAiMAX, LIPOFECTAMINE® LTX), SAINT-RED (Synvolux Therapeutics, Groningen Netherlands), DOPE, Cytofectin (Gilead Sciences, Foster City, Calif.), and Eufectins (JBL, San Luis Obispo, Calif.).

### Stable Nucleic-Acid-Lipid Particles (SNALPs)

In an embodiment, the lipid particles may be stable nucleic-acid-lipid particles (SNALPs). SNALPs may comprise an ionizable lipid (e.g., DLinDMA, which iscationic at low pH), a neutral helper lipid (e.g., cholesterol), a diffusible polyethylene glycol (PEG)-lipid, or any combination thereof. In some examples, SNALPs may comprise synthetic cholesterol, dipalmitoylphosphatidylcholine, 3-N-[(w-methoxy polyethylene glycol)2000)carbamoyl]-1,2-dimyrestyloxypropylamine, and cationic 1,2-dilinoleyloxy-3-N,Ndimethylaminopropane. In some examples, SNALPs may comprise synthetic cholesterol, 1,2-distearoyl-sn-glycero-3-phosphocholine, PEG-CDMA, and 1,2-dilinoleyloxy-3-(N,N-dimethyl)aminopropane (DLinDMAo).

Other non-limiting, exemplary SNALPs that can be used to deliver the engineered Acr delivery system or component(s) thereof and/or a CRISPR-Cas system and/or component(s) thereof described herein can be any such SNALPs as described in Morrissey et al., Nature Biotechnology, Vol. 23, No. 8, August 2005, Zimmerman et al., Nature Letters, Vol. 441, 4 May 2006; Geisbert et al., Lancet 2010; 375:1896-905; Judge, J. Clin. Invest. 119:661-673 (2009); and Semple et al., Nature Biotechnology, Volume 28 Number 2 Feb. 2010, pp. 172-177. In an embodiment, the engineered Acr delivery system or component(s) thereof and/or a CRISPR-Cas system and/or component(s) thereof is included as an RNP. In other embodiments, the engineered Acr delivery system or component(s) thereof and/or a CRISPR-Cas system and/or component(s) thereof is included as mRNA.

### Other Lipids

The lipid particles may also comprise one or more other types of lipids, e.g., cationic lipids, such as amino lipid 2,2-dilinoleyl-4-dimethylaminoethyl-[1,3]-dioxolane (DLin-KC2-DMA), DLin-KC2-DMA4, C12-200, and co-lipids disteroylphosphatidyl choline, cholesterol, and PEG-DMG.

In an embodiment, the delivery vehicle can be or include a lipidoid, such as any of those set forth in, for example, US20110293703.

In an embodiment, the delivery vehicle can be or include an amino lipid, such as any of those set forth in, for example, Jayaraman, Angew. Chem. Int. Ed. 2012, 51, 8529-8533.

In an embodiment, the delivery vehicle can be or include a lipid envelope, such as any of those set forth in, for example, Korman et al., 2011. Nat. Biotech. 29:154-157.

### Lipoplexes/Polyplexes

In an embodiment, the delivery vehicles comprise lipoplexes and/or polyplexes. Lipoplexes may bind to negatively charged cell membranes and induce endocytosis into the cells. Examples of lipoplexes may be complexes comprising lipid(s) and non-lipid components. Examples of lipoplexes and polyplexes include FuGENE-6 reagent, a non-liposomal solution containing lipids and other components, zwitterionic amino lipids (ZALs), Ca2p (e.g., forming DNA/Ca²⁺ microcomplexes), polyethenimine (PEI) (e.g., branched PEI), and poly(L-lysine) (PLL).

### Sugar-Based Particles

In an embodiment, the delivery vehicle can be a sugar-based particle. In an embodiment, the sugar-based particles can be or include GalNAc, such as any of those described in WO2014118272; US20020150626; Nair, J K et al., 2014, Journal of the American Chemical Society 136 (49), 16958-16961; Østergaard et al., Bioconjugate Chem., 2015, 26 (8), pp 1451-1455.

### Cell-Penetrating Peptides

In an embodiment, the delivery vehicles comprise cell-penetrating peptides (CPPs). CPPs are short peptides that facilitate cellular uptake of various molecular cargos (e.g., from nanosized particles to small chemical molecules and large fragments of DNA).

CPPs may be of different sizes, amino acid sequences, and charges. In some examples, CPPs can translocate the plasma membrane and facilitate the delivery of various molecular cargos to the cytosolor an organelle. CPPs may be introduced into cells via different mechanisms, e.g., direct penetration in the membrane, endocytosis-mediated entry, and translocation through the formation of a transitory structure.

CPPs may have an amino acid composition that either contains a high relative abundance of positively charged amino acids such as lysine or arginine or has sequences that contain an alternating pattern of polar/charged amino acids and non-polar, hydrophobic amino acids. These two types of structures are referred to as polycationic or amphipathic, respectively. A third class of CPPs is the hydrophobic peptides, containing only apolar residues, with low net charge or with hydrophobic amino acid groups that are crucial for cellular uptake. Another type of CPPs is the trans-activating transcriptional activator (Tat) from Human Immunodeficiency Virus 1 (HIV-1). Examples of CPPs include Penetratin, Tat (48-60), Transportan, and (R-AhX-R4) (Ahx refers to aminohexanoyl), Kaposi fibroblast growth factor (FGF) signal peptide sequence, integrin β3 signal peptide sequence, polyarginine peptide (poly-Arg) sequence, Guanine rich-molecular transporters, and sweet arrow peptide. In an embodiment, the CPP is a cyclic CPP (see e.g., Herce et al., Nat. Chem. 9:762-771 (2017)). Examples of CPPs and related applications also include those described in U.S. Pat. No. 8,372,951.

CPPs can be used for in vitro and ex vivo work quite readily, and extensive optimization for each cargo and cell type is usually required. In some examples, CPPs may be covalently attached to the Cas protein directly, which is then complexed with the gRNA and delivered to cells. See e.g., Ramakrishna et al. Genome Res. 2014. 24:1020-1027 and Staahl et al. Nature Biotechnology. 35:431-434 (2017). In some examples, separate delivery of CPP-Cas and CPP-gRNA to multiple cells may be performed. CPPs may also be used to deliver RNPs.

CPPs may be used to deliver the compositions and systems to plants. In some examples, CPPs may be used to deliver the components to plant protoplasts, which are then regenerated to plant cells and further to plants.

### DNA Nanoclews

In an embodiment, the delivery vehicles comprise DNA nanoclews. A DNA nanoclew refers to a sphere-like structure of DNA (e.g., with a shape of a ball of yarn). The nanoclew may be synthesized by rolling circle amplification with palindromic sequences that aid in the self-assembly of the structure. The sphere may then be loaded with a payload. An example of DNA nanoclew is described in Sun W et al, J Am Chem Soc. 2014 Oct. 22; 136 (42): 14722-5; and Sun W et al, Angew Chem Int Ed Engl. 2015 Oct. 5; 54 (41): 12029-33. A DNA nanoclew may have a palindromic sequence to be partially complementary to the gRNA within the Cas:gRNA ribonucleoprotein complex. A DNA nanoclew may be coated, e.g., coated with PEI to induce endosomal escape.

### Metal Nanoparticles

In an embodiment, the delivery vehicles comprise gold nanoparticles (also referred to AuNPs or colloidal gold). Gold nanoparticles may form a complex with cargos, e.g., Cas:gRNA RNP. Gold nanoparticles may be coated, e.g., coated in a silicate and an endosomal disruptive polymer, PAsp (DET). Examples of gold nanoparticles include AuraSense Therapeutics' Spherical Nucleic Acid (SNA™) constructs, and those described in Mout R, et al. (2017). ACS Nano 11:2452-8; Lee K, et al. (2017). Nat Biomed Eng 1:889-901. Other metal nanoparticles can also be complexed with cargo(s). Such metal particles include tungsten, palladium, rhodium, platinum, and iridium particles. Other non-limiting, exemplary metal nanoparticles are described in US20100129793.

iTOP

In an embodiment, the delivery vehicles comprise iTOP. iTOP refers to a combination of small molecules that drives the highly efficient intracellular delivery of native proteins, independent of any transduction peptide. iTOP may be used for induced transduction by osmocytosis and propanebetaine, using NaCl-mediated hyperosmolality together with a transduction compound (propanebetaine) to trigger macropinocytotic uptake into cells of extracellular macromolecules. Examples of iTOP methods and reagents include those described in D'Astolfo D S, Pagliero R J, Pras A, et al. (2015). Cell 161:674-690.

### Polymer-Based Particles

In an embodiment, the delivery vehicles may comprise polymer-based particles (e.g., nanoparticles). In an embodiment, the polymer-based particles may mimic a viral mechanism of membrane fusion. The polymer-based particles may be a synthetic copy of Influenza virus machinery and form transfection complexes with various types of nucleic acids (siRNA, miRNA, plasmid DNA, shRNA, or mRNA) that cells take up via the endocytosis pathway, a process that involves the formation of an acidic compartment. The low pH in late endosomes acts as a chemical switch that renders the particle surface hydrophobic and facilitates membrane crossing. Once in the cytosol, the particle releases its payload for cellular action. This Active Endosome Escape technology is safe and maximizes transfection efficiency as it is using a natural uptake pathway. In an embodiment, the polymer-based particles may comprise alkylated and carboxyalkylated branched polyethylenimine. In some examples, the polymer-based particles are or comprise Viromers, e.g., ViromerR RNAi, Viromer RED, Viromer mRNA, Viromer CRISPR. Example methods of delivering the systems and compositions herein include those described in Bawage S S et al., Synthetic mRNA expressed Cas13a mitigates RNA virus infections, www.biorxiv.org/content/10.1101/370460v1.full doi: doi.org/10.1101/370460, Viromer® RED, a powerful tool for transfection of keratinocytes. doi: 10.13140/RG.2.2.16993.61281, Viromer® Transfection—Factbook 2018: technology, product overview, users' data., doi: 10.13140/RG.2.2.23912.16642. Other exemplary and non-limiting polymeric particles are described in US20170079916, US20160367686, US 20110212179, US20130302401, U.S. Pat. Nos. 6,007,845, 5,855,913, 5,985,309, 5,543,158, WO2012135025, US20130252281, US20130245107, US20130244279; US20050019923, 20080267903.

### Streptolysin O (SLO)

The delivery vehicles may be streptolysin O (SLO). SLO is a toxin produced by Group A streptococci that works by creating pores in mammalian cell membranes. SLO may act in a reversible manner, which allows for the delivery of proteins (e.g., up to 100 kDa) to the cytosol of cells without compromising overall viability. Examples of SLO include those described in Sierig G, et al. (2003). Infect Immun 71:446-55; Walev I, et al. (2001). Proc. Natl. Acad. Sci U.S.A. 98:3185-90; Teng K W, et al. (2017). Elife 6: e25460.

### Multifunctional Envelope-Type Nanodevice (MEND)

The delivery vehicles may comprise multifunctional envelope-type nanodevices (MENDs). MENDs may comprise condensed plasmid DNA, a PLL core, and a lipid film shell. A MEND may further comprise a cell-penetrating peptide (e.g., stearyl octaarginine). The cell-penetrating peptide may be in the lipid shell. The lipid envelope may be modified with one or more functional components, e.g., one or more of: polyethylene glycol (e.g., to increase vascular circulation time), ligands for targeting specific tissues/cells, additional cell-penetrating peptides (e.g., for greater cellular delivery), lipids to enhance endosomal escape, and nuclear delivery tags. In some examples, the MEND may be a tetra-lamellar MEND (T-MEND), which may target the cellular nucleus and mitochondria. In certain examples, a MEND may be a PEG-peptide-DOPE-conjugated MEND (PPD-MEND), which may target bladder cancer cells. Examples of MENDs include those described in Kogure K, et al. (2004). J Control Release 98:317-23; Nakamura T, et al. (2012). Acc Chem Res 45:1113-21.

### Lipid-Coated Mesoporous Silica Particles

The delivery vehicles may comprise lipid-coated mesoporous silica particles. Lipid-coated mesoporous silica particles may comprise a mesoporous silica nanoparticle core and a lipid membrane shell. The silica core may have a large internal surface area, leading to high cargo loading capacities. In an embodiment, pore sizes, pore chemistry, and overall particle sizes may be modified for loading different types of cargo. The lipid coating of the particle may also be modified to maximize cargo loading, increase circulation times, and provide precise targeting and cargo release. Examples of lipid-coated mesoporous silica particles include those described in Du X, et al. (2014). Biomaterials 35:5580-90; Durfee P N, et al. (2016). ACS Nano 10:8325-45.

### Inorganic Nanoparticles

The delivery vehicles may comprise inorganic nanoparticles. Examples of inorganic nanoparticles include carbon nanotubes (CNTs) (e.g., as described in Bates K and Kostarelos K. (2013). Adv Drug Deliv Rev 65:2023-33), bare mesoporous silica nanoparticles (MSNPs) (e.g., as described in Luo G F, et al. (2014). Sci Rep 4:6064), and dense silica nanoparticles (SiNPs) (as described in Luo D and Saltzman W M. (2000). Nat Biotechnol 18:893-5).

### Exosomes

The delivery vehicles may comprise exosomes. Exosomes include membrane-bound extracellular vesicles, which can be used to contain and deliver various types of biomolecules, such as proteins, carbohydrates, lipids, nucleic acids, and complexes thereof (e.g., RNPs). Examples of exosomes include those described in Schroeder A, et al., J. Intern Med. 2010 January; 267 (1): 9-21; El-Andaloussi S, et al., Nat Protoc. 2012 December; 7 (12): 2112-26; Uno Y, et al., Hum Gene Ther. 2011 June; 22 (6): 711-9; Zou W, et al., Hum Gene Ther. 2011 April; 22 (4): 465-75.

In some examples, the exosome may form a complex (e.g., by binding directly or indirectly) to one or more components of the cargo. In certain examples, a molecule of an exosome may be fused with a first adapter protein and a component of the cargo may be fused with a second adapter protein. The first and the second adapter protein may specifically bind each other, thus associating the cargo with the exosome. Examples of such exosomes include those described in Ye Y, et al., Biomater Sci. 2020 Apr. 28. doi: 10.1039/d0bm00427h.

Other non-limiting, exemplary exosomes include any of those set forth in Alvarez-Erviti et al. 2011, Nat Biotechnol 29:341; El-Andaloussi et al. (Nature Protocols 7:2112-2126 (2012); and Wahlgren et al. (Nucleic Acids Research, 2012, Vol. 40, No. 17 e130).

### Spherical Nucleic Acids (SNAs)

In an embodiment, the delivery vehicle can be an SNA. SNAs are three-dimensional nanostructures that can be composed of densely functionalized and highly oriented nucleic acids that can be covalently attached to the surface of spherical nanoparticle cores. The core of the spherical nucleic acid can impart the conjugate with specific chemical and physical properties, and it can act as a scaffold for assembling and orienting the oligonucleotides into a dense spherical arrangement that gives rise to many of their functional properties, distinguishing them from all other forms of matter. In an embodiment, the core is a crosslinked polymer. Non-limiting, exemplary SNAs can be any of those set forth in Cutler et al., J. Am. Chem. Soc. 2011 133:9254-9257, Hao et al., Small. 2011 7:3158-3162, Zhang et al., ACS Nano. 2011 5:6962-6970, Cutler et al., J. Am. Chem. Soc. 2012 134:1376-1391, Young et al., Nano Lett. 2012 12:3867-71, Zheng et al., Proc. Natl. Acad. Sci. USA. 2012 109:11975-80, Mirkin, Nanomedicine 2012 7:635-638 Zhang et al., J. Am. Chem. Soc. 2012 134:16488-1691, Weintraub, Nature 2013 495: S14-S16, Choi et al., Proc. Natl. Acad. Sci. USA. 2013 110 (19): 7625-7630, Jensen et al., Sci. Transl. Med. 5, 209ra152 (2013) and Mirkin, et al., and Small, 10:186-192.

### Self-Assembling Nanoparticles

In an embodiment, the delivery vehicle is a self-assembling nanoparticle. The self-assembling nanoparticles can contain one or more polymers. The self-assembling nanoparticles can be PEGylated. Self-assembling nanoparticles are known in the art. Non-limiting, exemplary self-assembling nanoparticles can be any as set forth in Schiffelers et al., Nucleic Acids Research, 2004, Vol. 32, No. 19, Bartlett et al. Proc. Natl. Acad. Sci. USA. Sep. 25, 2007, vol. 104, no. 39; Davis et al., Nature, Vol 464, 15 Apr. 2010.

### Supercharged Proteins

In an embodiment, the delivery vehicle can be a supercharged protein. As used herein “Supercharged proteins” are a class of engineered or naturally occurring proteins with unusually high positive or negative net theoretical charge. Non-limiting, exemplary supercharged proteins can be any of those set forth in Lawrence et al., 2007, Journal of the American Chemical Society 129, 10110-10112 and Fuchs and Raines. ACS Chem. Biol. 2 (3): 167-170 (2007).

### Virus-Like Particles

In an embodiment, the delivery vehicle can be a virus like particles. VLPs is a term of art that refers to particles produced from virus proteins, such as capsid or other proteins, but that do not contain the native viral genetic materials. Exemplary VLPs and their production systems and vectors for delivery of an engineered Acr delivery system described herein are described in e.g., Bhat et al., Viruses 14 (2): 383 (2022) doi: 10.3390/v14020383; Hill et al., Curr Protein Pept Sci. (2018) 19 (1): 112-127; Schwarz B et al., Adv Virus Res. 2017. 97:1-60 doi: 10.1016/bs.aivir.2016.09.002; Banskota et al., Cell. 2022. 185 (2): 250-265; Ikwuagwu and Tullman-Ercek. Curr Opin Biotechnol. 2022. 78:102785 doi: 10.1016/j.copbio.2022.102785; Zdanowicz and Chroboczek. Acta Biochim Pol. 2016: 63 (3): 469-473; Suffian and Al-Jamal et al., Adv. Drug Deliv. Rev. 2022. 180:114030 doi: 10.1016/j.addr.2021.114030; and Segel et al., Science. 373:6557 (2021).

### Targeted Delivery

In an embodiment, the delivery vehicle can allow for targeted delivery to a specific cell, tissue, organ, or system. In such embodiments, the delivery vehicle can include one or more targeting moieties that can direct targeted delivery of the cargo(s). In an embodiment, the delivery vehicle comprises a targeting moiety.

Exemplary targeting moieties are described in greater detail elsewhere herein and are applicable to targeting moieties that can be included in a delivery vehicle.

### Responsive Delivery

In an embodiment, the delivery vehicle can allow for responsive delivery of the cargo(s). Responsive delivery, as used in this context herein, refers to delivery of cargo(s) by the delivery vehicle in response to an external stimuli. Examples of suitable stimuli include, without limitation, energy (light, heat, cold, and the like), chemical stimuli (e.g., chemical composition, etc.), and biologic or physiologic stimuli (e.g., environmental pH, osmolarity, salinity, biologic molecule, etc.). In an embodiment, the targeting moiety can be responsive to external stimuli and facilitate responsive delivery. In other embodiments, responsiveness is determined by a non-targeting moiety component of the delivery vehicle.

The delivery vehicle can be stimuli-sensitive, e.g., sensitive to externally applied stimuli, such as magnetic fields, ultrasound, or light; and pH-triggering can also be used, e.g., a labile linkage can be used between a hydrophilic moiety such as PEG and a hydrophobic moiety such as a lipid entity of the invention, which is cleaved only upon exposure to the relatively acidic conditions characteristic of a particular environment or microenvironment such as an endocytic vacuole or the acidotic tumor mass. pH-sensitive copolymers can also be incorporated in embodiments of the invention to provide shielding; diortho esters, vinyl esters, cysteine-cleavable lipopolymers, double esters, and hydrazones are a few examples of pH-sensitive bonds that are quite stable at pH 7.5, but are hydrolyzed relatively rapidly at pH 6 and below, e.g., a terminally alkylated copolymer of N-isopropylacrylamide and methacrylic acid that facilitates destabilization of a lipid entity of the invention and release in compartments with decreased pH value; or, the invention comprehends ionic polymers for generation of a pH-responsive lipid entity of the invention (e.g., poly(methacrylic acid), poly(diethylaminoethyl methacrylate), poly(acrylamide) and poly(acrylic acid)).

Temperature-triggered delivery is also within the ambit of the invention. Many pathological areas, such as inflamed tissues and tumors, show distinctive hyperthermia compared with normal tissues. Utilizing this hyperthermia is an attractive strategy in cancer therapy since hyperthermia is associated with increased tumor permeability and enhanced uptake. This technique involves local heating of the site to increase microvascular pore size and blood flow, which, in turn, can result in increased extravasation of embodiments of the invention. A temperature-sensitive lipid entity of the invention can be prepared from thermosensitive lipids or polymers with a low critical solution temperature. Above the low critical solution temperature (e.g., at a site such as the tumor site or inflamed tissue site), the polymer precipitates, disrupting the liposomes to release the cargo. Lipids with a specific gel-to-liquid phase transition temperature are used to prepare these lipid entities of the invention, and a lipid for a thermosensitive embodiment can be dipalmitoylphosphatidylcholine. Thermosensitive polymers can also facilitate destabilization followed by release, and a useful thermosensitive polymer is poly(N-isopropylacrylamide). Another temperature-triggered system can employ lysolipid temperature-sensitive liposomes.

The invention also comprehends redox-triggered delivery. The difference in redox potential between normal and inflamed or tumor tissues, and between the intra- and extra-cellular environments has been exploited for delivery, e.g., glutathione (GSH) is a reducing agent abundant in cells, especially in the cytosol, mitochondria, and nucleus. The GSH concentrations in blood and extracellular matrix are just one out of 100 to one out of 1000 of the intracellular concentration, respectively. This high redox potential difference caused by GSH, cysteine, and other reducing agents can break the reducible bonds, destabilize a lipid entity of the invention and result in the release of the payload. A disulfide bond can be used as the cleavable/reversible linker in a lipid entity of the invention, because it causes sensitivity to redox owing to the disulfide-to-thiol reduction reaction; a lipid entity of the invention can be made reduction sensitive by using two forms of a disulfide-conjugated multifunctional lipid where cleavage of the disulfide bond (e.g., via tris(2-carboxyethyl) phosphine, dithiothreitol, L-cysteine or GSH), can cause removal of the hydrophilic head group of the conjugate and alter the membrane organization leading to the release of the payload.

Enzymes can also be used as a trigger to release payload. Enzymes, including MMPs (e.g., MMP2), phospholipase A2, alkaline phosphatase, transglutaminase, or phosphatidylinositol-specific phospholipase C, have been found to be overexpressed in certain tissues, e.g., tumor tissues. In the presence of these enzymes, a specially engineered enzyme-sensitive lipid entity of the invention can be disrupted and release the payload. An MMP2-cleavable octapeptide (Gly-Pro-Leu-Gly-Ile-Ala-Gly-Gln) (SEQ ID NO: 107) can be incorporated into a linker, and can have an antibody targeting moiety, e.g., antibody 2C5.

The invention also comprehends light- or energy-triggered delivery, e.g., the lipid entity of the invention can be light-sensitive, such that light or energy can facilitate structural and conformational changes, which lead to direct interaction of the lipid entity of the invention with the target cells via membrane fusion, photo-isomerism, photofragmentation or photopolymerization; such a moiety therefore can be a benzoporphyrin photosensitizer. Ultrasound can be a form of energy to trigger delivery; a lipid entity of the invention with a small quantity of a particular gas, including air or a perfluorated hydrocarbon, can be triggered to release with ultrasound, e.g., low-frequency ultrasound (LFUS). Magnetic delivery: A lipid entity of the invention can be magnetized by incorporation of magnetites, such as Fe₃O₄ or γ-Fe₂O₃, e.g., those that are less than 10 nm in size. Triggered delivery then occurs via exposure to a magnetic field.

### Cells

Described in an example embodiment herein is a cell or cell population containing (a) an engineered Acr polypeptide of the present invention; (b) an engineered Acr polypeptide delivery system or component thereof of the present invention; (c) one or more polynucleotides of the present invention; (d) one or more vectors of the present invention; (e) a delivery vehicle of the present invention; or (f) any combination of (a)-(e). In an embodiment, the cell(s) also contain a CRISPR-Cas system or component thereof, such as a Cas polypeptide and/or guide RNA. Exemplary CRISPR-Cas systems and components thereof are described in greater detail elsewhere herein.

In an embodiment, the cell or cell population is a eukaryotic cell or cell population. In an embodiment, the eukaryotic cell or cell population is a mammalian cell or cell population. In an embodiment, the eukaryotic cell or cell population is a non-human mammalian cell or cell population. In an embodiment, the cell or cell population is a human cell or cell population. In an embodiment, the cell or cell population is a plant cell or cell population. In an embodiment, the cell or cell population is a fungal cell or cell population. In an embodiment, the cell or cell population is a prokaryotic cell or cell population. In an embodiment, the cell or cell population is part of an organism. In an embodiment, the organism is a non-human animal. In an embodiment, the organism is a human. In an embodiment, the cell or cell population is ex vivo or in vitro.

Exemplary non-human animal cell(s) are mammalian. Exemplary non-human mammals include, without limitation, non-human primates, canines, felines, swines, bovines, equines, ovines, camelids, ursids, leporids, murines, cricetids, cervids, giraffids, etc.,

In general, the term “plant” refers to any photosynthetic, eukaryotic, unicellular, or multicellular organism of the kingdom Plantae characteristically growing by cell division, containing chloroplasts, and having cell walls comprised of cellulose. The term plant encompasses monocotyledonous and dicotyledonous plants. Specifically, the plants are intended to comprise without limitation angiosperm and gymnosperm plants such as acacia, alfalfa, amaranth, apple, apricot, artichoke, ash tree, asparagus, avocado, banana, barley, beans, beet, birch, beech, blackberry, blueberry, broccoli, Brussel's sprouts, cabbage, canola, cantaloupe, carrot, cassava, cauliflower, cedar, a cereal, celery, chestnut, cherry, Chinese cabbage, citrus, clementine, clover, coffee, corn, cotton, cowpea, cucumber, cypress, eggplant, elm, endive, eucalyptus, fennel, figs, fir, geranium, grape, grapefruit, groundnuts, ground cherry, gum hemlock, hickory, kale, kiwifruit, kohlrabi, larch, lettuce, leek, lemon, lime, locust, pine, maidenhair, maize, mango, maple, melon, millet, mushroom, mustard, nuts, oak, oats, oil palm, okra, onion, orange, an ornamental plant or flower or tree, papaya, palm, parsley, parsnip, pea, peach, peanut, pear, peat, pepper, persimmon, pigeon pea, pine, pineapple, plantain, plum, pomegranate, potato, pumpkin, radicchio, radish, rapeseed, raspberry, rice, rye, sorghum, safflower, sallow, soybean, spinach, spruce, squash, strawberry, sugar beet, sugarcane, sunflower, sweet potato, sweet corn, tangerine, tea, tobacco, tomato, trees, triticale, turf grasses, turnips, vine, walnut, watercress, watermelon, wheat, yams, yew, and zucchini. The term plant also encompasses Algae, which are mainly photoautotrophs unified primarily by their lack of roots, leaves, and other organelles that characterize higher plants. Exemplary plant cells include, without limitation, those cells of monocotyledonous and dicotyledonous plants, such as crops including grain crops (e.g., wheat, maize, rice, millet, barley), fruit crops (e.g., tomato, apple, pear, strawberry, orange), forage crops (e.g., alfalfa), root vegetable crops (e.g., carrot, potato, sugar beets, yam), leafy vegetable crops (e.g., lettuce, spinach); flowering plants (e.g., petunia, rose, chrysanthemum), conifers and pine trees (e.g., pine fir, spruce); plants used in phytoremediation (e.g., heavy metal accumulating plants); oil crops (e.g., sunflower, rape seed) and plants used for experimental purposes (e.g., Arabidopsis). Plant cells and tissues that can include the Acr delivery compositions and/or systems of the present invention include, without limitation, roots, stems, leaves, flowers and reproductive structures, undifferentiated meristematic cells, parenchyma, collenchyma, sclerenchyma, xylem, phloem, epidermis, and germplasm. A part of a plant, e.g., a “plant tissue” may be treated according to the methods of the present invention to produce an improved plant. Plant tissue also encompasses plant cells. The term “plant cell” as used herein refers to individual units of a living plant, either in an intact whole plant or in an isolated form grown in in vitro tissue cultures, on media or agar, in suspension in a growth media or buffer or as a part of higher organized units, such as, for example, plant tissue, a plant organ, or a whole plant. A “protoplast” refers to a plant cell that has had its protective cell wall completely or partially removed using, for example, mechanical or enzymatic means resulting in an intact biochemical competent unit of living plant that can reform their cell wall, proliferate, and regenerate into a whole plant under proper growing conditions. This also includes the progeny of plant cells that include one or more of the Acr delivery compositions and/or systems of the present invention, such as the progeny of a transgenic plant, is one that is born of, begotten by, or derived from a plant to which an Acr delivery composition and/or system of the present invention is delivered.

Thus, it will be appreciated that the Acr delivery compositions and/or systems of the present invention can be used over a broad range of plants, such as for example with dicotyledonous plants belonging to the orders Magniolales, Illiciales, Laurales, Piperales, Aristochiales, Nymphaeales, Ranunculales, Papeverales, Sarraceniaceae, Trochodendrales, Hamamelidales, Eucomiales, Leitneriales, Myricales, Fagales, Casuarinales, Caryophyllales, Batales, Polygonales, Plumbaginales, Dilleniales, Theales, Malvales, Urticales, Lecythidales, Violales, Salicales, Capparales, Ericales, Diapensales, Ebenales, Primulales, Rosales, Fabales, Podostemales, Haloragales, Myrtales, Cornales, Proteales, San tales, Rafflesiales, Celastrales, Euphorbiales, Rhamnales, Sapindales, Juglandales, Geraniales, Polygalales, Umbellales, Gentianales, Polemoniales, Lamiales, Plantaginales, Scrophulariales, Campanulales, Rubiales, Dipsacales, and Asterales; monocotyledonous plants such as those belonging to the orders Alismatales, Hydrocharitales, Najadales, Triuridales, Commelinales, Eriocaulales, Restionales, Poales, Juncales, Cyperales, Typhales, Bromeliales, Zingiberales, Arecales, Cyclanthales, Pandanales, Arales, Lilliales, and Orchid ales, or with plants belonging to Gymnospermae, e.g., those belonging to the orders Pinales, Ginkgoales, Cycadales, Araucariales, Cupressales and Gnetales. It will also be appreciated that the Acr delivery compositions and/or systems of the present invention can be used over a broad range of plant species, included in the non-limitative list of dicot, monocot or gymnosperm genera hereunder: Atropa, Alseodaphne, Anacardium, Arachis, Beilschmiedia, Brassica, Carthamus, Cocculus, Croton, Cucumis, Citrus, Citrullus, Capsicum, Catharanthus, Cocos, Coffea, Cucurbita, Daucus, Duguetia, Eschscholzia, Ficus, Fragaria, Glaucium, Glycine, Gossypium, Helianthus, Hevea, Hyoscyamus, Lactuca, Landolphia, Linum, Litsea, Lycopersicon, Lupinus, Manihot, Majorana, Malus, Medicago, Nicotiana, Olea, Parthenium, Papaver, Persea, Phaseolus, Pistacia, Pisum, Pyrus, Prunus, Raphanus, Ricinus, Senecio, Sinomenium, Stephania, Sinapis, Solanum, Theobroma, Trifolium, Trigonella, Vicia, Vinca, Vilis, and Vigna; and the genera Allium, Andropogon, Aragrostis, Asparagus, Avena, Cynodon, Elaeis, Festuca, Festulolium, Heterocallis, Hordeum, Lemna, Lolium, Musa, Oryza, Panicum, Pannesetum, Phleum, Poa, Secale, Sorghum, Triticum, Zea, Abies, Cunninghamia, Ephedra, Picea, Pinus, and Pseudotsuga.

It will also be appreciated that the Acr delivery compositions and/or systems of the present invention can be used over a broad range of “algae” or “algae cells”; including for example algae selected from several eukaryotic phyla, including the Rhodophyta (red algae), Chlorophyta (green algae), Phaeophyta (brown algae), Bacillariophyta (diatoms), Eustigmatophyta and dinoflagellates as well as the prokaryotic phylum Cyanobacteria (blue-green algae). The term “algae” includes for example algae selected from: Amphora, Anabaena, Anikstrodesmis, Botryococcus, Chaetoceros, Chlamydomonas, Chlorella, Chlorococcum, Cyclotella, Cylindrotheca, Dunaliella, Emiliana, Euglena, Hematococcus, Isochrysis, Monochrysis, Monoraphidium, Nannochloris, Nannnochloropsis, Navicula, Nephrochloris, Nephroselmis, Nitzschia, Nodularia, Nostoc, Oochromonas, Oocystis, Oscillartoria, Pavlova, Phaeodactylum, Playtmonas, Pleurochrysis, Porhyra, Pseudoanabaena, Pyramimonas, Stichococcus, Synechococcus, Synechocystis, Tetraselmis, Thalassiosira, and Trichodesmium.

As used herein, the term “yeast cell” refers to any fungal cell within the phyla Ascomycota and Basidiomycota. Yeast cells may include budding yeast cells, fission yeast cells, and mold cells. Without being limited to these organisms, many types of yeast used in laboratory and industrial settings are part of the phylum Ascomycota. In an embodiment, the yeast cell is an S. cerevisiae, Kluyveromyces marxianus, or Issatchenkia orientalis cell. Other yeast cells may include without limitation Candida spp. (e.g., Candida albicans), Yarrowia spp. (e.g., Yarrowia lipolytica), Pichia spp. (e.g., Pichia pastoris), Kluyveromyces spp. (e.g., Kluyveromyces lactis and Kluyveromyces marxianus), Neurospora spp. (e.g., Neurospora crassa), Fusarium spp. (e.g., Fusarium oxysporum), and Issatchenkia spp. (e.g., Issatchenkia orientalis, a.k.a. Pichia kudriavzevii and Candida acidothermophilum). In an embodiment, the fungal cell is a filamentous fungal cell. As used herein, the term “filamentous fungal cell” refers to any type of fungal cell that grows in filaments, i.e., hyphae or mycelia. Examples of filamentous fungal cells may include without limitation Aspergillus spp. (e.g., Aspergillus niger), Trichoderma spp. (e.g., Trichoderma reesei), Rhizopus spp. (e.g., Rhizopus oryzae), and Mortierella spp. (e.g., Mortierella isabellina).

In an embodiment, the fungal cell is an industrial strain. As used herein, “industrial strain” refers to any strain of fungal cell used in or isolated from an industrial process, e.g., production of a product on a commercial or industrial scale. Industrial strain may refer to a fungal species that is typically used in an industrial process, or it may refer to an isolate of a fungal species that may be also used for non-industrial purposes (e.g., laboratory research). Examples of industrial processes may include fermentation (e.g., in production of food or beverage products), distillation, biofuel production, production of a compound, and production of a polypeptide. Examples of industrial strains may include, without limitation, JAY270 and ATCC4124.

In an embodiment, the fungal cell is a polyploid cell. As used herein, a “polyploid” cell may refer to any cell whose genome is present in more than one copy. A polyploid cell may refer to a type of cell that is naturally found in a polyploid state, or it may refer to a cell that has been induced to exist in a polyploid state (e.g., through specific regulation, alteration, inactivation, activation, or modification of meiosis, cytokinesis, or DNA replication). A polyploid cell may refer to a cell whose entire genome is polyploid, or it may refer to a cell that is polyploid in a particular genomic locus of interest.

In an embodiment, the fungal cell is a diploid cell. As used herein, a “diploid” cell may refer to any cell whose genome is present in two copies. A diploid cell may refer to a type of cell that is naturally found in a diploid state, or it may refer to a cell that has been induced to exist in a diploid state (e.g., through specific regulation, alteration, inactivation, activation, or modification of meiosis, cytokinesis, or DNA replication). For example, the S. cerevisiae strain S228C may be maintained in a haploid or diploid state. A diploid cell may refer to a cell whose entire genome is diploid, or it may refer to a cell that is diploid in a particular genomic locus of interest. In an embodiment, the fungal cell is a haploid cell. As used herein, a “haploid” cell may refer to any cell whose genome is present in one copy. A haploid cell may refer to a type of cell that is naturally found in a haploid state, or it may refer to a cell that has been induced to exist in a haploid state (e.g., through specific regulation, alteration, inactivation, activation, or modification of meiosis, cytokinesis, or DNA replication). For example, the S. cerevisiae strain S228C may be maintained in a haploid or diploid state. A haploid cell may refer to a cell whose entire genome is haploid, or it may refer to a cell that is haploid in a particular genomic locus of interest.

### Pharmaceutical Formulations

Also described herein are pharmaceutical formulations that can contain an amount, effective amount, and/or least effective amount, and/or therapeutically effective amount of one or more engineered Acr delivery compounds, molecules, compositions, systems, vectors, vector systems, systems, cells, or any combination thereof of the present invention, which are also referred to as the primary active agent or ingredient, and a pharmaceutically acceptable carrier or excipient. As used herein, “pharmaceutical formulation” refers to the combination of an active agent, compound, or ingredient with a pharmaceutically acceptable carrier or excipient, making the composition suitable for diagnostic, therapeutic, or preventive use in vitro, in vivo, or ex vivo. As used herein, “pharmaceutically acceptable carrier or excipient” refers to a carrier or excipient that is useful in preparing a pharmaceutical formulation that is generally safe, non-toxic, and is neither biologically or otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient. When present, a compound or composition can optionally be present in the pharmaceutical formulation as a pharmaceutically acceptable salt. In an embodiment, the pharmaceutical formulation can include, such as an additional active ingredient, a CRISPR-Cas system or component thereof described in greater detail elsewhere herein. In an embodiment, the pharmaceutical formulation can include, such as an active ingredient, a CRISPR-Cas system polynucleotide, protein, RNP complex, or any combination thereof described in greater detail elsewhere herein.

In an embodiment, the active ingredient is present as a pharmaceutically acceptable salt of the active ingredient. As used herein, “pharmaceutically acceptable salt” refers to any acid or base addition salt whose counter-ions are non-toxic to the subject to which they are administered in pharmaceutical doses of the salts. Suitable salts include hydrobromide, iodide, nitrate, bisulfate, phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, malonate, mandelate, malate, phthalate, and pamoate.

The pharmaceutical formulations described herein can be administered to a subject in need thereof via any suitable method or route. Suitable administration routes can include, but are not limited to auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratympanic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated and/or the active ingredient(s).

Where appropriate, the engineered Acr delivery and/or additional active agent compounds, molecules, compositions, vectors, vector systems, systems, cells, or any combination thereof of the present invention can be provided to a subject in need thereof as an ingredient, such as an active ingredient or agent, in a pharmaceutical formulation. As such, also described are pharmaceutical formulations containing one or more of the compounds and salts thereof, or pharmaceutically acceptable salts thereof described herein.

In an embodiment, the subject has a disease or disorder to be treated with a CRISPR-Cas system, such as a genetic disease or disorder. In an embodiment, it is desirable to also treat the subject with an engineered Acr polypeptide delivery system of the present invention. Without being bound by theory, it can be desirable to spatially control the activity of the CRISPR-Cas system and/or the amount of CRISPR-Cas system. As used herein, “agent” refers to any substance, compound, molecule, and the like, which can be biologically active or otherwise can induce a biological and/or physiological effect on a subject to which it is administered to. As used herein, “active agent” or “active ingredient” refers to a substance, compound, or molecule, which is biologically active or otherwise, induces a biological or physiological effect on a subject to which it is administered to. In other words, “active agent” or “active ingredient” refers to a component or components of a composition to which the whole or part of the effect of the composition is attributed. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed.

### Pharmaceutically Acceptable Carriers and Secondary Ingredients and Agents

The pharmaceutical formulation can include a pharmaceutically acceptable carrier. Suitable pharmaceutically acceptable carriers include, but are not limited to water, salt solutions, alcohols, gum arabic, vegetable oils, benzyl alcohols, polyethylene glycols, gelatin, carbohydrates (such as lactose, amylose, or starch), magnesium stearate, talc, silicic acid, viscous paraffin, perfume oil, fatty acid esters, hydroxy methylcellulose, and polyvinyl pyrrolidone, which do not deleteriously react with the active composition.

The pharmaceutical formulations can be sterilized, and if desired, mixed with agents, such as lubricants, preservatives, stabilizers, wetting agents, emulsifiers, salts for influencing osmotic pressure, buffers, coloring, flavoring and/or aromatic substances, and the like which do not deleteriously react with the active compound.

In an embodiment, the pharmaceutical formulation can also include an effective amount of secondary active agents, including but not limited to, biological agents or molecules including, but not limited to, e.g. polynucleotides, amino acids, peptides, polypeptides, antibodies, aptamers, ribozymes, hormones, immunomodulators, antipyretics, anxiolytics, antipsychotics, analgesics, antispasmodics, anti-inflammatories, anti-histamines, anti-infectives, chemotherapeutics, nucleic acid modification systems (e.g. CRISPR-Cas systems), and any combination thereof.

In an embodiment, the secondary agent included in the formulation is a performance modifier. In this context, a “performance modifier” is a compound, composition, or other ingredient that modifies the function and/or activity level of a primary or other secondary active agent. In an embodiment, the performance modifier is an anti-anti-CRISPR molecule, which is effective to regulate or otherwise modify the activity of the Acr, including but not limited to Acas (see e.g., Stanley et al., Cell. 178 (6): 1452-1464.e13 (2019)) and small molecules (see e.g., Nakamura et al., Nat. Comm. 10, Article number: 194 (2019)).

### Effective Amounts

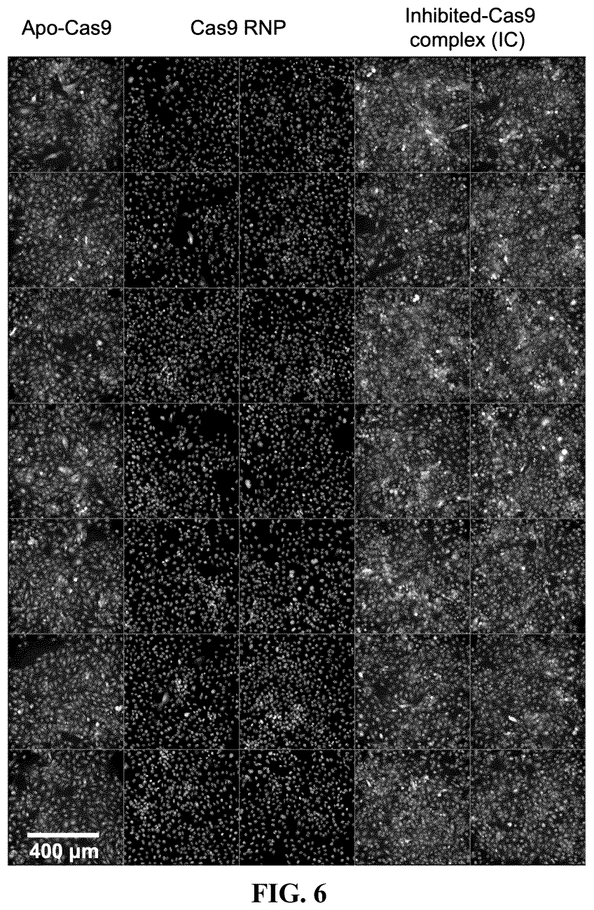

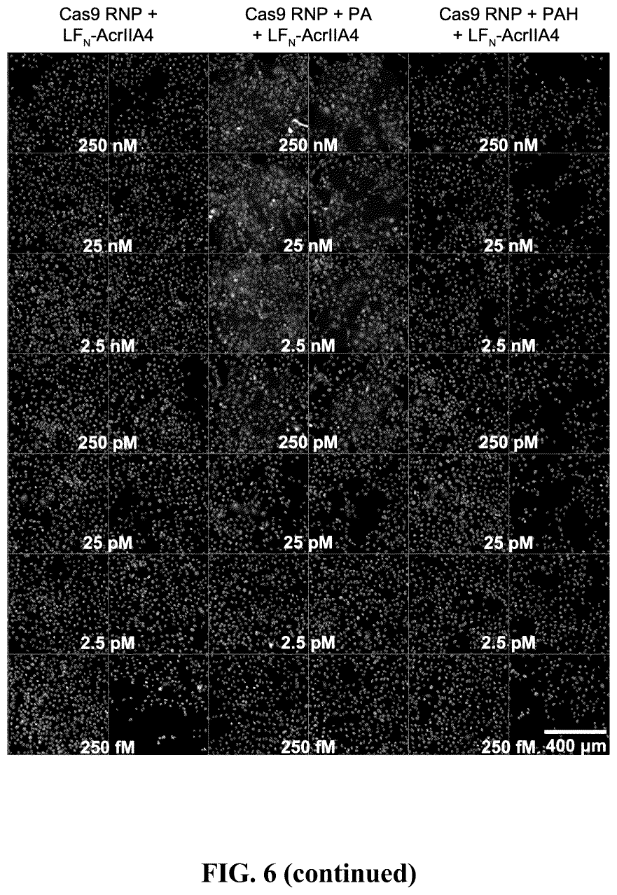

In an embodiment, the amount of the primary active agent and/or optional secondary agent can be an effective amount, least effective amount, and/or therapeutically effective amount. As used herein, “effective amount” refers to the amount of the primary and/or optional secondary agent included in the pharmaceutical formulation that achieves one or more desired effects. As used herein, “least effective” amount refers to the lowest amount of the primary and/or optional secondary agent that achieves one or more therapeutic or other desired effects. As used herein, “therapeutically effective amount” refers to the amount of the primary and/or optional secondary agent included in the pharmaceutical formulation that achieves one or more therapeutic effects. In an embodiment, one or more therapeutic effects of the engineered Acr delivery polypeptide or system thereof of the present invention are inhibiting the activity of a CRISPR-Cas system and/or reducing one or more off-target effects of a CRISPR-Cas system in a target cell. In an embodiment, the one or more therapeutic effects are cell-specific or tissue-specific polynucleotide modification, such as genome or transcript editing. In an embodiment, the one or more therapeutic effects is cell cycle-dependent polynucleotide modification, such as genome or transcript editing. See also e.g., Hoffmann et al., Nucleic Acids Research. 47 (13): e75 (2019) https://doi.org/10.1093/nar/gkz271; Lee et al., RNA 2019. 25:1421-1431; Matsumoto et al., Communications Biology volume 3, Article number: 601 (2020), Jia and Patel. Nat. Rev. Cell. Molec. 22:563-579 (2021), particularly FIG. 6; and Marino et al. Nat. Meth. 17:471-479 (2020). In an embodiment, the one or more therapeutic effects include reducing cell toxicity, restricting and/or controlling gene drive activity, controlling transcription, controlling epigenetic modulation, gene silencing, controlling gene imaging, detecting CRISPR-Cas complexes, or any combination hereof. In an embodiment, the system can be configured as or effective as a bacteriophage therapy.

The effective amount, least effective amount, and/or therapeutically effective amount of the primary and optional secondary active agent described elsewhere herein contained in the pharmaceutical formulation can be any non-zero amount ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pg, ng, μg, mg, or g or be any numerical value or subrange within any of these ranges.

In an embodiment, the effective amount, least effective amount, and/or therapeutically effective amount can be an effective concentration, least effective concentration, and/or therapeutically effective concentration, which can each be any non-zero amount ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pM, nM, μM, mM, or M or be any numerical value or subrange within any of these ranges. Similar to effective amount, least effective amount, and therapeutic effective amount, effective concentration, least effective concentration, and/or therapeutically effective concentration is the concentration where a desired effect is achieved, the least concentration at which a desired effect or effects are achieved, or the concentration at which one or more therapeutic effects are achieved, respectively. Exemplary effects and/or therapeutic effects are described in greater detail elsewhere herein.

In other embodiments, the effective amount, least effective amount, and/or therapeutically effective amount of the primary and optional secondary active agent can be any non-zero amount ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 international units (IU) or be any numerical value or subrange within any of these ranges.

In an embodiment, the primary and/or the optional secondary active agent present in the pharmaceutical formulation can be any non-zero amount ranging from about 0 to 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the pharmaceutical formulation or be any numerical value or subrange within any of these ranges.

In an embodiment where a cell or cell population is present in the pharmaceutical formulation (e.g., as a primary and/or secondary active agent), the effective amount of cells can be any amount ranging from about 1 or 2 cells to 1×10¹ cells/mL, 1×10²⁰ cells/mL or more, such as about 1×10¹ cells/mL, 1×10² cells/mL, 1×10³ cells/mL, 1×10⁴ cells/mL, 1×10⁵ cells/mL, 1×10⁶ cells/mL, 1×10⁷ cells/mL, 1×10⁸ cells/mL, 1×10⁹ cells/mL, 1×10¹⁰ cells/mL, 1×10¹¹ cells/mL, 1×10¹² cells/mL, 1×10¹³ cells/mL, 1×10¹⁴ cells/mL, 1×10¹⁵ cells/mL, 1×10¹⁶ cells/mL, 1×10¹⁷ cells/mL, 1×10¹⁸ cells/mL, 1×10¹⁹ cells/mL, to/or about 1×0²⁰/cells mL or any numerical value or subrange within any of these ranges.

In an embodiment, the amount or effective amount, particularly where an infective particle is being delivered (e.g., a virus particle having the primary or secondary agent as a cargo), the effective amount of virus particles can be expressed as a titer (plaque forming units per unit of volume) or as a MOI (multiplicity of infection). In an embodiment, the effective amount can be about 1×01 particles per pL, nL, μL, mL, or L to 1×10²⁰ particles per pL, nL, μL, mL, or L or more, such as about 1×10¹, 1×10², 1×10³, 1×10⁴, 1×0⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, to/or about 1×10²⁰ particles per pL, nL, μL, mL, or L. In an embodiment, the effective titer can be about 1×10¹ transforming units per pL, nL, μL, mL, or L to 1×10²⁰ transforming units per pL, nL, μL, mL, or L or more, such as about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹ to/or about 1×10²⁰ transforming units per pL, nL, μL, mL, or L or any numerical value or subrange within these ranges. In an embodiment, the MOI of the pharmaceutical formulation can range from about 0.1 to 10 or more, such as 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10 or more or any numerical value or subrange within these ranges.

In an embodiment, the amount or effective amount of one or more of the active agent(s) described herein contained in the pharmaceutical formulation can range from about 1 μg/kg to about 10 mg/kg based upon the bodyweight of the subject in need thereof or average bodyweight of the specific patient population to which the pharmaceutical formulation can be administered.

In embodiments where there is a secondary agent contained in the pharmaceutical formulation, the effective amount of the secondary active agent will vary depending on the secondary agent, the primary agent, the administration route, subject age, disease, stage of disease, among other things, which can be appreciated by one of ordinary skill in the art.

When optionally present in the pharmaceutical formulation, the secondary active agent can be included in the pharmaceutical formulation or can exist as a stand-alone compound or pharmaceutical formulation that can be administered contemporaneously or sequentially (e.g., before or after with the compound, derivative thereof, or pharmaceutical formulation thereof.

In an embodiment, the effective amount of the secondary active agent, when optionally present, is any non-zero amount ranging from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% w/w, v/v, or w/v of the total active agents present in the pharmaceutical formulation or any numerical value or subrange within these ranges. In additional embodiments, the effective amount of the secondary active agent is any non-zero amount ranging from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% w/w, v/v, or w/v of the total pharmaceutical formulation or any numerical value or subrange within these ranges.

### Dosage Forms

In an embodiment, the pharmaceutical formulations described herein can be provided in a dosage form. The dosage form can be administered to a subject in need thereof. The dosage form can be effective to generate a specific concentration, such as an effective concentration, at a given site in the subject in need thereof. As used herein, “dose,” “unit dose,” or “dosage” can refer to physically discrete units suitable for use in a subject, each unit containing a predetermined quantity of the primary active agent, and optionally present secondary active ingredient, and/or a pharmaceutical formulation thereof calculated to produce the desired response or responses in association with its administration. In an embodiment, the given site is proximal to the administration site. In an embodiment, the given site is distal to the administration site. In some cases, the dosage form contains a greater amount of one or more of the active ingredients present in the pharmaceutical formulation than the final intended amount needed to reach a specific region or location within the subject to account for loss of the active components such as via first and second pass metabolism.

The dosage forms can be adapted for administration by any appropriate route. Appropriate routes include, but are not limited to, oral (including buccal or sublingual), rectal, intraocular, inhaled, intranasal, topical (including buccal, sublingual, or transdermal), vaginal, parenteral, subcutaneous, intramuscular, intravenous, internasal, and intradermal. Other appropriate routes are described elsewhere herein. Such formulations can be prepared by any method known in the art.

Dosage forms adapted for oral administration can be discrete dosage units such as capsules, pellets or tablets, powders or granules, solutions, or suspensions in aqueous or non-aqueous liquids; edible foams or whips, or in oil-in-water liquid emulsions or water-in-oil liquid emulsions. In an embodiment, the pharmaceutical formulations adapted for oral administration also include one or more agents which flavor, preserve, color, or help disperse the pharmaceutical formulation. Dosage forms prepared for oral administration can also be in the form of a liquid solution that can be delivered as a foam, spray, or liquid solution. The oral dosage form can be administered to a subject in need thereof. Where appropriate, the dosage forms described herein can be microencapsulated.

The dosage form can also be prepared to prolong or sustain the release of any ingredient. In an embodiment, compounds, molecules, compositions, vectors, vector systems, cells, or a combination thereof described herein can be the ingredient whose release is delayed. In an embodiment, the primary active agent is the ingredient whose release is delayed. In an embodiment, an optional secondary agent can be the ingredient whose release is delayed. Suitable methods for delaying the release of an ingredient include, but are not limited to, coating or embedding the ingredients in materials, such as polymers, wax, gels, and the like. Delayed release dosage formulations can be prepared as described in standard references such as “Pharmaceutical dosage form tablets,” eds. Liberman et. al. (New York, Marcel Dekker, Inc., 1989), “Remington—The science and practice of pharmacy”, 20th ed., Lippincott Williams & Wilkins, Baltimore, MD, 2000, and “Pharmaceutical dosage forms and drug delivery systems”, 6th Edition, Ansel et al., (Media, PA: Williams and Wilkins, 1995). These references provide information on excipients, materials, equipment, and processes for preparing tablets and capsules and delayed release dosage forms of tablets and pellets, capsules, and granules. The delayed release can be anywhere from about an hour to about 3 months or more.

Examples of suitable coating materials to prolong the release of an ingredient include, but are not limited to, cellulose polymers such as cellulose acetate phthalate, hydroxypropyl cellulose, hydroxypropyl methylcellulose, hydroxypropyl methylcellulose phthalate, and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Coatings may be formed with a different ratio of water-soluble polymers, water-insoluble polymers, and/or pH-dependent polymers, with or without water-insoluble/water-soluble non-polymeric excipients, to produce the desired release profile. The coating is either performed on the dosage form (matrix or simple) which includes, but is not limited to, tablets (compressed with or without coated beads), capsules (with or without coated beads), beads, particle compositions, “ingredient as is” formulated as, but is not limited to, a suspension form or as a sprinkle dosage form.

Where appropriate, the dosage forms described herein can be a liposome. In these embodiments, primary active ingredient(s), and/or optional secondary active ingredient(s), and/or pharmaceutically acceptable salt thereof where appropriate are incorporated into a liposome. In embodiments where the dosage form is a liposome, the pharmaceutical formulation is thus a liposomal formulation. The liposomal formulation can be administered to a subject in need thereof.

Dosage forms adapted for topical administration can be formulated as ointments, creams, suspensions, lotions, powders, solutions, pastes, gels, sprays, aerosols, or oils. In an embodiment for treatments of the eye or other external tissues, for example, the mouth or the skin, the pharmaceutical formulations are applied as a topical ointment or cream. When formulated in an ointment, a primary active ingredient, optional secondary active ingredient, and/or pharmaceutically acceptable salt thereof where appropriate can be formulated with a paraffinic or water-miscible ointment base. In other embodiments, the primary and/or secondary active ingredient can be formulated in a cream with an oil-in-water cream base or a water-in-oil base. Dosage forms adapted for topical administration in the mouth include lozenges, pastilles, and mouth washes.

Dosage forms adapted for nasal or inhalation administration include aerosols, solutions, suspension drops, gels, or dry powders. In an embodiment, a primary active ingredient, optional secondary active ingredient, and/or pharmaceutically acceptable salt thereof where appropriate can be in a dosage form adapted for inhalation and is in a particle-size-reduced form that is obtained or obtainable by micronization. In an embodiment, the particle size of the size reduced (e.g., micronized) compound or salt or solvate thereof, is defined by a D₅₀ value of about 0.5 to about 10 microns as measured by an appropriate method known in the art. Dosage forms adapted for administration by inhalation also include particle dusts or mists. Suitable dosage forms wherein the carrier or excipient is a liquid for administration as a nasal spray or drops include aqueous or oil solutions/suspensions of an active (primary and/or secondary) ingredient, which may be generated by various types of metered dose pressurized aerosols, nebulizers, or insufflators. The nasal/inhalation formulations can be administered to a subject in need thereof.

In an embodiment, the dosage forms are aerosol formulations suitable for administration by inhalation. In some of these embodiments, the aerosol formulation contains a solution or fine suspension of a primary active ingredient, secondary active ingredient, and/or pharmaceutically acceptable salt thereof where appropriate and a pharmaceutically acceptable aqueous or non-aqueous solvent. Aerosol formulations can be presented in single or multi-dose quantities in sterile form in a sealed container. For some of these embodiments, the sealed container is a single-dose or multi-dose nasal or an aerosol dispenser fitted with a metering valve (e.g., metered dose inhaler), which is intended for disposal once the contents of the container have been exhausted.

Where the aerosol dosage form is contained in an aerosol dispenser, the dispenser contains a suitable propellant under pressure, such as compressed air, carbon dioxide, or an organic propellant, including but not limited to a hydrofluorocarbon. The aerosol formulation dosage forms in other embodiments are contained in a pump-atomizer. The pressurized aerosol formulation can also contain a solution or a suspension of a primary active ingredient, optional secondary active ingredient, and/or pharmaceutically acceptable salt thereof. In further embodiments, the aerosol formulation also contains co-solvents and/or modifiers incorporated to improve, for example, the stability and/or taste and/or fine particle mass characteristics (amount and/or profile) of the formulation. Administration of the aerosol formulation can be once daily or several times daily, for example, 2, 3, 4, or 8 times daily, in which 1, 2, 3, or more doses are delivered each time. The aerosol formulations can be administered to a subject in need thereof.

For some dosage forms suitable and/or adapted for inhaled administration, the pharmaceutical formulation is a dry powder inhalable formulation. In addition to a primary active agent, optional secondary active ingredient, and/or pharmaceutically acceptable salt thereof where appropriate, such a dosage form can contain a powder base such as lactose, glucose, trehalose, mannitol, and/or starch. In some of these embodiments, a primary active agent, secondary active ingredient, and/or pharmaceutically acceptable salt thereof where appropriate is in a particle-size reduced form.

In further embodiments, a performance modifier, such as L-leucine or another amino acid, cellobiose octaacetate, and/or metal salts of stearic acid, such as magnesium or calcium stearate. In an embodiment, the aerosol formulations are arranged so that each metered dose of aerosol contains a predetermined amount of an active ingredient, such as the one or more of the compositions, compounds, vector(s), molecules, cells, and combinations thereof described herein.

Dosage forms adapted for vaginal administration can be presented as pessaries, tampons, creams, gels, pastes, foams, or spray formulations. Dosage forms adapted for rectal administration include suppositories or enemas. The vaginal formulations can be administered to a subject in need thereof.

Dosage forms adapted for parenteral administration and/or adapted for injection can include aqueous and/or non-aqueous sterile injection solutions, which can contain antioxidants, buffers, bacteriostats, solutes that render the composition isotonic with the blood of the subject, and aqueous and non-aqueous sterile suspensions, which can include suspending agents and thickening agents. The dosage forms adapted for parenteral administration can be presented in single-unit dose or multi-unit dose containers, including but not limited to sealed ampoules or vials. The doses can be lyophilized and re-suspended in a sterile carrier to reconstitute the dose prior to administration. Extemporaneous injection solutions and suspensions can be prepared in an embodiment, from sterile powders, granules, and tablets. The parenteral formulations can be administered to a subject in need thereof.

For an embodiment, the dosage form contains a predetermined amount of a primary active agent, secondary active ingredient, and/or pharmaceutically acceptable salt thereof where appropriate per unit dose. In an embodiment, the predetermined amount of primary active agent, secondary active ingredient, and/or pharmaceutically acceptable salt thereof where appropriate can be an effective amount, a least effective amount, and/or a therapeutically effective amount. In other embodiments, the predetermined amount of a primary active agent, secondary active agent, and/or pharmaceutically acceptable salt thereof where appropriate, can be an appropriate fraction of the effective amount of the active ingredient.

### Co-Therapies and Combination Therapies

In an embodiment, the pharmaceutical formulation(s) described herein are part of a combination treatment or combination therapy. The combination treatment can include the pharmaceutical formulation described herein and an additional treatment modality. The additional treatment modality can be a chemotherapeutic, a genetic modifier, a biological therapeutic, surgery, radiation, diet modulation, environmental modulation, a physical activity modulation, and combinations thereof.

In an embodiment, the co-therapy or combination therapy additionally includes but is not limited to, polynucleotides, amino acids, peptides, polypeptides, antibodies, aptamers, ribozymes, hormones, immunomodulators, antipyretics, anxiolytics, antipsychotics, analgesics, antispasmodics, anti-inflammatories, anti-histamines, anti-infectives, chemotherapeutics, genetic modifiers (e.g., CRISPR-Cas systems), and combinations thereof.

In an embodiment, the co-therapy or combination therapy is a CRISPR-Cas system that comprises a Cas protein. In an embodiment, the co-therapy or combination therapy is a Class 2, Type II CRISPR-Cas system. In an embodiment, the co-therapy or combination therapy is a CRISPR-Cas9 system. In an embodiment, the co-therapy or combination therapy is a Class 2, Type V CRISPR-Cas system. In an embodiment, the co-therapy or combination therapy is a CRISPR-Cas12 system. In an embodiment, the co-therapy or combination therapy is a Class 2, Type VI CRISPR-Cas system. In an embodiment, the co-therapy or combination therapy is a CRISPR-Cas13 system. Other exemplary CRISPR-Cas systems that can be included in a co-therapy or combination therapy are described in greater detail elsewhere herein.

### Exemplary CRISPR-Cas Systems

As described elsewhere herein, the engineered Acr delivery compositions described herein can be provided to cells containing a CRISPR-Cas system or component thereof or delivered with the CRISPR-Cas system as an auxiliary agent in a pharmaceutical formulation or as a co- or combination therapy. In general, a CRISPR-Cas or CRISPR system as used herein and in documents, such as WO 2014/093622 (PCT/US2013/074667), refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g. CRISPR RNA and transactivating (tracr) RNA or a single guide RNA (sgRNA) (chimeric RNA)), or other sequences and transcripts from a CRISPR locus. In general, a CRISPR system is characterized by elements that promote the formation of a CRISPR complex at the site of a target sequence (also referred to as a protospacer in the context of an endogenous CRISPR system). See, e.g., Shmakov et al. (2015) “Discovery and Functional Characterization of Diverse Class 2 CRISPR-Cas Systems”, Molecular Cell, DOI: dx.doi.org/10.1016/j.molcel.2015.10.008. The term “CRISPR systems” includes any form such as polynucleotides, proteins, and complexes (e.g., RNPs), which are described in greater detail elsewhere herein. The terms “CRISPR-Cas system” and “CRISPR system” are used interchangeably herein.

### Class 1 Systems

The methods, systems, and tools provided herein may be designed for use with Class 1 CRISPR proteins. In an example embodiment, the Class 1 system may be Type I, Type III or Type IV Cas proteins as described in Makarova et al. “Evolutionary classification of CRISPR-Cas systems: a burst of class 2 and derived variants” Nature Reviews Microbiology, 18:67-81 (February 2020), incorporated in its entirety herein by reference, and particularly as described in FIG. 1, p. 326. The Class 1 systems typically use a multi-protein effector complex, which can, in an embodiment, include ancillary proteins, such as one or more proteins in a complex referred to as a CRISPR-associated complex for antiviral defense (Cascade), one or more adaptation proteins (e.g., Cas1, Cas2, RNA nuclease), and/or one or more accessory proteins (e.g. Cas 4, DNA nuclease, Cas3, etc.), CRISPR associated Rossmann fold (CARF) domain-containing proteins, and/or RNA transcriptase. Although Class 1 systems have limited sequence similarity, Class 1 system proteins can be identified by their similar architectures, including one or more Repeat-Associated Mysterious Protein (RAMP) family subunits, e.g., Cas 5, Cas6, Cas7. RAMP proteins are characterized by having one or more RNA recognition motif domains. Large subunits (for example, Cas8 or Cas10) and small subunits (for example, Cas11) are also typical of Class 1 systems. See, e.g., FIGS. 1 and 2. Koonin E V, Makarova K S. 2019 Origins and evolution of CRISPR-Cas systems. Phil. Trans. R. Soc. B 374:20180087, DOI: 10.1098/rstb.2018.0087. In one aspect, Class 1 systems are characterized by the signature protein Cas3. The Cascade in particular Class 1 proteins can comprise a dedicated complex of multiple Cas proteins that binds pre-crRNA and recruits an additional Cas protein, for example, Cas6 or Cas5, which is the nuclease directly responsible for processing pre-crRNA. In one aspect, the Type I CRISPR protein comprises an effector complex with one or more Cas5 subunits and two or more Cas7 subunits. Class 1 subtypes include Type I-A, I-B, I-C, I-U, I-D, I-E, and I-F, Type IV-A and IV-B, IV-C, and Type III-A, III-D, III-B, III-C, III-E, and III-F III-B. See e.g., Marakova et al., Nat. Rev. Microbiol. 18, pages 67-83 (2020). Class 1 systems also include CRISPR-Cas variants, including Type I-A, I-B, I-E, I-F, I-U, and Tye IV variants, which can include variants carried by transposons and plasmids, including versions of subtype I-F encoded by a large family of Tn7-like transposon and smaller groups of Tn7-like transposons that encode similarly degraded subtype I-B systems. Peters et al., PNAS 114 (35) (2017); DOI: 10.1073/pnas. 1709035114; see also, Makarova et al, the CRISPR Journal, v. 1, n5, FIG. 5; and Theoretical and Applied Genetics (2022) 135:367-387

### Class 2 Systems

The compositions, systems, and methods described in greater detail elsewhere herein can be designed and adapted for use with Class 2 CRISPR-Cas systems. Thus, in an embodiment, the CRISPR-Cas system is a Class 2 CRISPR-Cas system. Class 2 systems are distinguished from Class 1 systems in that they have a single, large, multi-domain effector protein. In an example embodiment, the Class 2 system can be a Type II, Type V, or Type VI system, which are described in Makarova et al. “Evolutionary classification of CRISPR-Cas systems: a burst of class 2 and derived variants” Nature Reviews Microbiology, 18:67-81 (February 2020), incorporated herein by reference. Each type of Class 2 system is further divided into subtypes. See Markova et al. 2020, particularly at FIG. 2. Class 2, Type II systems can be divided into 4 subtypes: II-A, II-B, II-C1, and II-C2. Class 2, Type V systems can be divided into 17 subtypes: V-A, V-B1, V-B2, V-C, V-D, V-E, V-F1, V-F1 (V-U3), V-F2, V-F3, V-G, V-H, V-I, V-K (V-U5), V-U1, V-U2, and V-U4. Class 2, Type VI systems can be divided into 5 subtypes: VI-A, VI-B1, VI-B2, VI-C, and VI-D.

The distinguishing feature of these types is that their effector complexes consist of a single, large, multi-domain protein. Type V systems differ from Type II systems (e.g., Cas9), which contain two nuclear domains (HNH and RuvC) that are each responsible for the cleavage of one strand of the target DNA. The Type V systems (e.g., Cas12) only contain a RuvC-like nuclease domain that cleaves both strands. Type VI (Cas13) are unrelated to the effectors of Type II and V systems and contain two HEPN domains and target RNA. Cas13 proteins also display collateral activity that is triggered by target recognition. Some Type V systems have also been found to possess this collateral activity with single-stranded DNA or RNA. See e.g., Tong et al., Front. Cell. Dev. Biol. 2021. https://doi.org/10.3389/fcell.2020.622103.

In an embodiment, the Class 2 system is a Type II system. In an embodiment, the Type II CRISPR-Cas system is a II-A CRISPR-Cas system. In an embodiment, the Type II CRISPR-Cas system is a II-B CRISPR-Cas system. In an embodiment, the Type II CRISPR-Cas system is a II-C1 CRISPR-Cas system. In an embodiment, the Type II CRISPR-Cas system is a II-C2 CRISPR-Cas system. In an embodiment, the Type II system is a Cas9 system. In an embodiment, the Type II system includes a Cas9.

In an embodiment, the Class 2 system is a Type V system. In an embodiment, the Type V CRISPR-Cas system is a V-A CRISPR-Cas system. In an embodiment, the Type V CRISPR-Cas system is a V-B1 CRISPR-Cas system. In an embodiment, the Type V CRISPR-Cas system is a V-B2 CRISPR-Cas system. In an embodiment, the Type V CRISPR-Cas system is a V-C CRISPR-Cas system. In an embodiment, the Type V CRISPR-Cas system is a V-D CRISPR-Cas system. In an embodiment, the Type V CRISPR-Cas system is a V-E CRISPR-Cas system. In an embodiment, the Type V CRISPR-Cas system is a V-F1 CRISPR-Cas system. In an embodiment, the Type V CRISPR-Cas system is a V-F1 (V-U3) CRISPR-Cas system. In an embodiment, the Type V CRISPR-Cas system is a V-F2 CRISPR-Cas system. In an embodiment, the Type V CRISPR-Cas system is a V-F3 CRISPR-Cas system. In an embodiment, the Type V CRISPR-Cas system is a V-G CRISPR-Cas system. In an embodiment, the Type V CRISPR-Cas system is a V-H CRISPR-Cas system. In an embodiment, the Type V CRISPR-Cas system is a V-I CRISPR-Cas system. In an embodiment, the Type V CRISPR-Cas system is a V-K (V-U5) CRISPR-Cas system. In an embodiment, the Type V CRISPR-Cas system is a V-U1 CRISPR-Cas system. In an embodiment, the Type V CRISPR-Cas system is a V-U2 CRISPR-Cas system. In an embodiment, the Type V CRISPR-Cas system is a V-U4 CRISPR-Cas system. In an embodiment, the Type V CRISPR-Cas system includes a Cas12a (Cpf1), Cas12b (C2c1), Cas12b2, Cas12c (C2c3), Cas12d (CasY), Cas12e (CasX), Cas12k, Cas14, Cas12f1 (Cas14a), Cas12f2 (Cas14b), Cas12g, Cas12h, Cas12i, C2c4, C2c8, C2c9, C2c10, and/or CasΦ.

In an embodiment the Class 2 system is a Type VI system. In an embodiment, the Type VI CRISPR-Cas system is a VI-A CRISPR-Cas system. In an embodiment, the Type VI CRISPR-Cas system is a VI-B1 CRISPR-Cas system. In an embodiment, the Type VI CRISPR-Cas system is a VI-B2 CRISPR-Cas system. In an embodiment, the Type VI CRISPR-Cas system is a VI-C CRISPR-Cas system. In an embodiment, the Type VI CRISPR-Cas system is a VI-D CRISPR-Cas system. In an embodiment, the Type VI CRISPR-Cas system includes a Cas13a (C2c2), Cas13b, Cas13c, and/or Cas13d.

### Guide Molecules

The CRISPR-Cas or Cas-Based system described herein can, in an embodiment, include one or more guide molecules. The terms guide molecule, guide sequence and guide polynucleotide refer to polynucleotides capable of guiding Cas to a target genomic locus and are used interchangeably as in foregoing cited documents such as International Patent Publication No. WO 2014/093622 (PCT/US2013/074667). In general, a guide sequence is any polynucleotide sequence having sufficient complementarity with a target polynucleotide sequence to hybridize with the target sequence and direct sequence-specific binding of a CRISPR complex to the target sequence. The guide molecule can be a polynucleotide.

The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by the Surveyor assay (Qui et al. 2004. BioTechniques. 36 (4) 702-707). Similarly, cleavage of a target nucleic acid sequence may be evaluated in a test tube by providing the target nucleic acid sequence, components of a nucleic acid-targeting complex, including the guide sequence to be tested and a control guide sequence different from the test guide sequence, and comparing binding or rate of cleavage at the target sequence between the test and control guide sequence reactions. Other assays are possible and will occur to those skilled in the art.

In an embodiment, the guide molecule is an RNA. The guide molecule(s) (also referred to interchangeably herein as guide polynucleotide and guide sequence) that are included in the CRISPR-Cas or Cas-based system can be any polynucleotide sequence having sufficient complementarity with a target nucleic acid sequence to hybridize with the target nucleic acid sequence and direct sequence-specific binding of a nucleic acid-targeting complex to the target nucleic acid sequence. In an embodiment, the degree of complementarity, when optimally aligned using a suitable alignment algorithm, can be about or more than about 50%, 60%, 75%, 80%, 85%, 90%, 95%, 97.5%, 99%, or more. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g., the Burrows-Wheeler Aligner), ClustalW, Clustal X, BLAT, Novoalign (Novocraft Technologies; available at www.novocraft.com), ELAND (Illumina, San Diego, CA), SOAP (available at soap.genomics.org.cn), and Maq (available at maq.sourceforge.net).

A guide sequence, and hence a nucleic acid-targeting guide, may be selected to target any target nucleic acid sequence. The target sequence may be DNA. The target sequence may be any RNA sequence. In an embodiment, the target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double-stranded RNA (dsRNA), non-coding RNA (ncRNA), long non-coding RNA (lncRNA), and small cytoplasmaic RNA (scRNA). In some preferred embodiments, the target sequence may be a sequence within an RNA molecule selected from the group consisting of mRNA, pre-mRNA, and rRNA. In some preferred embodiments, the target sequence may be a sequence within an RNA molecule selected from the group consisting of ncRNA, and lncRNA. In some more preferred embodiments, the target sequence may be a sequence within an mRNA molecule or a pre-mRNA molecule.

In an embodiment, a nucleic acid-targeting guide is selected to reduce the degree of secondary structure within the nucleic acid-targeting guide. In an embodiment, about or less than about 75%, 50%, 40%, 30%, 25%, 20%, 15%, 10%, 5%, 1%, or fewer of the nucleotides of the nucleic acid-targeting guide participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106 (1): 23-24; and P A Carr and G M Church, 2009, Nature Biotechnology 27 (12): 1151-62).

In certain embodiments, a guide RNA or CRISPR RNA (crRNA) may comprise, consist essentially of, or consist of a direct repeat (DR) sequence and a guide sequence or spacer sequence. In certain embodiments, the guide RNA or crRNA may comprise, consist essentially of, or consist of a direct repeat sequence fused or linked to a guide sequence or spacer sequence. In certain embodiments, the direct repeat sequence may be located upstream (i.e., 5′) from the guide sequence or spacer sequence. In other embodiments, the direct repeat sequence may be located downstream (i.e., 3′) from the guide sequence or spacer sequence.

In certain embodiments, the crRNA comprises a stem-loop, preferably a single stem-loop. In certain embodiments, the direct repeat sequence forms a stem-loop, preferably a single stem-loop.

In certain embodiments, the spacer length of the guide RNA is from 15 to 35 nucleotides (nt). In certain embodiments, the spacer length of the guide RNA is at least 15 nt. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27 to 30 nt, e.g., 27, 28, 29, or 30 nt, from 30 to 35 nt, e.g., 30, 31, 32, 33, 34, or 35 nt, or 35 nt or longer.

The “tracrRNA” sequence or analogous terms includes any polynucleotide sequence that has sufficient complementarity with a crRNA sequence to hybridize. In an embodiment, the degree of complementarity between the tracrRNA sequence and crRNA sequence along the length of the shorter of the two when optimally aligned is about or more than about 25%, 30%, 40%, 50%, 60%, 70%, 80%, 90%, 95%, 97.5%, 99%, or higher. In an embodiment, the tracr sequence is about or more than about 5, 6, 7, 8, 9, 10, 11, 12, 13, 14, 15, 16, 17, 18, 19, 20, 25, 30, 40, 50, or more nucleotides in length. In an embodiment, the tracr sequence and crRNA sequence are contained within a single transcript, such that hybridization between the two produces a transcript having a secondary structure, such as a hairpin.

In general, the degree of complementarity is with reference to the optimal alignment of the guide sequence and tracr sequence, along the length of the shorter of the two sequences. Optimal alignment may be determined by any suitable alignment algorithm and may further account for secondary structures, such as self-complementarity within either the guide sequence or tracr sequence. In an embodiment, the degree of complementarity between the tracr sequence and guide sequence along the length of the shorter of the two when optimally aligned is about or more than about 25%, 30%, 40%, 50%, 60%, 70%, 80%, 90%, 95%, 97.5%, 99%, or higher.

In an embodiment, the degree of complementarity between a guide sequence and its corresponding target sequence can be about or more than about 50%, 60%, 75%, 80%, 85%, 90%, 95%, 97.5%, 99%, or 100%. In an embodiment, a guide RNA or sgRNA can be about or more than about 5, 10, 11, 12, 13, 14, 15, 16, 17, 18, 19, 20, 21, 22, 23, 24, 25, 26, 27, 28, 29, 30, 35, 40, 45, 50, 75, 76, 77, 78, 79, 80, 81, 82, 83, 84, 85, 86, 87, 88, 89, 90, 91, 92, 93, 94, 95, 96, 97, 98, 99, 100 or more nucleotides in length. In an embodiment, a guide RNA or sgRNA can be less than about 100, 75, 50, 45, 40, 35, 30, 25, 20, 15, 12, or fewer nucleotides in length; and a tracr RNA can be 30 or 50 nucleotides in length. In an embodiment, the degree of complementarity between a guide sequence and its corresponding target sequence is greater than 94.5% or 95% or 95.5% or 96% or 96.5% or 97% or 97.5% or 98% or 98.5% or 99% or 99.5% or 99.9%, or 100%. Off target is less than 100% or 99.9% or 99.5% or 99% or 99% or 98.5% or 98% or 97.5% or 97% or 96.5% or 96% or 95.5% or 95% or 94.5% or 94% or 93% or 92% or 91% or 90% or 89% or 88% or 87% or 86% or 85% or 84% or 83% or 82% or 81% or 80% complementarity between the sequence and the guide, with it being advantageous that there is 100% or 99.9% or 99.5% or 99% or 99% or 98.5% or 98% or 97.5% or 97% or 96.5% or 96% or 95.5% or 95% or 94.5% complementarity between the sequence and the guide.

In an embodiment according to the invention, the guide RNA (capable of guiding Cas to a target locus) may comprise (1) a guide sequence capable of hybridizing to a genomic target locus in the eukaryotic cell; (2) a tracr sequence; and (3) a tracr mate sequence. All (1) to (3) may reside in a single RNA, i.e., an sgRNA (arranged in a 5′ to 3′ orientation), or the tracr RNA may be a different RNA than the RNA containing the guide and tracr mate sequence. The tracr hybridizes to the tracr mate sequence and directs the CRISPR/Cas complex to the target sequence. Where the tracr RNA is on a different RNA than the RNA containing the guide and tracr mate sequence, the length of each RNA may be optimized to be shortened from their respective native lengths, and each may be independently chemically modified to protect from degradation by cellular ribonucleases or otherwise increase stability.

Many modifications to guide sequences are known in the art and are further contemplated within the context of this invention. Various modifications may be used to increase the specificity of binding to the target sequence and/or increase the activity of the Cas protein and/or reduce off-target effects. Example guide sequence modifications are described in International Patent Application No. PCT US2019/045582, specifically paragraphs [0178]-[0333]. which is incorporated herein by reference.

### Target Sequences, PAMs, and PFSs

In the context of the formation of a CRISPR complex, “target sequence” refers to a sequence to which a guide sequence is designed to have complementarity, where hybridization between a target sequence and a guide sequence promotes the formation of a CRISPR complex. It will be appreciated that “CRISPR complex” generally refers to a Cas complexed with a guide RNA and optionally a target polynucleotide, and/or other molecules involved in activity of the CRISPR-Cas system. Such a term includes RNPs formed of a Cas protein complexed with a gRNA and those otherwise formed. A target sequence may comprise RNA polynucleotides. The term “target RNA” refers to an RNA polynucleotide being or comprising the target sequence. In other words, the target polynucleotide can be a polynucleotide or a part of a polynucleotide to which a part of the guide sequence is designed to have complementarity with and to which the effector function mediated by the complex comprising the CRISPR effector protein and a guide molecule is to be directed. In an embodiment, a target sequence is located in the nucleus or cytosol of a cell.

The guide sequence can specifically bind a target sequence in a target polynucleotide. The target polynucleotide may be DNA. The target polynucleotide may be RNA. The target polynucleotide can have one or more (e.g., 1, 2, 3, 4, 5, 6, 7, 8, 9, 10, etc. or more) target sequences. The target polynucleotide can be on a vector. The target polynucleotide can be genomic DNA. The target polynucleotide can be episomal. Other forms of the target polynucleotide are described elsewhere herein.

The target sequence may be DNA. The target sequence may be any RNA sequence. In an embodiment, the target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double-stranded RNA (dsRNA), non-coding RNA (ncRNA), long non-coding RNA (lncRNA), and small cytoplasmatic RNA (scRNA). In some preferred embodiments, the target sequence (also referred to herein as a target polynucleotide) may be a sequence within an RNA molecule selected from the group consisting of mRNA, pre-mRNA, and rRNA. In some preferred embodiments, the target sequence may be a sequence within an RNA molecule selected from the group consisting of ncRNA, and lncRNA. In some more preferred embodiments, the target sequence may be a sequence within an mRNA molecule or a pre-mRNA molecule.

### PAM and PFS Elements

PAM (protospacer adjacent motif) elements are sequences that can be recognized and bound by Cas proteins. Cas proteins/effector complexes can then unwind the dsDNA at a position adjacent to the PAM element. It will be appreciated that RNA-targeting Cas proteins and systems do not require PAM sequences (Marraffini et al. 2010. Nature. 463:568-571). Instead, many rely on PFSs (protospacer flanking sequence or site), which are discussed elsewhere herein. In certain embodiments, the target sequence should be associated with a PAM or PFS, that is, a short sequence recognized by the CRISPR complex. Depending on the nature of the CRISPR-Cas protein, the target sequence should be selected, such that its complementary sequence in the DNA duplex (also referred to herein as the non-target sequence) is upstream or downstream of the PAM. In an embodiment, the complementary sequence of the target sequence is downstream (3′ of the PAM) or upstream (5′ of the PAM). The precise sequence and length requirements for the PAM differ depending on the Cas protein used, but PAMs are typically 2-5 base pair sequences adjacent to the protospacer (that is, the target sequence). Examples of the natural PAM sequences for different Cas proteins are provided herein below and the skilled person will be able to identify further PAM sequences for use with a given Cas protein.

The ability to recognize different PAM sequences depends on the Cas polypeptide(s) included in the system. See e.g., Gleditzsch et al. 2019. RNA Biology. 16 (4): 504-517. Table 5 (from Gleditzsch et al. 2019) below shows several Cas polypeptides and the PAM sequence they recognize.

TABLE 5
Example PAM Sequences

| Cas Protein | PAM Sequence |
|---|---|
| SpCas9 | NGG/NRG |
| SaCas9 | NGRRT or NGRRN |
| NmeCas9 | NNNNGATT |
| CjCas9 | NNNNRYAC |
| StCas9 | NNAGAAW |
| Cas12a (Cpf1) | TTTV |
| (including | |
| LbCpf1 and AsCpf1) | |
| Cas12b (C2c1) | TTT, TTA, and TTC |
| Cas12c (C2c3) | TA |
| Cas12d (CasY) | TA |
| Cas12e (CasX) | TTCN |

In an embodiment, the CRISPR effector protein may recognize a 3′ PAM. In certain embodiments, the CRISPR effector protein may recognize a 3′ PAM which is 5′H, wherein H is A, C or U. In an embodiment, the CRISPR effector protein may recognize a 5′ PAM.

Further, engineering of the PAM Interacting (PI) domain on the Cas protein may allow the programming of PAM specificity to improve target site recognition fidelity, and increase the versatility of the CRISPR-Cas protein, for example as described for Cas9 in Kleinstiver B P et al. Engineered CRISPR-Cas9 nucleases with altered PAM specificities. Nature. 2015 Jul. 23; 523 (7561): 481-5. doi: 10.1038/nature14592. As further detailed herein, the skilled person will understand that Cas 12 proteins may be modified analogously. Gao et al, “Engineered Cpf1 Enzymes with Altered PAM Specificities,” bioRxiv 091611; doi: http://dx.doi.org/10.1101/091611 (Dec. 4, 2016) and Gao et al. Nat. Biotechnol. 35, 789-792 (2017). Doench et al. Nat Biotechnol. 2016 February; 34 (2): 184-191 created a pool of sgRNAs, tiling across all possible target sites of a panel of six endogenous mice and three endogenous human genes and quantitatively assessed their ability to produce null alleles of their target gene by antibody staining and flow cytometry. The authors showed that optimization of the PAM improved activity and also provided an online tool for designing sgRNAs. In an embodiment, the CRISPR-Cas system recognizes such an optimized PAM.

PAM sequences can be identified in a polynucleotide using appropriate design tools, which are commercially available as well as online. Such freely available tools include, but are not limited to, CRISPRFinder and CRISPRTarget. Mojica et al. 2009. Microbiol. 155 (Pt. 3): 733-740; Atschul et al. 1990. J. Mol. Biol. 215:403-410; Biswass et al. 2013 RNA Biol. 10:817-827; and Grissa et al. 2007. Nucleic Acid Res. 35: W52-57. Experimental approaches to PAM identification can include, but are not limited to, plasmid depletion assays (Jiang et al. 2013. Nat. Biotechnol. 31:233-239; Esvelt et al. 2013. Nat. Methods. 10:1116-1121; Kleinstiver et al. 2015. Nature. 523:481-485), screening by a high-throughput in vivo model called PAM-SCNAR (Pattanayak et al. 2013. Nat. Biotechnol. 31:839-843 and Leenay et al. 2016. Mol. Cell. 16:253), and negative screening (Zetsche et al. 2015. Cell. 163:759-771).

As previously mentioned, CRISPR-Cas systems that target RNA do not typically rely on PAM sequences. Instead, such systems typically recognize protospacer flanking sites (PFSs) instead of PAMs. Thus, Type VI CRISPR-Cas systems typically recognize protospacer flanking sites (PFSs) instead of PAMs. PFSs represent an analog to PAMs for RNA targets. Type VI CRISPR-Cas systems employ a Cas13. Some Cas13 proteins analyzed to date, such as Cas13a (C2c2) identified from Leptotrichia shahii (LshCas13a) have a specific discrimination against G at the 3′end of the target RNA. The presence of a C at the corresponding crRNA repeat site can indicate that nucleotide pairing at this position is rejected. However, some Cas13 proteins (e.g., LwaCas13a and PspCas13b) do not seem to have a PFS preference. See e.g., Gleditzsch et al. 2019. RNA Biology. 16 (4): 504-517.

Some Type VI proteins, such as subtype B, have 5′-recognition of D (G, T, A) and a 3′-motif requirement of NAN or NNA. One example is the Cas13b protein identified in Bergeyella zoohelcum (BzCas13b). See e.g., Gleditzsch et al. 2019. RNA Biology. 16 (4): 504-517.

Overall Type VI CRISPR-Cas systems appear to have less restrictive rules for substrate (e.g., target sequence) recognition than those that target DNA (e.g., Type V and type II).

### Specialized Cas-Based Systems

In an embodiment, the system is a Cas-based system that is capable of performing a specialized function or activity. For example, the Cas protein may be fused, operably coupled to, or otherwise associated with one or more functional domains. In an example embodiment, the Cas protein may be a catalytically dead Cas protein (“dCas”) and/or have nickase activity. A nickase is a Cas protein that cuts only one strand of a double-stranded target. In such embodiments, the dCas or nickase provides a sequence-specific targeting functionality that positions the functional domain to or proximate to a target sequence. Example functional domains that may be fused to, operably coupled to, or otherwise associated with a Cas protein can be or include, but are not limited to a nuclear localization signal (NLS) domain, a nuclear export signal (NES) domain, a translational activation domain, a transcriptional activation domain (e.g. VP64, p65, MyoD1, HSF1, RTA, and SET7/9), a translation initiation domain, a transcriptional repression domain (e.g., a KRAB domain, NuE domain, NcoR domain, and a SID domain such as a SID4X domain), a nuclease domain (e.g., FokI), a histone modification domain (e.g., a histone acetyltransferase), a light-inducible/controllable domain, a chemically-inducible/controllable domain, a transposase domain, a homologous recombination machinery domain, a recombinase domain, an integrase domain, a deaminase domain, and combinations thereof. Methods for generating catalytically dead Cas9 or a nickase Cas9 (WO 2014/204725, Ran et al. Cell. 2013 Sep. 12; 154 (6): 1380-1389), Cas12 (Liu et al. Nature Communications, 8, 2095 (2017), and Cas13 (International Patent Publication Nos. WO 2019/005884 and WO2019/060746) are known in the art and incorporated herein by reference.

In an embodiment, the functional domains can have one or more of the following activities: methylase activity, demethylase activity, translation activation activity, translation initiation activity, translation repression activity, transcription activation activity, transcription repression activity, transcription release factor activity, histone modification activity, nuclease activity, single-strand RNA cleavage activity, double-strand RNA cleavage activity, single-strand DNA cleavage activity, double-strand DNA cleavage activity, molecular switch activity, chemical inducibility, light inducibility, and nucleic acid binding activity. In an embodiment, one or more functional domains may comprise epitope tags or reporters. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporters include, but are not limited to, glutathione-S-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and auto-fluorescent proteins including blue fluorescent protein (BFP) and mCherry.

One or more functional domain(s) may be positioned at, near, in between, and/or in proximity to a terminus of the effector protein (e.g., a Cas protein). In embodiments having two or more functional domains, each of the two can be positioned at or near or in proximity to a terminus of the effector protein (e.g., a Cas protein). In an embodiment, such as those where the functional domain is operably coupled to the effector protein, one or more functional domains can be tethered or linked via a suitable linker (including, but not limited to, GlySer linkers) to the effector protein (e.g., a Cas protein). When there is more than one functional domain, the functional domains can be the same or different. In an embodiment, all the functional domains are the same. In an embodiment, all of the functional domains are different from each other. In an embodiment, at least two of the functional domains are different from each other. In an embodiment, at least two of the functional domains are the same as each other.

Other suitable functional domains can be found, for example, in International Patent Publication No. WO 2019/018423.

### Split CRISPR-Cas Systems

In an embodiment, the CRISPR-Cas system is a split CRISPR-Cas system. See e.g., Zetsche et al., 2015. Nat. Biotechnol. 33 (2): 139-142 and International Patent Publication WO 2019/018423, the compositions and techniques of which can be used in and/or adapted for use with the present invention. Split CRISPR-Cas proteins are set forth herein and in documents incorporated herein by reference in further detail elsewhere herein. In certain embodiments, each part of a split CRISPR protein is attached to a member of a specific binding pair, and when bound with each other, the members of the specific binding pair maintain the parts of the CRISPR protein in proximity. In certain embodiments, each part of a split CRISPR protein is associated with an inducible binding pair. An inducible binding pair is one which is capable of being switched “on” or “off” by a protein or small molecule that binds to both members of the inducible binding pair. In an embodiment, CRISPR proteins may be preferably split between domains, leaving domains intact. In particular embodiments, said Cas split domains (e.g., RuvC and HNH domains in the case of Cas9) can be simultaneously or sequentially introduced into the cell such that said split Cas domain(s) process the target nucleic acid sequence in the cell. The reduced size of the split Cas compared to the wild-type Cas allows other methods of delivery of the systems to the cells, such as the use of cell-penetrating peptides as described herein.

### DNA and RNA Base Editing

In an embodiment, a polynucleotide can be modified using a base editing system. In an embodiment, a Cas protein is connected or fused to a nucleotide deaminase. Thus, in an embodiment, the Cas-based system can be a base editing system. As used herein, “base editing” refers generally to the process of polynucleotide modification via a CRISPR-Cas-based or Cas-based system that does not include excising nucleotides to make the modification. Base editing can convert base pairs at precise locations without generating excess undesired editing byproducts that can be made using traditional CRISPR-Cas systems.

In an example embodiment, the nucleotide deaminase may be a DNA base editor used in combination with a DNA binding Cas protein such as, but not limited to, Class 2 Type II and Type V systems. Two classes of DNA base editors are generally known: cytosine base editors (CBEs) and adenine base editors (ABEs). CBEs convert a C·G base pair into a T·A base pair (Komor et al. 2016. Nature. 533:420-424; Nishida et al. 2016. Science. 353; and Li et al. Nat. Biotech. 36:324-327) and ABEs convert an A·T base pair to a G·C base pair. Collectively, CBEs and ABEs can mediate all four possible transition mutations (C to T, A to G, T to C, and G to A). View Rees and Liu. 2018. Nat. Rev. Genet. 19 (12): 770-788, particularly at FIGS. 1b, 2a-2c, 3a-3f, and Table 1. In an embodiment, the base editing system includes a CBE and/or an ABE. In an embodiment, a base editor can modify a polynucleotide. See e.g., Rees and Liu. 2018. Nat. Rev. Gent. 19 (12): 770-788. Base editors also generally do not need a DNA donor template and/or rely on homology-directed repair. Komor et al. 2016. Nature. 533:420-424; Nishida et al. 2016. Science. 353; and Gaudeli et al. 2017. Nature. 551:464-471. Upon binding to a target locus in the DNA, base pairing between the guide RNA of the system and the target DNA strand leads to displacement of a small segment of ssDNA in an “R-loop”. View Nishimasu et al. 2014. Cell. 156:935-949, Lapinaite et al., Science. 369 (6503): 566-572 (2020). DNA bases within the ssDNA bubble are modified by the enzyme component, such as a deaminase. In some systems, the catalytically disabled Cas protein can be a variant or a modified Cas with nickase functionality and can generate a nick in the non-edited DNA strand to induce cells to repair the non-edited strand using the edited strand as a template. Komor et al. 2016. Nature. 533:420-424; Nishida et al. 2016. Science. 353; and Gaudeli et al. 2017. Nature. 551:464-471.

Other Example Type V base editing systems are described in International Patent Publication Nos. WO 2018/213708, WO 2018/213726, and International Patent Applications No. PCT/US2018/067207, PCT/US2018/067225, and PCT/US2018/067307, each of which is incorporated herein by reference.

In an example embodiment, the base editing system may be an RNA base editing system. As with DNA base editors, a nucleotide deaminase capable of converting nucleotide bases may be fused to a Cas protein. However, in these embodiments, the Cas protein will need to be capable of binding RNA. Example RNA binding Cas proteins include, but are not limited to, RNA-binding Cas9s such as Francisella novicida Cas9 (“FnCas9”), Class 2 Type VI Cas systems, and Cas7-11 (see e.g., Özcan et al., Nature. 597:720-725 (2021)). The nucleotide deaminase may be a cytidine deaminase or an adenosine deaminase, or an adenosine deaminase engineered to have cytidine deaminase activity. In an example embodiment, the RNA base editor may be used to delete or introduce a post-translational modification site in the expressed mRNA. In contrast to DNA base editors, whose edits are permanent in the modified cell, RNA base editors can provide edits where finer, temporal control may be needed, for example in modulating a particular immune response. Example Type VI RNA-base editing systems are described in Cox et al. 2017. Science 358:1019-1027, International Patent Publication Nos. WO 2019/005884, WO 2019/005886, and WO 2019/071048, and International Patent Application Nos. PCT/US20018/05179 and PCT/US2018/067207, which are incorporated herein by reference. An example FnCas9 system that may be adapted for RNA base editing purposes is described in International Patent Publication No. WO 2016/106236, which is incorporated herein by reference.

An example method for delivery of base-editing systems, including use of a split-intein approach to divide CBE and ABE into reconstitutable halves, is described in Levy et al. Nature Biomedical Engineering doi.org/10.1038/s41441-019-0505-5 (2019), which is incorporated herein by reference.

In an embodiment, the base editor is inhibited by an engineered Acr delivery system or an Acr thereof. In an embodiment, the engineered Acr delivery system of the present invention or an Acr thereof reduces the off-target effects of a base editor system. See e.g., Cells 2020, 9, 1786; doi: 10.3390/cells9081786

### Prime Editors

In an embodiment, a polynucleotide can be modified using a prime editing system. See e.g. Anzalone et al. 2019. Nature. 576:149-157. Like base editing systems, prime editing systems can be capable of targeted modification of a polynucleotide without generating double-stranded breaks and does not require donor templates. Further, prime editing systems can be capable of all 12 possible combinations of transition and transversion mutations (i.e., A to C, A to T, A to G, C to A, C to T, C to G, T to A, T to G, T to C, G to A, G to T, G to C). Prime editing can operate via a “search-and-replace” methodology and can mediate targeted insertions, deletions, all 12 possible base-to-base conversions and combinations thereof. Generally, a prime editing system, as exemplified by PE1, PE2, and PE3 (Id.), can include a reverse transcriptase fused or otherwise coupled or associated with an RNA-programmable nickase and a prime-editing extended guide RNA (pegRNA) to facilitate direct copying of genetic information from the extension on the pegRNA into the target polynucleotide. Embodiments that can be used with the present invention include these and variants thereof. Prime editing can have the advantage of lower off-target activity than traditional CRISPR-Cas systems along with few byproducts and greater or similar efficiency as compared to traditional CRISPR-Cas systems.

In an embodiment, the prime editing guide molecule can specify both the target polynucleotide information (e.g., sequence) and contain a new polynucleotide cargo that replaces target polynucleotides. To initiate transfer from the guide molecule to the target polynucleotide, the PE system can nick the target polynucleotide at a target side to expose a 3′hydroxyl group, which can prime reverse transcription of an edit-encoding extension region of the guide molecule (e.g., a prime editing guide molecule or peg guide molecule) directly into the target site in the target polynucleotide. See e.g., Anzalone et al. 2019. Nature. 576:149-157, particularly at FIGS. 1b, 1c, related discussion, and Supplementary discussion.

In an embodiment, a prime editing system can be composed of a Cas polypeptide having nickase activity, a reverse transcriptase, and a prime editing guide molecule. The Cas polypeptide can lack nuclease activity. The guide molecule can include a target binding sequence as well as a primer binding sequence and a template containing the edited polynucleotide sequence. The guide molecule, Cas polypeptide, and/or reverse transcriptase can be coupled together or otherwise associate with each other to form an effector complex and edit a target sequence. In an embodiment, the Cas polypeptide is a Class 2, Type V or Type II Cas polypeptide. In an embodiment, the Cas polypeptide is a Cas9 polypeptide (e.g., is a Cas9 nickase). In an embodiment, the Cas polypeptide is fused to the reverse transcriptase. In an embodiment, the Cas polypeptide is linked to the reverse transcriptase.

In an embodiment, the prime editing system can be a PE system or variant thereof, a PE2 system or variant thereof, or a PE3 (e.g. PE3, PE3b) system. See e.g., Anzalone et al. 2019. Nature. 576:149-157, particularly at pgs. 2-3, FIGS. 2a, 3a-3f, 4a-4b, Extended data FIGS. 3a-3b, 4,

The peg guide molecule can be about 10 to about 200 or more nucleotides in length, such as 10 to/or 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 or more nucleotides in length. Optimization of the peg guide molecule can be accomplished as described in Anzalone et al. 2019. Nature. 576:149-157, particularly at pg. 3, FIG. 2a-2b, and Extended Data FIGS. 5a-c.

### CRISPR Associated Transposase (CAST) Systems

In an embodiment, a polynucleotide of the present invention described elsewhere herein can be modified using a CRISPR Associated Transposase (“CAST”) system. A CAST system can include a Cas protein that is catalytically inactive, or engineered to be catalytically active (e.g., have nickase or nuclease activity), and further comprises a transposase (or subunits thereof) that catalyze RNA-guided DNA transposition. Such systems are able to insert DNA sequences at a target site in a DNA molecule without relying on host cell repair machinery. CAST systems can be Class1 or Class 2 CAST systems. An example Class 1 system is described in Klompe et al. Nature, doi: 10.1038/s41586-019-1323, which is incorporated herein by reference. An example Class 2 system is described in Strecker et al. Science. 10/1126/science.aax9181 (2019), and PCT/US2019/066835 which are incorporated herein by reference.

### Administration of the Pharmaceutical Formulations

The pharmaceutical formulations or dosage forms thereof described herein can be administered one or more times hourly, daily, monthly, or yearly (e.g., 1, 2, 3, 4, 5, 6, 7, 8, 9, 10, 11, 12, 13, 14, 15, 16, 17, 18, 19, 20, or more times hourly, daily, monthly, or yearly). In an embodiment, the pharmaceutical formulations or dosage forms thereof described herein can be administered continuously over a period of time ranging from minutes to hours to days. Devices and dosage forms are known in the art and described herein that are effective at providing continuous administration of the pharmaceutical formulations described herein. In an embodiment, the first one or a few initial amount(s) administered can be a higher dose than subsequent doses. This is typically referred to in the art as a loading dose or doses and a maintenance dose, respectively. In an embodiment, the pharmaceutical formulations can be administered such that the doses over time are tapered (increased or decreased) so as to wean a subject gradually off of a pharmaceutical formulation or gradually introduce a subject to the pharmaceutical formulation.

As used herein, “administering” refers to any suitable administration of the agent(s) being delivered to the subject receiving said agent(s) and can be oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravitreal, intracerebral, and intracerebroventricular, intratympanic, intracochlear, rectal, vaginal, by inhalation, by catheters, stents or via an implanted reservoir or another device that administers, either actively or passively (e.g. by diffusion) a composition to the perivascular space and adventitia. For example, a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration routes can be, for instance, auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratympanic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated, subject being treated, and/or agent(s) being administered.

As previously discussed, the pharmaceutical formulation can contain a predetermined amount of a primary active agent, secondary active agent, and/or pharmaceutically acceptable salt thereof where appropriate. In some of these embodiments, the predetermined amount can be an appropriate fraction of the effective amount of the active ingredient. Such unit doses may therefore be administered once or more than once a day, month, or year (e.g., 1, 2, 3, 4, 5, 6, or more times per day, month, or year). Such pharmaceutical formulations may be prepared by any of the methods well-known in the art.

Where co-therapies or multiple pharmaceutical formulations are to be delivered to a subject, the different therapies or formulations can be administered sequentially or simultaneously. Sequential administration is an administration where an appreciable amount of time occurs between administrations, such as more than about 15, 20, 30, 45, 60 minutes or more. The time between administrations in sequential administration can be on the order of hours, days, months, or even years, depending on the active agent present in each administration. Simultaneous administration refers to an administration of two or more formulations at the same time or substantially at the same time (e.g., within seconds or just a few minutes apart), where the intent is that the formulations be administered together at the same time.

### Kits

Any of the Acr delivery compounds, compositions, systems, formulations, delivery vehicles, cells, etc. of the present invention described herein or a combination thereof can be presented as a combination kit. As used herein, the terms “combination kit” or “kit of parts” refers to the compounds, compositions, formulations, particles, cells, and any additional components that are used to package, sell, market, deliver, and/or administer the combination of elements or a single element, such as the active ingredient, contained therein. Such additional components include, but are not limited to, packaging, syringes, blister packages, bottles, and the like. When one or more of the compounds, compositions, formulations, particles, cells, described herein or a combination thereof (e.g., agents) contained in the kit are administered simultaneously, the combination kit can contain the active agents in a single formulation, such as a pharmaceutical formulation, (e.g., a tablet) or in separate formulations. When the compounds, compositions, formulations, particles, and cells described herein or a combination thereof and/or kit components are not administered simultaneously, the combination kit can contain each agent or other component in separate pharmaceutical formulations. The separate kit components can be contained in a single package or in separate packages within the kit.

In an embodiment, the combination kit also includes instructions printed on or otherwise contained in a tangible medium of expression. The instructions can provide information regarding the content of the compounds, compositions, formulations, particles, cells, described herein or a combination thereof contained therein, safety information regarding the content of the compounds, compositions, formulations (e.g., pharmaceutical formulations), particles, and cells described herein or a combination thereof contained therein, information regarding the dosages, indications for use, and/or recommended treatment regimen(s) for the compound(s) and/or pharmaceutical formulations contained therein. In an embodiment, the instructions can provide directions for administering the compounds, compositions, formulations, particles, and cells described herein or a combination thereof to a subject in need thereof. In an embodiment, the subject in need thereof is in need of genetic or nucleic acid modification. In an embodiment, the subject in need thereof is receiving or going to receive a CRISPR-Cas system therapy. In an embodiment, the subject in need thereof is receiving or going to receive a CRISPR-Cas system therapy and is in need of inhibiting the activity of the CRISPR-Cas system, for example, to reduce off-target effects and/or restrict polynucleotide modification by the CRISPR-Cas system to a particular cell state, cell type, and/or point in the cell cycle.

### Methods of Delivery of the Acr Polypeptide

The engineered Acr delivery polypeptides and systems thereof of the present invention can be used to deliver an Acr polypeptide to a cell. Without being bound by theory, in an embodiment and as shown in FIG. 1A-1B, the targeting moiety or targeting domain, which is part of a pore-forming polypeptide or is operatively coupled to a pore-forming polypeptide can target pore-forming polypeptides to a target cell or cells expressing on their surface a targeting moiety binding partner. In FIG. 1A the pore-forming polypeptide comprises a targeting domain. In FIG. 1B, the exemplary targeting moiety of an antibody is shown. In an embodiment, as a sufficient number of pore-forming proteins are attached and in proximity, the pore-forming proteins oligomerize and form a pre-pore. In an embodiment, the engineered Acr polypeptide(s) bind or otherwise interact with the pre-pore. The complex that is formed is then endocytosed into the cell which delivers the Acr to the cell, via e.g., an endosome release mechanism. In an embodiment, the Acr is cleaved from the cargo delivery molecule in the endosome. In some embodiments, the Acr is cleaved from the cargo delivery molecule after release from the endosome. Without being bound by theory, the engineered Acr polypeptide is then released from the endosome and into the cytosol. In an embodiment, the engineered Acr polypeptide or component thereof (e.g., an Acr polypeptide) is unfolded prior to release from the endosome. In an embodiment, an acidic environment of an endocytosed pore can result in unfolding of an Acr polypeptide such that the Acr polypeptide can fit through the pore from the N to the C terminus. See also e.g., Young and Collier. Annu. Rev. Biochem. (2007) 76:243-265, which discusses the mechanism of PA-mediated cargo delivery via pore formation in the context of the native anthrax toxin. In an embodiment, the engineered Acr polypeptide is translocated into the nucleus of the cell. In an embodiment, the engineered Acr polypeptide is translocated into another intracellular compartment. In an embodiment, the Acr polypeptide is not cleaved from the cargo delivery molecule. In an embodiment, the engineered Acr polypeptide is cleaved from the cargo delivery molecule. In an embodiment, the engineered Acr polypeptide or component thereof can interact with and/or inhibit the activity of a CRISPR-Cas system or component thereof in the cytosol and/or nucleus of a target cell containing a CRISPR-Cas system or component thereof.

In certain embodiments, a method of delivering an anti-CRISPR (Acr) polypeptide to a cell comprises providing, to a cell or cell population, one or more engineered Acr polypeptides of the present invention; one or more engineered Acr polypeptide delivery systems or a component thereof of the present invention; one or more polynucleotides of the present invention; one or more vectors of the present invention; a delivery vehicle of the present invention; a cell or cell population of the present invention; a pharmaceutical formulation of the present invention; or any combination thereof.

In an embodiment, the method further includes binding the targeting moiety of a plurality of pore-forming polypeptides (e.g., PA₈₃) of an engineered Acr delivery system of the present invention to the targeting moiety binding partner on the cell surface (e.g., TEM8 and CMG2 receptors) thereby tethering the pore-forming protein (e.g., PA₈₃) to the cell surface; and forming a pre-pore at the cell membrane surface formed from a plurality of the pore-forming polypeptides (e.g., PA₆₃ after a furin family protease cleaves PA₈₃) tethered to the cell membrane surface. In an embodiment, the method further includes coupling the engineered Acr polypeptide to a pore-forming polypeptide in the pre-pore via binding of the cargo delivery molecule (e.g., LF_{N}) to the pore-forming polypeptide(s) (e.g., PA₆₃) in the pre-pore. In an embodiment, the method further includes, transporting the pre-pore and the engineered Acr polypeptide coupled thereto into the cell via endocytosis. In an embodiment, the pre-pore turns into a pore after acidification of the endosome.

In an embodiment, the method further includes releasing the engineered Acr polypeptide from the pre-pore or from the pore.

In an embodiment, the method further includes releasing the engineered Acr polypeptide from an endosome into the cytosol of the cell.

Also described in example embodiments herein are methods of inhibiting the activity of a CRISPR-Cas system in a cell comprising delivering an anti-CRISPR (Acr) polypeptide to the cell by the method described herein, whereby the Acr polypeptide inhibits the activity of a CRISPR-Cas system or a component thereof in the cell.

In an embodiment, the activity of the CRISPR-Cas system or component thereof is reduced by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 fold or more. In an embodiment, the activity of the CRISPR-Cas system or component thereof is reduced by any non-zero percent to/or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent. In an embodiment, the activity of the CRISPR-Cas system or component thereof is reduced to below the level of detection as measured by an indel assay or other suitable assay.

In an embodiment, delivery is in vivo, in vitro, in situ, or ex vivo.

In an embodiment, delivery is to a target cell type and/or cell state. Target cell types and states are described in greater detail elsewhere herein. In an embodiment, the target cell contains or will contain a CRISPR-Cas system or component thereof. Exemplary CRISPR-Cas systems are described in greater detail elsewhere herein. In an embodiment, the cell comprises a targeting moiety-binding partner on the cell membrane surface.

Delivery can be simultaneous or sequential with a CRISPR-Cas system or component thereof. In an embodiment, the engineered delivery system is delivered before or after the CRISPR-Cas system. In an embodiment, the engineered Acr delivery polypeptide or system thereof is in the same formulation as a CRISPR-Cas system or component thereof.

In an embodiment, the engineered Acr delivery polypeptide is delivered to a cell simultaneously with a pore-forming polypeptide. In an embodiment, the engineered Acr delivery polypeptide is delivered to a cell sequentially or separately from a pore-forming polypeptide.

In an embodiment, two or more (e.g., 2, 3, 4, 5, 6, 7, 8, 9, 10, or more) different engineered Acr delivery polypeptide systems are delivered to a subject in need thereof. In an embodiment, two or more (e.g., 2, 3, 4, 5, 6, 7, 8, 9, 10, or more) engineered Acr delivery polypeptides targeting different CRISPR-Cas systems can be delivered to a subject in need thereof. In an embodiment, two or more engineered Acr delivery systems each comprising pore-forming-proteins targeting different cell types and/or cell states can be delivered to a subject in need thereof.

In an embodiment, the engineered Acr delivery polypeptide systems or components thereof can be delivered to a subject in need thereof. In an embodiment, the subject in need thereof is receiving or is in need of a CRISPR-Cas based treatment. In an embodiment, the subject in need thereof has a disease and is a target or can be a target of a CRISPR-Cas based treatment. In an embodiment, the subject in need thereof has a genetic disease.

In other embodiments, the pore is formed in an endosomal membrane so as to release an engineered Acr delivery system or component(s) thereof, such as a cargo, into the cytosol or other intracellular compartment. In these embodiments, a targeting moiety can target a cell surface receptor, that once bound, stimulates endocytosis. Endocytosis results in capture of one or more engineered pore-forming polypeptides and cargo coupled thereto, in an endosome. A pore is then formed by the pore-forming polypeptides in the membrane of the endosome. Cargo can then be delivered to the cytosol or other intracellular compartment in which the endosome is present. In an embodiment, the cargo is cleaved from the engineered pore-forming protein in the endosome. In an embodiment, the cargo is not cleaved from the engineered pore-forming protein in the endosome. See e.g., Murphy, J. Toxins. 2011. 3 (3), 294-308 and Tian et al., Cell Reports. 38 (10): 110476 (2022).

Further embodiments are illustrated in the following Examples which are given for illustrative purposes only and are not intended to limit the scope of the invention.

### EXAMPLES

### Example 1—Protective Antigen Delivery of Acrs

This example demonstrates the delivery of an engineered Acr via a protective antigen (PA) strategy (see e.g., FIG. 1A-1B). An exemplary engineered Acr fusion polypeptide was generated by fusing the Anthrax lethal factor N-terminus (LF_{N}) to a representative Acr (AcrIIA4). In this example, a non-cleavable GS linker with a cysteine in between the LEN and the Acr was used. This left open the opportunity for bioconjugation. That said, a cleavable linker could be used. Exemplary cleavable linkers are described in greater detail elsewhere herein. The construct also included an SV40 nuclear localization signal (NLS) at the C-terminus of the Acr. See e.g., FIG. 2. An exemplary full-length plasmid sequence that includes a polynucleotide encoding the engineered Acr fusion polypeptide of FIG. 2 is shown below. As shown, the full sequence of the plasmid encoding 10×His-MBP-TEV-LF_{N}-G₄CG₄S-AcrIIA4-GS-NLS is in uppercase, and its domains are underlined differently: 10×His-MBP tag, TEV site, LF_{N}, G₄CG₄S linker (SEQ ID NO: 1), AcrIIA4, GS linker, SV40 NLS. The MBP-TEV backbone plasmid is Addgene plasmid #115670, Watters et al., Science 2018, 362 (6411), 236-239. The AcrIIA4 sequence was originally reported in Rauch et al., Cell 2017, 168, 150-158.

Full sequence of the plasmid encoding 10×His-MBP-TEV-LF_{N}-G₄CG₄S-AcrIIA4-GS-NLS:

(SEQ ID NO: 106)
tcccccggccacggggcctgccaccatacccacgccgaaacaagcgctcatgagcccgaagtggcgagcccgat
cttccccatcggtgatgtcggcgatataggcgccagcaaccgcacctgtggcgccggtgatgccggccacgatgcgtccggcgtag
aggatcgagatctcgatcccgcgaaattaatacgactcactatagggagaccacaacggtttccctctagtgccggctccggagagct
ctttaattaagcggccgccctgcaggactcgagttctagaaataattttgtttaactttaagaaggagatatacatATGAAATCTT

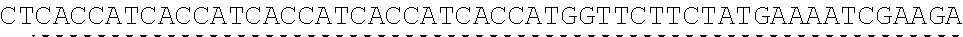

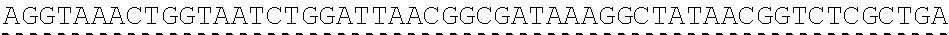

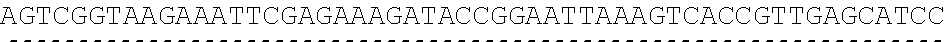

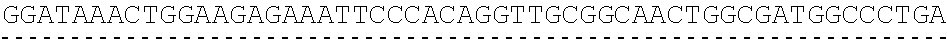

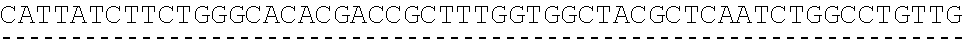

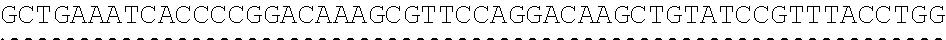

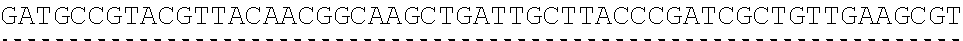

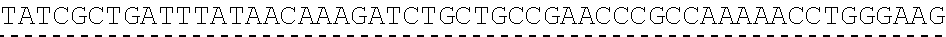

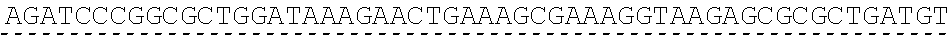

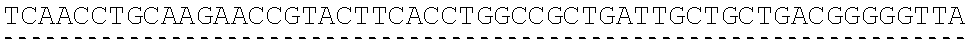

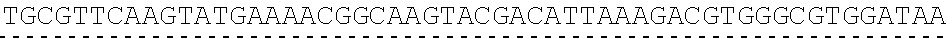

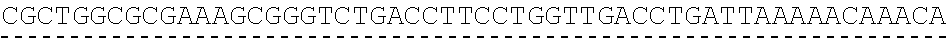

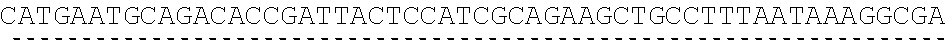

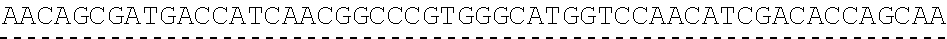

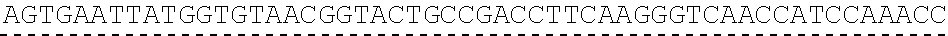

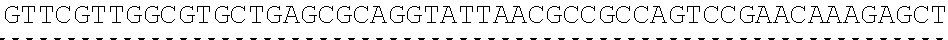

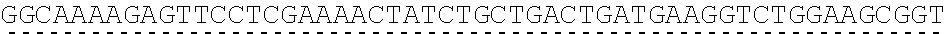

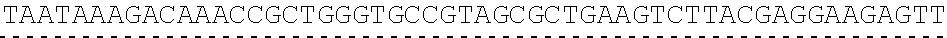

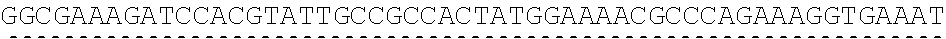

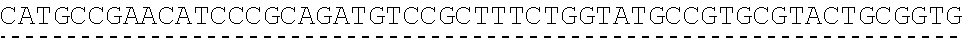

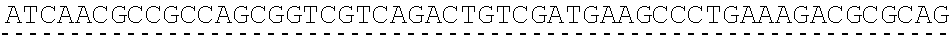

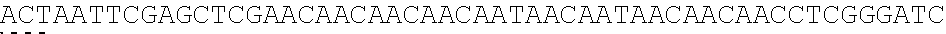

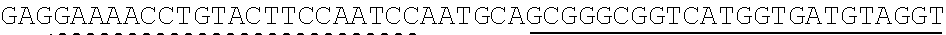

ATGCACGTAAAAGAGAAAGAGAAAAATAAAGATGAGAATAAGAGAAAAGATGA
AGAACGAAATAAAACACAGGAAGAGCATTTAAAGGAAATCATGAAACACATTG
TAAAAATAGAAGTAAAAGGGGAGGAAGCTGTTAAAAAAGAGGCAGCAGAAAAG
CTACTTGAGAAAGTACCATCTGATGTTTTAGAGATGTATAAAGCAATTGGAGGAA
AGATATATATTGTGGATGGTGATATTACAAAACATATATCTTTAGAAGCATTATC
TGAAGATAAGAAAAAAATAAAAGACATTTATGGGAAAGATGCTTTATTACATGA
ACATTATGTATATGCAAAAGAAGGATATGAACCCGTACTTGTAATCCAATCTTCG
GAAGATTATGTAGAAAATACTGAAAAGGCACTGAACGTTTATTATGAAATAGGT
AAGATATTATCAAGGGATATTTTAAGTAAAATTAATCAACCATATCAGAAATTTT
TAGATGTATTAAATACCATTAAAAATGCATCTGATTCAGATGGACAAGATCTTTT
ATTTACTAATCAGCTTAAGGAACATCCCACAGACTTTTCTGTAGAATTCTTGGAA
CAAAATAGCAATGAGGTACAAGAAGTATTTGCGAAAGCTTTTGCATATTATATCG
AGCCACAGCATCGTGATGTTTTACAGCTTTATGCACCGGAAGCTTTTAATTACAT
GGATAAATTTAACGAACAAGAAATAAATCTATCCTTGGAAGAACTTAAAGATCA

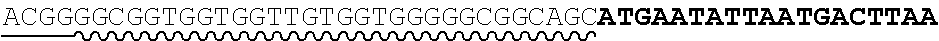

TTAGAGAAATCAAAAACAAAGATTACACAGTGAAATTGAGTGGTACGGATA
GCAATAGTATCACACAGCTAATTATTCGCGTTAATAATGATGGCAACGAGTA
TGTAATTTCTGAAAGTGAAAATGAATCAATCGTTGAAAAATTCATCTCTGCA
TTCAAAAACGGTTGGAATCAAGAATACGAGGATGAAGAAGAATTTTATAATG

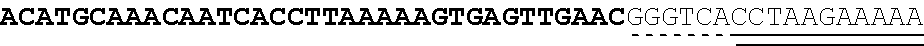

ACGAAAAGTTtaataaatattggaagtggataacggatccgcgatcgcggcgcgccacctggtggccggccggtaccacg
cgtgcgcgctgatccggctgctaacaaagcccgaaaggaagctgagttggctgctgccaccgctgagcaataactagcataacccct
tggggcctctaaacgggtcttgaggggttttttgctgaaaggaggaactatatccggatatccacaggacgggtgtggtcgccatgatc
gcgtagtcgatagtggctccaagtagcgaagcgagcaggactgggcggcggccaaagcggtcggacagtgctccgagaacgggt
gcgcatagaaattgcatcaacgcatatagcgctagcagcacgccatagtgactggcgatgctgtcggaatggacgatatcccgcaag
aggcccggcagtaccggcataaccaagcctatgcctacagcatccagggtgacggtgccgaggatgacgatgagcgcattgttaga
tttcatacacggtgcctgactgcgttagcaatttaactgtgataaactaccgcattaaagcttatcgatgataagctgtcaaacatgagaatt
cttgaagacgaaagggcctcgtgatacgcctatttttataggttaatgtcatgataataatggtttcttagacgtcaggtggcacttttcggg
gaaatgtgcgcggaacccctatttgtttatttttctaaatacattcaaatatgtatccgctcatgagacaataaccctgataaatgcttcaata
acattgaaaaaggaagagtatgagtattcaacatttccgtgtcgcccttattcccttttttgcggcattttgccttcctgtttttgctcacccag
aaacgctggtgaaagtaaaagatgctgaagatcagttgggtgcacgagtgggttacatcgaactggatctcaacagcggtaagatcct
tgagagttttcgccccgaagaacgttttccaatgatgagcacttttaaagttctgctatgtggcgcggtattatcccgtgttgacgccgggc
aagagcaactcggtcgccgcatacactattctcagaatgacttggttgagtactcaccagtcacagaaaagcatcttacggatggcatg
acagtaagagaattatgcagtgctgccataaccatgagtgataacactgcggccaacttacttctgacaacgatcggaggaccgaagg
agctaaccgcttttttgcacaacatgggggatcatgtaactcgccttgatcgttgggaaccggagctgaatgaagccataccaaacgac
gagcgtgacaccacgatgcctgcagcaatggcaacaacgttgcgcaaactattaactggcgaactacttactctagcttcccggcaac
aattaatagactggatggaggcggataaagttgcaggaccacttctgcgctcggcccttccggctggctggtttattgctgataaatctg
gagccggtgagcgtgggtctcgcggtatcattgcagcactggggccagatggtaagccctcccgtatcgtagttatctacacgacggg
gagtcaggcaactatggatgaacgaaatagacagatcgctgagataggtgcctcactgattaagcattggtaactgtcagaccaagttt
actcatatatactttagattgatttaaaacttcatttttaatttaaaaggatctaggtgaagatcctttttgataatctcatgaccaaaatccctta
acgtgagttttcgttccactgagcgtcagaccccgtagaaaagatcaaaggatcttcttgagatcctttttttctgcgcgtaatctgctgctt
gcaaacaaaaaaaccaccgctaccagcggtggtttgtttgccggatcaagagctaccaactctttttccgaaggtaactggcttcagca
gagcgcagataccaaatactgtccttctagtgtagccgtagttaggccaccacttcaagaactctgtagcaccgcctacatacctegctc
tgctaatcctgttaccagtggctgctgccagtggcgataagtcgtgtcttaccgggttggactcaagacgatagttaccggataaggcg
cagcggtcgggctgaacggggggttcgtgcacacagcccagcttggagcgaacgacctacaccgaactgagatacctacagcgtg
agctatgagaaagcgccacgcttcccgaagggagaaaggcggacaggtatccggtaagcggcagggtcggaacaggagagcgc
acgagggagcttccagggggaaacgcctggtatctttatagtcctgtcgggtttcgccacctctgacttgagcgtcgatttttgtgatgct
cgtcaggggggcggagcctatggaaaaacgccagcaacgcggcctttttacggttcctggccttttgctggccttttgctcacatgttctt
tcctgcgttatcccctgattctgtggataaccgtattaccgcctttgagtgagctgataccgctcgccgcagccgaacgaccgagcgca
gcgagtcagtgagcgaggaagcggaagagcgcctgatgcggtattttctccttacgcatctgtgcggtatttcacaccgcaatggtgca
ctctcagtacaatctgctctgatgccgcatagttaagccagtatacactccgctatcgctacgtgactgggtcatggctgcgccccgaca
cccgccaacacccgctgacgcgccctgacgggcttgtctgctcccggcatccgcttacagacaagctgtgaccgtctccgggagctg
catgtgtcagaggttttcaccgtcatcaccgaaacgcgcgaggcagctgcggtaaagctcatcagcgtggtcgtgaagcgattcacag
atgtctgcctgttcatccgcgtccagctcgttgagtttctccagaagcgttaatgtctggcttctgataaagcgggccatgttaagggcgg
ttttttcctgtttggtcactgatgcctccgtgtaagggggatttctgttcatgggggtaatgataccgatgaaacgagagaggatgctcacg
atacgggttactgatgatgaacatgcccggttactggaacgttgtgagggtaaacaactggcggtatggatgcggcgggaccagaga
aaaatcactcagggtcaatgccagcgcttcgttaatacagatgtaggtgttccacagggtagccagcagcatcctgcgatgcagatcc
ggaacataatggtgcagggcgctgacttccgcgtttccagactttacgaaacacggaaaccgaagaccattcatgttgttgctcaggtc
gcagacgttttgcagcagcagtcgcttcacgttcgctcgcgtatcggtgattcattctgctaaccagtaaggcaaccccgccagcctag
ccgggtcctcaacgacaggagcacgatcatgcgcacccgtggccaggacccaacgctgcccgagatgcgccgcgtgcggctgct
ggagatggcggacgcgatggatatgttctgccaagggttggtttgcgcattcacagttctccgcaagaattgattggctccaattcttgg
agtggtgaatccgttagcgaggtgccgccggcttccattcaggtcgaggtggcccggctccatgcaccgcgacgcaacgcgggga
ggcagacaaggtatagggcggcgcctacaatccatgccaacccgttccatgtgctcgccgaggcggcataaatcgccgtgacgatc
agcggtccaatgatcgaagttaggctggtaagagccgcgagcgatccttgaagctgtccctgatggtcgtcatctacctgcctggaca
gcatggcctgcaacggggcatcccgatgccgccggaagcgagaagaatcataatggggaaggccatccagcctcgcgtcgcga
acgccagcaagacgtagcccagcgcgtcggccgccatgccggcgataatggcctgcttctcgccgaaacgtttggtggcgggacca
gtgacgaaggcttgagcgagggcgtgcaagattccgaataccgcaagcgacaggccgatcatcgtcgcgctccagcgaaagcggt
cctcgccgaaaatgacccagagcgctgccggcacctgtcctacgagttgcatgataaagaagacagtcataagtgcggcgacgata
gtcatgccccgcgcccaccggaaggagctgactgggttgaaggctctcaagggcatcggtcgacgctctcccttatgcgactcctgc
attaggaagcagcccagtagtaggttgaggccgttgagcaccgccgccgcaaggaatggtgcatgcaaggagatggcgcccaacag

The engineered Acr fusion polypeptide was produced and purified in E. coli. See e.g., FIG. 3A-3C. This approach yielded about 5 mg of purified LF_{N}-AcrIIA4 fusion polypeptide per liter of E. coli culture.

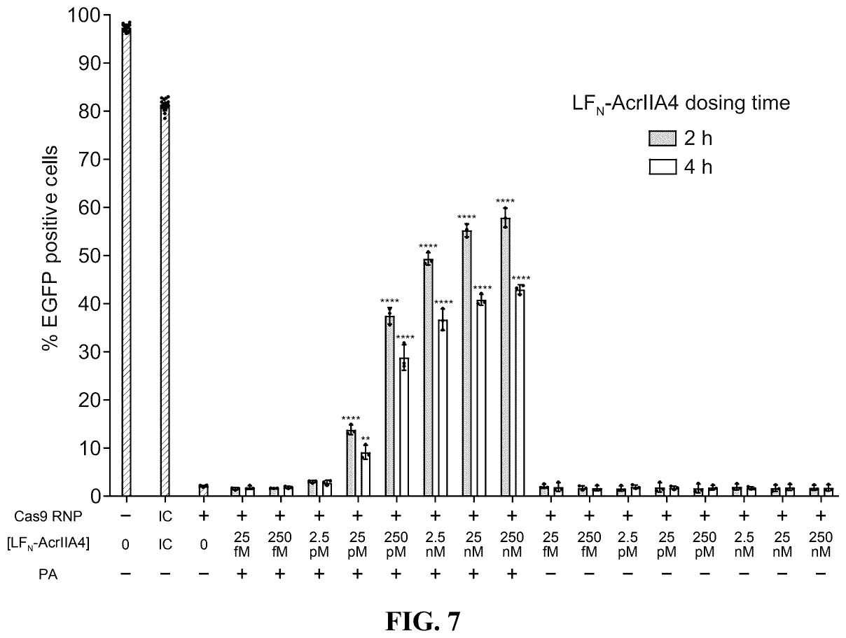

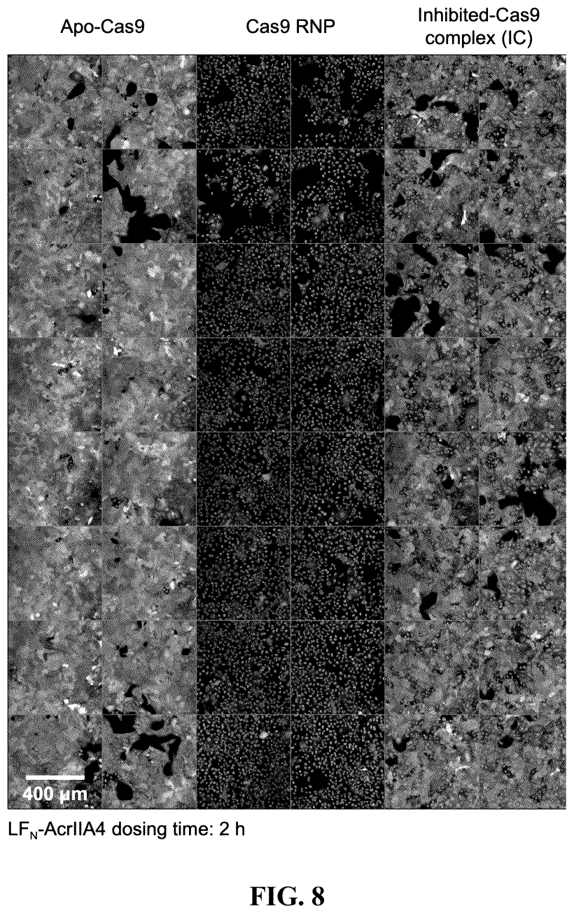

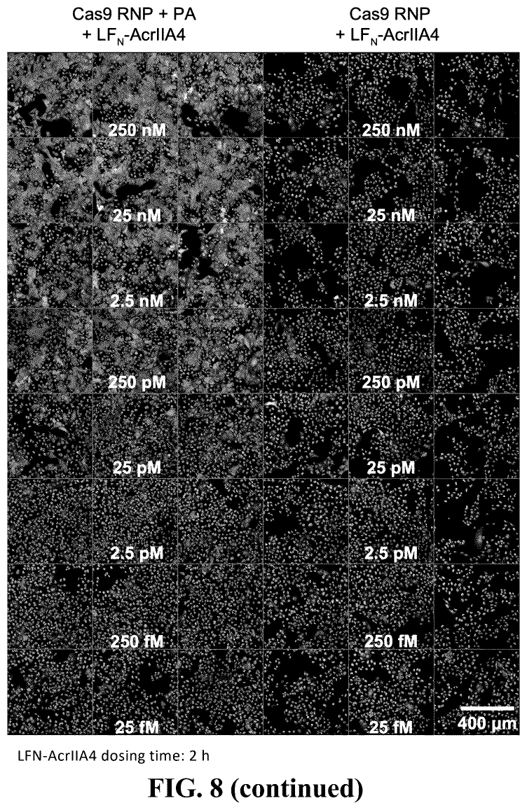

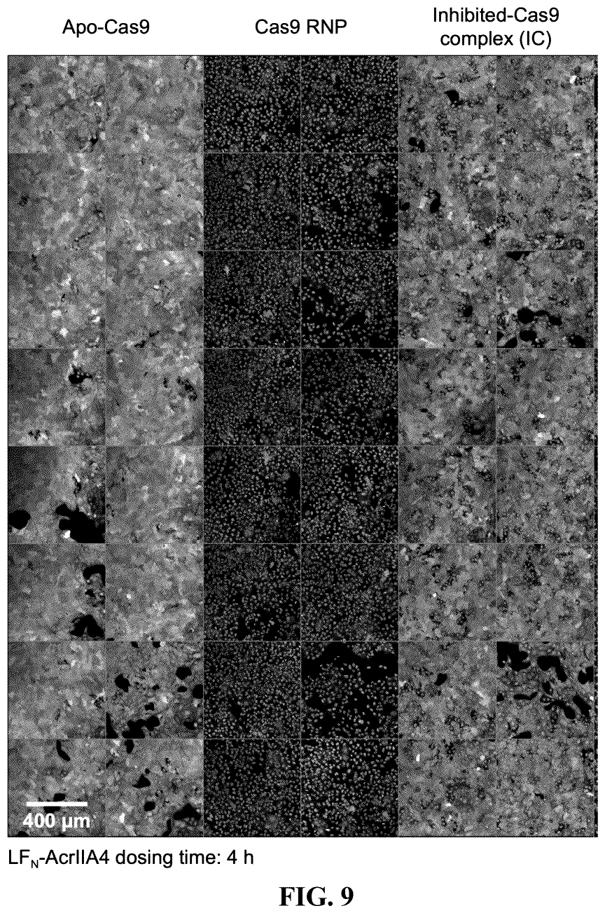

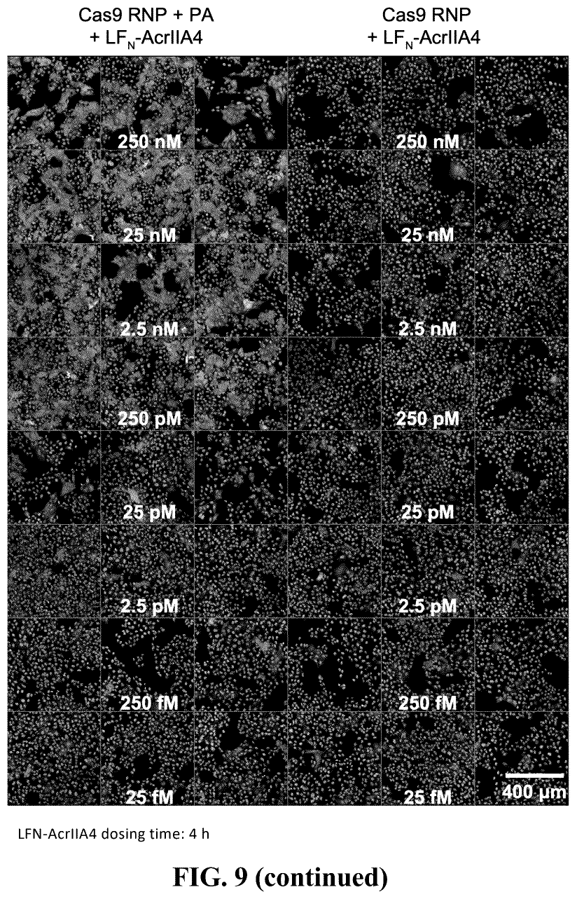

An EGFP disruption assay was utilized to assess the activity and efficiency of delivery of the engineered Acr polypeptide, LF_{N}-AcrIIA4, via the PA method in human cells (U2OS cells stably expressing EGFP: U2OS.EGFP.PEST cells, developed by Joung and co-workers, Nat. Biotechnol. 2013, 31 (9), 822-826 and Nat. Biotechnol. 2015, 33 (12), 1293-1298). See e.g., FIG. 4. In brief, inhibition of Cas activity is directly proportional to EGFP fluorescence. Results from this assay are shown in FIGS. 5-9. As shown in FIGS. 7-9, timing of the delivery of and/or controlling the dose of the engineered Acr influences its activity and ability to inhibit Cas9.

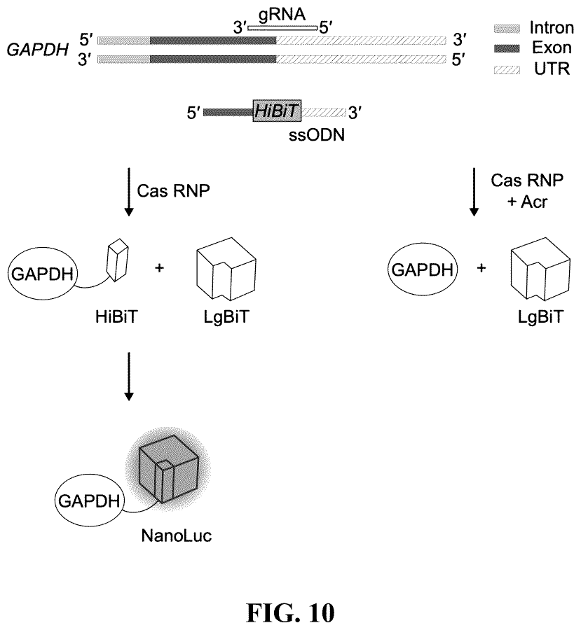

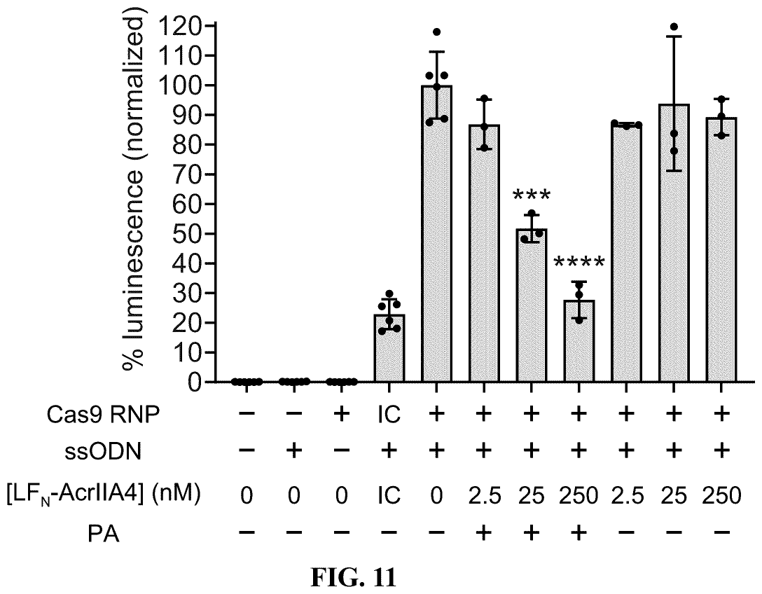

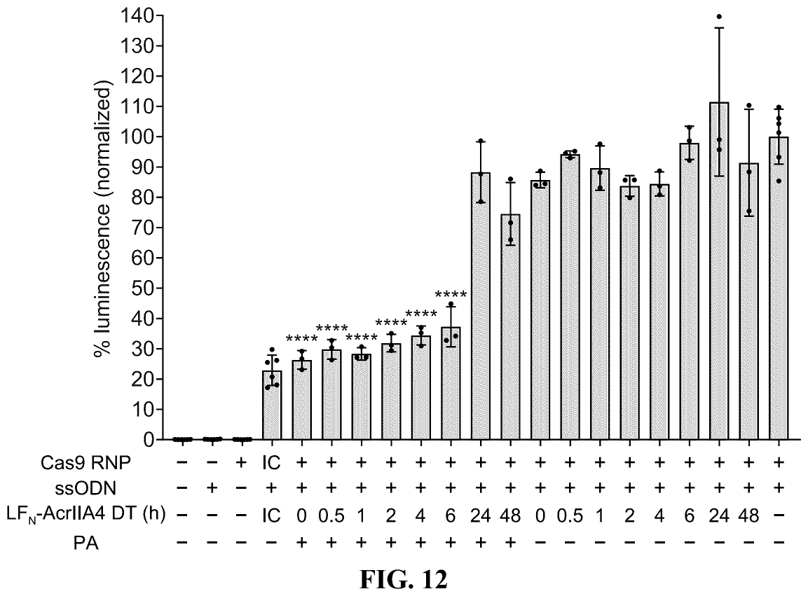

A HiBiT assay was used for validation of delivery of the engineered Acr polypeptide via the PA method in human cells. See e.g., FIG. 10. In brief, inhibition of Cas activity decreases the luminescence signal. Results from this assay are shown in FIGS. 11-12 and demonstrate that the PA method delivered the engineered Acr polypeptide (e.g., LF_{N}-AcrIIA4) into human cells (HEK293T) in a dose-dependent manner (see e.g., FIG. 11) and that timing the delivery of the engineered Acr influences its activity and ability to inhibit Cas9 (FIG. 12).

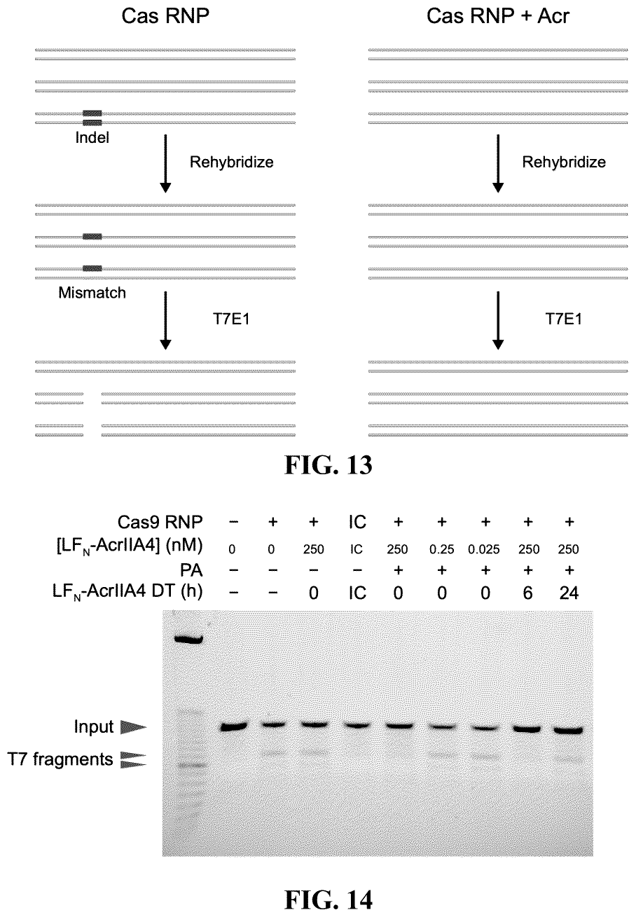

A T7 endonuclease assay was used to validate the delivery of the engineered Acr polypeptide (e.g., LF_{N}-AcrIIA4) via the PA strategy in human cells (HEK293T). See e.g., FIG. 13. In brief, inhibition diminishes the intensity of T7 fragments and increases the intensity of the input DNA substrate band in the agarose gel. FIG. 14 shows results from the T7 endonuclease validation assay described in FIG. 13 demonstrating that concentration and time of addition control the efficiency of delivery of an engineered Acr polypeptide (e.g., LF_{N}-AcrIIA4) and its ability to inhibit Cas9.

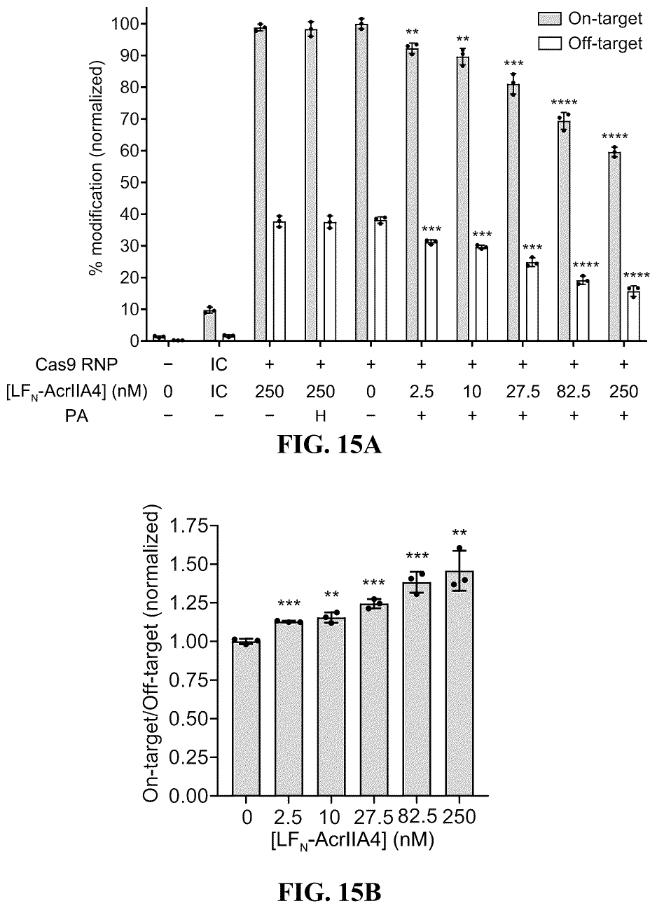

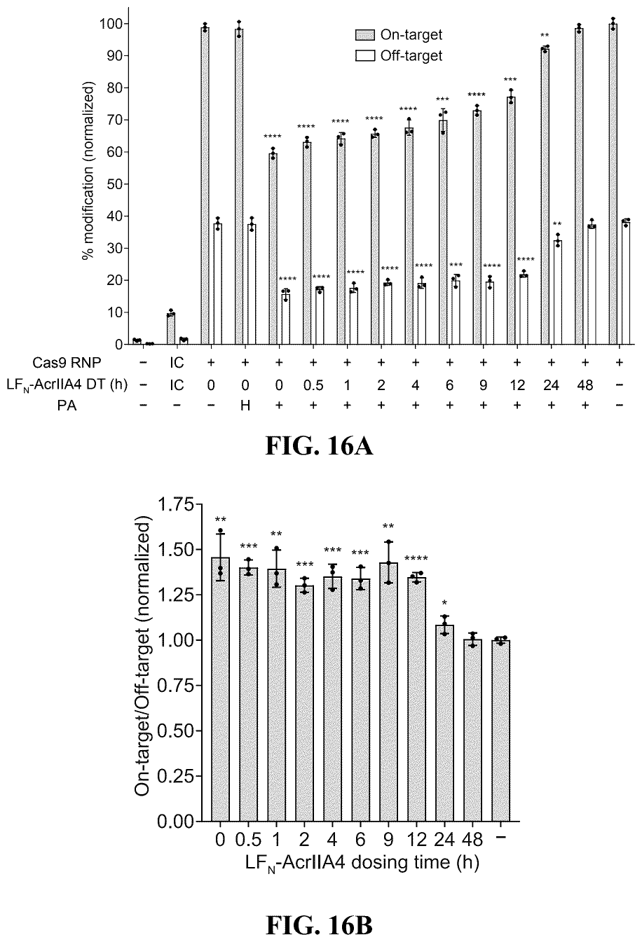

Next-generation sequencing (NGS) was used to confirm that delivery of the engineered Acr polypeptide (e.g., LF_{N}-AcrIIA4) using the PA method increases Cas9 specificity in human cells (HEK293T). Results are demonstrated in FIGS. 15A-15B and 16A-16B. FIG. 15A-15B shows that delivery of an engineered Acr polypeptide (e.g., LF_{N}-AcrIIA4) using the PA method increases Cas9 specificity in human cells in a dose-dependent manner. FIG. 16A-16B shows that timing the delivery of an engineered Acr polypeptide (e.g., LF_{N}-AcrIIA4) using the PA method increases Cas9 specificity in human cells.

For some experiments, 300,000 cells in 20 μL of buffer were used for Cas9 nucleofection (1.5×10⁷ cells/mL). Then, LF_{N}-AcrIIA4 (25 fM-250 nM final) with PA (20 nM final) was delivered to 10,000-30,000 cells (96-well plate) in 100 μL of media (100,000-300,000 cells/mL) followed by a 1-, 2-, and 3-day incubation. For a 24-well plate format, LF_{N}-AcrIIA4 (2.5 nM-250 nM)/PA (20 nM) was delivered to 120,000-150,000 cells in 600 μL media (200,000-250,000 cells/mL) followed by a three-day incubation.

Applicant notes that the data was generated in U2OS and HEK293T cells. Thus, wild-type PA₈₃ was used to target these cells and demonstrate proof of concept, which can be extended to other PA variants and other pore-forming proteins described elsewhere herein. As such, the TEM8 or CMG2 receptors present on these cell types were targeted. The Protein Atlas database (www.proteinatlas.org) provides that the normalized transcript per million (nTPM) of TEM8 (ANTXR1) expression is 52.3 in U2OS cells and 24.4 in HEK293 cells. The Protein Atlas database provides that the nTPM of CMG2 (ANTXR2) expression is 6.6 in U2OS cells and 4.2 in HEK293 cells. Without being bound by theory, the difference in receptor expression between cell lines can explain differences observed in delivery efficiency results presented in this Example.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

Further attributes, features, and embodiments of the present invention can be understood by reference to the following numbered aspects of the disclosed invention. Reference to disclosure in any of the preceding aspects is applicable to any preceding numbered aspect and to any combination of any number of preceding aspects, as recognized by appropriate antecedent disclosure in any combination of preceding aspects that can be made. The following numbered aspects are provided:

1. An engineered Anti-CRISPR (Acr) polypeptide comprising:
an Acr polypeptide operatively coupled to a cargo delivery molecule, wherein the cargo delivery molecule is capable of binding or otherwise interacting with a pore-forming polypeptide.

2. The engineered Acr polypeptide of aspect 1, wherein the cargo delivery molecule is a bacterial exotoxin, optionally a Bacillus anthracis lethal factor (LF) or edema factor (EF) or a derivative thereof or a Corynebacterium diphtheriae catalytic domain or derivative thereof.

3. The engineered Acr polypeptide of any one of aspects 1-2, wherein the cargo delivery molecule is engineered to comprise a pore-forming polypeptide interaction molecule or domain, optionally wherein the pore-forming polypeptide interaction molecule or domain is operatively coupled to an N-terminus, a C-terminus, at a location between the N-terminus and the C-terminus, or any combination thereof of the cargo delivery molecule.

4. The engineered Acr polypeptide of any one of aspects 1-3, wherein the cargo delivery molecule is cleavably coupled to the Acr polypeptide.

5. The engineered Acr polypeptide of aspect 4, wherein the cargo delivery molecule comprises a cleavable domain or wherein the cargo delivery molecule is linked via cleavable linker to the Acr polypeptide.

6. The engineered Acr polypeptide of any one of aspects 1-5, wherein the pore-forming polypeptide is an alpha pore-forming polypeptide a beta pore-forming polypeptide, or both.

7. The engineered Acr polypeptide of any one aspects 1-6, wherein the pore-forming polypeptide is a Bacillus anthracis protective antigen polypeptide or a derivative thereof, or is a Corynebacterium diphtheriae translocation polypeptide or a derivative thereof.

8. The engineered Acr polypeptide of any one of aspects 1-7, wherein the Acr polypeptide inhibits a Type I, Type II, Type III, Type V, or Type VI CRISPR-Cas system or component or activity thereof.

9. The engineered Acr polypeptide of any one of aspects 1-8, wherein the Acr polypeptide is selected from an AcrIE8.2, AcrIE9, AcrIF1, AcrIF2, AcrIF3, AcrIF4, AcrIF5, AcrIF6, ArcIF7, AcrIF8, AcrIF9, AcrIF10, AcrIF11, AcrIF11.1, AcrIF11.2, AcrIF12, AcrIF13, AcrIF14, AcrIF15, AcrIF16, AcrIF17, AcrIF18, AcrIF19, AcrIF20, AcrIF21, AcrIF22, AcrIF23, AcrIF24, AcrIE4-F7, AcrIAI, AcrIB1, AcrIC1, AcrIF2/C2, AcrIC3, AcrIC4, AcrIC5, AcrIC6, AcrIC7, AcrIC8, AcrIC9, AcrIC10, AcrID1, AcrIIA1, AcrIIA2, AcrIIA2-1, AcrIIA2-2, AcrIIA2b, AcrIIA3, AcrIIA4, AcrIIA4-2, AcrIIA4-3, AcrIIA4 variant Ins. 5, AcrIIA4 variant N39A, AcrIIA4 variant D14A/G38A, AcrIIA5, AcrIIA5-2, AcrIIA6, AcrIIA7, AcrIIA8, AcrIIA9, AcrIIA10, AcrIIA11, AcrIIA12, AcrIIA13, AcrIIA13b, AcrIIA14, AcrIIA15, AcrIIA16, AcrIIA17, AcrIIA18, AcrIIA19, AcrIIA20, AcrIIA21, AcrIIA22, AcrIIA23, AcrIIA24, AcrIIA25, AcrIIA26, AcrIIA27, AcrIIA28, AcrIIA29, AcrIIA30, AcrIIA31, AcrIIA32, AcrIIC1, AcrIIC1-1, AcrIIC2, AcrIIC2-1, AcrIIC2-2, AcrIIC3, AcrIIC4, AcrIIC5, AcrIIC6, AcrIII-1, AcrIIIB1, AcrVA1, AcrVA2, AcrVA3, AcrVA3.1, AcrVA4, AcrVA5, AcrVIA1 (Lse), AcrVIA1 (Lwa) AcrVIA2, AcrVIA3, AcrVIA4, AcrVIA5, AcrVIA6, AcrVIA7, AcrVIB, Csx27, a homologue thereof, or any combination thereof.

10. The engineered Acr polypeptide of any one of aspects 1-9, further comprising a reporter molecule operatively coupled to the cargo delivery molecule, the Acr polypeptide, or both.

11. The engineered Acr polypeptide of any one of aspects 1-10, wherein the pore-forming polypeptide comprises a targeting moiety or a targeting domain.

12. The engineered Acr polypeptide of any one of aspects 1-10, wherein the pore-forming polypeptide is operatively coupled to a targeting moiety.

13. The engineered Acr polypeptide of any one of aspects 11-12, wherein the targeting moiety is an antibody or fragment thereof.

14. The engineered Acr polypeptide of aspect 3, wherein the pore-forming polypeptide interaction molecule or domain comprises a charged polypeptide.

15. The engineered Acr polypeptide of aspect 14, wherein the charged polypeptide is or comprises a polybasic polypeptide.

16. An engineered Acr polypeptide delivery system comprising:
a plurality of pore-forming polypeptides, wherein one or more of the plurality of pore-forming polypeptides are operatively coupled to a targeting moiety; and
an engineered Acr polypeptide of any one of aspects 1-15,
wherein the cargo delivery molecule of the engineered Acr polypeptide is capable of binding or otherwise interacting with the pore-forming polypeptide thereby transporting the Acr polypeptide through a pore formed from the pore-forming polypeptide.

17. The engineered Acr polypeptide delivery system of aspect 16, wherein the targeting moiety is an antibody or fragment thereof.

18. The engineered Acr polypeptide delivery system of any one of aspects 16-17, wherein the pore-forming polypeptide is or comprises an alpha pore-forming polypeptide, a beta pore-forming polypeptide, or both.

19. The engineered Acr polypeptide delivery system of any one of aspects 16-18, wherein the pore-forming polypeptide is or comprises a Bacillus anthracis protective antigen polypeptide or a derivative thereof or is or comprises a Corynebacterium diphtheriae translocation polypeptide or a derivative thereof.

20. A polynucleotide encoding an engineered Acr polypeptide of any one of aspects 1-15; and/or an engineered Acr polypeptide delivery system of any one of aspects 16-19.

21. A vector system comprising: on one or more vectors, one or more polynucleotides of claim 20.

22. The vector system of claim 21, further comprising one or more regulatory elements operatively coupled to the one or more polynucleotides.

23. A delivery vehicle comprising:
(a) an engineered Acr polypeptide as in any one of claims 1-15;
(b) an engineered Acr polypeptide delivery system comprising: a plurality of any one of aspects 16-19;
(c) one or more polynucleotides of aspect 20;
(d) one or more vector systems of any one of aspects 21-22; or
(e) any combination of (a)-(d).

24. A cell or cell population comprising:
(a) an engineered Acr polypeptide as in any one of claims 1-15;
(b) an engineered Acr polypeptide delivery system comprising: a plurality of any one of aspects 16-19;
(c) one or more polynucleotides of aspect 20;
(d) one or more vector systems of any one of aspects 21-22;
(e) a delivery vehicle of aspect 23; or
(f) any combination of (a)-(e).

25. A pharmaceutical formulation comprising:
(a) an engineered Acr polypeptide as in any one of claims 1-15;
(b) an engineered Acr polypeptide delivery system comprising: a plurality of any one of aspects 16-19;
(c) one or more polynucleotides of aspect 20;
(d) one or more vector systems of any one of aspects 21-22;
(e) a delivery vehicle of aspect 23;
(f) a cell or cell population of aspect 24; or
(g) any combination of (a)-(f); and
(h) a pharmaceutically acceptable carrier.

26. A kit comprising:
(a) an engineered Acr polypeptide as in any one of claims 1-15;
(b) an engineered Acr polypeptide delivery system comprising: a plurality of any one of aspects 16-19;
(c) one or more polynucleotides of aspect 20;
(d) one or more vector systems of any one of aspects 21-22;
(e) a delivery vehicle of aspect 23;
(f) a cell or cell population of aspect 24; or
(g) a pharmaceutical formulation of aspect 25; or
(h) any combination of (a)-(g).

27. A method of delivering an anti-CRISPR (Acr) polypeptide to a cell comprising: providing, to a cell or cell population,
(a) an engineered Acr polypeptide as in any one of claims 1-15;
(b) an engineered Acr polypeptide delivery system comprising: a plurality of any one of aspects 16-19;
(c) one or more polynucleotides of aspect 20;
(d) one or more vector systems of any one of aspects 21-22;
(e) a delivery vehicle of aspect 23;
(f) a cell or cell population of aspect 24; or
(g) a pharmaceutical formulation of aspect 25; or
(h) any combination of (a)-(g).

28. The method of claim 27, wherein the cell comprises a targeting moiety binding partner on a cell membrane surface.

29. The method of claim 27, further comprising
binding a targeting moiety or a targeting domain of a plurality of pore-forming polypeptides of an engineered Acr delivery system to the targeting moiety binding partner on the cell membrane surface thereby tethering the pore-forming polypeptide to the cell membrane surface; and
forming a pre-pore at the cell membrane surface formed from a plurality of the pore-forming polypeptides tethered to the cell membrane surface,
wherein the engineered Acr delivery system comprises the plurality of pore-forming polypeptides, wherein one or more of the plurality of pore-forming polypeptides are operatively coupled to a targeting moiety or a targeting domain; and an engineered Acr polypeptide of any one of claims 1-15, wherein the cargo delivery molecule of the engineered Acr polypeptide is capable of binding or otherwise interacting with the pore-forming polypeptide thereby transporting the Acr polypeptide through a pore formed from the pore-forming polypeptide.

30. The method of claim 29, further comprising coupling the engineered Acr polypeptide to one or more pore-forming polypeptides in the pre-pore via binding of the cargo delivery molecule to the one or more pore-forming polypeptides in the pre-pore.

31. The method of claim 30, further comprising transporting the pre-pore and the engineered Acr polypeptide coupled thereto into the cell via endocytosis whereby the pre-pore becomes a pore.

32. The method of claim 31, further comprising releasing the engineered Acr polypeptide from the pore.

33. The method of claim 31, further comprising releasing the engineered Acr polypeptide from an endosome into an intracellular compartment of the cell.

34. The method of claim 33, wherein the intracellular compartment is a cytosol or a nucleus.

35. A method of inhibiting activity of a CRISPR-Cas system in a cell comprising:
delivering an anti-CRISPR (Acr) polypeptide to the cell by the method as in any one of aspects 27-34, whereby the Acr polypeptide inhibits activity of a CRISPR-Cas system or a component thereof in the cell.

1. An engineered Anti-CRISPR (Acr) polypeptide comprising:
an Acr polypeptide operatively coupled to a cargo delivery molecule, wherein the cargo delivery molecule is capable of binding or otherwise interacting with a pore-forming polypeptide.

2. The engineered Acr polypeptide of claim 1, wherein the cargo delivery molecule is a bacterial exotoxin, optionally a Bacillus anthracis lethal factor (LF) or edema factor (EF) or a derivative thereof or a Corynebacterium diphtheriae catalytic domain or derivative thereof.

3. The engineered Acr polypeptide of claim 1, wherein the cargo delivery molecule is engineered to comprise a pore-forming polypeptide interaction molecule or domain,
optionally wherein the pore-forming polypeptide interaction molecule or domain is operatively coupled to an N-terminus, a C-terminus, at a location between the N-terminus and the C-terminus, or any combination thereof of the cargo delivery molecule;
optionally wherein the pore-forming polypeptide interaction molecule or domain comprises a charged polypeptide; and
optionally wherein the charged polypeptide is or comprises a polybasic polypeptide.

4. The engineered Acr polypeptide of claim 1, wherein the cargo delivery molecule is cleavably coupled to the Acr polypeptide, wherein the cargo delivery molecule comprises a cleavable domain or wherein the cargo delivery molecule is linked via cleavable linker to the Acr polypeptide.

5. (canceled)

6. The engineered Acr polypeptide of claim 1, wherein the pore-forming polypeptide is an alpha pore-forming polypeptide, a beta pore-forming polypeptide, or both.

7. The engineered Acr polypeptide of claim 1, wherein the pore-forming polypeptide is a Bacillus anthracis protective antigen polypeptide or a derivative thereof, or is a Corynebacterium diphtheriae translocation polypeptide or a derivative thereof.

8. The engineered Acr polypeptide of claim 1, wherein the Acr polypeptide inhibits a Type I, Type II, Type III, Type V, or Type VI CRISPR-Cas system or component or activity thereof.

9. The engineered Acr polypeptide of claim 1, wherein the Acr polypeptide is selected from an AcrIE8.2, AcrIE9, AcrIF1, AcrIF2, AcrIF3, AcrIF4, AcrIF5, AcrIF6, ArcIF7, AcrIF8, AcrIF9, AcrIF10, AcrIF11, AcrIF11.1, AcrIF11.2, AcrIF12, AcrIF13, AcrIF14, AcrIF15, AcrIF16, AcrIF17, AcrIF18, AcrIF19, AcrIF20, AcrIF21, AcrIF22, AcrIF23, AcrIF24, AcrIE4-F7, AcrIAI, AcrIB1, AcrIC1, AcrIF2/C2, AcrIC3, AcrIC4, AcrIC5, AcrIC6, AcrIC7, AcrIC8, AcrIC9, AcrIC10, AcrID1, AcrIIA1, AcrIIA2, AcrIIA2-1, AcrIIA2-2, AcrIIA2b, AcrIIA3, AcrIIA4, AcrIIA4-2, AcrIIA4-3, AcrIIA4 variant Ins. 5, AcrIIA4 variant N39A, AcrIIA4 variant D14A/G38A, AcrIIA5, AcrIIA5-2, AcrIIA6, AcrIIA7, AcrIIA8, AcrIIA9, AcrIIA10, AcrIIA11, AcrIIA12, AcrIIA13, AcrIIA13b, AcrIIA14, AcrIIA15, AcrIIA16, AcrIIA17, AcrIIA18, AcrIIA19, AcrIIA20, AcrIIA21, AcrIIA22, AcrIIA23, AcrIIA24, AcrIIA25, AcrIIA26, AcrIIA27, AcrIIA28, AcrIIA29, AcrIIA30, AcrIIA31, AcrIIA32, AcrIIC1, AcrIIC1-1, AcrIIC2, AcrIIC2-1, AcrIIC2-2, AcrIIC3, AcrIIC4, AcrIIC5, AcrIIC6, AcrIII-1, AcrIIIB1, AcrVA1, AcrVA2, AcrVA3, AcrVA3.1, AcrVA4, AcrVA5, AcrVIA1(Lse), AcrVIA1(Lwa) AcrVIA2, AcrVIA3, AcrVIA4, AcrVIA5, AcrVIA6, AcrVIA7, AcrVIB, Csx27, a homologue thereof, or any combination thereof.

10. The engineered Acr polypeptide of claim 1, further comprising a reporter molecule operatively coupled to the cargo delivery molecule, the Acr polypeptide, or both.

11. The engineered Acr polypeptide of claim 1, wherein the pore-forming polypeptide comprises a targeting moiety or a targeting domain.

12. The engineered Acr polypeptide of claim 1, wherein the pore-forming polypeptide is operatively coupled to a targeting moiety, optionally wherein the targeting moiety is an antibody or fragment thereof.

13-15. (canceled)

16. An engineered Acr polypeptide delivery system comprising:
a plurality of pore-forming polypeptides, wherein one or more of the plurality of pore-forming polypeptides are operatively coupled to a targeting moiety; and
an engineered Acr polypeptide of claim 1,
wherein the cargo delivery molecule of the engineered Acr polypeptide is capable of binding or otherwise interacting with the pore-forming polypeptide thereby transporting the Acr polypeptide through a pore formed from the pore-forming polypeptide.

17. The engineered Acr polypeptide delivery system of claim 16, wherein the targeting moiety is an antibody or fragment thereof.

18. The engineered Acr polypeptide of claim 16, wherein the pore-forming polypeptide is or comprises an alpha pore-forming polypeptide, a beta pore-forming polypeptide, or both.

19. The engineered Acr polypeptide delivery system of claim 16, wherein the pore-forming polypeptide is or comprises a Bacillus anthracis protective antigen polypeptide or a derivative thereof or is or comprises a Corynebacterium diphtheriae translocation polypeptide or a derivative thereof.

20. A polynucleotide encoding an engineered Acr polypeptide of claim 1; and/or
an engineered Acr polypeptide delivery system comprising
a plurality of pore-forming polypeptides, wherein one or more of the plurality of pore-forming polypeptides are operatively coupled to a targeting moiety; and the engineered Acr polypeptide or a component thereof.

21. A vector system comprising: on one or more vectors, one or more polynucleotides of claim 20.

22. The vector system of claim 21, further comprising one or more regulatory elements operatively coupled to the one or more polynucleotides.

23. A delivery vehicle comprising:
(a) an engineered Acr polypeptide as in claim 1;
(b) an engineered Acr polypeptide delivery system comprising: a plurality of pore-forming polypeptides, wherein one or more of the plurality of pore-forming polypeptides are operatively coupled to a targeting moiety; and an engineered Acr polypeptide of claim 1, wherein the cargo delivery molecule of the engineered Acr polypeptide is capable of binding or otherwise interacting with the pore-forming polypeptide thereby transporting the Acr polypeptide through a pore formed from the pore-forming polypeptide;
(c) one or more polynucleotides encoding (a), (b), or both;
(d) one or more vector systems comprising (c), wherein (c) is operatively coupled to one or more regulatory polynucleotides; or
(e) any combination of (a)-(d).

24. A cell or cell population comprising:
(a) an engineered Acr polypeptide as in claim 1;
(b) an engineered Acr polypeptide delivery system comprising: a plurality of pore-forming polypeptides, wherein one or more of the plurality of pore-forming polypeptides are operatively coupled to a targeting moiety; and an engineered Acr polypeptide of claim 1, wherein the cargo delivery molecule of the engineered Acr polypeptide is capable of binding or otherwise interacting with the pore-forming polypeptide thereby transporting the Acr polypeptide through a pore formed from the pore-forming polypeptide;
(c) one or more polynucleotides encoding (a), (b), or both;
(d) one or more vector systems comprising (c), wherein (c) is operatively coupled to one or more regulatory polynucleotides;
(e) a delivery vehicle of comprising (a)-(d) or any combination thereof; or
(f) any combination of (a)-(e).

25. A pharmaceutical formulation comprising:
(a) an engineered Acr polypeptide as in claim 1;
(b) an engineered Acr polypeptide delivery system comprising: a plurality of pore-forming polypeptides, wherein one or more of the plurality of pore-forming polypeptides are operatively coupled to a targeting moiety; and an engineered Acr polypeptide of claim 1, wherein the cargo delivery molecule of the engineered Acr polypeptide is capable of binding or otherwise interacting with the pore-forming polypeptide thereby transporting the Acr polypeptide through a pore formed from the pore-forming polypeptide;
(c) one or more polynucleotides encoding (a), (b), or both;
(d) one or more vector systems comprising (c), wherein (c) is operatively coupled to one or more regulatory polynucleotides;
(e) a delivery vehicle of comprising (a)-(d) or any combination thereof;
(f) a cell or cell population comprising (a)-(e) or any combination thereof; or
(g) any combination of (a)-(f); and
(h) a pharmaceutically acceptable carrier.

26. A kit comprising:
(a) an engineered Acr polypeptide as in claim 1;
(b) an engineered Acr polypeptide delivery system comprising: a plurality of pore-forming polypeptides, wherein one or more of the plurality of pore-forming polypeptides are operatively coupled to a targeting moiety; and an engineered Acr polypeptide of claim 1, wherein the cargo delivery molecule of the engineered Acr polypeptide is capable of binding or otherwise interacting with the pore-forming polypeptide thereby transporting the Acr polypeptide through a pore formed from the pore-forming polypeptide;
(c) one or more polynucleotides encoding (a), (b), or both;
(d) one or more vector systems comprising (c), wherein (c) is operatively coupled to one or more regulatory polynucleotides;
(e) a delivery vehicle of comprising (a)-(d) or any combination thereof;
(f) a cell or cell population comprising (a)-(e) or any combination thereof; or
(g) a pharmaceutical formulation comprising (a)-(f) or any combination thereof; and a pharmaceutical acceptable carrier; or
(h) any combination of (a)-(g).

27. A method of delivering an anti-CRISPR (Acr) polypeptide to a cell comprising:
providing, to a cell or cell population,
(a) an engineered Acr polypeptide as in claim 1;
(b) an engineered Acr polypeptide delivery system comprising: a plurality of pore-forming polypeptides, wherein one or more of the plurality of pore-forming polypeptides are operatively coupled to a targeting moiety; and an engineered Acr polypeptide of claim 1, wherein the cargo delivery molecule of the engineered Acr polypeptide is capable of binding or otherwise interacting with the pore-forming polypeptide thereby transporting the Acr polypeptide through a pore formed from the pore-forming polypeptide;
(c) one or more polynucleotides encoding (a), (b), or both;
(d) one or more vector systems comprising (c), wherein (c) is operatively coupled to one or more regulatory polynucleotides;
(e) a delivery vehicle of comprising (a)-(d) or any combination thereof;
(f) a cell or cell population comprising (a)-(e) or any combination thereof; or
(g) a pharmaceutical formulation comprising (a)-(f) or any combination thereof; and a pharmaceutical acceptable carrier; or
(h) any combination of (a)-(g).

28. The method of claim 27, wherein the cell comprises a targeting moiety binding partner on a cell membrane surface.

29. The method of claim 28, further comprising
binding a targeting moiety or a targeting domain of a plurality of pore-forming polypeptides of an engineered Acr delivery system to the targeting moiety binding partner on the cell membrane surface thereby tethering the pore-forming polypeptide to the cell membrane surface; and
forming a pre-pore at the cell membrane surface formed from a plurality of the pore-forming polypeptides tethered to the cell membrane surface,
wherein the engineered Acr delivery system comprises the plurality of pore-forming polypeptides, wherein one or more of the plurality of pore-forming polypeptides are operatively coupled to a targeting moiety or a targeting domain; and an engineered Acr polypeptide of claim 1, wherein the cargo delivery molecule of the engineered Acr polypeptide is capable of binding or otherwise interacting with the pore-forming polypeptide thereby transporting the Acr polypeptide through a pore formed from the pore-forming polypeptide.

30. The method of claim 29, further comprising coupling the engineered Acr polypeptide to one or more pore-forming polypeptides in the pre-pore via binding of the cargo delivery molecule to the one or more pore-forming polypeptides in the pre-pore.

31. The method of claim 30, further comprising transporting the pre-pore and the engineered Acr polypeptide coupled thereto into the cell via endocytosis whereby the pre-pore becomes a pore, optionally further comprising releasing the engineered Acr polypeptide from the pore or from an endosome into an intracellular compartment of the cell, optionally wherein the intracellular compartment is a cytosol or a nucleus.

32-34. (canceled)

35. A method of inhibiting activity of a CRISPR-Cas system in a cell comprising:
delivering an anti-CRISPR (Acr) polypeptide to the cell by the method as in claim 27, whereby the Acr polypeptide inhibits activity of a CRISPR-Cas system or a component thereof in the cell.


SEQUENCE LISTING (107 sequences)

SEQ ID NO: 1 - protein, 10 aa, synthetic construct
  REGION 1..10: note = Synthetic
GGGGCGGGGS

SEQ ID NO: 2 - protein, 119 aa, synthetic construct
  REGION 1..119: note = Acr Polypeptide
MSTQYTYQQIAEDFRLWSEYVDTAGEMSKDEFNSLSTEDKVRLQVEAFGEEKSPKFSTKVTTKPDFDGFQFYIEAGRDFDGDAYTEAYGVAVPTNIAARIQAQAAELNAGEWLLVEHEA

SEQ ID NO: 3 - protein, 63 aa, synthetic construct
  REGION 1..63: note = Acr Polypeptide
MTTITINTYDPEARFNMSGEEAKEFFAFVEEQAKVSGFDVYYDSCTYVDEESERFVEKCFQNY

SEQ ID NO: 4 - protein, 287 aa, synthetic construct
  REGION 1..287: note = Acr Polypeptide
MRSKMIKKEEKDNKIYITVKDEETGIEWTAVVEKVEFEWCVKQKEELEVEDAEKSVMLDYALFGNCAIPKVTAEEYKNSLTKYTGEKMSRLLHILYNYEIVSQNDTKNIWVTELSRCLRRSYLMRKEGKTKVGLNEAMKMHIGSGLHMRLQSLLRKHGFETEVRVQRKTALGFQIVGRIDVYDKEENVIYELKYTHNDKLDSVRLNNYLRQLNYYIEMANAMKGYLVIVHADGSVEEIKRDWAETDLEKRANAFGIYVEENTLPPKKSRPDAECIECPFYNFCWGKL

SEQ ID NO: 5 - protein, 97 aa, synthetic construct
  REGION 1..97: note = Acr Polypeptide
MATKTAQMIAQQHKDTVAACEAAEAIAIAKDQVWDGEGYTKYTFDDNSVLIQSGTTQYAMDADDADSIKGYADWLDDEARSAEASEIERLLESVEEE

SEQ ID NO: 6 - protein, 168 aa, synthetic construct
  REGION 1..168: note = Acr Polypeptide
MVGKSKIDWQSIDWTKTNAQIAQECGRAYNTVCKMRGKLGKSHQGAKSPRKDKGISRPQPHLNRLEYQALATAKAKASPKAGRFETNTKAKTWTLKSPDNKTYTFTNLMHFVRTNPHLFDPDDVVWRTKSNGVEWCRASSGLALLAKRKKAPLSWKGWRLISLTKDNK

SEQ ID NO: 7 - protein, 83 aa, synthetic construct
  REGION 1..83: note = Acr Polypeptide
MSKTMYKNDVIELIKNAKTNNEELLFTSVERNTREAATQYFRCPEKHVSDAGVYYGEDFEFDGFEIFEDDLIYTRSYDKEELN

SEQ ID NO: 8 - protein, 170 aa, synthetic construct
  REGION 1..170: note = Acr Polypeptide
MRKTIERLLNSELSSNSIAVRTGVSQAVISKLRNGKKELGNLTLNSAEKLFEYQKEMEKVDTWIVYRGRTADMNKSYIAEGSTYEEVYNNFVDKYGYDVLDEDIYEIQLLKKNGENLDDYDVDSDGINNYDKLDEFRESDYVDLEDYDYRELFENSSSQVYYHEFEITHE

SEQ ID NO: 9 - protein, 202 aa, synthetic construct
  REGION 1..202: note = Acr Polypeptide
MGYIGTKRSERSQDAIEDYEVPLNHFNKDLIQAFIDENEAYDTLKTKKVRLWKFVAPRAGATSWHHTGTYYNKTDHYSLEKVADELLQNGDEWEEQFKAYVKEEQETATSEPVFLSVIKVQIWGGSMKRPKLVGHEVVMGVKKEGWLHAVSKATQSKYKLSANKVEMQKHYSLEDYSALTKDFPEFKAQKRAINKKMKEMYN

SEQ ID NO: 10 - protein, 64 aa, synthetic construct
  REGION 1..64: note = Acr Polypeptide
MKNYEVTNEVKNLNTQVETIGQAVDLYKEYGSNTIVWSIDKNEDLIDEVTELVAEYAEKGTVIK

SEQ ID NO: 11 - protein, 108 aa, synthetic construct
  REGION 1..108: note = Acr Polypeptide
MDYDNENYLIPKILLQDDFYSSLSAKDILVYAVLKDRQIEALEKGWIDTDGSIYLNFKLIELAKMFSCSRTTMIDVMQRLEEVNLIERERVDVFYGYSLPYKTYINEV

SEQ ID NO: 12 - protein, 54 aa, synthetic construct
  REGION 1..54: note = Acr Polypeptide
MVVEETRDLAETADCVVIEAILVDDGLRYRQLSVGIKDENGDIIRIVPISTVLI

SEQ ID NO: 13 - protein, 93 aa, synthetic construct
  REGION 1..93: note = Acr Polypeptide
MFIYVIRRNKMEYGNKIFEIYNKPFKYRNSSSTNYNKVRASGIEPNTKFVVNKTANINCAVYPRHGSIEKVFYWGDRKITQATAEKRCGYFKG

SEQ ID NO: 14 - protein, 104 aa, synthetic construct
  REGION 1..104: note = Acr Polypeptide
MKKAQQLLKEIKTNNVSYAIMDEDNEIYCNKETNNIMDIYGYDNENGHFYGVYGDVVDGQIDSRYFSDDAILNAIDKLLFLGDPIKRTDLPSDADFKRTFFFEE

SEQ ID NO: 15 - protein, 96 aa, synthetic construct
  REGION 1..96: note = Acr Polypeptide
MKNRLLGSRYTDAIKNDCGTANKMSNIYNKLNKDSLREIHSALYGLLTAGYDISNMRNIEELEKYVNLKKSRGQLLNVSSDDIKLYHKLFVIRFGK

SEQ ID NO: 16 - protein, 183 aa, synthetic construct [sequence omitted]

SEQ ID NO: 17 - protein, 79 aa, synthetic construct [sequence omitted]

SEQ ID NO: 18 - protein, 88 aa, synthetic construct
  REGION 1..88: note = Acr Polypeptide
MKTIFTKKQTEELLNDISIEKQKELFNSMHDFRSQHAKEARIPGWSDKYNKLEKKMLSDFEEVTGIKYDTLESELIWDNLSNKFLYNS

SEQ ID NO: 19 - protein, 241 aa, synthetic construct
  REGION 1..241: note = Acr Polypeptide
MKPSQKIKWLLTATGITTYKIGKDIEESTQFLDRYKNDPEKIGGMRLEKAEKLLEYISNLRQEDVIKTNWNNQQILVQNSTEKEITKYFNSYPFAIKLNWIKPHKEMFIVNFDTTSNKTFRKYPYDLKNLYFLVDKNRDKMSQFAEFLIICGRKSHFGGSRVLYEVEGKKYQIIFSIKRPSELGPTIRLINVVETDTYRDDLVPKISEEESILRSEDLDLKGKRVSIKDSELLELMSIIDN

SEQ ID NO: 20 - protein, 73 aa, synthetic construct
  REGION 1..73: note = Acr Polypeptide
MITANEIVKTHKGIRLVQRKNESWEEFKERIQEVIAKQGDNYLTQTKPVHEIKNKGTRNIRRTYVNILLKEGA

SEQ ID NO: 21 - protein, 69 aa, synthetic construct
  REGION 1..69: note = Acr Polypeptide
MVTEEQLKEVLVGIYETEYKDEQTFEEYADGWDFWIDKDGDILIEGRGMKPIDGVQKVGHVDNGVIYAY

SEQ ID NO: 22 - protein, 141 aa, synthetic construct
  REGION 1..141: note = Acr Polypeptide
MKNEDGKLVVSKAHFGNMIRNCQSVEDFKKSFERLTYYSSENRESTVRQRLKIAEKEYNFKAGVKEDLEIKNTTDKEILDYVRNELSKIDSKKQADKNWSEKNREHRNYLSKRSSARSFINNNATHEDLLELKKIIEEKLK

SEQ ID NO: 23 - protein, 76 aa, synthetic construct
  REGION 1..76: note = Acr Polypeptide
MKTLKTNLFVLEQTSQNTFNVYRNHYDINISTPYGIVKLSDEVIAGLEKKPHSGYWSEVVRQTVEQNGALYEKHKI

SEQ ID NO: 24 - protein, 6 aa, synthetic construct
  REGION 1..6: note = Synthetic
GGGLLK

SEQ ID NO: 25 - protein, 6 aa, synthetic construct
  MOD_RES 5: note = L is D-Leu
  MOD_RES 4: note = L is D-Leu
  REGION 1..6: note = Synthetic
GGGLLK

SEQ ID NO: 26 - protein, 25 aa, synthetic construct
  REGION 1..25: note = Synthetic
GGGGGSGGSGGSGGSGGSGGSGGSK

SEQ ID NO: 27 - protein, 25 aa, synthetic construct
  MOD_RES 18: note = S is L-Ser
  MOD_RES 25: note = K is L-Lys
  REGION 1..25: note = Synthetic
  MOD_RES 15: note = S is L-Ser
  MOD_RES 12: note = S is L-Ser
  MOD_RES 6: note = S is L-Ser
  MOD_RES 24: note = S is L-Ser
  MOD_RES 21: note = S is L-Ser
  MOD_RES 9: note = S is L-Ser
GGGGGSGGSGGSGGSGGSGGSGGSK

SEQ ID NO: 28 - protein, 7 aa, synthetic construct
  REGION 1..7: note = Synthetic
LPSTGGK

SEQ ID NO: 29 - protein, 10 aa, synthetic construct
  REGION 1..10: note = Synthetic
GGGGCGGGGS

SEQ ID NO: 30 - protein, 33 aa, synthetic construct
  REGION 1..33: note = Synthetic
GAAPAAAPAKQEAAAPAPAAKAEAPAAAPAAKA

SEQ ID NO: 31 - protein, 15 aa, synthetic construct
  REGION 1..15: note = Synthetic
GGGGSGGGGSGGGGS

SEQ ID NO: 32 - protein, 8 aa, synthetic construct
  REGION 1..8: note = Synthetic
GGGGGGGG

SEQ ID NO: 33 - protein, 6 aa, synthetic construct
  REGION 1..6: note = Synthetic
GGGGGG

SEQ ID NO: 34 - protein, 15 aa, synthetic construct
  REGION 1..15: note = Synthetic
EAAAKEAAAKEAAAK

SEQ ID NO: 35 - protein, 5 aa, synthetic construct
  REGION 1..5: note = Synthetic
EAAAK

SEQ ID NO: 36 - protein, 10 aa, synthetic construct
  REGION 1..10: note = Synthetic
EAAAKEAAAK

SEQ ID NO: 37 - protein, 46 aa, synthetic construct
  REGION 1..46: note = Synthetic
AEAAAKEAAAKEAAAKEAAAKALEAEAAAKEAAAKEAAAKEAAAKA

SEQ ID NO: 38 - protein, 5 aa, synthetic construct
  REGION 1..5: note = Synthetic
GGGGS

SEQ ID NO: 39 - protein, 5 aa, synthetic construct
  REGION 1..5: note = Synthetic
PAPAP

SEQ ID NO: 40 - protein, 12 aa, synthetic construct
  REGION 1..12: note = Synthetic
AEAAAKEAAAKA

SEQ ID NO: 41 - protein, 10 aa, synthetic construct
  REGION 1..10: note = Synthetic
GGGGSGGGGS

SEQ ID NO: 42 - protein, 20 aa, synthetic construct
  REGION 1..20: note = Synthetic
GGGGSGGGGSGGGGSGGGGS

SEQ ID NO: 43 - protein, 25 aa, synthetic construct
  REGION 1..25: note = Synthetic
GGGGSGGGGSGGGGSGGGGSGGGGS

SEQ ID NO: 44 - protein, 30 aa, synthetic construct
  REGION 1..30: note = Synthetic
GGGGSGGGGSGGGGSGGGGSGGGGSGGGGS

SEQ ID NO: 45 - protein, 35 aa, synthetic construct
  REGION 1..35: note = Synthetic
GGGGSGGGGSGGGGSGGGGSGGGGSGGGGSGGGGS

SEQ ID NO: 46 - protein, 40 aa, synthetic construct
  REGION 1..40: note = Synthetic
GGGGSGGGGSGGGGSGGGGSGGGGSGGGGSGGGGSGGGGS

SEQ ID NO: 47 - protein, 45 aa, synthetic construct
  REGION 1..45: note = Synthetic
GGGGSGGGGSGGGGSGGGGSGGGGSGGGGSGGGGSGGGGSGGGGS

SEQ ID NO: 48 - protein, 50 aa, synthetic construct
  REGION 1..50: note = Synthetic
GGGGSGGGGSGGGGSGGGGSGGGGSGGGGSGGGGSGGGGSGGGGSGGGGS

SEQ ID NO: 49 - protein, 20 aa, synthetic construct
  REGION 1..20: note = Synthetic
APAPAPAPAPAPAPAPAPAP

SEQ ID NO: 50 - protein, 22 aa, synthetic construct
  REGION 1..22: note = Synthetic
APAPAPAPAPAPAPAPAPAPAP

SEQ ID NO: 51 - protein, 24 aa, synthetic construct
  REGION 1..24: note = Synthetic
APAPAPAPAPAPAPAPAPAPAPAP

SEQ ID NO: 52 - protein, 26 aa, synthetic construct
  REGION 1..26: note = Synthetic
APAPAPAPAPAPAPAPAPAPAPAPAP

SEQ ID NO: 53 - protein, 28 aa, synthetic construct
  REGION 1..28: note = Synthetic
APAPAPAPAPAPAPAPAPAPAPAPAPAP

SEQ ID NO: 54 - protein, 30 aa, synthetic construct
  REGION 1..30: note = Synthetic
APAPAPAPAPAPAPAPAPAPAPAPAPAPAP

SEQ ID NO: 55 - protein, 32 aa, synthetic construct
  REGION 1..32: note = Synthetic
APAPAPAPAPAPAPAPAPAPAPAPAPAPAPAP

SEQ ID NO: 56 - protein, 34 aa, synthetic construct
  REGION 1..34: note = Synthetic
APAPAPAPAPAPAPAPAPAPAPAPAPAPAPAPAP

SEQ ID NO: 57 - protein, 36 aa, synthetic construct
  REGION 1..36: note = Synthetic
APAPAPAPAPAPAPAPAPAPAPAPAPAPAPAPAPAP

SEQ ID NO: 58 - protein, 38 aa, synthetic construct
  REGION 1..38: note = Synthetic
APAPAPAPAPAPAPAPAPAPAPAPAPAPAPAPAPAPAP

SEQ ID NO: 59 - protein, 40 aa, synthetic construct
  REGION 1..40: note = Synthetic
APAPAPAPAPAPAPAPAPAPAPAPAPAPAPAPAPAPAPAP

SEQ ID NO: 60 - protein, 42 aa, synthetic construct
  REGION 1..42: note = Synthetic
APAPAPAPAPAPAPAPAPAPAPAPAPAPAPAPAPAPAPAPAP

SEQ ID NO: 61 - protein, 44 aa, synthetic construct
  REGION 1..44: note = Synthetic
APAPAPAPAPAPAPAPAPAPAPAPAPAPAPAPAPAPAPAPAPAP

SEQ ID NO: 62 - protein, 46 aa, synthetic construct
  REGION 1..46: note = Synthetic
APAPAPAPAPAPAPAPAPAPAPAPAPAPAPAPAPAPAPAPAPAPAP

SEQ ID NO: 63 - protein, 48 aa, synthetic construct
  REGION 1..48: note = Synthetic
APAPAPAPAPAPAPAPAPAPAPAPAPAPAPAPAPAPAPAPAPAPAPAP

SEQ ID NO: 64 - protein, 50 aa, synthetic construct
  REGION 1..50: note = Synthetic
APAPAPAPAPAPAPAPAPAPAPAPAPAPAPAPAPAPAPAPAPAPAPAPAP

SEQ ID NO: 65 - protein, 52 aa, synthetic construct
  REGION 1..52: note = Synthetic
APAPAPAPAPAPAPAPAPAPAPAPAPAPAPAPAPAPAPAPAPAPAPAPAPAP

SEQ ID NO: 66 - protein, 54 aa, synthetic construct
  REGION 1..54: note = Synthetic
APAPAPAPAPAPAPAPAPAPAPAPAPAPAPAPAPAPAPAPAPAPAPAPAPAPAP

SEQ ID NO: 67 - protein, 56 aa, synthetic construct
  REGION 1..56: note = Synthetic
APAPAPAPAPAPAPAPAPAPAPAPAPAPAPAPAPAPAPAPAPAPAPAPAPAPAPAP

SEQ ID NO: 68 - protein, 58 aa, synthetic construct
  REGION 1..58: note = Synthetic
APAPAPAPAPAPAPAPAPAPAPAPAPAPAPAPAPAPAPAPAPAPAPAPAPAPAPAPAP

SEQ ID NO: 69 - protein, 60 aa, synthetic construct
  REGION 1..60: note = Synthetic
APAPAPAPAPAPAPAPAPAPAPAPAPAPAPAPAPAPAPAPAPAPAPAPAPAPAPAPAPAP

SEQ ID NO: 70 - protein, 62 aa, synthetic construct
  REGION 1..62: note = Synthetic
APAPAPAPAPAPAPAPAPAPAPAPAPAPAPAPAPAPAPAPAPAPAPAPAPAPAPAPAPAPAP

SEQ ID NO: 71 - protein, 64 aa, synthetic construct
  REGION 1..64: note = Synthetic
APAPAPAPAPAPAPAPAPAPAPAPAPAPAPAPAPAPAPAPAPAPAPAPAPAPAPAPAPAPAPAP

SEQ ID NO: 72 - protein, 17 aa, synthetic construct
  REGION 1..17: note = Synthetic
  SITE 9..10: note = Protease-sensitive cleavage site
VSQTSKLTRAETVFPDV

SEQ ID NO: 73 - protein, 6 aa, synthetic construct
  REGION 1..6: note = Synthetic
  SITE 3..4: note = Protease-sensitive cleavage site
PLGLWA

SEQ ID NO: 74 - protein, 6 aa, synthetic construct
  REGION 1..6: note = Synthetic
  SITE 3..4: note = Protease-sensitive cleavage site
RVLAEA

SEQ ID NO: 75 - protein, 10 aa, synthetic construct
  SITE 6..7: note = Protease-sensitive cleavage site
  REGION 1..10: note = Synthetic
EDVVCCSMSY

SEQ ID NO: 76 - protein, 8 aa, synthetic construct
  SITE 6..7: note = Protease-sensitive cleavage site
  REGION 1..8: note = Synthetic
GGIEGRGS

SEQ ID NO: 77 - protein, 10 aa, synthetic construct
  SITE 7..8: note = Protease-sensitive cleavage site
  REGION 1..10: note = Synthetic
TRHRQPRGWE

SEQ ID NO: 78 - protein, 10 aa, synthetic construct
  REGION 1..10: note = Synthetic
  SITE 7..8: note = Protease-sensitive cleavage site
AGNRVRRSVG

SEQ ID NO: 79 - protein, 9 aa, synthetic construct
  SITE 8..9: note = Protease-sensitive cleavage site
  REGION 1..9: note = Synthetic
  SITE 7..8: note = Protease-sensitive cleavage site
RRRRRRRRR

SEQ ID NO: 80 - protein, 4 aa, synthetic construct
  SITE >4: note = Protease-sensitive cleavage site
  REGION 1..4: note = Synthetic
GFLG

SEQ ID NO: 81 - protein, 20 aa, synthetic construct
  REGION 1..20: note = Synthetic
  SITE 11..12: note = Protease-sensitive cleavage site
LEAGCKNFFPRSFTSCGSLE

SEQ ID NO: 82 - protein, 12 aa, synthetic construct
  REGION 1..12: note = Synthetic
CRRRRRREAEAC

SEQ ID NO: 83 - protein, 7 aa, synthetic construct
  VARIANT 7: note = X is S, G, A, M, C, or H
  SITE 6..7: note = Protease-sensitive cleavage site
  REGION 1..7: note = Synthetic
ENLYFQX

SEQ ID NO: 84 - protein, 7 aa, unidentified
  source 1..7: note = Simian virus 40
PKKKRKV

SEQ ID NO: 85 - protein, 10 aa, unidentified
  source 1..10: note = Simian virus 40
PKKKRKVEAS

SEQ ID NO: 86 - protein, 16 aa, synthetic construct
  REGION 1..16: note = Synthetic
KRPAATKKAGQAKKKK

SEQ ID NO: 87 - protein, 9 aa, Homo sapiens
PAAKRVKLD

SEQ ID NO: 88 - protein, 11 aa, Homo sapiens
RQRRNELKRSP

SEQ ID NO: 89 - protein, 38 aa, Homo sapiens
NQSSNFGPMKGGNFGGRSSGPYGGGGQYFAKPRNQGGY

SEQ ID NO: 90 - protein, 42 aa, Homo sapiens
RMRIZFKNKGKDTAELRRRRVEVSVELRKAKKDEQILKRRNV

SEQ ID NO: 91 - protein, 8 aa, Homo sapiens
VSRKRPRP

SEQ ID NO: 92 - protein, 8 aa, Homo sapiens
PPKKARED

SEQ ID NO: 93 - protein, 8 aa, Homo sapiens
PQPKKKPL

SEQ ID NO: 94 - protein, 12 aa, Mus sp.
SALIKKKKKMAP

SEQ ID NO: 95 - protein, 5 aa, unidentified
  source 1..5: note = Influenza virus
DRLRR

SEQ ID NO: 96 - protein, 7 aa, unidentified
  source 1..7: note = Influenza virus
PKQKKRK

SEQ ID NO: 97 - protein, 10 aa, unidentified
  source 1..10: note = Hepatitus D virus
RKLKKKIKKL

SEQ ID NO: 98 - protein, 10 aa, Mus sp.
REKKKFLKRR

SEQ ID NO: 99 - protein, 20 aa, Homo sapiens
KRKGDEVDGVDEVAKKKSKK

SEQ ID NO: 100 - protein, 17 aa, Homo sapiens
RKCLQAGMNLEARKTKK

SEQ ID NO: 101 - protein, 12 aa, Human immunodeficiency virus 1
GRKKRRQRRRPQ

SEQ ID NO: 102 - protein, 10 aa, synthetic construct
  VARIANT 2..4: note = X is any amino acid
  VARIANT 9: note = X is any amino acid
  REGION 1..10: note = Synthetic
  VARIANT 6..7: note = X is any amino acid
LXXXLXXLXL

SEQ ID NO: 103 - protein, 4 aa, synthetic construct
  REGION 1..4: note = Synthetic
KDEL

SEQ ID NO: 104 - protein, 15 aa, synthetic construct
  REGION 1..15: note = Synthetic
GGGGGGGGGGGGGGG

SEQ ID NO: 105 - protein, 5 aa, synthetic construct
  REGION 1..5: note = Synthetic
TEFCA

SEQ ID NO: 106 - DNA, 7077 bp, synthetic construct
  source 1..7077: mol_type = other DNA
  misc_feature 1..7077: note = Synthetic
tcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactatagggagaccacaacggtttccctctagtgccggctccggagagctctttaattaagcggccgccctgcaggactcgagttctagaaataattttgtttaactttaagaaggagatatacatatgaaatcttctcaccatcaccatcaccatcaccatcaccatggttcttctatgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggatcgaggaaaacctgtacttccaatccaatgcagcgggcggtcatggtgatgtaggtatgcacgtaaaagagaaagagaaaaataaagatgagaataagagaaaagatgaagaacgaaataaaacacaggaagagcatttaaaggaaatcatgaaacacattgtaaaaatagaagtaaaaggggaggaagctgttaaaaaagaggcagcagaaaagctacttgagaaagtaccatctgatgttttagagatgtataaagcaattggaggaaagatatatattgtggatggtgatattacaaaacatatatctttagaagcattatctgaagataagaaaaaaataaaagacatttatgggaaagatgctttattacatgaacattatgtatatgcaaaagaaggatatgaacccgtacttgtaatccaatcttcggaagattatgtagaaaatactgaaaaggcactgaacgtttattatgaaataggtaagatattatcaagggatattttaagtaaaattaatcaaccatatcagaaatttttagatgtattaaataccattaaaaatgcatctgattcagatggacaagatcttttatttactaatcagcttaaggaacatcccacagacttttctgtagaattcttggaacaaaatagcaatgaggtacaagaagtatttgcgaaagcttttgcatattatatcgagccacagcatcgtgatgttttacagctttatgcaccggaagcttttaattacatggataaatttaacgaacaagaaataaatctatccttggaagaacttaaagatcaacggggcggtggtggttgtggtgggggcggcagcatgaatattaatgacttaattagagaaatcaaaaacaaagattacacagtgaaattgagtggtacggatagcaatagtatcacacagctaattattcgcgttaataatgatggcaacgagtatgtaatttctgaaagtgaaaatgaatcaatcgttgaaaaattcatctctgcattcaaaaacggttggaatcaagaatacgaggatgaagaagaattttataatgacatgcaaacaatcaccttaaaaagtgagttgaacgggtcacctaagaaaaaacgaaaagtttaataaatattggaagtggataacggatccgcgatcgcggcgcgccacctggtggccggccggtaccacgcgtgcgcgctgatccggctgctaacaaagcccgaaaggaagctgagttggctgctgccaccgctgagcaataactagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataacattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgcaaggagatggcgcccaacag

SEQ ID NO: 107 - protein, 8 aa, synthetic construct
  REGION 1..8: note = Synthetic
GPLGIAGQ